SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  Synthetic variable light chain CDR3 peptide
QRYNRAPYT

SEQ ID NO: 4 - protein, 12 aa, Artificial Sequence
  Synthetic variable heavy chain CDR3 peptide
VSYLSTASSLDY

SEQ ID NO: 5 - protein, 7 aa, Artificial Sequence
  Synthetic variable light chain CDR2 peptide
AASTLQS

SEQ ID NO: 6 - protein, 17 aa, Artificial Sequence
  Synthetic variable heavy chain CDR2 peptide
AITWNSGHIDYADSVEG

SEQ ID NO: 7 - protein, 11 aa, Artificial Sequence
  Synthetic variable light chain CDR1 peptide
RASQGIRNYLA

SEQ ID NO: 8 - protein, 5 aa, Artificial Sequence
  Synthetic variable heavy chain CDR1 peptide
DYAMH

SEQ ID NO: 9 - protein, 214 aa, Artificial Sequence
  Synthetic adalimumab light chain polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, Artificial Sequence
  Synthetic adalimumab heavy chain polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 16 aa, Artificial Sequence
  Synthetic peptide
AKTTPKLEEGEFSEAR

SEQ ID NO: 12 - protein, 17 aa, Artificial Sequence
  Synthetic peptide
AKTTPKLEEGEFSEARV

SEQ ID NO: 13 - protein, 9 aa, Artificial Sequence
  Synthetic peptide
AKTTPKLGG

SEQ ID NO: 14 - protein, 10 aa, Artificial Sequence
  Synthetic peptide
SAKTTPKLGG

SEQ ID NO: 15 - protein, 6 aa, Artificial Sequence
  Synthetic peptide
SAKTTP

SEQ ID NO: 16 - protein, 6 aa, Artificial Sequence
  Synthetic peptide
RADAAP

SEQ ID NO: 17 - protein, 9 aa, Artificial Sequence
  Synthetic peptide
RADAAPTVS

SEQ ID NO: 18 - protein, 12 aa, Artificial Sequence
  Synthetic peptide
RADAAAAGGPGS

SEQ ID NO: 19 - protein, 27 aa, Artificial Sequence
  Synthetic peptide
RADAAAAGGGGSGGGGSGGGGSGGGGS

SEQ ID NO: 20 - protein, 18 aa, Artificial Sequence
  Synthetic peptide
SAKTTPKLEEGEFSEARV

SEQ ID NO: 21 - protein, 5 aa, Artificial Sequence
  Synthetic peptide
ADAAP

SEQ ID NO: 22 - protein, 12 aa, Artificial Sequence
  Synthetic peptide
ADAAPTVSIFPP

SEQ ID NO: 23 - protein, 5 aa, Artificial Sequence
  Synthetic peptide
TVAAP

SEQ ID NO: 24 - protein, 12 aa, Artificial Sequence
  Synthetic peptide
TVAAPSVFIFPP

SEQ ID NO: 25 - protein, 6 aa, Artificial Sequence
  Synthetic peptide
QPKAAP

SEQ ID NO: 26 - protein, 13 aa, Artificial Sequence
  Synthetic peptide
QPKAAPSVTLFPP

SEQ ID NO: 27 - protein, 6 aa, Artificial Sequence
  Synthetic peptide
AKTTPP

SEQ ID NO: 28 - protein, 13 aa, Artificial Sequence
  Synthetic peptide
AKTTPPSVTPLAP

SEQ ID NO: 29 - protein, 6 aa, Artificial Sequence
  Synthetic peptide
AKTTAP

SEQ ID NO: 30 - protein, 13 aa, Artificial Sequence
  Synthetic peptide
AKTTAPSVYPLAP

SEQ ID NO: 31 - protein, 6 aa, Artificial Sequence
  Synthetic peptide
ASTKGP

SEQ ID NO: 32 - protein, 13 aa, Artificial Sequence
  Synthetic peptide
ASTKGPSVFPLAP

SEQ ID NO: 33 - protein, 15 aa, Artificial Sequence
  Synthetic peptide
GGGGSGGGGSGGGGS

SEQ ID NO: 34 - protein, 15 aa, Artificial Sequence
  Synthetic peptide
GENKVEYAPALMALS

SEQ ID NO: 35 - protein, 15 aa, Artificial Sequence
  Synthetic peptide
GPAKELTPLKEAKVS

SEQ ID NO: 36 - protein, 15 aa, Artificial Sequence
  Synthetic peptide
GHEAAAVMQVQYPAS

SEQ ID NO: 37 - protein, 10 aa, Artificial Sequence
  Synthetic peptide
GGGGSGGGGS

(19) United States
(12) Patent Application Publication
(10) Pub. No.: US 2016/0228371 A1
(43) Pub. Date: Aug. 11, 2016

(54) STABLE SOLID UNITS AND METHODS OF MAKING THE SAME

(71) Applicant: ABBVIE INC., North Chicago, IL (US)

(72) Inventors: Steven G. Schultz, Wadsworth, IL (US); Sherry G. Rusev, Morton Grove, IL (US)

(21) Appl. No.: 15/029,947

(22) PCT Filed: Oct. 18, 2014
(86) PCT No.: PCT/US14/61279

§ 371 Date: Apr. 15, 2016

Related U.S. Application Data
(60) Provisional application No. 61/893,123, filed on Oct. 18, 2013.

(51) Int. Cl. A61K 9/16; A61J 1/20; A61K 9/19; C07K 16/28; A61K 9/48; C07K 16/24

(52) U.S. Cl. CPC A61K 9/1623; A61K 9/4808; C07K 16/241; A61K 9/19; A61K 9/1652; A61K 9/1635; C07K 16/244; C07K 16/245; C07K 16/283; A61J 1/2089; C07K 2317/21; C07K 2317/76; C07K 2317/94; C07K 2317/31

(57) ABSTRACT

The invention provides stable solid compositions of a therapeutic agent, such as a protein, (e.g., an antibody, a peptide, or a DVD-Ig protein), and a stabilizer, such as a sugar, and methods of preparing and using the same. The invention further provides a generalized therapeutic agent delivery form wherein the active components are in a lyophilized, stable configuration, and, in some embodiments, prepared independently from the primary container.

### RELATED APPLICATIONS

This application claims priority to U.S. Provisional Application No. 61/893,123, filed Oct. 18, 2013, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Oct. 18, 2014, is named 117813-31020_SL.txt and is 15,369 bytes in size.

### BACKGROUND OF THE INVENTION

A major challenge in the pharmaceutical industry is the ability to stably maintain a formulation comprising a therapeutic agent, particularly a biologic, under room temperature conditions. Lyophilization is well known in the art to provide the required stability, in most cases. While lyophilized formulations are used in the industry, these formulations come with unique challenges, including manufacturing difficulties and added complexity for patients. Traditional lyophilization requires that critical freeze drying steps be carried out in the primary drug container, which works well for vials. However, reconstitution within vials is a multi-step process and not applicable for home administration. Prefilled syringes are the preferred dosage form for home delivery, but are cumbersome to handle during the lyophilization process.

Aside from lyophilization, other currently known processes that provide stable formulations include spray drying. Spray drying, however, incorporates high shear and high temperatures, either of which may be detrimental to the stability and potentially the effectiveness of a therapeutic agent, especially a therapeutic protein. Formulations obtained using spray drying often have low density and can be difficult to handle due to dusting, settling, and large volume requirements. Spray-freeze drying may reduce shearing and eliminates the high temperature requirement associated with spray-drying, thus improving storage stability relative to spray-drying. Nevertheless, both spray-drying and spray-freeze drying techniques result in varying particle sizes, irregular surface geometry, clumping and settling, which bring added complexity to the manufacturing process and eventual use of the formulation for therapeutic purposes.

A basic principle of pharmaceutical protein formulations is that certain instabilities must be overcome. Degradation pathways of proteins, e.g., antibodies, can be separated into two distinct classes, involving chemical instability and physical instability. Chemical instabilities lead to the modification of the protein through bond formation or cleavage. Examples of chemical instability problems include deamidation, racemization, hydrolysis, oxidation, beta elimination and disulfide exchange. Physical instabilities, on the other hand, do not lead to covalent changes in proteins. Rather, they involve changes in the higher order structure (secondary and above) of proteins. These include denaturation, adsorption to surfaces, aggregation and precipitation (Manning et al., Pharm. Res. 6, 903 (1989)).

It is generally accepted that these instabilities, which can have great effect on the commercial viability and efficacy of pharmaceutical protein formulations, can be overcome by including additional molecules in the formulation. Protein stability can be improved by including excipients that interact with the protein in solution to keep the protein stable, soluble and unaggregated. For example, salt compounds and other ionic species are common additives to protein formulations. They assist in helping to prevent denaturation of proteins by binding to proteins in a non-specific fashion and increasing thermal stability. Salt compounds (e.g., NaCl, KCl) have been used successfully in commercial insulin preparations to fight aggregation and precipitation (ibid. at 911). Amino acids (e.g., histidine, arginine) have been shown to reduce alterations in proteins' secondary structures when used as formulation additives (Tian et al., Int'l J. Pharm. 355, 20 (2007)). Other examples of commonly used additives include polyalcohol materials such as glycerol and sugars, and surfactants such as detergents, both non-ionic (e.g., Tween, Pluronic) and anionic (sodium dodecyl sulfate). The near universal prevalence of additives in all liquid commercial protein formulations indicates that protein solutions without such compounds may encounter challenges with degradation due to instabilities.

Therapeutic proteins, such as antibodies, may generate a variety of degradants during production, processing, and storage both in liquid and solid states (see Wang et al. J. Pharmac. Sci. 96(1):1 (2007)). Liquid dosage forms are usually preferable to lyophilized products as they are easier to administer and less expensive to manufacture. Lyophilization is often used, however, for therapeutic proteins which are not stable in liquid formulations. A lyophilized product must be reconstituted prior to use, however, which may delay administration and add to the overall burden of the patient. Administration of the lyophilized product is usually performed under doctor supervision where the doctor can ensure the protein is reconstituted to the proper concentration and can confirm that the proper amount of drug is administered to the patient. In addition to patient convenience, long term stability of the formulation is also an important feature which can present a challenge due to chemical and physical instabilities that are often encountered over time.

While small molecule drugs are commonly administered orally, therapeutic proteins are usually administered by injection (e.g., subcutaneously or intravenously). Oral administration of therapeutic proteins presents numerous challenges given their physicochemical properties, including susceptibility to enzymatic degradation (see review described in Shaji and Patole Indian J Pharm Sci 70(3):269 (2008)). While few studies have been described that successfully targeted therapeutic proteins to the gastrointestinal tract via oral delivery, Zhu et al. (Nat Med 18(8) (2012)) described oral delivery of a nanoparticle-releasing oral vaccine in mice by encapsulating a peptide into PLGA nanoparticles.

Given the complexities of therapeutic agents, particularly therapeutic proteins, and the competing needs in formulating these molecules (e.g., long-term storage vs. patient convenience), there remains a need in the art for a solid formulation which provides long term storage stability, e.g., low aggregate formation, and fast reconstitution times. There also remains a need in the art for therapeutic protein formulations that improve convenience, e.g., through self-administration, and patient compliance.

### SUMMARY OF THE INVENTION

The present invention provides, at least in part, a lyophilization process which is separate from a primary container. Thus, the present invention provides a lyophilized formulation (solid units) that is not only stable, but also provides patient convenience given that the lyophilized formulations or solid units may be placed in non-vial primary containers, such as syringes, patch pumps, or dual-chambered cartridges or patch pumps.

The present invention provides, at least in part, a lyophilization method which produces solid units having a well controlled geometry, size, and density. When taken collectively, particularly in spherical form, the solid units of the invention are freely flowing. The free flowing solid units may be contained in a bulk form for later filling into a delivery primary container of choice, such as a dual chambered syringe. Such filling may occur at times and locations separable from where the lyophilization process takes place.

The present invention is based, at least in part, on a holistic manufacturing system for the delivery of pharmaceutical drug products comprising a lyophilization process enabling controlled nucleation producing uniform, free flowing solid units capable of maintaining stability at room temperature and/or accelerated storage conditions for a therapeutic agent, such as a therapeutic protein, e.g., a peptide, DVD-Ig protein, or an antibody, or antigen-binding portion thereof.

The present invention is based, at least in part, on the discovery of stable compositions of a therapeutic agent (particularly a therapeutic protein such as an antibody, DVD-Ig protein, or peptide) and a stabilizer, referred to herein as solid units. Specifically, it has been discovered that despite having a high proportion of sugar, the solid units of the invention maintain structural rigidity and resist changes in shape and/or volume when stored under ambient conditions, e.g., room temperature and humidity, for extended periods of time and maintain long-term physical and chemical stability of the protein without significant degradation and/or aggregate formation. Moreover, despite having a high proportion of sugar, the solid units of the invention remain free-flowing when stored under ambient conditions, e.g., room temperature and humidity, for extended periods of time, and yet are easily dissolved in a diluent, e.g., water (e.g., the solid units require minimal mixing when contacted with a diluent for reconstitution). The solid units of the invention offer advantages over conventional lyophilized formulations because they are uniform in shape and free-flowing, making them easier to reconstitute, manipulate and use to manufacture a drug product. Furthermore, the solid units of the invention are versatile in that they can be combined with certain polymers for numerous modes of administration, such as parenteral and oral administration.

The invention features, in one embodiment, a container comprising a plurality of lyophilized solid units which are free-flowing and geometrically uniform, wherein the plurality of solid units comprises a therapeutic agent and a stabilizer. In one embodiment, the plurality of solid units are spherical. Examples of diameters of speheres of the invention include, but are not limited to about 0.1 to about 4 mm; about 0.1 to about 3 mm; about 0.1 to about 2 mm; about 0.1 to about 1 mm; and about 0.1 to about 0.5 mm.

In one embodiment, the container of the invention is not the same container used to lyophilize the solid units.

Examples of types of containers that may be used to contain the solid unit or units of the invention include intermediate container or a primary container. Examples of primary containers include, but are not limited to, an ampule, a bag, a blister, a bottle, a cartridge, an injection needle, an injection syringe, a single-dose container, a strip, a dual chamber syringe, a dual chamber cartridge, a patch pump, a dual chamber patch pump, and a vial.

The invention further features a drug product comprising a plurality of lyophilized, spherical solid units which are free-flowing and geometrically uniform, wherein the plurality of solid units comprises a therapeutic agent and a sugar. Examples of amounts of therapeutic agent that may be inside the solid unit include 0.01 μg to 6.0 mg of the therapeutic agent or 15 μg to 4.0 mg of the therapeutic agent, e.g., a therapeutic protein (such as a DVD-Ig protein, an antibody, or a peptide).

In one embodiment, the invention features a drug product comprising solid units each have a diameter selected from the group consisting of about 0.1 to about 4 mm; about 0.1 to about 3 mm; about 0.1 to about 2 mm; about 0.1 to about 1 mm; and about 0.1 to about 0.5 mm. In one embodiment, the solid unit is a sphere having a diameter which is greater than 1 mm and less than 4 mm.

In a further embodiment, the solid unit(s) described in the invention, including a drug product, does not contain casein, a preservative (e.g., sodium azide), albumin, or tromethamine.

In a further embodiment, the invention features a capsule comprising a drug product comprising a solid unit(s). The capsule may, in some embodiments, comprise a second therapeutic agent.

The invention further includes a primary container comprising the drug product described herein. Examples of primary containers include, but are not limited to, an ampule, a bag, a blister, a bottle, a cartridge, an injection needle, an injection syringe, a single-dose container, a strip, a dual chamber syringe, a dual chamber cartridge, a patch pump, a dual chamber patch pump, and a vial.

In one embodiment, the solid unit described herein (or the plurality thereof) does not contain albumin, e.g., bovine serum albumin (BSA), or tromethamine. In a further embodiment, the solid unit described herein (or the plurality thereof) does not contain casein. In a further embodiment, the solid unit described herein (or the plurality thereof) does not contain a preservative, such as sodium azide.

In one embodiment, the solid unit described herein (or the plurality thereof) comprises 0.01 μg to 6.0 mg of the therapeutic agent. In a separate embodiment, the solid units comprises 15 μg to 4.0 mg of the therapeutic agent.

In one embodiment, the invention features a plurality of solid units comprises 10 or less solid units; 50 or less solid units; 100 or less solid units; 1,000 or less solid units; or 5,000 or less solid units; 10,000 or less solid units; 50,000 or less solid units; 100,000 or less solid units; 500,000 or less solid units; 1,000,000 or less solid units; or more than 1,000,000 solid units.

In one embodiment of the invention, the solid unit is free flowing when placed in a plurality of the solid units.

In a further embodiment of the invention, the solid unit(s) is not prepared by spray-drying or spray-freeze drying.

In one embodiment, the invention features a solid unit (or plurality thereof) comprising a stabilizer which is a sugar. Nonlimiting examples of sugars that may be included in the solid unit(s) of the invention include sucrose, mannitol, and trehalose. In one embodiment, the solid unit(s) of the invention include sorbitol. In one embodiment, the solid unit(s) of the invention include mannitol. In one embodiment, the solid unit(s) of the invention include sucrose. In one embodiment, the solid unit(s) of the invention include trehalose.

The invention further includes a stable solid unit comprising a lyophilized mixture of a therapeutic agent, such as a therapeutic protein (e.g., an antibody, peptide, or DVD-Ig protein) and an amount of sucrose, sorbitol, or trehalose which prevents or reduces chemical or physical instability of the antibody upon lyophilization and subsequent storage, wherein the solid unit is free flowing when placed in a plurality of the solid units.

The invention features, in one embodiment, a stable solid unit suitable for pharmaceutical administration, comprising a therapeutic agent, such as a therapeutic protein, e.g., a DVD-Ig protein, a peptide, or an antibody, or antigen-binding portion thereof, and sucrose or trehalose, wherein the amount of sucrose or trehalose is sufficient to maintain the stability of the a protein, e.g., peptide or antibody, or antigen-binding portion thereof, for at least 12 months of storage at about 25° C. storage or for at least 3 months of storage at about 40° C. and wherein the solid unit is free flowing when placed in a plurality of the solid units.

In one embodiment, stability of the therapeutic agent, e.g., a DVD-Ig protein, a peptide, or an antibody, or antigen-binding portion thereof, includes a dissolving the solid unit in water following storage wherein stability is determined by a result of 90% or more monomer therapeutic agent as determined by size exclusion chromatography (SEC), wherein the solid unit is free flowing when placed in a plurality of the solid units. In one embodiment, stability of the therapeutic agent, e.g., a DVD-Ig protein, a peptide, or an antibody, or antigen-binding portion thereof, includes a dissolving the solid unit in water following storage wherein stability is determined by a result of 95% or more monomer therapeutic agent as determined by size exclusion chromatography (SEC), wherein the solid unit is free flowing when placed in a plurality of the solid units. In one embodiment, stability of the therapeutic agent, e.g., a DVD-Ig protein, a peptide, or an antibody, or antigen-binding portion thereof, includes a dissolving the solid unit in water following storage wherein stability is determined by a result of 98% or more monomer therapeutic agent as determined by size exclusion chromatography (SEC), wherein the solid unit is free flowing when placed in a plurality of the solid units. In one embodiment, stability of the therapeutic agent, such as a therapeutic protein, e.g., a DVD-Ig protein, a peptide, or an antibody, or antigen-binding portion thereof, includes a dissolving the solid unit in water following storage wherein stability is determined by therapeutic efficacy.

The invention also features a stable solid unit suitable for pharmaceutical administration, comprising a therapeutic agent, such as a therapeutic protein, e.g., a peptide, DVD-Ig protein, or an antibody, or antigen-binding portion thereof, and sucrose, wherein the sucrose:therapeutic protein e.g., peptide or antibody, or antigen-binding portion thereof, ratio ranges from about 0.8 to 3.5:1 weight/weight (w/w). In one embodiment, the sucrose:therapeutic protein, e.g., peptide or antibody, or antigen-binding portion thereof, ratio ranges from about 0.9 to 2.0:1 w/w. In one embodiment, the sucrose:therapeutic protein, e.g., peptide or antibody, or antigen-binding portion thereof, ratio ranges from about 0.1 to 10.0:1 w/w. In one embodiment, the sucrose:therapeutic protein, e.g., peptide or antibody, or antigen-binding portion thereof, ratio ranges from about 0.1 to 3.5.0:1 w/w. In a further embodiment, the sucrose:therapeutic protein, e.g., peptide or antibody, or antigen-binding portion thereof, ratio is about 1:1 w/w.

In one embodiment, the concentration of sucrose in a solution for preparation of the solid unit is selected from the group consisting of about 10 mg/ml, about 20 mg/ml, about 30 mg/ml to about 100 mg/ml; about 40 mg/ml to about 90 mg/ml; about 40 mg/ml to about 80 mg/ml; about 40 mg/ml to about 70 mg/ml; about 40 mg/ml to about 60 mg/ml; and about 40 mg/ml to about 50 mg/ml. In one embodiment, the concentration of sucrose in a solution for preparation of the solid unit is about 10 mg/ml to about 200 mg/ml.

In one embodiment, the solid unit(s) of the invention are prepared from a solution comprising about 10 to about 40 mg/mL of mannitol and about 60 mg/mL to about 80 mg/mL of sucrose.

In a further embodiment, the concentration of sucrose in a solution for preparation of the solid unit is less than 20%, less than 15%, less than 10%, less than 7% or about 1% to about 7% sucrose.

The invention further features a stable solid unit suitable for pharmaceutical administration, comprising a therapeutic agent, such as a therapeutic protein, e.g., a peptide, DVD-Ig protein, or an antibody, or antigen-binding portion thereof, and sorbitol or trehalose, wherein the sorbitol or trehalose:therapeutic protein, e.g., peptide or antibody, or antigen-binding portion thereof, ratio ranges from about 0.8 to 3.5:1 weight/weight (w/w). In one embodiment, the sorbitol or trehalose:therapeutic protein, e.g., DVD-Ig protein, peptide or antibody, or antigen-binding portion thereof, ratio ranges from about 0.1 to 10:1 weight/weight (w/w), 0.1 to 3.5:1 w/w, or 0.9 to 2.0:1 w/w. In a further embodiment, the sorbitol or trehalose:therapeutic protein, e.g., DVD-Ig protein, peptide or antibody, or antigen-binding portion thereof, ratio is about 1:1 w/w.

The invention also includes a stable solid unit comprising a lyophilized mixture of a therapeutic agent, such as a therapeutic protein, e.g., a peptide, DVD-Ig protein, or an antibody, or antigen-binding portion thereof, and an amount of sucrose, sorbitol, or trehalose which prevents or reduces chemical or physical instability of the therapeutic agent, such as a therapeutic protein, e.g., a peptide, DVD-Ig protein, or an antibody, or antigen-binding portion thereof, upon lyophilization and subsequent storage.

The invention also features a stable solid unit suitable for oral administration to a human subject, said solid unit comprising therapeutic agent, such as a therapeutic protein, e.g., a peptide, DVD-Ig protein, or an antibody, or antigen-binding portion thereof, a stabilizer, and a polymer selected from the group consisting of an enteric protectant, a non-pH-sensitive polymer, a slow-release polymer, a bioadhesive polymer, or any combination thereof. In one embodiment, the stabilizer is a sugar. Examples of sugars include sorbitol, sucrose, and trehalose. In one embodiment, the solid unit is stable for at least 12 months of storage at about 25° C. or for at least 3 months or storage at about 40° C. Stability may be determined by dissolving the solid unit in water following storage wherein 90%, 91%, 92%, 93%, 94%, 95%, 96%, 97%, 98% or more monomer antibody, or antigen-binding portion thereof, as determined by size exclusion chromatography (SEC) indicates stability.

The invention also includes, in one embodiment, a lyophilized, stable solid unit suitable for pharmaceutical administration, said solid unit comprising an anti-human TNFα antibody, or an antigen-binding portion thereof, and a stabilizer, wherein the stabilizer prevents or reduces chemical or physical instability of the antibody, or antigen-binding portion thereof, upon lyophilizing and subsequent storage. In one embodiment, the stabilizer is a sugar, e.g., sorbitol, sucrose, and trehalose. In one embodiment, the stabilizer:antibody, or antigen-binding portion thereof, ratio ranges from about 0.8 to 3.5:1 weight/weight (w/w); about 0.9 to 2.0:1 w/w; about 0.1 to 3.5:1 w/w; about 0.1 to 10:1 w/w; or about 1:1 w/w. In one embodiment, the solid unit comprises a polymer selected from the group consisting of an enteric protectant, a non-pH-sensitive polymer, a slow-release polymer, a bioadhesive polymer, or any combination thereof; and/or an enteric coating.

In one embodiment, the solid unit of the invention has a volume ranging from about 0.1 μl to about 20 μl and/or a stabilizer:therapeutic protein e.g., DVD-Ig protein, peptide or antibody, or antigen binding portion thereof, ratio ranging from 0.8 to 3.5:1 w/w; alternatively about 0.9 to 2.0:1 w/w; about 0.1 to 3.5:1 w/w; about 0.1 to 10:1 w/w; or alternatively about 1:1 w/w.

In one embodiment of the invention, the solid unit comprises a polymer. Examples of polymers include, but are not limited to, an enteric protectant, a non-pH-sensitive polymer, a slow-release polymer, a bioadhesive polymer, or any combination thereof. The polymer may be contained within the solid unit or may be on the outside of the solid unit (or both). In one embodiment of the invention, the solid unit has an enteric coating in addition to a polymer. In one embodiment of the invention, the slow release polymer may be a carbopol, a cellulose derivative, and a poly(acrylic acid) polymer. In one embodiment of the invention, the cellulose derivative may be hydroxypropylmethylcellulose (HPMC). In one embodiment of the invention, the enteric protectant is selected from the group consisting of a polymethacrylate (e.g., methacrylic acid/ethyl acrylate), a cellulose ester, and a polyvinyl derivative (e.g., polyvinyl acetate phthalate (PVAP)). In one embodiment, the enteric protectant is selected from the group consisting of poly(acrylic acid) polymer, a poly(sulfonic acid) polymer, a poly(vinylamine) polymer, a poly[2-(dimethylamino)ethyl methacrylate] polymer, copolymers, and derivatives thereof. In one embodiment, the non-pH sensitive polymer is copovidone. In one embodiment, the solid unit(s) of the invention comprises a polymer selected from the group consisting of an enteric protectant, a non-pH-sensitive polymer, a slow-release polymer, a bioadhesive polymer, or any combination thereof; and/or an enteric coating.

In a further embodiment of the invention, the solid unit has an enteric coating.

The invention features, in one embodiment, a stable solid unit suitable for oral administration to a human subject, said solid unit comprising a therapeutic agent, such as a therapeutic protein (e.g., a peptide, a DVD-Ig protein, or an antibody), a stabilizer, e.g., a sugar, and a polymer selected from the group consisting of an enteric protectant, a non-pH-sensitive polymer, a slow-release polymer, a bioadhesive polymer, or any combination thereof, wherein the solid unit is free flowing when placed in a plurality of the solid units.

In one embodiment of the invention, the solid unit comprises two or more therapeutic proteins, e.g., peptides, or antibodies, or antigen-binding portions thereof, directed to distinct molecular targets.

In one embodiment, the solid unit of the invention comprises a surfactant.

In one embodiment, the solid unit of the invention is in a shape selected from the group consisting of a sphere, a cube, a cylinder, or a pyramid.

In a further embodiment, the shape of the solid unit is a sphere. In one embodiment, the sphere has a diameter selected from the group consisting of about 0.1 to about 4 mm; about 0.1 to about 3 mm; about 0.1 to about 2 mm; about 0.1 to about 1 mm; and about 0.1 to about 0.5 mm.

In one embodiment, the sphere comprises an amount of therapeutic agent, such as a therapeutic protein, e.g., a peptide, DVD-Ig protein, or an antibody, or antigen-binding portion thereof, and a diameter selected from the group consisting of an amount of therapeutic agent, such as a therapeutic protein, e.g., a peptide, DVD-Ig protein, or an antibody, or antigen-binding portion thereof, of about 0.02 μg and 2.0 mg and a diameter of about 0.1 mm to about 4 mm; an amount of therapeutic agent, such as a therapeutic protein, e.g., a peptide, DVD-Ig protein, or an antibody, or antigen-binding portion thereof, of about 0.02 μg and 1.5 mg and a diameter of about 0.1 mm to about 3 mm; an amount of therapeutic agent, such as a therapeutic protein, e.g., a peptide, DVD-Ig protein, or an antibody, or antigen-binding portion thereof, of about 0.02 μg and 500 μg and a diameter of about 0.1 mm to about 2 mm; an amount of therapeutic agent, such as a therapeutic protein, e.g., a peptide, DVD-Ig protein, or an antibody, or antigen-binding portion thereof, of about 0.02 μg and 50 μg and a diameter of about 0.1 mm to about 1 mm; an amount of therapeutic agent, such as a therapeutic protein, e.g., a peptide, DVD-Ig protein, or an antibody, or antigen-binding portion thereof, of about 0.02 μg and 6 μg and a diameter of about 0.1 mm to about 0.5 mm.

In one embodiment of the invention, in the solid unit is stable for at least 12 months at about 25° C. or for at least 3 months at about 40° C.

In a further embodiment of the invention, the solid unit is suitable for parenteral or oral administration.

In a further embodiment of the invention, the solid unit is aseptic or is manufactured under aseptic conditions.

In yet another embodiment of the invention, the solid unit comprises a therapeutic protein, such an antibody, or antigen-binding portion thereof, having a post-translational modification selected from the group consisting of glycosylation, oxidation, phosphorylation, sulphation, lipidation, disulphide bond formation, and deamidation, conversion of an N-terminal amino acid deletion of a C-terminal amino acid, attachment of a chemical moiety to the amino acid backbone, N-terminal glutamate converted to pyroglutamate, and addition or deletion of an N-terminal methionine residue.

Another aspect of the invention is a plurality of the solid units described here. In one embodiment, the solid unit or plurality of solid units include solid units having a uniform size distribution and/or a volume ranging from about 0.0005 μl to about 40 μl; about 0.1 μl to about 20 μl; a volume ranging from about 0.5 μl to about 10 μl.

In one embodiment, the plurality of solid units are free-flowing.

Another aspect of the invention is a plurality of solid units, wherein the solid units comprise a therapeutic agent, such as a therapeutic protein, e.g., a peptide, DVD-Ig protein, or an antibody, or antigen-binding portion thereof, to at least two distinct molecular targets. In one embodiment, the solid units within the plurality further comprise an additional therapeutic agent.

A further feature of the invention is a plurality of solid units comprising two or more populations of solid units. The populations may differ in the type of therapeutic agent, such as a therapeutic protein, e.g., a peptide, DVD-Ig protein, or an antibody, or antigen-binding portion thereof, contained within. For example, in one embodiment, the plurality of solid units comprises one population of solid units having a first peptide or first antibody, or an antigen binding portion thereof, and a second population of solid units having a second peptide or a second antibody, or antigen binding portion thereof, wherein the second peptide or second antibody, or antigen-binding portion thereof, is directed to a different molecular target than the first peptide or the first antibody, or antigen-binding portion thereof. Alternatively, the populations within a plurality may differ by size. For example, in one embodiment, the two or more populations of solid units comprise a first population of solid units having substantially similar volumes and a second population of solid units having substantially similar volumes, wherein the first population and the second population have different volumes. In another embodiment, the two or more populations of solid units comprise one population of solid units having a first peptide or antibody, or a first antigen binding portion thereof, and a second population of solid units comprising an additional therapeutic agent. In one embodiment, the two populations of solid units make up at least about 70% of the plurality; at least about 80% of the plurality; at least about 90% of the plurality; or at least about 95% of the plurality.

The invention also includes a pharmaceutical composition comprising the plurality of solid units described herein. In one embodiment, the pharmaceutical composition is a tablet, which, in one alternative, has an enteric coating.

The invention also includes a capsule for oral administration comprising the plurality of solid units described here. In one embodiment, the capsule has an enteric coating.

The invention further features a dual-chambered delivery device comprising the plurality of solid units of the invention. In one embodiment, the device comprises one chamber comprising the plurality of solid units and one chamber comprising a diluent, which may, in one alternative, comprise a therapeutic agent.

The invention further features a method of treating subject in need thereof, comprising administering to the subject a therapeutically effective amount of the solid unit of the invention or the plurality of solid units of the invention. Such administration may be, in one embodiment, parenteral or oral.

The invention also features a method of treating a subject having a disorder, said method comprising combining a drug product comprising the solid unit of the invention or the plurality of solid units of the invention with a diluent to form a reconstituted solution; and administering the reconstituted solution to the subject having the disorder.

In one embodiment, a certain number of solid units is reconstituted in accordance with the dose required by the subject for treatment.

In one embodiment, the reconstituted solution is prepared in a dual chamber syringe. In another embodiment, the reconstituted solution is prepared in a dual chamber patch pump.

Also included within the scope of the invention is a method for preparing a stable solid unit suitable for pharmaceutical administration comprising a peptide or an antibody, or antigen-binding portion thereof, said method comprising providing a solution comprising about 20 mg/ml to about 200 mg/ml of the peptide or antibody, or antigen-binding portion thereof, and about 30 mg/ml to about 100 mg/ml of sorbitol, sucrose, or trehalose; freezing the solution at a temperature of about −5° C. or colder, thereby obtaining a first composition; and subjecting the first composition to vacuum sublimation, thereby preparing a stable solid unit of the peptide or antibody, or antigen-binding portion thereof. In one embodiment, the solution is frozen at a temperature of about −30° C. to about −200° C. In one embodiment, the solution further comprises a polymer selected from the group consisting of an enteric coating, a slow release polymer, a non-pH sensitive polymer, a bioadhesive polymer, or any combination thereof. In one embodiment, the polymer is selected from the group consisting of an enteric coating, a slow release polymer, a non-pH sensitive polymer, a bioadhesive polymer, or any combination thereof.

In one embodiment, the invention includes a method of preparing an intermediate container comprising a bulk intermediate, said method comprising lyophilizing a solution comprising a therapeutic agent and a stabilizer under conditions suitable for controlling nucleation of the solution during freezing, such that a bulk intermediate comprising a plurality of solid units is obtained; placing the bulk intermediate in an intermediate container, thereby preparing the intermediate container comprising the bulk intermediate, wherein the plurality of solid units are free flowing and geometrically uniform. The bulk intermediate may be stored for a period of time selected from the group consisting of about 1 month, about 3 months, about 1 year, or greater than 1 year.

In another embodiment, the invention provides a method for preparing a plurality of solid units comprising a therapeutic agent, said method comprising freezing at least two droplets of a solution comprising the therapeutic agent and a stabilizer under conditions suitable for controlling nucleation of the solution, thereby obtaining a plurality of droplets; and subjecting the plurality of droplets to vacuum sublimation; thereby preparing the plurality of solid units, wherein the plurality of solid units are free-flowing and geometrically uniform. Examples of stabilizers that may be used include, but are not limited to, sorbitol, sucrose, mannitol, and trehalose.

In one embodiment, the methods of the invention include controlled nucleation. Examples of how to achieve controlled nucleation include the use of liquid nitrogen or Freon. Alternatively, controlled nucleation could also be achieved using cold nitrogen gas.

In one embodiment, the at least two droplets are frozen sequentially.

In another embodiment, the plurality of droplets is placed in a primary container prior to vacuum sublimation.

In still another embodiment, the invention features a methods resulting in a plurality of solid units having 10 or less solid units; 50 or less solid units; 100 or less solid units; 1,000 or less solid units; or 5,000 or less solid units; 10,000 or less solid units; 50,000 or less solid units; 100,000 or less solid units; 500,000 or less solid units; 1,000,000 or less solid units; or more than 1,000,000 solid units.

In one embodiment, the invention provides a method of making a plurality of solid units are prepared under aseptic conditions.

In still another embodiment, the invention includes contacting the plurality of solid units with a polymer selected from the group consisting of an enteric coating, a slow release polymer, a non-pH sensitive polymer, a bioadhesive polymer, and any combination thereof.

The invention further features a method for preparing a stable solid unit suitable for pharmaceutical administration comprising a therapeutic agent, for example a therapeutic protein (e.g., a peptide, a DVD-Ig protein, an antibody), said method comprising providing a solution comprising about 20 mg/ml to about 200 mg/ml of the therapeutic protein, about 30 mg/ml to about 100 mg/ml of sucrose or trehalose; freezing the solution at a temperature of about −5° C. or colder using controlled nucleation, thereby obtaining a first composition; and subjecting the first composition to vacuum sublimation, thereby preparing a stable solid unit of the therapeutic agent, such as a protein (e.g., an antibody, peptide, or DVD-Ig protein). In one embodiment, the solution is frozen at a temperature of about −30° C. to about −200° C. In another embodiment, the freezing is performed in liquid nitrogen, cold nitrogen gas, or Freon. In one embodiment, the method includes the used of a solution comprising a therapeutic agent, for example a therapeutic protein (e.g., a peptide, a DVD-Ig protein, an antibody) and about 10 mg/ml to about 200 mg/ml of sucrose.

In one embodiment, the solid unit or plurality of solid units of the invention comprise a therapeutic agent which is a therapeutic protein. Examples of therapeutic proteins that may be used in certain embodiments of the invention include, but are not limited to, a peptide, a DVD-Ig protein, and an antibody, or an antigen-binding portion thereof.

The solid units and plurality of solid units of the invention, in one embodiment, comprise an anti-human Tumor Necrosis Factor alpha (hTNFα) antibody, or antigen-binding portion thereof. Methods of the invention may also be used to make solid unit(s) comprising an anti-hTNFα antibody, or antigen-binding portion thereof.

In one embodiment, the solid unit and plurality of solid units of the invention (or methods of making the same) comprise an anti-human Tumor Necrosis Factor alpha (hTNFα) antibody, or antigen-binding portion thereof, comprising a light chain variable region comprising a CDR3 domain comprising an amino acid sequence set forth as SEQ ID NO: 3, a CDR2 domain comprising an amino acid sequence set forth as SEQ ID NO: 5, and a CDR1 domain comprising an amino acid sequence set forth as SEQ ID NO: 7, and a heavy chain variable region comprising a CDR3 domain comprising an amino acid sequence set forth as SEQ ID NO: 4, a CDR2 domain comprising an amino acid sequence set forth as SEQ ID NO: 6, and a CDR1 domain comprising an amino acid sequence set forth as SEQ ID NO: 8. In one embodiment, the solid unit or plurality of solid units comprises less than 15% of acidic species of the antibody, or antigen-binding portion thereof. In one embodiment, the acidic species comprises AR1, AR2, or both AR1 and AR2. In another embodiment, the solid unit or the plurality of solid units of the invention comprises about 70% lysine variant species of the antibody, or antigen-binding portion thereof, that have two C-terminal lysines (Lys 2) of the antibody, or antigen-binding portion thereof. In a further embodiment, the antibody comprises a PGPK modification.

In one embodiment, the solid unit and plurality of solid units of the invention (or methods of making the same) comprise an anti-human Tumor Necrosis Factor alpha (hTNFα) antibody, or antigen-binding portion thereof, comprising a light chain variable region comprising the amino acid sequence set forth as SEQ ID NO: 1, and a heavy chain variable region of the antibody, or antigen-binding portion thereof, comprising the amino acid sequence set forth as SEQ ID NO: 2. In one embodiment, the solid unit or plurality of solid units comprises less than 15% of acidic species of the antibody, or antigen-binding portion thereof. In one embodiment, the acidic species comprises AR1, AR2, or both AR1 and AR2. In another embodiment, the solid unit or the plurality of solid units of the invention comprises about 70% lysine variant species of the antibody, or antigen-binding portion thereof, that have two C-terminal lysines (Lys 2) of the antibody, or antigen-binding portion thereof. In a further embodiment, the antibody comprises a PGPK modification.

In one embodiment, the solid unit and plurality of solid units of the invention (or methods of making the same) comprise an anti-human Tumor Necrosis Factor alpha (hTNFα) antibody, or antigen-binding portion thereof, comprising the amino acid sequence set forth as SEQ ID NO: 9 and a heavy chain comprising the amino acid sequence set forth as SEQ ID NO: 10. In one embodiment, the solid unit or plurality of solid units comprises less than 15% of acidic species of the antibody, or antigen-binding portion thereof. In one embodiment, the acidic species comprises AR1, AR2, or both AR1 and AR2. In another embodiment, the solid unit or the plurality of solid units of the invention comprises about 70% lysine variant species of the antibody, or antigen-binding portion thereof, that have two C-terminal lysines (Lys 2) of the antibody, or antigen-binding portion thereof. In a further embodiment, the antibody comprises a PGPK modification.

In one embodiment, the solid unit and plurality of solid units of the invention (or methods of making the same) comprise adalimumab, or a biosimilar thereof. In one embodiment, the solid unit or plurality of solid units comprises less than 15% of acidic species of adalimumab, or antigen-binding portion thereof. In one embodiment, the acidic species comprises AR1, AR2, or both AR1 and AR2. In another embodiment, the solid unit or the plurality of solid units of the invention comprises about 70% lysine variant species of the antibody, or antigen-binding portion thereof, that have two C-terminal lysines (Lys 2) of the antibody, or antigen-binding portion thereof. In a further embodiment, the antibody comprises a PGPK modification.

### BRIEF DESCRIPTION OF THE DRAWINGS

FIG. 1 is a graph depicting SEC-HPLC results describing the stability of adalimumab formulated in a solid unit containing various concentrations of sucrose (alone or in combination with glycine) for up to 18 months at 25° C.

FIG. 2 is a graph depicting CEX-HPLC results showing the sum of lysines (indicating the stability of adalimumab) from adalimumab formulated in a solid unit containing various concentrations of sucrose (alone or in combination with glycine) for up to 18 months at 25° C.

FIG. 3 is a graph depicting SEC-HPLC results (% monomer vs. time) describing the stability of adalimumab formulated in a solid unit containing various concentrations of sucrose (alone or in combination with glycine) for up to 9 months at 40° C. (accelerated conditions).

FIG. 4 is a graph depicting the sum of the lysine variants (determined by CEX HPLC) over time for adalimumab formulated in a solid unit containing various concentrations of sucrose (alone or in combination with glycine) for up to 9 months at 40° C. (accelerated conditions).

FIG. 5 is a graph depicting the serum concentration of adalimumab over time when prepared with different formulations (frozen solution, solid units with adalimumab in Bulk drug substance (BDS), and solid units with adalimumab in BDS with sucrose) upon subcutaneous administration in rats.

FIGS. 6A and 6B are graphs depicting the comparison between Adalimumab prepared in fresh solid units, 23 month old solid units, and liquid standards by differential scanning calorimetry.

FIG. 7 graphically depicts the comparison between Adalimumab prepared in fresh solid units, 23 month old solid units, and liquid standards by circular dichronism.

FIG. 8 graphically depicts results from the intact and reduced mass spec analysis of fresh solid units and 23 month old solid units.

FIG. 9 graphically depicts results from dynamic light scattering analysis of fresh solid units and 23 month old solid units.

FIG. 10 graphically depicts results from weak cation exchange chromatography analysis of fresh solid units and 23 month old solid units.

FIG. 11 is a graph depicting the reconstitution times of various solid units comprising Antibody A and traditionally lyophilized cakes comprising Antibody A.

FIG. 12A provides graphically displays prediction results for monomer content, lysine content, and desirability based on a spherical solid unit comprising 50 mg/ml adalimumab, 12 mg/ml mannitol, 65 mg/ml sucrose, and 1 mg/ml Tween 80 stored for 2 months; FIG. 12B graphically depicts a contour profiler for monomer and lysine data provided in FIG. 12A.

FIG. 13A provides graphically displays prediction results for monomer content, lysine content, and desirability based on a spherical solid unit comprising 50 mg/ml adalimumab, 12 mg/ml mannitol, 65 mg/ml sucrose, and no Tween 80 stored for 2 months; FIG. 13B graphically depicts a contour profiler for monomer and lysine data provided in FIG. 13A.

FIG. 14A provides graphically displays prediction results for monomer content, lysine content, and desirability based on a spherical solid unit comprising 50 mg/ml adalimumab, 12 mg/ml mannitol, 65 mg/ml sucrose, and 0.05 mg/ml Tween 80 stored for 2 months; FIG. 14B graphically depicts a contour profiler for monomer and lysine data provided in FIG. 14A.

FIG. 15A provides graphically displays prediction results for monomer content, lysine content, and desirability based on a spherical solid unit comprising 50 mg/ml low acidic adalimumab, 12 mg/ml mannitol, 65 mg/ml sucrose, and 1 mg/ml Tween 80 stored for 2 months; FIG. 15B graphically depicts a contour profiler for monomer and lysine data provided in FIG. 15A.

FIG. 16A provides graphically displays prediction results for monomer content, lysine content, and desirability based on a spherical solid unit comprising 50 mg/ml low acidic adalimumab, 12 mg/ml mannitol, 65 mg/ml sucrose, and no Tween 80 stored for 2 months; FIG. 16B graphically depicts a contour profiler for monomer and lysine data provided in FIG. 16A.

FIG. 17A provides graphically displays prediction results for monomer content, lysine content, and desirability based on a spherical solid unit comprising 50 mg/ml low acidic adalimumab, 12 mg/ml mannitol, 65 mg/ml sucrose, and 0.05 mg/ml Tween 80 stored for 2 months; FIG. 17B graphically depicts a contour profiler for monomer and lysine data provided in FIG. 17A.

FIG. 18A graphically depicts the least square fit response for monomer percentage at 2 months (predicted plot). FIG. 18B graphically depicts the predicted response for the sum of lysines (percentage) at 2 months (predicted plot). FIG. 18 represents predictions for low acidic adalimumab.

FIG. 19A graphically depicts the least square fit response for monomer percentage at 2 months (predicted plot). FIG. 19B graphically depicts the predicted response for the sum of lysines (percentage) at 2 months (predicted plot). FIG. 19 represents predictions for adalimumab.

### DETAILED DESCRIPTION OF THE INVENTION

The present invention is based on the discovery of stable solid compositions of a protein (preferably a therapeutic protein) and a stabilizer, referred to herein as solid units. Specifically, it has been discovered that despite having a high proportion of sugar relative to the protein, the solid units of the invention maintain structural rigidity and resist changes in shape and/or volume when stored under ambient conditions, e.g., room temperature and humidity, for extended periods of time. The solid units of the invention remain free-flowing and are able to maintain long-term physical and chemical stability of the protein without significant degradation and/or aggregate formation. The solid units of the invention have many advantages over the art, including that they can be formulated for oral delivery and are easily reconstituted in a diluent, such as water. Because the solid units are readily dissolved, they may be used in dual chamber delivery devices and may be prepared directly in a device for patient use.

In order that the present invention may be more readily understood, certain terms are first defined.

### I. DEFINITIONS

As used herein, the term “solid unit,” refers to a composition which is suitable for pharmaceutical administration and comprises a therapeutic agent, such as a protein, e.g., an antibody or peptide, and a stabilizer, e.g., a sugar. The solid unit has a structural rigidity and resistance to changes in shape and/or volume. In a preferred embodiment, the solid unit is obtained by lyophilizing a pharmaceutical formulation of a therapeutic agent, e.g., a therapeutic protein. The solid unit may be any shape, e.g., geometric shape, including, but not limited to, a sphere, a cube, a pyramid, a hemisphere, a cylinder, a teardrop, and so forth, including irregularly shaped units. In one embodiment, the solid unit has a volume ranging from about 1 μl to about 20 μl. In one embodiment, the solid unit is not obtained using spray drying techniques, e.g., the solid unit is not a powder or granule.

The term “pharmaceutical composition,” as used herein, refers to a composition, e.g., a plurality of solid units, that it is useful for treating a disease or disorder in a subject, e.g., a human subject. In one embodiment, the pharmaceutical composition comprises a plurality of solid units that comprise a therapeutic agent, e.g., a therapeutic protein, such as an antibody, DVD-Ig protein, or peptide.

The term “pharmaceutical administration” refers to the delivery of a composition comprising a therapeutic agent (e.g., a composition comprising a plurality of solid units comprising a therapeutic protein, such as an antibody, DVD-Ig protein, or peptide) to a subject for treating a disease or disorder. Thus, “suitable for pharmaceutical administration” describes a composition comprising a therapeutic agent which may be used to treat a disease or disorder in a subject. A pharmaceutical composition is suitable for pharmaceutical administration.

As used herein, the phrase “a plurality of solid units” refers to a collection or population of solid units, wherein the collection comprises two or more solid units having a substantially uniform shape, e.g., sphere, and/or volume distribution. In one embodiment, the plurality of solid units is free-flowing. A plurality of solid units, as used herein, is not a powder (a dry, bulk solid composed of a large number of very fine particles that may flow freely when shaken or tilted).

As used herein, the term “geometrically uniform” refers to a plurality of lyophilized solid units having substantially uniform shape and size. In one embodiment, a plurality of solid units that are geometrically uniform are spheres and have substantially similar diameters and protein concentrations.

As used herein, the term “free-flowing” refers to the ability of the plurality of solid units to move in unbroken continuity, similar to a fluid (e.g., the individual solid units within a plurality of solid units do not significantly adhere or stick to one another), prior to reconstitution in a diluent.

The term “substantially similar” or “substantially uniform,” as used herein, denotes a sufficiently high degree of similarity between two numeric values (for example, one associated with a first solid unit of the invention and the other associated with a second solid unit of the invention), such that one of skill in the art would consider the difference between the two values to be of little or no statistical significance within the context of the characteristic measured by said values (e.g., diameter of a sphere). The difference between said two values is, for example, less than about 50%, less than about 40%, less than about 30%, less than about 20%, and/or less than about 10% as a function of the reference/comparator value.

The terms “freeze-drying” and “lyophilizing”, used interchangeably herein, refer to a process in which a solution comprising a therapeutic agent, e.g., a therapeutic protein, (e.g., a peptide, a DVD-Ig protein, or an antibody, or antigen-binding fragment thereof), is frozen and subsequently vacuum sublimated.

The term “nucleation” refers to a physical process in which a change of state, e.g., liquid to solid, occurs in a substance around certain focal points, known as nuclei. “Controlled nucleation” refers to nucleation of a substance under conditions that provide for homogeneous nucleation of a population of substances undergoing a physical process in which a change of state occurs. For example, freezing a plurality of solid units using controlled nucleation results in a population of solid units that are substantially homogenous, e.g., have similar pore size within each solid unit. Controlled nucleation can be achieved by instantaneously freezing a solution.

A “reconstituted” formulation is one which has been prepared by dissolving a lyophilized formulation containing a solid unit in a diluent such that the solid unit (and protein contained therein) is dispersed in the reconstituted formulation. The reconstituted formulation is suitable for administration (e.g. intravenous administration) to a subject to be treated with the therapeutic agent, e.g., the protein of interest (e.g., anti-TNF-alpha antibody, or antigen-binding portion thereof).

A “diluent” as used herein refers to a liquid which is pharmaceutically acceptable (safe and non-toxic for administration to a human) and is useful for the preparation of a liquid formulation, such as a solid unit reconstituted after lyophilization. Exemplary diluents include sterile water, bacteriostatic water for injection (BWFI), a pH buffered solution (e.g. phosphate-buffered saline), sterile saline solution, Ringer's solution or dextrose solution. In an alternative embodiment, diluents can include aqueous solutions of, for example, salts and/or buffers and/or polymers.

As used herein, the term “enteric protectant” refers to a barrier applied to or within a solid unit to facilitate oral delivery of a therapeutic agent, such as a therapeutic protein, e.g., a peptide, DVD-Ig protein, or antibody. An enteric protectant can be found within the solid unit or on the outside of the solid unit. In one embodiment, an enteric protectant is an enteric coating. As used herein, the term “enteric coating” refers to a barrier applied to the outer surface of a solid unit composition for oral administration. An enteric protectant controls the location in the digestive system where the protein within the solid unit is absorbed. An enteric protectant is stable (e.g., acid-resistant) at high acidic pH, e.g., the pH found in the stomach, but breaks down in a less acidic environment than that of the stomach. For example, enteric protectants will not dissolve in the acidic juices of the stomach (pH about 3), but will dissolve in the alkaline (pH about 7 to about 9) environment present in the small intestine. Suitable enteric protectants are polymers, including, but not limited to, polymers having free carboxylic acid groups on the polymer backbone including, for example, polymethacrylates (such as methacrylic acid/ethyl acrylate), cellulose esters (such as cellulose acetate phthalate (CAP), cellulose acetate trimellitate (CAT), and hydroxypropylmethylcellulose acetate succinate (HPMCAS)), and polyvinyl derivatives (such as Polyvinyl acetate phthalate (PVAP)).

A “non-pH-sensitive polymer” refers to a polymer suitable for inclusion in a solid unit of the invention for oral administration that releases the therapeutic agent, such as a protein, from the solid unit composition in the digestive system regardless of the pH. An example of a non-pH sensitive polymer is copovidone.

As used interchangeably herein, the terms “slow-release polymer,” “extended-release polymer,”, or “sustained-release polymer” refer to a polymer suitable for inclusion in a solid unit for oral administration that facilitates release of the therapeutic agent, such as a protein from the solid unit over time. A slow-release polymer allows the protein to be released in a slow, consistent manner.

As used herein, the term “bioadhesive polymer” refers to a polymer that can adhere to the mucin/epithelial surface. Through adhesion, bioadhesive polymers may be used to facilitate absorption of a therapeutic agent, such as a therapeutic protein, by the intestinal tract.

As used herein, the term “stable” solid unit refers to a solid unit in which the therapeutic agent, such as a protein, (e.g., antibody, peptide, or DVD-Ig protein), therein essentially retains its physical stability and/or chemical stability and/or biological activity. Various analytical techniques for measuring stability of the composition and the protein therein are available in the art and are reviewed in Peptide and Protein Drug Delivery 247-301, Vincent Lee Ed., Marcel Dekker, Inc., New York, N.Y., Pubs. (1991); and Jones, A. (1993) Adv. Drug Delivery Rev. 10: 29-90 (both incorporated by reference). For example, in one embodiment, the stability of a protein within a solid unit is determined using size exclusion chromatography (SEC) to determine the percentage of monomer protein in a reconstituted solution, where the protein is considered stable if there is a low percentage of degraded (e.g., fragmented) and/or aggregated protein or a high level (e.g., 98%, 99%, or 99.5% or more) of monomer protein.

A solid unit and/or a protein within the solid unit “retains its physical stability” if it shows substantially no signs of, e.g., aggregation, precipitation and/or denaturation upon visual examination of color and/or clarity, or as measured by UV light scattering or by SEC. Aggregation of a protein is a process whereby individual molecules or complexes associate covalently or non-covalently to form aggregates. Aggregation can proceed to the extent that a visible precipitate is formed.

Stability, such as physical stability of a composition and/or protein within the compositions, may be assessed by methods well-known in the art, including measurement of a sample's apparent attenuation of light (absorbance, or optical density). Such a measurement of light attenuation relates to the turbidity of a solid unit composition when reconstituted. The turbidity of a composition is partially an intrinsic property of the protein dissolved in solution and is commonly determined by nephelometry, and measured in Nephelometric Turbidity Units (NTU).

The degree of turbidity, e.g., as a function of the concentration of one or more of the components in the solution, e.g., protein and/or salt concentration, is also referred to as the “opalescence” or “opalescent appearance” of a composition. The degree of turbidity can be calculated by reference to a standard curve generated using suspensions of known turbidity. Reference standards for determining the degree of turbidity for pharmaceutical compositions can be based on the European Pharmacopeia criteria (European Pharmacopoeia, Fourth Ed., Directorate for the Quality of Medicine of the Council of Europe (EDQM), Strasbourg, France). According to the European Pharmacopeia criteria, a clear solution is defined as one with a turbidity less than or equal to a reference suspension which has a turbidity of approximately 3 according to European Pharmacopeia standards. Nephelometric turbidity measurements can detect Rayleigh scatter, which typically changes linearly with concentration, in the absence of association or nonideality effects. Other methods for assessing physical stability are well-known in the art.

A protein “retains its chemical stability” in a solid unit, if the chemical stability at a given time is such that the protein is considered to still retain its biological activity (e.g., when reconstituted). Chemical stability can be assessed by, e.g., detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation or oxidation) which can be evaluated by ion-exchange chromatography, for example.

A protein “retains its biological activity” in a solid unit, if the protein in a composition is biologically active for its intended purpose (e.g., when reconstituted). For example, biological activity is retained if the biological activity of an antibody in the composition is within about 30%, about 20%, or about 10% (within the errors of the assay) of the biological activity exhibited at the time the composition was prepared (e.g., as determined in an antigen binding assay).

As used herein, the term “protein” refers to compounds composed, at least in part, of amino acid residues linked by amide bonds (i.e., peptide bonds). The term “protein” is intended to encompass peptides, polypeptides, and proteins, including antibodies, and antigen-binding portions thereof. Typically, a peptide will be composed of less than about 100 amino acids, more typically less than about 50 amino acid residues and even more typically, less than about 25 amino acid residues. The term “protein” is further intended to encompass peptide analogues, peptide derivatives and peptidomimetics that mimic the chemical structure of a peptide composed of naturally-occurring amino acids. Examples of peptide analogues include peptides comprising one or more non-natural amino acids. Examples of peptide derivatives include peptides in which an amino acid side chain, the peptide backbone, or the amino- or carboxy-terminus has been derivatized (e.g., peptidic compounds with methylated amide linkages). Examples of peptidomimetics include proteins in which the peptide backbone is substituted with one or more benzodiazepine molecules (see e.g., James, G. L. et al. (1993) Science 260:1937-1942), “inverso” peptides in which all L-amino acids are substituted with the corresponding D-amino acids, “retro-inverso” peptides (see U.S. Pat. No. 4,522,752 by Sisto) in which the sequence of amino acids is reversed (“retro”) and all L-amino acids are replaced with D-amino acids)“inverso”) and other isosteres, such as peptide back-bone (i.e., amide bond) mimetics, including modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets. Other possible modifications include an N-alkyl (or aryl) substitution (ψ[CONR]), backbone crosslinking to construct lactams and other cyclic structures, and other derivatives including C-terminal hydroxymethyl derivatives, O-modified derivatives and N-terminally modified derivatives including substituted amides such as alkylamides and hydrazides.

As used herein, the term “therapeutic protein” is intended to refer to a protein that exhibits pharmacologic activity, either in its present form or upon processing in vivo (i.e., therapeutic proteins include proteins with constitutive pharmacologic activity and proteins in a “prodrug” form that have to be metabolized or processed in some way in vivo following administration in order to exhibit pharmacologic activity). It should be noted that while therapeutic proteins may be used for treatment purposes, the invention is not limited to such use, as said proteins may also be used for in vitro studies. Preferably, a therapeutic protein is produced recombinantly or synthetically. Examples of therapeutic proteins include, but are not limited to, peptides, antibodies, and DVD-Ig proteins.

The term “antibody” broadly refers to an immunoglobulin (Ig) molecule, generally comprised of four polypeptide chains, two heavy (H) chains and two light (L) chains, or any functional fragment, mutant, variant, or derivative thereof, that retains the essential target binding features of an Ig molecule. In one embodiment of the invention, the solid unit contains an antibody with CDR1, CDR2, and CDR3 sequences of adalimumab (D2E7), as described in U.S. Pat. Nos. 6,090,382 and 6,258,562, each incorporated by reference herein.

The terms “antigen-binding portion” or “antigen-binding fragment”, used interchangeably throughout, of an antibody (or simply “antibody portion”), as used herein, refers to one or more fragments of an antibody that retain the ability to specifically bind to an antigen (e.g., hTNF-alpha). It has been shown that the antigen-binding function of an antibody can be performed by fragments of a full-length antibody. In one embodiment, the antigen-binding portion is an antigen-binding portion comprising the Fc region or the CH3 domain of an antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). In one embodiment of the invention, the composition contains an antigen-binding portions described in U.S. Pat. Nos. 6,090,382 and 6,258,562, each incorporated by reference herein.

As used herein, the term “CDR” refers to the complementarity determining region within an antibody variable sequence. There are three CDRs in each of the variable regions of the heavy chain and the light chain, which are designated CDR1, CDR2 and CDR3, for each of the heavy and light chain variable regions. The exact boundaries of these CDRs have been defined differently according to different systems. The system described by Kabat (Kabat et al. (1991) Sequences of Proteins of Immunological Interest, Fifth Edition, U.S. Department of Health and Human Services, NIH Publication No. 91-3242) not only provides an unambiguous residue numbering system applicable to any variable region of an antibody, but also provides precise residue boundaries defining the three CDRs. These CDRs may be referred to as Kabat CDRs. Chothia et al. found that certain sub-portions within Kabat CDRs adopt nearly identical peptide backbone conformations, despite having great diversity at the level of amino acid sequence (Chothia et al. (1987) Mol. Biol. 196:901-917; Chothia et al. (1989) Nature 342:877-883) These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45. Still other CDR boundary definitions may not strictly follow one of the herein described systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs. In one embodiment, the antibody used in the methods and compositions of the invention includes the six CDRs from the antibody adalimumab.

The phrase “recombinant antibody” refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of particular immunoglobulin gene sequences (such as human immunoglobulin gene sequences) to other DNA sequences. Examples of recombinant antibodies include recombinant human, chimeric, CDR-grafted and humanized antibodies.

The term “human antibody,” as used herein, is intended to include antibodies having variable and constant regions derived from human germline immunoglobulin sequences. The human antibodies used in the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

An “isolated antibody, or antigen-binding portion thereof,” as used herein, is intended to refer to an antibody that is substantially free of other antibodies, or antigen-binding portions thereof, having different antigenic specificities (e.g., an isolated antibody, or antigen-binding portion thereof, that specifically binds hTNF-alpha is substantially free of antibodies, or antigen-binding portions thereof, that specifically bind antigens other than hTNF-alpha). An isolated antibody, or antigen-binding portion thereof, that specifically binds an antigen, such as hTNF-alpha may, however, have cross-reactivity to other antigens, such as TNF-alpha molecules from other species. Moreover, an isolated antibody, or antigen-binding portion thereof, may be substantially free of other cellular material and/or chemicals.

A “neutralizing antibody, or antigen-binding portion thereof,” as used herein (e.g., an “antibody, or antigen-binding portion thereof, that neutralizes hTNF-alpha activity”), is intended to refer to an antibody, or antigen-binding portion thereof, e.g., an anti-hTNF-alpha antibody, or antigen-binding portion thereof, whose binding to antigen results in inhibition of the biological activity of hTNF-alpha. This inhibition of the biological activity of, e.g., hTNF-alpha, can be assessed by measuring one or more indicators of biological activity. For example, when the antibody, or antigen-binding portion thereof, is a human anti-TNF-alpha antibody, or antigen-binding portion thereof, a biological activity that can be measured includes hTNF-alpha-induced cytotoxicity (either in vitro or in vivo), hTNF-alpha-induced cellular activation and hTNF-alpha binding to hTNF-alpha receptors. These indicators of hTNF-alpha biological activity can be assessed by one or more of several standard in vitro or in vivo assays known in the art, and described in U.S. Pat. Nos. 6,090,382 and 6,258,562, each incorporated by reference herein. In one embodiment, the ability of an antibody, or antigen-binding portion thereof, to neutralize hTNF-alpha activity is assessed by inhibition of hTNF-alpha-induced cytotoxicity of L929 cells. As an additional or alternative parameter of hTNF-alpha activity, the ability of an antibody, or antigen-binding portion thereof, to inhibit hTNF-alpha-induced expression of ELAM-1 on HUVEC, as a measure of hTNF-alpha-induced cellular activation, can be assessed.

The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “kₒₙ,” as used herein, is intended to refer to the on rate constant for association of a binding protein (e.g., an antibody, or antigen-binding portion thereof) to the antigen to form the, e.g., antibody, or antigen-binding portion thereof/antigen complex as is known in the art.

The term “k_{off},” as used herein, is intended to refer to the off rate constant for dissociation of an antibody, or antigen-binding portion thereof, from the antibody, or antigen-binding portion thereof/antigen complex.

The term “K_{d},” as used herein, is intended to refer to the dissociation constant of a particular antibody, or antigen-binding portion thereof-antigen interaction and refers to the value obtained in a titration measurement at equilibrium, or by dividing the dissociation rate constant (k_{off}) by the association rate constant (kₒₙ).

As used herein, the term “biosimilar” (of an approved reference product/biological drug, such as a therapeutic protein, e.g., an antibody, or antigen-binding portion thereof) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biological product. In one embodiment, the biosimilar and reference product utilize the same mechanism or mechanisms of action for the condition or conditions of use prescribed, recommended, or suggested in the proposed labeling, but only to the extent the mechanism or mechanisms of action are known for the reference product. In one embodiment, the condition or conditions of use prescribed, recommended, or suggested in the labeling proposed for the biological product have been previously approved for the reference product. In one embodiment, the route of administration, the dosage form, and/or the strength of the biosimilar are the same as those of the reference product. In one embodiment, the facility in which the biosimilar is manufactured, processed, packed, or held meets standards designed to assure that the biosimilar continues to be safe, pure, and potent. The reference product may be approved in at least one of the U.S., Europe, or Japan.

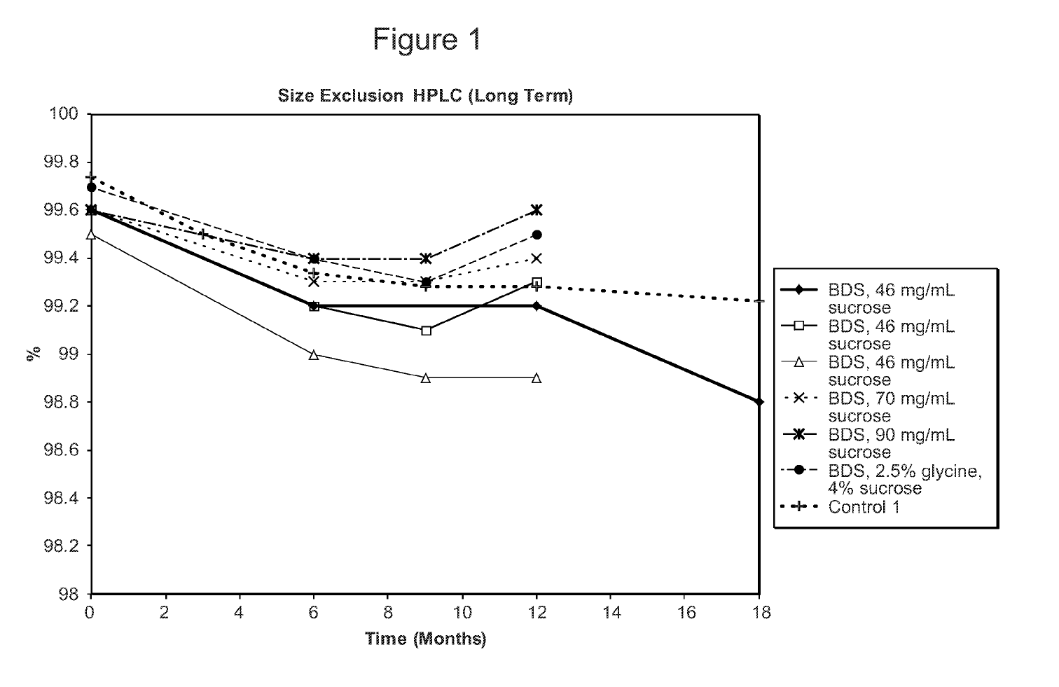

As used herein, the terms “Dual Variable Domain Immunoglobulin” or “DVD-Ig™” are understood to include immunoglobulin molecules having the structure schematically represented in FIG. 1A provided in US Patent Publications 20100260668 and 20090304693 both of which are incorporated herein by reference. A DVD-Ig™ comprises a paired heavy chain DVD polypeptide and a light chain DVD polypeptide with each paired heavy and light chain providing two antigen binding sites. Each binding site includes a total of 6 CDRs involved in antigen binding per antigen binding site. A DVD-Ig™ is typically has two arms (is divalent), with each arm of the DVD being dual-specific, providing an immunoglobulin with four binding sites.

The term “excipient” refers to an agent that may be added to a solid unit to provide a desired characteristic, e.g., consistency, improving stability, and/or to adjust osmolality. Examples of commonly used excipients include, but are not limited to, sugars, polyols, amino acids, surfactants, and polymers.

A “stabilizer,” as used herein, refers to an excipient, particularly a pharmaceutically acceptable excipient, which inhibits, prevents, slows down, or reduces the degradation of a protein in a solid unit as compared to the protein in the solid unit in the absence of the stabilizer. In one embodiment, the stabilizer is a lyoprotectant.

As used herein, the term “lyoprotectant” refers to a stabilizer that is used to protect a protein during the lyophilization process, particularly during the drying stages of lyophilization. An example of a lyoprotectant is a sugar.

The term “sugar” is meant to refer to as used herein denotes a monosaccharide or an oligosaccharide. A monosaccharide is a monomeric carbohydrate which is not hydrolysable by acids, including simple sugars and their derivatives, e.g. amino sugars. Examples of monosaccharaides include, but are not limited to, glucose, fructose, galactose, mannose, sorbose, ribose, deoxyribose, neuraminic acid. An oligosaccharide is a carbohydrate consisting of more than one monomeric saccharide unit connected via glycosidic bond(s) either branched or in a chain. The monomeric saccharide units within an oligosaccharide can be identical or different. Depending on the number of monomeric saccharide units the oligosaccharide is a di-, tri-, tetra-, penta- and so forth saccharide. Examples of oligosaccharides include, but are not limited to, maltose, sucrose, lactose, melezitose, trehalose, sorbose, and raffinose. In contrast to polysaccharides, monosaccharides and oligosaccharides are water soluble. Examples of sugars include, but not limited to, a reducing sugar, a nonreducing sugar, a sugar alcohol, and a sugar acid. A “reducing sugar” is a sugar that contains a free aldehyde or ketone group and can reduce metal ions or react covalently with lysine and other amino groups in proteins. A “nonreducing sugar” is a sugar that lacks a free aldehyde or ketonic group and is not oxidized by mild oxidizing agents such as Fehling's or Benedict's solutions. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. In one embodiment of the invention, the sugar is sucrose or trehalose. In another embodiment of the invention, the formulation comprises about 40-90 mg/ml of a sugar, e.g., sucrose. In another further embodiment of the invention, the formulation comprises about 45-80 mg/ml of a sugar, e.g., sucrose.

As used herein, the term “buffer” refers to an agent(s) in a solution that allows the solution to resist changes in pH by the action of its acid-base conjugate components. Examples of buffers include acetate (e.g. sodium acetate), succinate (such as sodium succinate), gluconate, histidine, methionine, citrate, phosphate, citrate/phosphate, imidazole, combinations thereof, and other organic acid buffers. In one embodiment, a buffer is not a protein. A buffer may provide a solution with a pH in the range from about 4 to about 8; from about 4.5 to about 7; or from about 5.0 to about 6.5.

As used herein, the term “surfactant” generally includes an agent that protects a protein, e.g., antibody, or antigen-binding portion thereof, from air/solution interface-induced stresses, solution/surface induced-stresses, to reduce aggregation of the protein, or to minimize the formation of particulates in the formulation. Exemplary surfactants include, but are not limited to, nonionic surfactants such as polysorbates (e.g. polysorbates 20 and 80) or poloxamers (e.g. poloxamer 188). The term “surfactant” or “detergent” includes nonionic surfactants such as, but not limited to, polysorbates. In one embodiment, a surfactant includes poloxamers, e.g., Poloxamer 188, Poloxamer 407; polyoxyethylene alkyl ethers, e.g., Brij 35®, Cremophor A25, Sympatens ALM/230; and polysorbates/Tweens, e.g., Polysorbate 20 (Tween 20), Polysorbate 80 (Tween 80), Mirj, and Poloxamers, e.g., Poloxamer 188.

As used herein, the terms “therapeutically effective amount” or “effective amount” of a protein, e.g., an antibody, or antigen-binding portion thereof, refers to an amount used in the prevention or treatment or alleviation of a symptom of a disorder for the treatment of which the protein is effective.

The term “human TNF-alpha” (abbreviated herein as hTNF-alpha, TNFα, or hTNFa), as used herein, is intended to refer to a human cytokine that exists as a 17 kDa secreted form and a 26 kDa membrane associated form, the biologically active form of which is composed of a trimer of noncovalently bound 17 kDa molecules. The structure of hTNF-alpha is described further in, for example, Pennica, D., et al. (1984) Nature 312:724-729; Davis, J. M., et al. (1987) Biochem 26:1322-1326; and Jones, E. Y., et al. (1989) Nature 338:225-228. The term human TNF-alpha is intended to include recombinant human TNF-alpha (rhTNF-alpha), which can be prepared by standard recombinant expression methods or purchased commercially (R & D Systems, Catalog No. 210-TA, Minneapolis, Minn.).

The term “subject” or “patient” is intended to include mammalian organisms. Examples of subjects/patients include humans and non-human mammals, e.g., non-human primates, dogs, cows, horses, pigs, sheep, goats, cats, mice, rabbits, rats, and transgenic non-human animals. In specific embodiments of the invention, the subject is a human.

As used herein, the term “drug product” generally refers to a composition that comprises a solid unit (or a plurality of solid units) comprising a therapeutic agent, e.g., a small molecule or a protein (e.g., a peptide, an antibody, or a DVD-Ig protein). The drug product is suitable for administration to a subject, e.g., a human subject, in need of the therapeutic agent. In one embodiment, the drug product comprises a plurality of solid units which are free-flowing. In one embodiment, the drug product comprises a plurality of solid units that are further geometrically uniform and/or uniform in size.

As used herein, the term “drug substance” refers to a composition that comprises a solid unit (or a plurality of solid units) comprising a therapeutic agent, e.g., a small molecule or a protein (e.g., a peptide, an antibody, or a DVD-Ig protein), that requires further processing to become a drug product. For example, a drug substance may be a plurality of solid units that comprise a therapeutic agent and a buffer and/or excipient, but is not suitable for administration for therapeutic purposes and requires further processing to become a drug product. In another example, a drug substance is a plurality of solid units comprising a therapeutic agent which may be further processed by coating with an enteric coating to become a drug product. In one embodiment, the drug substance comprises a plurality of solid units which are free-flowing. In one embodiment, the drug substance comprises a plurality of solid units that are uniform in size.

As used herein, the term “bulk intermediate” refers to a drug product or a drug substance.

The term “primary container,” as used herein, refers to an article which holds or is intended to contain a drug product suitable for the intended use of the drug product. In one embodiment, a primary container is a dual chamber syringe. In another embodiment, a primary container is a dual chamber cartridge. In another embodiment, a primary container is a dual chamber patch pump.

The term “intermediate container,” as used herein, refers to an article which holds or is intended to contain a bulk intermediate. An intermediate container is not a primary container.

Various aspects of the invention are described in further detail in the following subsections.

### II. SOLID UNITS OF THE INVENTION

The present invention creates a holistic manufacturing system for the delivery of any agent, but most especially pharmaceutical drug products such as therapeutic proteins. The system incorporates a lyophilization process, enabling controlled nucleation, to produce uniform, free flowing solid units.

Under typical lyophilization methods, a liquid solution is placed into a final primary container prior to lyophilization, resulting in a lyophilized cake. While traditional lyophilization is performed in the container in which the lyo-cake will be stored and eventually reconstituted, the current invention provides a lyophilization process which can be independent of the primary container. Indeed, the invention provides stable solid units which can be manufactured, subsequently stored, and then further processed according to specific needs. The free flowing solid units of the invention are of uniform geometry, volume, and composition, and are capable of being stored and managed as a large bulk volume, or as a single dose in a primary drug container without impact to the lyophilization process parameters.

The invention is a platform technology, applicable to a broad range of antibody, protein-based, small molecule, or combinations of pharmaceutical products with minimal changes to critical process parameters. The invention provides solid units that may be used both as oral and injectable dosage forms.

In one embodiment, the invention features a drug product comprising a plurality of lyophilized, spherical solid units which are free-flowing and geometrically uniform, wherein the plurality of solid units comprises a therapeutic protein and a sugar. The solid units within the drug product may be produced using a controlled nucleation.

A further advantage of the methods and compositions of the invention, is that they present the opportunity to combine distinct agents that have separate formulation stability needs and are otherwise incompatible as far as combining in a single formulation. In cases where a biopharmaceutical product is made from a combination of two or more active agents, e.g., two antibodies having distinct antigen specificity, the agents must be able to be co-formulated in order to be lyophilized collectively as one product. This can be a challenge given that a common formulation must be identified in which both agents are stable and still biologically active. The present invention does not require a common formulation in order to provide a combination of therapeutic agents in one dosage form. For example, a plurality of solid units comprising two distinct antibody populations, i.e. solid units comprising a first antibody having specific to antigen 1 and solid units comprising a second antibody having a specificity to antigen 2, may be combined as free flowing spherical solid units which may be combined upon reconstitution in water or a buffered solution such that the resulting liquid formulation is stable for a given period of time sufficient for administration of the reconstituted formulation to a subject in need. Thus, the present invention provides a process whereby each active therapeutic agent can be lyophilized in its preferred formulation, and then combined as a plurality of solid units until reconstitution is warranted.

A broad range of formulations described herein and in the Examples shows that the process produces stable drug product examples for many active pharmaceutical substances, including stability at room temperature and/or accelerated storage conditions for a protein, including a peptide, a DVD-Ig protein, and an antibody, or antigen-binding portion thereof.

Thus, the present invention provides stable solid units (and pluralities thereof) containing a therapeutic agent, such as a protein (e.g., an antibody, or antigen-binding portion thereof), and a stabilizer, e.g., a sugar such as sorbitol, mannitol, sucrose or trehalose. The present invention is directed to a stable solid unit suitable for pharmaceutical administration where the solid unit comprises a protein, such as, but not limited to a peptide, an antibody, or antigen-binding portion thereof, or a DVD-Ig protein, and a stabilizer, such as a lyoprotectant, e.g., a sugar.

The solid units of the invention provide many advantages over the art due to their stability and the ability to make homogenous populations of solid units having similar sizes and agent (e.g., protein) content. The solid units of the invention, when considered together, are free flowing. Further, solid units of the invention may be geometrically uniform. The solid units of the invention are not produced using spray-drying or sprayfreeze drying techniques. Such techniques do not result in a population of solid units having consistent features, e.g., geometric uniformity, in contrast to the solid units made according to the methods described herein.

The solid units described herein are stable, in that they can maintain stability of a therapeutic agent, e.g., a protein, (e.g., antibody or antigen-binding portion thereof), over time, including at high temperatures. In one embodiment, the invention provides a stable solid unit suitable for pharmaceutical administration, said lyophilized solid unit comprising a mixture of an anti-human TNFα antibody, or an antigen-binding portion thereof, and a stabilizer, wherein the stabilizer prevents or reduces chemical or physical instability of the antibody, or antigen-binding portion thereof, upon freeze-drying and subsequent storage.

The solid unit of the invention may include a polymer within the solid unit and/or as a coating on the outside of the solid unit. Polymers that may be combined with the solid unit of the invention include, but are not limited to, a bioadhesive polymer, an enteric protectant, a non-pH sensitive polymer, and a sustained-release polymer (and combinations thereof).

In one embodiment, the solid unit of the invention is made under aseptic conditions.

A solid unit of the invention may have a volume ranging from about 0.0005 μl to about 20 μl, about 0.005 μl to about 20 μl, 0.001 μl to about 20 μl, 0.05 μl to about 20 μl, 0.01 μl to about 20 μl, 0.0005 μl to about 10 μl, about 0.005 μl to about 10 μl, 0.001 μl to about 10 μl, 0.05 μl to about 10 μl, 0.01 μl to about 10 μl, 0.0005 μl to about 5 μl, about 0.005 μl to about 5 μl, 0.001 μl to about 5 μl, 0.05 μl to about 5 μl, 0.01 μl to about 5 μl, 0.0005 μl to about 1 μl, about 0.005 μl to about 1 μl, 0.001 μl to about 1 μl, 0.05 μl to about 1 μl, 0.01 μl to about 1 μl, about 0.1 μl to about 20 μl, about 0.5 to about 20, about 1 to about 20, about 1.5 to about 20, about 2 to about 20, about 2.5 to about 20, about 3 to about 20, about 3.5 to about 20, about 4 to about 20, about 4.5 to about 20, about 5 to about 20, about 5.5 to about 20, about 6 to about 20, about 6.5 to about 20, about 7 to about 20, about 7.5 to about 20, about 8 to about 20, about 8.5 to about 20, about 9 to about 20, about 9.5 to about 20, about 10 to about 20, about 15 to about 20, about 12 to about 20, about 13 to about 20, about 14 to about 20, about 15 to about 20, about 16 to about 20, about 17 to about 20, about 18 to about 20, about 19 to about 20, about 0.5 to about 15, about 1 to about 15, 1.5 to about 15, about 2 to about 15, about 2.5 to about 15, about 3 to about 15, about 3.5 to about 15, about 4 to about 15, about 4.5 to about 15, about 5 to about 15, about 5.5 to about 15, about 6 to about 15, about 6.5 to about 15, about 7 to about 15, about 7.5 to about 15, about 8 to about 15, about 8.5 to about 15, about 9 to about 15, about 9.5 to about 15, or about 10 μl to about 15 μl. In one embodiment, the solid unit of the invention has a volume of between about 9 μl and 15 μl. Volumes and ranges intermediate to the above recited volumes and ranges are also intended to be part of this invention (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 52.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or about 20.0 μl).

A solid unit of the invention may be any suitable shape. In one embodiment, the solid unit is a geometric shape, e.g., a sphere, a cube, a cylinder, or a pyramid. In one embodiment, a solid unit is a sphere having a diameter of about 0.1 to about 4 mm; about 0.1 to about 3.5 mm; about 0.1 to about 3 mm; about 0.1 to about 2.5 mm; about 0.1 to about 2 mm; about 0.1 to about 1.5 mm; about 0.1 to about 1 mm; or about 0.1 to about 0.5 mm. Diameters and ranges intermediate to the above recited diameters and ranges are also intended to be part of this invention (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0 mm). Other exemplary ranges include about 0.1 to about 4 mm; about 0.1 to about 3 mm; about 0.1 to about 2 mm; about 0.1 to about 1 mm; and about 0.1 to about 0.5 mm.

In one embodiment, the invention features solid units that are spherical in shape. A solid unit that is spherical in shape has approximately the same diameter regardless of the point at which the calculation is taken on the outside of the solid unit. Thus, a sphere does not include a partial sphere, i.e., a sphere with a flat surface(s).

In one embodiment, the solid unit is a sphere having a diameter which is greater than 1 mm and less than 4 mm.

In one embodiment, the solid unit of the invention contains a protein, e.g., a peptide, a DVD-Ig protein, or an antibody or antigen-binding portion thereof, and an additional therapeutic agent.

Solid units of the invention are particularly useful in providing a consistent means for measuring a dose of a therapeutic agent, such as a therapeutic agent, such as a protein (e.g., an antibody, antigen-binding portion thereof, or a DVD-Ig protein). As a plurality of the solid units may have substantially the same shape, the solid units in turn have substantially similar amounts of therapeutic agent, e.g., protein. Thus, the amount of agent, such as a protein, (e.g., antibody, or antigen-binding portion thereof), in a solid unit, (such as a sphere shaped solid unit), may be between about 0.02 μg and 6.0 mg, about 0.05 μg to about 6.0 mg, about 0.1 μg to about 6.0 mg, about 0.2 μg to about 6.0 mg, about 0.5 μg to about 6.0 mg, about 1 μg to about 6.0 mg, about 5 μg to about 6.0 mg, about 10 μg to about 6.0 mg, about 15 μg to about 6.0 mg, 0.02 μg and 4.0 mg, about 0.05 μg to about 4.0 mg, about 0.1 μg to about 4.0 mg, about 0.2 μg to about 4.0 mg, about 0.5 μg to about 4.0 mg, about 1 μg to about 4.0 mg, about 5 μg to about 4.0 mg, about 10 μg to about 4.0 mg, about 15 μg to about 4.0 mg, 0.02 μg and 2.0 mg, about 0.05 μg to about 2.0 mg, about 0.1 μg to about 2.0 mg, about 0.2 μg to about 2.0 mg, about 0.5 μg to about 2.0 mg, about 1 μg to about 2.0 mg, about 5 μg to about 2.0 mg, about 10 μg to about 2.0 mg, about 15 μg to about 2.0 mg, about 0.02 μg and 1.0 mg, about 0.05 μg to about 1.0 mg, about 0.02 μg and 1.0 mg, about 0.05 μg to about 1.0 mg, about 0.1 μg to about 1.0 mg, about 0.2 μg to about 1.0 mg, about 0.5 μg to about 1.0 mg, about 1 μg to about 1.0 mg, about 5 μg to about 1.0 mg, about 10 μg to about 1.0 mg, about 15 μg to about 1.0 mg, about 0.02 μg and 0.5 mg, about 0.05 μg to about 0.5 mg, about 0.1 μg to about 0.5 mg, about 0.2 μg to about 0.5 mg, about 0.5 μg to about 0.5 mg, about 1 μg to about 0.5 mg, about 5 μg to about 0.5 mg, about 10 μg to about 0.5 mg, about 15 μg to about 0.5 mg, about 0.02 μg and 0.25 mg, about 0.05 μg to about 0.25 mg, about 0.1 μg to about 0.25 mg, about 0.2 μg to about 0.25 mg, about 0.5 μg to about 0.25 mg, about 1 μg to about 0.52 mg, about 5 μg to about 0.25 mg, about 10 μg to about 0.25 mg, about 15 μg to about 0.25 mg, about 0.02 μg and 0.1 mg, about 0.05 μg to about 0.1 mg, about 0.1 μg to about 0.1 mg, about 0.2 μg to about 0.1 mg, about 0.5 μg to about 0.1 mg, about 1 μg to about 0.1 mg, about 5 μg to about 0.1 mg, about 10 μg to about 0.1 mg, about 15 μg to about 0.1 mg, about 0.02 μg and 0.05 mg, about 0.05 μg to about 0.05 mg, about 0.1 μg to about 0.05 mg, about 0.2 μg to about 0.05 mg, about 0.5 μg to about 0.05 mg, about 1 μg to about 0.05 mg, about 5 μg to about 0.05 mg, about 10 μg to about 0.05 mg, or about 15 μg to about 0.05 mg. Amounts and ranges intermediate to the above recited amounts and ranges are also intended to be part of this invention. Other exemplary ranges of agent, e.g., protein, amount include 0.02 μg to 6.0 mg or 15 μg to 4.0 mg of therapeutic protein.

In certain embodiments of the invention, the amount of therapeutic agent, such as a protein (e.g., an antibody, or antigen-binding portion thereof, peptide, or DVD-Ig protein) in a solid unit, such as a sphere shaped solid unit, may be between about 0.02 μg and 2.0 mg and the diameter of the sphere may be between about 0.1 mm to about 4 mm. In other embodiments, the amount of therapeutic agent, such as a protein (e.g., an antibody, or antigen-binding portion thereof, peptide, or a DVD-Ig protein) in a solid unit, such as a sphere shaped solid unit, may be between about 0.02 μg and 1.5 mg and the diameter of the sphere may be between about 0.1 mm to about 3 mm. In yet other embodiments, the amount of therapeutic agent, such as a protein (e.g., an antibody, or antigen-binding portion thereof, peptide, or a DVD-Ig protein) in a solid unit, such as a sphere shaped solid unit, may be between about 0.02 μg and 500 μg and the diameter of the sphere may be between about 0.1 mm to about 2 mm. In some embodiments, the amount of therapeutic agent, such as a protein (e.g., an antibody, or antigen-binding portion thereof, peptide, or a DVD-Ig protein) in a solid unit, such as a sphere shaped solid unit, may be between about 0.02 μg and 50 μg and the diameter of the sphere may be between about 0.1 mm to about 1 mm. In other embodiments, the amount of therapeutic agent, such as a protein (e.g., an antibody, or antigen-binding portion thereof, peptide, or a DVD-Ig protein) in a solid unit, such as a sphere shaped solid unit, may be between about 0.02 μg and 6 μg and the diameter of the sphere may be between about 0.1 mm to about 0.5 mm. Amounts, diameters and ranges intermediate to the above recited amounts, diameters and ranges are also intended to be part of this invention.

In one embodiment, the solid unit of the invention contains a therapeutic agent, such as a protein (e.g., an antibody, or an antigen-binding portion thereof, peptide, or a DVD-Ig protein) and sorbitol, sucrose or trehalose, where the amount of sorbitol, sucrose or trehalose is sufficient to maintain the stability of the therapeutic agent, such as a protein (e.g., peptide, DVD-Ig protein, or antibody, or antigen-binding portion thereof), for at least 12 months of storage at about 25° C. storage. Alternatively, the amount of sorbitol, sucrose or trehalose in the solid unit is sufficient to maintain stability of the therapeutic agent, such as a protein (e.g., an antibody, or antigen-binding portion thereof, peptide, or DVD-Ig protein) for at least 3 months of storage at about 40° C.

In one embodiment, the solid unit of the invention contains a therapeutic agent, such as a protein (e.g., an antibody, or an antigen-binding portion thereof, peptide, or a DVD-Ig protein) and mannitol, where the amount of mannitol is sufficient to maintain the stability of the agent, such as a protein (e.g., an antibody, or an antigen-binding portion thereof, peptide, or a DVD-Ig protein), for at least 12 months of storage at about 25° C. Alternatively, the amount of mannitol in the solid unit is sufficient to maintain stability of the agent, such as a protein (e.g., an antibody, or an antigen-binding portion thereof, peptide, or a DVD-Ig protein), or for at least 3 months of storage at about 40° C.

Stability of the therapeutic agent, such as a protein (e.g., an antibody, or antigen-binding portion thereof, peptide, or DVD-Ig protein) may be determined according to any method known in the art, including those described in the Examples herein. Size exclusion chromatography (SEC) may be used to determine fragment and monomer (aggregation) content for protein, such as antibodies, within a solid unit. In one embodiment, stability of the therapeutic agent, such as a protein (e.g., an antibody, peptide, or DVD-Ig protein) is determined by dissolving the solid unit containing the therapeutic agent, such as a protein (e.g., an antibody or antigen-binding portion thereof, peptide, or DVD-Ig protein), in water following storage (e.g., 12 months of storage at about 25° C. storage or 3 months of storage at about 40° C.) and performing SEC. In one embodiment, storage of the solid unit is performed at 25° C. under 55-65% relative humidity in a closed container. Alternatively, storage of the solid unit may be performed at 40° C. under 70-80% relative humidity in a closed container.

In one embodiment, SEC results indicating 90% or more monomer antibody, or antigen-binding portion thereof, indicates stability of the solid unit and antibody or antigen-binding portion thereof, contained therein. In one embodiment, SEC results indicating 95% or more monomer antibody, or antigen-binding portion thereof, indicates stability of the solid unit and antibody or antigen-binding portion thereof, contained therein. In one embodiment, SEC results indicating 96% or more monomer antibody, or antigen-binding portion thereof, indicates stability of the solid unit and antibody or antigen-binding portion thereof, contained therein. In one embodiment, SEC results indicating 97% or more monomer antibody, or antigen-binding portion thereof, indicates stability of the solid unit and antibody or antigen-binding portion thereof, contained therein. In one embodiment, SEC results indicating 98% or more monomer antibody, or antigen-binding portion thereof, indicates stability of the solid unit and antibody or antigen-binding portion thereof, contained therein. In one embodiment, SEC results indicating 99% or more monomer antibody, or antigen-binding portion thereof, indicates stability of the solid unit and antibody or antigen-binding portion thereof, contained therein. In one embodiment, SEC results indicating 99.5% or more monomer antibody, or antigen-binding portion thereof, indicates stability of the solid unit and antibody or antigen-binding portion thereof, contained therein. The monomer percentages described above also relate to solid units comprising DVD-Ig proteins.

Monomer percentages may also be described in terms of percent (%) aggregate. For example, in one embodiment, the invention features a plurality of solid units having less than 30% aggregate protein (e.g., peptide, antibody or DVD-Ig protein) as determined by SEC, less than 25% aggregate protein (e.g., peptide, antibody or DVD-Ig protein) as determined by SEC, less than 20% aggregate protein (e.g., peptide, antibody or DVD-Ig protein) as determined by SEC, less than 15% aggregate protein (e.g., peptide, antibody or DVD-Ig protein) as determined by SEC, less than 10% aggregate protein (e.g., peptide, antibody or DVD-Ig protein) as determined by SEC, less than 5% aggregate protein (e.g., peptide, antibody or DVD-Ig protein) as determined by SEC, less than 4% aggregate protein (e.g., peptide, antibody or DVD-Ig protein) as determined by SEC, less than 3% aggregate protein (e.g., peptide, antibody or DVD-Ig protein) as determined by SEC, less than 2% aggregate protein (e.g., peptide, antibody or DVD-Ig protein) as determined by SEC, less than 1% aggregate protein (e.g., peptide, antibody or DVD-Ig protein) as determined by SEC.

A solid unit of the invention may have a stabilizer:protein ratio ranging from about 0.8 to about 3.5:1.0 w/w, from about 0.8 to about 3.0:1.0 w/w, from about 0.8 to about 2.5:1.0 w/w, from about 0.8 to about 2.0:1.0 w/w, from about 0.8 to about 1.5:1.0 w/w, from about 0.9 to about 2.0:1 w/w, from about 0.9 to about 1.5:1.0 w/w, from about 0.1 to 3.5:1 w/w, from about 0.1 to 10:1 w/w, or from about 1.0:1.0 w/w. Examples of proteins having these exemplary stabilizer:protein ratios include, but are not limited to, peptide, antibodies, and DVD-Ig proteins. Values and ratios intermediate to the above recited values and ratios are also intended to be part of this invention.

In one embodiment, the ranges of molar ratios of stabilizer (sugar):antibody are 284:1 to 638:1. Alternatively, the range of molar ratio of stabilizer (sugar):antibody is 511:1 to 638:1; 520:1 to 638:1; 530:1 to 638:1, and so forth.

The present invention also features a stable solid unit suitable for pharmaceutical administration, comprising protein e.g., a peptide, DVD-Ig protein, or an antibody, or antigen-binding portion thereof, and sucrose, wherein the sucrose:peptide, DVD-Ig protein, or antibody, or antigen-binding portion thereof, ratio ranges from about 0.8 to 3.5:1 weight/weight (w/w), from about 0.8 to about 3.0:1.0 w/w, from about 0.8 to about 2.5:1.0 w/w, from about 0.8 to about 2.0:1.0 w/w, from about 0.8 to about 1.5:1.0 w/w, from about 0.9 to about 2.0:1 w/w, from about 0.9 to about 1.5:1.0 w/w, from about 0.1 to 3.5:1 w/w, from about 0.1 to 10:1 w/w; or from about 1.0:1.0 w/w. Values and ratios intermediate to the above recited values and ratios are also intended to be part of this invention.

The present invention further features a stable solid unit suitable for pharmaceutical administration, comprising a protein (e.g., a peptide, DVD-Ig protein, or an antibody, or antigen-binding portion thereof), and sorbitol, wherein the sorbitol:peptide, DVD-Ig protein, or antibody, or antigen-binding portion thereof, ratio ranges from about 0.8 to 3.5:1 weight/weight (w/w), from about 0.8 to about 3.0:1.0 w/w, from about 0.8 to about 2.5:1.0 w/w, from about 0.8 to about 2.0:1.0 w/w, from about 0.8 to about 1.5:1.0 w/w, from about 0.9 to about 2.0:1 w/w, from about 0.9 to about 1.5:1.0 w/w, from about 0.1 to 3.5:1 w/w, from about 0.1 to 10:1 w/w, or from about 1.0:1.0 w/w. Values and ratios intermediate to the above recited values and ratios are also intended to be part of this invention.

The present invention further features a stable solid unit suitable for pharmaceutical administration, comprising a protein, (e.g., a peptide, a DVD-Ig protein, or an antibody, or antigen-binding portion thereof (and trehalose, wherein the trehalose:peptide, DVD-Ig protein, or antibody, or antigen-binding portion thereof, ratio ranges from about 0.8 to 3.5:1 weight/weight (w/w), from about 0.8 to about 3.0:1.0 w/w, from about 0.8 to about 2.5:1.0 w/w, from about 0.8 to about 2.0:1.0 w/w, from about 0.8 to about 1.5:1.0 w/w, from about 0.9 to about 2.0:1 w/w, from about 0.9 to about 1.5:1.0 w/w, from about 0.1 to 3.5:1 w/w, from about 0.1 to 10:1 w/w, or from about 1.0:1.0 w/w. Values and ratios intermediate to the above recited values and ratios are also intended to be part of this invention.

In one embodiment, the concentration of sucrose in a solution for preparation of the solid unit is selected from the group consisting of about 10 mg/ml, about 20 mg/ml, about 30 mg/ml to about 100 mg/ml; about 40 mg/ml to about 90 mg/ml; about 40 mg/ml to about 80 mg/ml; about 40 mg/ml to about 70 mg/ml; about 40 mg/ml to about 60 mg/ml; and about 40 mg/ml to about 50 mg/ml. In one embodiment, the concentration of sucrose in a solution for preparation of the solid unit is about 10 mg/ml to about 200 mg/ml.

In one embodiment, the solid unit(s) of the invention are prepared from a solution comprising about 10 to about 40 mg/mL of mannitol and about 60 mg/mL to about 80 mg/mL of sucrose.

In one embodiment, the solid unit of the invention comprises a surfactant, e.g., a polysorbate.

In one embodiment, the solid unit of the invention does not include specific agents known to be traditional carriers for protein formulations. For example, in one embodiment, the solid unit does not comprise albumin, e.g., bovine serum albumin (BSA), or milk. Both albumin and milk, for example, are carriers used traditionally in protein formulations but are preferably excluded from the solid units of the invention, including solid units comprising a therapeutic protein (such as a peptide, DVD-Ig protein, or an antibody, or antigen-binding portion thereof).

In one embodiment, the solid unit of the invention does not comprise tromethamine. Thus, included in the invention is a solid unit (or plurality of solid units) comprising a therapeutic agent, such as a therapeutic protein, (e.g., a peptide, DVD-Ig protein, or an antibody, or antigen-binding portion thereof), and excluding tromethamine. In a further embodiment, the solid unit described herein (or the plurality thereof) does not contain casein. In a further embodiment, the solid unit described herein (or the plurality thereof) does not contain a preservative, such as sodium azide. Such solid units may also be free flowing and have geometric uniformity.

In one embodiment, the solid unit of the invention contains more than one type of protein, e.g., two antibodies that bind distinct epitopes.

The solid units of the invention are further stable in that they are free-flowing and are able to be stored in humid conditions despite containing sugars. For example, the solid units of the invention, in one embodiment, have a low moisture content, e.g., 2% or less moisture, 1% or less moisture, 0.9% or less moisture, 0.8% or less moisture, 0.7% or less moisture, 0.6% or less moisture, 0.5% or less moisture, 0.4% or less moisture, 0.3% or less moisture, 0.1% to 3% moisture, 0.1% to 2% moisture, or 1% to 2% moisture, even in humid conditions, e.g., 60% or more humidity.

In one embodiment, the protein population within a solid unit comprising a therapeutic protein (e.g., a peptide, antibody, or DVD-Ig protein) is at least 90% the therapeutic protein, at least 95% the therapeutic protein, at least 96% the therapeutic protein, at least 97% the therapeutic protein, at least 98% the therapeutic protein, or at least 99% the therapeutic protein.

In certain embodiments, the present invention encompasses post-translationally modified proteins, such as an antibody, or antigen-binding fragment thereof, as disclosed herein. For example, during post-translational processing, proteins are modified (e.g., chemical modification and folding) to produce a mature product (see, e.g., Berkowitz et al., Nat Rev Drug Discov. 11(7): 527-40, 2012, and references cited therein). Generally, modification is achieved by one or more events characterized broadly as the addition of biochemical functional groups (e.g., acetate, phosphate, lipids, and carbohydrates), modification of the chemical nature of an amino acid (e.g., citrullination), or structural modifications (e.g., formation of disulphide bridges). One of the most common post-translational modification to proteins involves glycosylation, which include, e.g., galactosylation, fucosylation, high mannose derivatives, and sialylation, e.g., N-linked or O-linked carbohydrate chains, processing of N-terminal or C-terminal ends. Additional post-translational modifications encompassed by the invention include, for example, oxidation, phosphorylation, sulphation, lipidation, disulphide bond formation, and deamidation, conversion of an N-terminal glutamate to pyroglutamate, deletion of a C-terminal amino acid, e.g., a C-terminal lysine), attachment of chemical moieties to the amino acid backbone, addition or deletion of an N-terminal methionine residue as a result of prokaryotic host cell expression. The proteins may also be modified with a detectable label, such as an enzymatic, fluorescent, isotopic or affinity label to allow for detection and isolation of the protein.

In one embodiment, solid units of the invention can be characterized according to the Table below, which describes the expected amount of protein given a spherical solid unit produced from a solution having either a 50 mg/ml protein concentration or 100 mg/ml protein. Thus, in one embodiment, the solid unit of the invention is a spherical solid unit having a diameter ranging from 0.1 mm to 3 mm, having a protein content of 0.00005 mg to 0.71 mg, and having a volume of 0.0005 microliters to 14.14 microliters.

Characterization of protein solid units

| Diameter | Volume | Area | Protein mg | Protein mg | Protein mg |
|---|---|---|---|---|---|
| mm | microliter | mm(2) | mg (100) | mg (50) | mg (25) |
| 0.1 | 0.000524 | 0.031416 | 0.00005236 | 0.00002618 | 0.00001309 |
| 0.2 | 0.004189 | 0.125664 | 0.00041888 | 0.00020944 | 0.00010472 |
| 0.5 | 0.065 | 0.785 | 0.0065 | 0.0033 | 0.00164 |
| 0.8 | 0.268 | 2.011 | 0.0268 | 0.0134 | 0.00670 |
| 1 | 0.524 | 3.142 | 0.0524 | 0.0262 | 0.01309 |
| 1.5 | 1.767 | 7.069 | 0.1767 | 0.0884 | 0.04418 |
| 2 | 4.189 | 12.566 | 0.4189 | 0.2094 | 0.10472 |
| 2.5 | 8.181 | 19.635 | 0.8181 | 0.4091 | 0.20453 |
| 3 | 14.137 | 28.274 | 1.4137 | 0.7069 | 0.35343 |
| 3.5 | 22.449 | 38.485 | 2.2449 | 1.1225 | 0.56123 |
| 4 | 33.510 | 50.266 | 3.3510 | 1.6755 | 0.83776 |

In one embodiment, the invention includes a lyophilized solid unit of an antibody, or antigen-binding portion thereof, and an amount of sorbitol, sucrose or trehalose which prevents or reduces chemical or physical instability of the antibody, or antigen-binding portion thereof, upon lyophilizing and subsequent storage.

It should be noted that whenever a value or range of values of a parameter are recited, it is intended that values and ranges intermediate to the recited values are also intended to be part of this invention.

In one embodiment, the solid units comprise an anti-human Tumor Necrosis Factor alpha (hTNFα) antibody, or antigen-binding portion thereof, comprising a light chain variable region comprising a CDR3 domain comprising an amino acid sequence set forth as SEQ ID NO: 3, a CDR2 domain comprising an amino acid sequence set forth as SEQ ID NO: 5, and a CDR1 domain comprising an amino acid sequence set forth as SEQ ID NO: 7, and a heavy chain variable region comprising a CDR3 domain comprising an amino acid sequence set forth as SEQ ID NO: 4, a CDR2 domain comprising an amino acid sequence set forth as SEQ ID NO: 6, and a CDR1 domain comprising an amino acid sequence set forth as SEQ ID NO: 8.

In one embodiment, the solid units comprise an anti-human Tumor Necrosis Factor alpha (hTNFα) antibody, or antigen-binding portion thereof, comprising a light chain variable region comprising the amino acid sequence set forth as SEQ ID NO: 1, and a heavy chain variable region of the antibody, or antigen-binding portion thereof comprising the amino acid sequence set forth as SEQ ID NO: 2.

In one embodiment, the solid units comprise an anti-human Tumor Necrosis Factor alpha (hTNFα) antibody, or antigen-binding portion thereof, comprising a light chain comprising the amino acid sequence set forth as SEQ ID NO: 9 and a heavy chain comprising the amino acid sequence set forth as SEQ ID NO: 10. In one embodiment, the acidic species of the antibody, or antigen-binding portion thereof, comprises AR1, AR2, or both AR1 and AR2.

In another embodiment, the solid unit of the invention comprises adalimumab, (or an antigen binding portion thereof), or a biosimilar thereof.

In one embodiment, the solid units of the invention comprise less than 15% of acidic species of the antibody, or antigen-binding portion thereof. In one embodiment, the acidic species of the antibody, or antigen-binding portion thereof, comprises AR1, AR2, or both AR1 and AR2. Alternatively, or in combination, the solid unit of the invention comprises about 70% lysine variant species of the antibody, or antigen-binding portion thereof, that have two C-terminal lysines (Lys 2) of the antibody, or antigen-binding portion thereof.

Notably, while therapeutic agents are described herein, it is also a feature of the invention that the methods and compositions described herein could be used for any agent, including small molecules. Further, the methods and compositions described herein may be used for non-therapeutic use, e.g., in vitro analysis.

The present invention also provides a plurality of solid units described herein. A plurality of solid units may, in some embodiments, have a substantially uniform size distribution and/or a volume distribution. In some instances, the plurality of solid units comprises populations of solid units having substantially uniform size distribution and/or a volume distribution. Notably, the plurality of solid units of the invention are not a powder (a power being a dry, bulk solid composed of a large number of very fine particles that may flow freely when shaken or tilted). Indeed, the plurality of the solid units described herein provide advantages over powders in that they provide consistency, for example in the size and uniformity of the plurality of solid units.

In one embodiment, a substantially uniform size distribution is intended to mean that the individual shapes and/or volumes of the solid units are substantially similar and not greater than a 20% standard deviation in volume. For example, a plurality of solid units which are spherical in shape would include a collection of solid units having no greater than 20% standard deviation from an average volume of the spheres. Alternatively, a substantially uniform size distribution indicates that the individual volumes of the solid units in a population are substantially similar and not greater than a 20% relative standard deviation in volume. Alternatively, a substantially uniform size distribution indicates that the individual volumes of the solid units in a population are substantially similar and not greater than a 15% standard deviation (or relative standard deviation) in volume; not greater than a 10% standard deviation (or relative standard deviation) in volume; or not greater than a 5% standard deviation (or relative standard deviation) in volume.

In one embodiment, each of the individual units within the plurality of units may have a substantially uniform volume, ranging from about 0.0005 μl to about 20 μl, about 0.005 μl to about 20 μl, 0.001 μl to about 20 μl, 0.05 μl to about 20 μl, 0.01 μl to about 20 μl, 0.0005 μl to about 10 about 0.005 μl to about 10 μl, 0.001 μl to about 10 μl, 0.05 μl to about 10 μl, 0.01 μl to about 10 μl, 0.0005 μl to about 5 about 0.005 μl to about 5 μl, 0.001 μl to about 5 μl, 0.05 μl to about 5 μl, 0.01 μl to about 5 μl, 0.0005 μl to about 1 about 0.005 μl to about 1 μl, 0.001 μl to about 1 μl, 0.05 μl to about 1 μl, 0.01 μl to about 1 μl, 0.1 μl to about 20 from about 0.5 μl to about 10 μl, about 0.5 to about 20, about 1 to about 20, about 1.5 to about 20, about 2 to about 20, about 2.5 to about 20, about 3 to about 20, about 3.5 to about 20, about 4 to about 20, about 4.5 to about 20, about 5 to about 20, about 5.5 to about 20, about 6 to about 20, about 6.5 to about 20, about 7 to about 20, about 7.5 to about 20, about 8 to about 20, about 8.5 to about 20, about 9 to about 20, about 9.5 to about 20, about 10 to about 20, about 15 to about 20, about 12 to about 20, about 13 to about 20, about 14 to about 20, about 15 to about 20, about 16 to about 20, about 17 to about 20, about 18 to about 20, about 19 to about 20, about 0.5 to about 15, about 1 to about 15, 1.5 to about 15, about 2 to about 15, about 2.5 to about 15, about 3 to about 15, about 3.5 to about 15, about 4 to about 15, about 4.5 to about 15, about 5 to about 15, about 5.5 to about 15, about 6 to about 15, about 6.5 to about 15, about 7 to about 15, about 7.5 to about 15, about 8 to about 15, about 8.5 to about 15, about 9 to about 15, about 9.5 to about 15, or about 10 μl to about 15 μl. In addition, a plurality of solid units may be substantially all spheres and have a volume ranging from any of the aforementioned volumes, including 0.0005 μl to about 20 μl, about 0.005 μl to about 20 μl, 0.001 μl to about 20 μl, 0.05 μl to about 20 μl, 0.01 μl to about 20 μl, 0.0005 μl to about 10 μl, about 0.005 μl to about 10 μl, 0.001 μl to about 10 μl, 0.05 μl to about 10 μl, 0.01 μl to about 10 μl, 0.0005 μl to about 5 μl, about 0.005 μl to about 5 μl, 0.001 μl to about 5 μl, 0.05 μl to about 5 μl, 0.01 μl to about 5 μl, 0.0005 μl to about 1 μl, about 0.005 μl to about 1 μl, 0.001 μl to about 1 μl, 0.05 μl to about 1 μl, 0.01 μl to about 1 μl, about 0.1 μl to about 20 μl or from about 0.5 μl to about 10 μl. Volumes and ranges intermediate to the above recited volumes and ranges are also intended to be part of this invention (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0, 2.1, 2.2, 2.3, 2.4, 2.5, 2.6, 2.7, 2.8, 2.9, 3.0 μl). For spherical solid units, volume is related to diameter. For example, a spherical solid unit having a volume of about 0.05 μl has a diameter of about 0.2 mm, and a spherical solid unit having a volume of about 0.0005 μl has a diameter of about 0.1 mm

In one embodiment, each of the solid units within the plurality of units may be substantially all spheres and have a diameter of about 0.1 to about 4 mm; about 0.1 to about 3 mm; about 0.1 to about 2 mm; about 0.1 to about 1 mm; or about 0.1 to about 0.5 mm. Diameters and ranges intermediate to the above recited diameters and ranges are also intended to be part of this invention (e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and 4.0 mm).

In a one embodiment, the plurality of subunits is suitable for pharmaceutical administration. The plurality of subunits may be manufactured under aseptic conditions.

One benefit of the plurality of solid units of the invention is that they remain free-flowing at room temperature and humidity, e.g., for at least 12 months at about 25° C. In one embodiment, the plurality of subunits is made of solid units having low moisture content, e.g., 1% or less moisture, 0.9% or less moisture, 0.8% or less moisture, 0.7% or less moisture, 0.6% or less moisture, 0.5% or less moisture, 0.4% or less moisture. Under certain conditions, e.g., under sealed containers, the solid units are able to maintain the low moisture content even in humid conditions, e.g., 60% or more humidity.

In some embodiments of the invention, a plurality of solid units are encapsulated within a shell or capsule, allowing them to, for example, be taken orally or be used as suppositories. Suitable capsules may be hard-shelled capsules or soft-shelled capsules, single-piece capsules or two-piece capsules. The solid units of the invention may also be pressed into a tablet which, in one embodiment, may be coated with an enteric coating.

An important feature of the plurality of solid units is that the plurality may, in certain embodiments, include two or more populations of solid units. For example, a plurality of solid units of the invention may include populations of different therapeutic proteins, solid units having different sizes, solid units having different enteric protectants or enteric coatings allowing for release at different points of the GI tract, etc. The plurality of solid units may include solid units containing antibodies, or antigen binding portions thereof, directed to at least two distinct molecular targets. Thus, the plurality of solid units allows for combinations of solid units, e.g., solid units within the plurality containing different antibodies.

In one embodiment, the invention features a pharmaceutical composition comprising a plurality of solid units composed of at least two different populations of solid units. The populations may be distinct based on any parameter, e.g., size, amount of therapeutic agent, the type of therapeutic agent, or any combinations thereof. Notably, the solid units are stable and remain free flowing even when combined in heterogeneous populations.

In one embodiment, the invention features a plurality of solid units having at least two populations of solid units specific to different molecular targets, e.g., a peptide and/or an antibody, or antigen binding portion thereof, that bind at least two distinct molecular targets. The term “distinct molecular target” indicates that within a population two or more binding proteins are specific for distinct molecules, e.g., TNF and EGFR, or alternatively, are specific for specific epitopes within one molecule, e.g., epitopes one and two on TNF. Thus, the plurality of solid units of the invention may include two or more populations of solid units comprising one population of solid units having a first peptide or first antibody, or an antigen binding portion thereof, and a second population of solid units having a second peptide or a second antibody, or antigen binding portion thereof, wherein the second peptide or second antibody, or antigen-binding portion thereof, is directed to a different molecular target or epitope than the first peptide or the first antibody, or antigen-binding portion thereof.

In one embodiment, the invention features a plurality of solid units having at least two populations of solid units having substantially similar volumes and a second population of solid units having substantially similar volumes, wherein the first population and the second population have different volumes.

In one embodiment, the invention features a plurality of solid units having two or more populations of solid units comprising one population of solid units having a first peptide or antibody, or a first antigen binding portion thereof, and a second population of solid units comprising an additional therapeutic agent.

In one embodiment, the two populations of solid units within the plurality make up at least about 70% of the plurality; at least about 80% of the plurality; at least about 90% of the plurality; at least about 95% of the plurality; at least 96%; at least 97%; at least 98%; or at least 99% of the overall population of solid units.

Combinations of the aforementioned populations are also within the scope of the invention, e.g., two populations of solid units within a plurality where each population has a unique size which is substantially similar within the population and also contains antibodies or peptides to different molecular targets.

In some embodiments, the uniform, free flowing stable solid units may be combined with other uniform, free flowing, stable solid units of a different composition or molecule that can be combined to produce multiple API final drug products for parenteral or oral administration.

In one embodiment, the plurality of solid units contains an anti-human Tumor Necrosis Factor alpha (hTNFα) antibody, or antigen-binding portion thereof, comprising a light chain variable region comprising a CDR3 domain comprising an amino acid sequence set forth as SEQ ID NO: 3, a CDR2 domain comprising an amino acid sequence set forth as SEQ ID NO: 5, and a CDR1 domain comprising an amino acid sequence set forth as SEQ ID NO: 7, and a heavy chain variable region comprising a CDR3 domain comprising an amino acid sequence set forth as SEQ ID NO: 4, a CDR2 domain comprising an amino acid sequence set forth as SEQ ID NO: 6, and a CDR1 domain comprising an amino acid sequence set forth as SEQ ID NO: 8.

In another embodiment, the plurality of solid units contains an anti-human Tumor Necrosis Factor alpha (hTNFα) antibody, or antigen-binding portion thereof, comprising a light chain variable region comprising the amino acid sequence set forth as SEQ ID NO: 1, and a heavy chain variable region of the antibody, or antigen-binding portion thereof. Variable and CDR sequences of the antibody D2E7 are described in U.S. Pat. No. 6,090,382, which is incorporated by reference herein.

In another embodiment, the plurality of solid units contains an anti-human Tumor Necrosis Factor alpha (hTNFα) antibody, or antigen-binding portion thereof, comprising a light chain comprising the amino acid sequence set forth as SEQ ID NO: 9 and a heavy chain comprising the amino acid sequence set forth as SEQ ID NO: 10, wherein In one embodiment, the acidic species of the antibody, or antigen-binding portion thereof, comprises AR1, AR2, or both AR1 and AR2.

In another embodiment, the plurality of solid units contains adalimumab, (or an antigen binding portion thereof), or a biosimilar thereof.

In one embodiment, the plurality of solid units comprises less than 15% of acidic species of the antibody, or antigen-binding portion thereof. In one embodiment, the acidic species of the antibody, or antigen-binding portion thereof, comprises AR1, AR2, or both AR1 and AR2. Alternatively, or in combination, the plurality comprises about 70% lysine variant species of the antibody, or antigen-binding portion thereof, that have two C-terminal lysines (Lys 2) of the antibody, or antigen-binding portion thereof.

Polymers for Use in Combination with the Solid Units of the Invention

In certain embodiments of the invention, the solid unit of the invention may also include a polymer, including, but not limited to, polymers suitable for oral or parenteral administration of the solid unit (or plurality of solid units). Thus, in some embodiments, the uniform, free flowing, stable solid units can be coated with polymer for oral administration.

Polymers may be included within the solid unit and/or coated on the outside of the solid unit, e.g., an enteric coating. Polymers may be used in accordance with the mode of delivery. For example, oral delivery generally requires the solid unit to have an enteric coating such that the solid unit (or plurality thereof) can withstand pH extremes in the stomach and the protein can be absorbed in the intestines.

In one embodiment, the solid unit of the invention includes a peptide or antibody, or antigen-binding portion thereof, a stabilizer, and a polymer selected from the group consisting of an enteric protectant, a non-pH-sensitive polymer, a slow-release polymer, a bioadhesive polymer, or any combination thereof. Other examples of polymers that may be combined with the solid units of the invention include hydrophilic polymers (e.g., poly(2-hydroxyethyl methacrylate), biodegradable polymers (e.g., poly(vinyl pyrrolidone), poly(lactic acid), poly(glycolic acid), and collagen), swellable polymers (e.g., ethylene/vinyl alcohol and HPMC), ion-exchange polymers (e.g., polystyrene sulfonic acid), and hydrophobic polymers (e.g., polydimethylysiloxane, polyethylene, ethylene/vinyl acetate, and polyurethane).

In one embodiment, the polymer used in the invention is a polyvinyl alcohol, an ethyl vinyl acetate, or ethyl cellulose.

Polymers may be added to the solid unit during the lyophilization process, e.g., polymers may be added to the initial protein solution which is subsequently lyophilized. Alternatively, polymers may be added to the diluent in which the solid unit of the invention is dissolved, e.g., a diluent in which the solid unit is dissolved prior to administration to a human subject.

In one embodiment, the solid unit contains a slow-release polymer within the solid unit and has an enteric protectant, e.g., an enteric coating. Alternatively, the solid unit may be coated with both an enteric coating and a slow-release polymer.

Exemplary enteric protectants (which may also be used as enteric coatings) include, for example, polymers having free carboxylic acid groups on the polymer backbone including, for example, polymethacrylates (such as methacrylic acid/ethyl acrylate), cellulose esters (such as cellulose acetate phthalate (CAP), cellulose acetate trimellitate (CAT), and hydroxypropylmethylcellulose acetate succinate (HPMCAS)), polyvinyl derivatives (such as Polyvinyl acetate phthalate (PVAP)), and copolymers (such as half esters of the copolymerisate of styrene and maleic acid, half esters of the copolymerisate of vinyl ether and maleic acid, and a copolymerisate of vinyl acetate and crotonic acid).

Exemplary slow-release polymers include polyacrylic acid, cellulose derivatives, chitosan and various gums such as guar, xanthan, poly(vinylpyrrolidone), and poly(vinyl alcohol). Examples of polyacrylic acid-based polymers are carbopol, polycarbophil, polyacrylic acid (PAAc), polyacrylate, poly (methylvinylether-co-methacrylic acid), poly (2-hydroxyethyl methacrylate), poly(methacrylate), poly(alkylcyanoacrylate), poly(isohexylcyanoacrylate) and poly(isobutylcyanoacrylate), polystyrene, poly(lactic-co-glycolic acid) (PLGA), chitosan, polycaprolactone (PCL), poly(butylcyanoacrylate) (PBCA), poly(lactic acid) (PLA), poly)hexyl cyanoacrylate) (PHCA), poly(acrylic acid) (PAA). Other examples include, but are not limited to, poly(itaconic acid) (PIA), poly(isobutyl cyanoacrylate) (PIBCA), poly(methylmethacrylate-co-sulfopropylmethacrylate) (PMS), poly(ethyl cyanoacrylate) (PECA), Eudragit RS100 (ERS) or Eudragit RL100 (ERL), or poly(methoxypolyethyleneglycol cyanoacrylate-co-hexadecyl cyanoacrylate) (PMHCH). Cellulose derivatives include carboxymethyl cellulose, hydroxyethyl cellulose, hydroxypropyl cellulose, sodium carboxymethyl cellulose, methylcellulose, and methylhydroxyethyl cellulose. Carbopol is also an example of a slow-release polymer that may be combined with the solid units of the invention. Carbopol homopolymers are polymers of acrylic acid crosslinked with allyl sucrose or allyl pentaerythritol. Carbopol copolymers are polymers of acrylic acid and C10-C30 alkyl acrylate crosslinked with allyl pentaerythritol. Carbopol interpolymers are a carbomer homopolymer or copolymer that contains a block copolymer of polyethylene glycol and a long chain alkyl acid ester

In one embodiment, the slow release polymer is a cellulose derivative or a poly(acrylic acid) polymer. Examples of a cellulose derivative include, but are not limited to, hydroxypropylmethylcellulose (HPMC), hydroxypropylcellulose (HPC), hydroxyethylcellulose (HEC), ethylcellulose, and methylcellulose.

In one embodiment, the enteric coating is a polymethacrylate, a cellulose ester, or a polyvinyl derivative.

In one embodiment, the polymethacrylate is methacrylic acid/ethyl acrylate. In one embodiment, the cellulose ester is cellulose acetate phthalate (CAP), cellulose acetate triemellitate (CAT), or hydroxypropylmethylcellulose acetate succinate (HPMCAS).

In one embodiment, the polyvinyl derivative is polyvinyl acetate phthalate (PVAP).

In one embodiment, the enteric coating is a poly(acrylic acid) polymer, a poly(sulfonic acid) polymer, a poly(vinylamine) polymer, a poly[2-(dimethylamino)ethyl methacrylate] polymer, copolymers, or derivatives thereof.

In one embodiment, the solid unit comprises one of the following polymers: copovidone, methocel, kollicoat, hydroxypropyl cellulose, (MW 80,000 to 100,000), ethyl cellulose, AnyCoat-P (hypromellose phthalate), Edragit 5100, Edragit L100, and HPMC AS-LF.

In one embodiment, the bioadhesive polymer is fumaric acid or sebacic acid. In a further embodiment, the bioadhesive polymer used in the compositions of the invention is a hydrophilic polymer or a hydrogel. An examples of a hydrophilic polymer includes, but is not limited to, polymers containing carboxylic groups (e.g., poly[acrylic acid]). Alternative bioadhesive polymers include sodium alginate, polycarbophil, fibronectin segment, carboxymethylcellulose, hydroxymethylcellulose and methylcellulose.

Rapidly bioerodible polymers such as poly[lactide-co-glycolide], polyanhydrides, and polyorthoesters, whose carboxylic groups are exposed on the external surface as their smooth surface erodes, are also examples of bioadhesive polymers that can be used in the solid units of the invention.

Further representative bioadhesive polymers include, but are not limited to, synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide), poly(lactide-co-caprolactone), poly(ethylene-co-maleic anhydride), poly(ethylene maleic anhydride-co-L-dopamine), poly(ethylene maleic anhydride-co-phenylalanine), poly(ethylene maleic anhydride-co-tyrosine), poly(butadiene-co-maleic anhydride), poly(butadiene maleic anhydride-co-L-dopamine) (pBMAD), poly(butadiene maleic anhydride-co-phenylalanine), poly(butadiene maleic anhydride-co-tyrosine), poly(fumaric-co-sebacic)anhydride (P(FA:SA)), poly(bis carboxy phenoxy propane-co-sebacic anhydride) (20:80) (poly(CCP:SA)), as well as blends comprising these polymers; and copolymers comprising the monomers of these polymers, and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers, blends and mixtures thereof. In general, these materials degrade either by enzymatic hydrolysis or exposure to water in vivo, by surface or bulk erosion.

In one embodiment, the polymers described herein are included in the diluent used to resuspend the solid unit of the invention.

Solvents may also be used in combination with the solid units of the invention to provide solid units that are able to be orally administered. Examples of solvents include, but are not limited to, chloroform, methanol, isopropanol, ethanol, acetone, petroleum ether, tert-butanol, and reagent alcohol.

Suitable polymers for oral delivery of the protein, e.g., antibody, or antigen-binding portion thereof, include an enteric protectant, non-pH-sensitive polymers, slow-release polymers, bioadhesive polymers, or any combination thereof, e.g., a slow-release polymer and an enteric protectant, examples of which are described above. In one embodiment, the slow release polymer may be included within the solid unit or coated on the solid unit. Similarly, a non-pH-sensitive polymer may be included within the solid unit or coated on the solid unit.

### Antibodies for Use in Compositions and Methods of Invention

Antibodies, or antigen-binding fragments thereof, (and post-translationally modified forms of the antibody, or antigen-binding fragment thereof) may be used in the solid units and methods of the invention. Examples of antibodies, or antigen-binding fragments thereof, that may be used in the invention include, but are not limited to, human antibodies, chimeric antibodies, humanized antibodies, and antigen-binding fragments of said antibodies.

In certain embodiments, the antibody comprises a heavy chain constant region, such as an IgG1, IgG2, IgG3, IgG4, IgA, IgE, IgM or IgD constant region. In certain embodiments, the heavy chain constant region is an IgG1 heavy chain constant region or an IgG4 heavy chain constant region. Furthermore, the antibody can comprise a light chain constant region, either a kappa light chain constant region or a lambda light chain constant region. In one embodiment, the antibody or antigen-binding portion thereof, comprises a kappa light chain constant region. Alternatively, the antibody portion can be, for example, a Fab fragment or a single chain Fv fragment.

In one embodiment, an antibody, or antigen-binding portion thereof, suitable for use in the compositions and methods of the invention is an antibody, or antigen-binding portion thereof, which binds human TNFα, including, for example, adalimumab (also referred to as Humira or D2E7; AbbVie). The anti-TNF-alpha antibody, or antigen-binding portion thereof, used in the invention preferably binds to human TNF-alpha with high affinity and a low off rate, and also has a high neutralizing capacity.

Suitable antibodies, or antigen-binding portions thereof, for use in the present invention may comprise an antibody or antigen-binding portion thereof, that binds the same epitope as adalimumab, such as, but not limited to, an adalimumab biosimilar antibody. In one embodiment, the antibody is a human IgG1 antibody having six CDRs corresponding to those of the light and heavy chain of adalimumab.

In one aspect, the invention pertains to solid units containing adalimumab antibodies and antibody portions, adalimumab-related antibodies and antibody portions, and other human antibodies and antibody portions with equivalent properties to adalimumab, such as high affinity binding to hTNFα with low dissociation kinetics and high neutralizing capacity. In one embodiment, the antibody, or antigen-binding fragment thereof, is defined according to dissociation and binding characteristics similar to adalimumab. For example, the formulation may include a human antibody that dissociates from human TNFα with a K_{d} of 1×10⁻⁸ M or less, and a k_{off} rate constant of 1×10⁻³ s⁻¹ or less, both determined by surface plasmon resonance. In another embodiment, the human antibody or antigen-binding portion thereof, dissociates from human TNFα with a K_{d} of 1×10⁻⁹ M or less.

In one embodiment, the antibody, or antigen-binding fragment thereof, is a human antibody that dissociates from human TNFα with a K_{d} of 1×10⁻⁸ M or less, and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. Examples and methods for making human, neutralizing antibodies which have a high affinity for human TNFα, including sequences of the antibodies, are described in U.S. Pat. No. 6,090,382 (referred to as D2E7), incorporated by reference herein.

In one embodiment, the antibody or antigen-binding portion thereof, used in the formulation of the invention is D2E7, also referred to as HUMIRA™ or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein.

In one embodiment, the human TNF-alpha, or an antigen-binding portion thereof, dissociates from human TNF-alpha with a K_{d} of 1×10⁻⁸ M or less and a k_{off} rate constant of 1×10⁻³ s⁻¹ or less, both determined by surface plasmon resonance, and neutralizes human TNF-alpha cytotoxicity in a standard in vitro L929 assay with an IC₅₀ of 1×10⁻⁷ M or less. In one embodiment, the isolated human antibody, or antigen-binding portion thereof, dissociates from human TNF-alpha with a k_{off} of 5×10⁻⁴ s⁻¹ or less; or, in one embodiment, with a k_{off} of 1×10⁻⁴ s⁻¹ or less. In one embodiment, the isolated human antibody, or antigen-binding portion thereof, neutralizes human TNF-alpha cytotoxicity in a standard in vitro L929 assay with an IC₅₀ of 1×10⁻⁸ M or less; or, in one embodiment, with an IC₅₀ of 1×10⁻⁹ M or less; or, in one embodiment, with an IC₅₀ of 1×10⁻¹⁰ M or less. In one embodiment, the antibody or antigen-binding portion thereof, is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the antibody or antigen-binding portion thereof, used in the compositions and methods of the invention has slow dissociation kinetics for association with hTNF-alpha and has light and heavy chain CDR3 domains that structurally are identical to or related to those of adalimumab. Position 9 of the adalimumab VL CDR3 can be occupied by Ala or Thr without substantially affecting the Koff. Accordingly, a consensus motif for the adalimumab VL CDR3 comprises the amino acid sequence: Q-R-Y-N-R-A-P-Y-(T/A) (SEQ ID NO: 3). Additionally, position 12 of the adalimumab VH CDR3 can be occupied by Tyr or Asn, without substantially affecting the k_{off}. Accordingly, a consensus motif for the adalimumab VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the adalimumab heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the k_{off}. Still further, the skilled artisan will appreciate that, given the amenability of the adalimumab VL and VH CDR3 domains to substitutions by alanine, substitution of other amino acids within the CDR3 domains may be possible while still retaining the low off rate constant of the antibody, in particular substitutions with conservative amino acids. In one embodiment, no more than one to five conservative amino acid substitutions are made within the adalimumab VL and/or VH CDR3 domains. In one embodiment, no more than one to three conservative amino acid substitutions are made within the adalimumab VL and/or VH CDR3 domains. Additionally, conservative amino acid substitutions should not be made at amino acid positions critical for binding to hTNF alpha. Positions 2 and 5 of the adalimumab VL CDR3 and positions 1 and 7 of the adalimumab VH CDR3 appear to be critical for interaction with hTNF alpha, and thus, conservative amino acid substitutions preferably are not made at these positions (although an alanine substitution at position 5 of the adalimumab VL CDR3 is acceptable, as described above) (see U.S. Pat. No. 6,090,382).

Accordingly, in one embodiment, the antibody, or antigen-binding portion thereof, used in the compositions and methods of the invention contains the following characteristics:

a) dissociates from human TNFα with a k_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In certain embodiments, the antibody or antigen-binding portion thereof, dissociates from human TNF-alpha with a k_{off} of 5×10⁻⁴ s⁻¹ or less. In certain embodiments, the antibody or antigen-binding portion thereof, dissociates from human TNF-alpha with a k_{off} of 1×10⁻⁴ s⁻¹ or less.

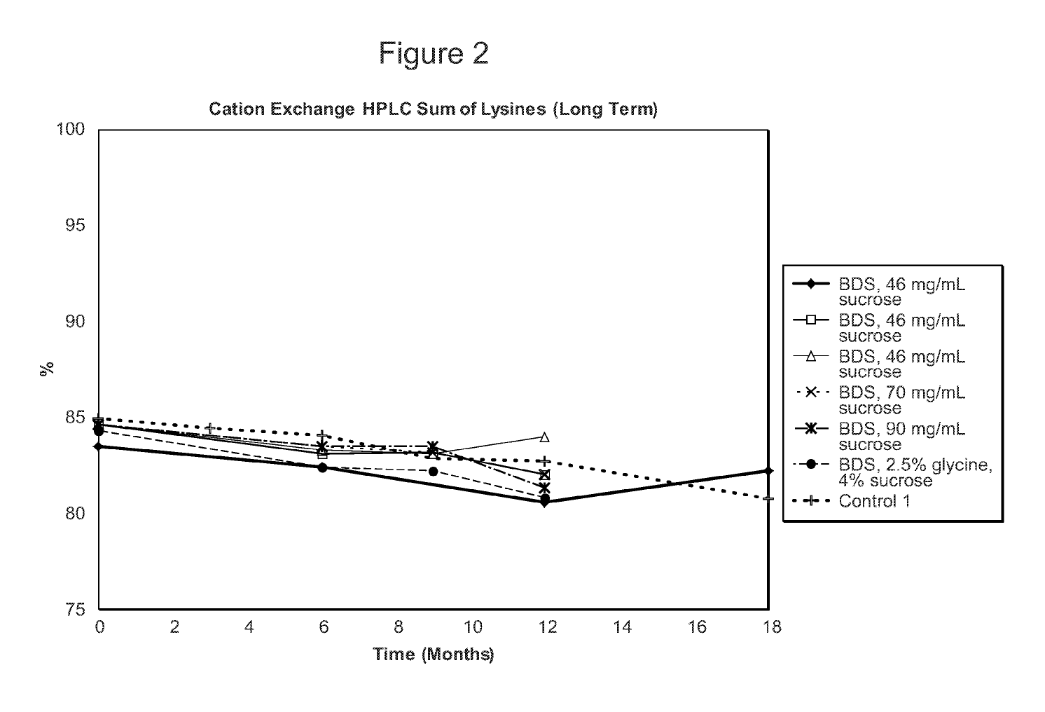

In yet another embodiment, the antibody or antigen-binding portion thereof contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. In one embodiment, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). In one embodiment, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL may be from the VκI human germline family, or from the A20 human germline Vk gene, or from the adalimumab VL framework sequences shown in FIGS. 1A and 1B of U.S. Pat. No. 6,090,382. The framework regions for VH may be from the VH3 human germline family, or from the DP-31 human germline VH gene, or from the D2E7 VH framework sequences shown in FIGS. 2A and 2B of U.S. Pat. No. 6,090,382.

Accordingly, in another embodiment, the antibody or antigen-binding portion thereof, contains a light chain variable region (LCVR) comprising the amino acid sequence of SEQ ID NO: 1 (i.e., the adalimumab VL) and a heavy chain variable region (HCVR) comprising the amino acid sequence of SEQ ID NO: 2 (i.e., the adalimumab VH). In another embodiment, the antibody or antigen-binding portion thereof, contains a complete light chain comprising the amino acid sequence of SEQ ID NO: 9 (i.e., the adalimumab L chain) and a complete heavy chain comprising the amino acid sequence of SEQ ID NO: 10 (i.e., the adalimumab H chain).

As described in U.S. Provisional Patent Application 61/893,068, entitled “LOW ACIDIC SPECIES COMPOSITIONS AND METHODS FOR PRODUCING THE SAME”, filed on Oct. 18, 2013, and U.S. Provisional Patent Application 61/892,710, entitled “ANTI-TNFα ANTIBODIES AND METHODS OF USE THEREOF”, filed on Oct. 18, 2013, weak cation-exchange chromatography (WCX) analysis of a human anti-TNF-alpha antibody, has shown that it has three main basic charge variants (i.e., Lys 0, Lys 1, and Lys 2). These variants, or charged isomers, are the result of incomplete post-translational cleavage of the C-terminal lysine residues on the heavy chains of the antibody. In addition to the lysine variants, the WCX-10 analysis measures the presence of acidic regions and/or acidic species. These acidic species and/or regions (including acidic regions AR1 and AR2), AR1 and AR2, are classified as product-related impurities that are relatively acidic when compared to the lysine variants and elute before the Lys 0 peak in the chromatogram. The entire contents of U.S. Provisional Patent Application 61/893,068, entitled “LOW ACIDIC SPECIES COMPOSITIONS AND METHODS FOR PRODUCING THE SAME”, filed on Oct. 18, 2013, U.S. patent application Ser. No. 14/077,871, filed on Nov. 12, 2013, U.S. Provisional Patent Application 61/892,710, entitled “ANTI-TNFα ANTIBODIES AND METHODS OF USE THEREOF”, filed on Oct. 18, 2013, and U.S. Patent Publication No. 20140271626, filed on Mar. 12, 2014, are incorporated herein by reference.

The terms “acidic species”, “acidic region” or “AR,” as used herein, refer to the variants of a protein, e.g., an antibody or antigen-binding portion thereof, (e.g., adalimumab) which are characterized by an overall acidic charge. For example, in monoclonal antibody (mAb) preparations, such acidic species can be detected by various methods, such as, for example, WCX-10 HPLC (a weak cation exchange chromatography), or IEF (isoelectric focusing). Acidic species of an antibody, e.g., an anti-TNFα antibody like those described herein, include charge variants, structure variants, and/or fragmentation variants. Exemplary charge variants include, but are not limited to, deamidation variants, afucosylation variants, methylglyoxal (MGO) variants, glycation variants, and citric acid variants. Exemplary structure variants include, but are not limited to, glycosylation variants and acetonation variants. Exemplary fragmentation variants include any truncated protein species from the target molecule due to dissociation of peptide chain, enzymatic and/or chemical modifications, including, but not limited to, Fc and Fab fragments, fragments missing a Fab, fragments missing a heavy chain variable domain, C-terminal truncation variants, variants with excision of N-terminal Asp in the light chain, and variants having N-terminal truncation of the light chain.

The term “acidic species” does not include process-related impurities. The term “process-related impurity,” as used herein, refers to impurities that are present in a composition comprising a protein but are not derived from the protein itself. Process-related impurities include, but are not limited to, host cell proteins (HCPs), host cell nucleic acids, chromatographic materials, and media components.” A “low process-related impurity composition,” as used herein, refers to a composition comprising reduced levels of process-related impurities as compared to a composition wherein the impurities were not reduced. For example, a low process-related impurity composition may contain about 10%, 9%, 8%, 7%, 6%, 5%, 4%, 3%, 2%, 1%, 0.5%, or less of process-related impurities. In one embodiment, a low process-related impurity composition is free of process-related impurities or is substantially free of process-related impurities.

The acidic species may be the result of product preparation (referred to herein as “preparation-derived acidic species”), or the result of storage (referred to herein as “storage-derived acidic species”). Preparation-derived acidic species are acidic species that are formed during the preparation (upstream and/or downstream processing) of the protein, e.g., the antibody or antigen-binding portion thereof. For example, preparation-derived acidic species can be formed during cell culture (“cell culture-derived acidic species”). Storage-derived acidic species are acidic species that are not present in the population of proteins directly after preparation, but are formed while the sample is being stored. The type and amount of storage-derived acidic species can vary based on the formulation of the sample. Formation of storage-derived acidic species can be partially or completely inhibited when the preparation is stored under particular conditions. For example, an aqueous formulation can be stored at a particular temperature to partially or completely inhibit AR formation. For example, formation or storage-derived AR can be partially inhibited in an aqueous formulation stored at between about 2° C. and 8° C., and completely inhibited when stored at −80° C. In addition, a low AR composition can be lyophilized to partially or completely inhibit the formation of storage-derived AR, making the present invention well suited for use with low AR compositions.

In certain embodiments, a solid unit of the invention can comprise more than one type of acidic species variant. For example, but not by way of limitation, the total acidic species can be divided based on chromatographic residence time. For example, the total acidic species associated with the expression of adalimumab may be divided into a first acidic species region (AR1) and a second acidic species region (AR2). AR1 may comprise, for example, charge variants such as deamidation variants, MGO modified species, glycation variants, and citric acid variants, structural variants such as glycosylation variants and acetonation variants, and/or fragmentation variants. Other acidic variants such as host cells and unknown species may also be present. AR2 may comprise, for example, charge variants such as glycation variants and deamidation variants.

The term “low acidic species solid unit” or “low AR solid unit,” as used herein, refers to a solid unit comprising an antibody or antigen-binding portion thereof, wherein the solid unit contains less than about 15% acidic species. As used herein, the percent AR in the low AR solid unit refers to the weight of the acidic species in a sample in relation to the weight of the total antibodies contained in the sample. For example, the percent AR can be calculated using weak cation exchange chromatography such as WCX-10. In one embodiment, the invention features a plurality of low acidic solid units.

In one embodiment, a solid unit may comprise about 15% or less AR, 14% or less AR, 13% or less AR, 12% or less AR, 11% or less AR, 10% or less AR, 9% or less AR, 8% or less AR, 7% or less AR, 6% or less AR, 5% or less AR, 4.5% or less AR, 4% or less AR, 3.5% or less AR, 3% or less AR, 2.5% or less AR, 2% or less AR, 1.9% or less AR, 1.8% or less AR, 1.7% or less AR, 1.6% or less AR, 1.5% or less AR, 1.4% or less AR, 1.3% or less AR, 1.2% or less AR, 1.1% or less AR, 1% or less AR, 0.9% or less AR, 0.8% or less AR, 0.7% or less AR, 0.6% or less AR, 0.5% or less AR, 0.4% or less AR, 0.3% or less AR, 0.2% or less AR, 0.1% or less AR, or 0.0% AR, and ranges within one or more of the preceding. A low AR solid unit of the invention may also comprise about 0.0% to about 10% AR, about 0.0% to about 5% AR, about 0.0% to about 4% AR, about 0.0% to about 3% AR, about 0.0% to about 2% AR, about 3% to about 5% AR, about 5% to about 8% AR, or about 8% to about 10% AR, or about 10% to about 15% AR, and ranges within one or more of the preceding.

A low AR solid unit of the invention may comprise about 15% or less AR1, 14% or less AR1, 13% or less AR1, 12% or less AR1, 11% or less AR1, 10% or less AR1, 9% or less AR1, 8% or less AR1, 7% or less AR1, 6% or less AR1, 5% or less AR1, 4.5% or less AR1, 4% or less AR1, 3.5% or less AR1, 3% or less AR1, 2.5% or less AR1, 2% or less AR1, 1.9% or less AR1, 1.8% or less AR1, 1.7% or less AR1, 1.6% or less AR1, 1.5% or less AR1, 1.4% or less AR1, 1.3% or less AR1, 1.2% or less AR1, 1.1% or less AR1, 1% or less AR1, 0.9% or less AR1, 0.8% or less AR1, 0.7% or less AR1, 0.6% or less AR1, 0.5% or less AR1, 0.4% or less AR1, 0.3% or less AR1, 0.2% or less AR1, 0.1% or less AR1, or 0.0% AR1, and ranges within one or more of the preceding. A low AR composition of the invention may also comprise about 0.0% to about 10% AR1, about 0.0% to about 5% AR1, about 0.0% to about 4% AR1, about 0.0% to about 3% AR1, about 0.0% to about 2% AR1, about 3% to about 5% AR1, about 5% to about 8% AR1, or about 8% to about 10% AR1, or about 10% to about 15% AR1, and ranges within one or more of the preceding.

A low AR solid unit of the invention may also comprise about 15% or less AR2, 14% or less AR2, 13% or less AR2, 12% or less AR2, 11% or less AR2, 10% or less AR2, 9% or less AR2, 8% or less AR2, 7% or less AR2, 6% or less AR2, 5% or less AR2, 4.5% or less AR2, 4% or less AR2, 3.5% or less AR2, 3% or less AR2, 2.5% or less AR2, 2% or less AR2, 1.9% or less AR2, 1.8% or less AR2, 1.7% or less AR2, 1.6% or less AR2, 1.5% or less AR2, 1.4% or less AR2, 1.3% or less AR2, 1.2% or less AR2, 1.1% or less AR2, 1% or less AR2, 0.9% or less AR2, 0.8% or less AR2, 0.7% or less AR2, 0.6% or less AR2, 0.5% or less AR2, 0.4% or less AR2, 0.3% or less AR2, 0.2% or less AR2, 0.1% or less AR2, or 0.0% AR2, and ranges within one or more of the preceding. A low AR composition of the invention may also comprise about 0.0% to about 10% AR2, about 0.0% to about 5% AR2, about 0.0% to about 4% AR2, about 0.0% to about 3% AR2, about 0.0% to about 2% AR2, about 3% to about 5% AR2, about 5% to about 8% AR2, or about 8% to about 10% AR2, or about 10% to about 15% AR2, and ranges within one or more of the preceding.

In one embodiment, a low AR solid unit comprises between about 0.0% and about 3% AR1. In another embodiment, a low AR solid unit comprises about between about 0.0% and about 3% AR2. In still another embodiment, a low acidic comprises about 3% or less AR2.

In another embodiment, the low AR solid unit comprises about 1.4% or less AR. For example, in one embodiment, the composition comprises about 1.4% AR2 and about 0.0% AR1.

In one embodiment, a low AR solid unit of the invention may comprise about 15% or less, 14% or less, 13% or less, 12% or less, 11% or less, 10% or less, 9% or less, 8% or less, 7% or less, 6% or less, 5% or less, 4.5% or less, 4% or less, 3.5% or less, 3% or less, 2.5% or less, 2% or less, 1.9% or less, 1.8% or less, 1.7% or less, 1.6% or less, 1.5% or less, 1.4% or less, 1.3% or less, 1.2% or less, 1.1% or less, 1% or less, 0.9% or less, 0.8% or less, 0.7% or less, 0.6% or less, 0.5% or less, 0.4% or less, 0.3% or less, 0.2% or less, 0.1% or less, or 0.0% of one or more of a deamidation variant, an afucosylation variant, an MGO variant, a glycation variant, a citric acid variant, a glycosylation variant, an acetonation variant, or a fragmentation variant, and ranges within one or more of the preceding. In one aspect of this embodiment, a low AR solid unit of the invention may also comprise about 0.0% to about 10%, about 0.0% to about 5%, about 0.0% to about 4%, about 0.0% to about 3%, about 0.0% to about 2%, about 3% to about 5%, about 5% to about 8%, or about 8% to about 10%, or about 10% to about 15%, of one or more of a deamidation variant, an afucosylation variant, an MGO variant, a glycation variant, a citric acid variant, a glycosylation variant, an acetonation variant, or a fragmentation variant, and ranges within one or more of the preceding. For example, a low AR solid unit of the invention may comprise less than 15% of a deamidation variant, while each of the other acidic variants, alone or in combination, are at a percentage that is greater than 15%.

The term “non-low acidic species composition,” as used herein, refers to a composition comprising an antibody or antigen-binding portion thereof, which contains more than about 16% acidic species. For example, a non-low acidic species composition may contain about 16% or more, 17% or more, 18% or more, 19% or more, 20% or more, 21% or more, 22% or more, 23% or more, 24% or more, or 25% or more acidic species. In one embodiment, a non-low acidic species composition can comprise about 16% or more, 17% or more, 18% or more, 19% or more, 20% or more, 21% or more, 22% or more, 23% or more, 24% or more, or 25% or more of AR1. In another embodiment, a non-low acidic species composition can comprise about 16% or more, 17% or more, 18% or more, 19% or more, 20% or more, 21% or more, 22% or more, 23% or more, 24% or more, or 25% or more of AR2, and ranges within one or more of the preceding.

In one embodiment, a low AR solid unit has improved biological and functional properties, including increased efficacy in the treatment or prevention of a disorder in a subject, e.g., a disorder in which TNFα activity is detrimental, as compared to a non-low acidic species composition. In one embodiment, a low AR solid unit comprising an antibody, or antigen-binding portion thereof, exhibits increased cartilage penetration, decreased bone erosion, and/or reduced cartilage destruction, as compared to a non-low acidic species composition comprising the same antibody or antigen binding portion thereof, when administered to a subject suffering from a disorder in which TNFα activity is detrimental.

In another embodiment, a low AR solid unit comprising an antibody, or antigen-binding portion thereof, exhibits increased protection against the development of arthritic scores and/or histopathology scores as compared to a non-low acidic species composition when administered to an animal model of arthritis, e.g., the TNF-Tg197 model of arthritis. As used herein, “arthritic scores” refer to signs and symptoms of arthritis in an animal model of arthritis. As used herein, “histopathology scores” refer to radiologic damage involving cartilage and bone as well as local inflammation.

In another embodiment, a low AR solid unit comprising an antibody, or antigen-binding portion thereof, exhibits reduced synovial proliferation, reduced cell infiltration, reduced chondrocyte death, and/or reduced proteoglycan loss as compared to a non-low acidic species composition. In another embodiment, a low AR composition comprising an anti-TNFα antibody, or antigen-binding portion thereof, exhibits increased TNFα affinity as compared to a non-low acidic species composition.

Accordingly, in one embodiment, a solid unit (or plurality of solid units) of the invention may include a low acidic species of an anti-human Tumor Necrosis Factor alpha (hTNFα) antibody, or antigen-binding portion thereof, e.g., a solid unit that comprises a population of anti-human Tumor Necrosis Factor alpha (hTNFα) antibodies, or antigen-binding portions thereof (and post-translationally modified forms of the antibody, or antigen-binding fragment thereof), that includes less than 15% of acidic species. In one embodiment, the low acid species of the anti-human Tumor Necrosis Factor alpha (hTNFα) antibody, or antigen-binding portion thereof, comprises a light chain variable region comprising a CDR3 domain comprising an amino acid sequence set forth as SEQ ID NO: 3, or modified from SEQ ID NO: 3 by a single alanine substitution at position 1, 4, 5, 7 or 8, a CDR2 domain comprising an amino acid sequence set forth as SEQ ID NO: 5, and a CDR1 domain comprising an amino acid sequence set forth as SEQ ID NO: 7, and a heavy chain variable region comprising a CDR3 domain comprising an amino acid sequence set forth as SEQ ID NO: 4, or modified from SEQ ID NO: 4 by a single alanine substitution at position 2, 3, 4, 5, 6, 8, 9, 10 or 11, a CDR2 domain comprising an amino acid sequence set forth as SEQ ID NO: 6, and a CDR1 domain comprising an amino acid sequence set forth as SEQ ID NO: 8.

In another embodiment, the low AR solid unit comprises an anti-human Tumor Necrosis Factor alpha (hTNFα) antibody, or antigen-binding portion thereof, comprises the amino acid sequence set forth as SEQ ID NO: 1 and a heavy chain variable region of the antibody, or antigen-binding portion thereof, comprising the amino acid sequence set forth as SEQ ID NO: 2. In another embodiment, the low AR solid unit comprises an anti-human Tumor Necrosis Factor alpha (hTNFα) antibody, or antigen-binding portion thereof, comprising a complete light chain comprising the amino acid sequence of SEQ ID NO: 9 (i.e., the adalimumab light chain) and a complete heavy chain comprising the amino acid sequence of SEQ ID NO: 10 (i.e., the adalimumab heavy chain). In other embodiments, the low AR solid unit comprises adalimumab, or a biosimilar thereof.

AR1 charge variants of adalimumab (and antibodies sharing certain structural characteristics of adalimumab, e.g., one or more CDR and/or heavy and light chain variable regions of adalimumab) can comprise, but are not limited to, deamidation variants, glycation variants, afucosylation variants, MGO variants or citric acid variants. In one embodiment, deamidation variants result from deamidation occurring at asparagine residues comprising Asn393 and Asn329 and at glutamine residues comprising Gln3 and Gln6. In another embodiment, the glycation variants result from glycation occurring at Lys98 and Lys151. AR1 structure variants can comprise, but are not limited to, glycosylation variants or acetonation variants. AR1 fragmentation variants can comprise Fc and Fab fragments, fragments missing a Fab, fragments missing a heavy chain variable domain, C-terminal truncation variants, variants with excision of N-terminal Asp in the light chain, and variants having N-terminal truncation of the light chain. AR2 charge variants can comprise, but are not limited to, deamidation variants or glycation variants, wherein the deamidation variants can result from deamidation occurring at asparagine residues comprising Asn393 and Asn329 and at glutamine residues comprising Gln3 and Gln6. In another aspect of this embodiment, when the low AR solid unit comprises adalimumab, the glycation variants can result from glycation occurring at Lys98 and Lys151 of adalimumab.

The preparation of low AR variants of a protein, e.g., an antibody, or antigen binding portion thereof, such as adalimumab, can be produced by modulating conditions during upstream protein production, such as cell culture and/or during downstream process technologies (e.g., purification following cell culture of a protein).

As used herein, the term “upstream process technology,” in the context of protein, e.g., antibody, preparation, refers to activities involving the production and collection of proteins (e.g. antibodies) from cells (e.g., during cell culture of a protein of interest). As used herein, the term “cell culture” refers to methods for generating and maintaining a population of host cells capable of producing a recombinant protein of interest, as well as the methods and techniques for optimizing the production and collection of the protein of interest. For example, once an expression vector has been incorporated into an appropriate host, the host can be maintained under conditions suitable for expression of the relevant nucleotide coding sequences, and the collection and purification of the desired recombinant protein.

When using the cell culture techniques of the instant invention, the protein of interest can be produced intracellularly, in the periplasmic space, or directly secreted into the medium. In embodiments where the protein of interest is produced intracellularly, the particulate debris, either host cells or lysed cells (e.g., resulting from homogenization) can be removed by a variety of means, including but not limited to, centrifugation or ultrafiltration. Where the protein of interest is secreted into the medium, supernatants from such expression systems can be first concentrated using a commercially available protein concentration filter, e.g., an Amicon™ or Millipore Pellicon™ ultrafiltration unit.

As used herein, the term “downstream process technology” refers to one or more techniques used after the upstream process technologies to purify the protein, e.g., antibody, of interest. For example, downstream process technology includes purification of the protein product, using, for example, affinity chromatography, including Protein A affinity chromatography, ion exchange chromatography, such as anion or cation exchange chromatography, hydrophobic interaction chromatography, or displacement chromatography.

In one embodiment, the preparation of low AR variants of a protein includes lowering the amount of acidic species variants or process-related impurities expressed by host cells producing a protein of interest including an antibody or antigen-binding portion thereof during an upstream process technology (e.g., during cell culture).

For example, the low acidic species may be produced by culturing cells expressing the antibody, or antigen binding portion thereof, in a cell culture media comprising an increased concentration of one or more amino acids; by culturing cells expressing the antibody, or antigen binding portion thereof, in a cell culture media comprising an increased concentration of calcium (e.g., as calcium chloride dihydrate); by culturing cells expressing the antibody, or antigen binding portion thereof, in a cell culture media comprising an increased concentration of niacinamide; by culturing cells in media supplemented with one or more amino acids, calcium (e.g., as calcium chloride dihydrate) and/or niacinamide, and combinations thereof.

In certain embodiments, a low acidic species may be produced by culturing host cells in a culture wherein process parameters, such as pH or dissolved oxygen (DO), are modulated, e.g., lowered to decrease the amount of acidic species produced by the host cells and/or reduce the conversion of the product to the acidic species variants.

Furthermore, a continuous or perfusion technology can utilized to obtain low AR. In certain embodiments, reduction of acidic species is obtained by modulating the medium exchange rate during cell culture.

Still further, one or more of the above supplements and modifications can be combined and used during cell culture of one protein, e.g., antibody, composition.

In one embodiment, the preparation of low AR variants and/or process related impurities of a protein includes the purification of a protein, such as an antibody or antigen-binding portion thereof, by, for example, chromatography, such as multimodal (MM) chromatography, wherein the MM media comprises both ion exchange and hydrophobic interaction functional groups, and an aqueous salt solution. In one embodiment, the same or substantially the same aqueous salt solution is used as a loading buffer and a wash buffer.

In further embodiments, the preparation of low AR variants of a protein includes the purification of a protein, such as an antibody or antigen-binding portion thereof, by, for example, chromatography comprising an anion exchange (AEX) resin and an aqueous salt solution. In one embodiment, the same or substantially the same aqueous salt solution is used as a loading buffer and a wash buffer.

In yet further embodiments, the preparation of low AR variants of a protein includes the purification of a protein, such as an antibody or antigen-binding portion thereof, by, for example, chromatography comprising a cation exchange (CEX) adsorbent resin and an aqueous salt solution. In one embodiment, the same or substantially the same aqueous salt solution is used as a loading buffer and a wash buffer, and the target protein bound to the CEX adsorbent resin is eluted with a buffer having a higher conductivity and/or pH than the loading/wash buffer.

In still further embodiments, the preparation of low AR variants of a protein includes the purification of a protein, such as an antibody or antigen-binding portion thereof, by, for example, a combination of several media, for example by using an anion exchange (AEX) resin, and chromatography using a cation exchange (CEX) adsorbent resin, in a suitable buffer, such as, for example, a Tris/Formate buffer system. In one embodiment, the sample is purified affinity chromatography media, e.g., Protein A, prior to the ion chromatography resins.

In one embodiment, the method for producing a low AR antibody, or antigen binding portion thereof, comprises contacting a first sample comprising the antibody, or antigen binding portion thereof, to affinity chromatography media in a load buffer (for example a low concentration Tris/Formate buffer), and eluting the sample from the affinity chromatography media as a first eluted sample, contacting the first eluted sample to a first chromatography media, such as an AEX chromatography resin, in a load buffer, and eluting the sample from the AEX chromatography resin as a second eluted sample. The second eluted sample is then contacted with a second chromatography media, such as a CEX chromatography resin, in a load buffer, and the sample is eluted from the CEX chromatography resin as a third eluted sample. In one embodiment, the CEX chromatography resin is eluted one, two, three or more times. In one embodiment, the process optionally includes one or more intermediate filtration steps, pH adjustment steps and/or inactivation steps. Weak cation-exchange chromatography (WCX) analysis of adalimumab has shown that it has three main basic charge variants (i.e., Lys 0, Lys 1, and Lys 2). These variants, or charged isomers, are the result of incomplete post-translational cleavage of the C-terminal lysine residues on the heavy chains of the antibody. In addition to the lysine variants, the WCX-10 analysis measures the presence acidic species. These acidic species regions (i.e., acidic species), AR1 and AR2, are classified as product-related impurities that are relatively acidic when compared to the lysine variants and elute before the Lys 0 peak in a chromatogram.

As used herein, the term “lysine variant species” refers to an antibody, or antigen-binding portion thereof, (e.g., adalimumab) comprising heavy chains with either zero, one or two C-terminal lysines. For example, the “Lys 0” variant comprises an antibody, or antigen-binding portion thereof, with heavy chains that do not comprise a C-terminal lysine. The “Lys 1” variant comprises an antibody, or antigen-binding portion thereof, with one heavy chain that comprises a C-terminal lysine. The “Lys 2” variant comprises an antibody, or antigen-binding portion thereof, with both heavy chains comprising a C-terminal lysine. Lysine variants can be detected by weak cation exchange chromatography, for example, WCX, of the expression product of a host cell expressing the antibody, or antigen-binding portion thereof.

A solid unit of the invention may comprise more than one lysine variant species of an antibody, or antigen-binding portion thereof, e.g., an anti-TNFα antibody. For example, in one embodiment, the solid unit may comprise a Lys 2 variant of an antibody, or antigen-binding portion thereof. The solid unit may comprise a Lys 1 variant of an antibody, or antigen-binding portion thereof. The solid unit may comprise a Lys 0 variant of an antibody, or antigen-binding portion thereof. In another embodiment, the solid unit may comprise both Lys 1 and Lys 2, or Lys 1 and Lys 0, or Lys 2 and Lys 0 variants of an antibody, or antigen-binding portion thereof. In another embodiment, the solid unit may comprise all three lysine variant species, i.e., Lys 0, Lys 1 and Lys 2, of an antibody, or antigen-binding portion thereof.

In one embodiment, the invention comprises a solid unit comprising an antibody, or antigen-binding portion thereof, wherein the composition comprises less than about 50% lysine variant species that lack a C-terminal lysine (Lys 0). In another embodiment, the solid unit comprises less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11% 10%, 9%, 8%, 7%, 6%, 5%, 4%, 3%, 2% or 1% lysine variant species that lack a C-terminal lysine (“Lys 0”). In another embodiment, the solid unit comprises about 50% to about 0%, about 40% to about 10%, about 30% to about 20%, about 40% to about 20%, or about 30% to about 15% lysine variant species that lack a C-terminal lysine (Lys 0). In one embodiment, the solid unit comprises 0% lysine variant species that lack a C-terminal lysine (Lys 0). As used herein, the percent lysine variant species in the solid unit refers to the weight of the specific lysine variant species in a sample in relation to the weight of the total lysine variant species sum (i.e., the sum of Lys 0, Lys 1 and Lys 2) contained in the solid unit. For example, the percent lysine variant species can be calculated using weak cation exchange chromatography such as WCX-10, as described herein.

In another embodiment, the solid unit comprises less than about 25% lysine variant species that have one C-terminal lysine (Lys 1). In another embodiment, the composition comprises less than about 24%, 23%, 22%, 21%, 20%, 19%, 18%, 17%, 16%, 15%, 14%, 13%, 12%, 11% 10%, 9%, 8%, 7%, 6%, 5%, 4%, 3%, 2% or 1% lysine variant species that have one C-terminal lysine (Lys 1). In another embodiment, the solid unit comprises about 25% to about 0%, about 20% to about 5%, about 15% to about 10%, about 20% to about 10%, about 15% to about 5%, or about 25% to about 5% lysine variant species that have one C-terminal lysine (Lys 1). In one embodiment, the solid unit comprises 0% lysine variant species that have one C-terminal Lysine (Lys 1).

In another embodiment, the solid unit comprises at least about 70% lysine variant species that have two C-terminal lysines (Lys 2). In another embodiment, the composition comprises at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% lysine variant species that have two C-terminal lysines (Lys 2). In one embodiment, the solid unit comprises about 70% to about 100%, about 70% to about 90%, about 70% to about 80%, about 80% to about 100%, about 85% to about 100%, about 90% to about 100%, about 95% to about 100%, about 80% to about 90%, about 85% to about 95%, about 75% to about 85%, or about 97% to about 100% lysine variant species that have two C-terminal lysines (Lys 2). In one embodiment, the solid unit comprises 100% lysine variant species that have two C-terminal lysines (Lys 2).

In one embodiment of the invention, an anti-TNFα antibody, or antigen-binding portion thereof, the antibody, or antigen-binding portion thereof, contains a PGPK modification at the C-terminal of the antibody heavy chain. For example, an anti-TNFα antibody, or antigen-binding portion thereof, comprising the full-length heavy and light chain sequences of adalimumab may be modified such that the C-terminal three amino acids of the heavy chain sequences are modified from the native IgG1 sequence of PGK to include a proline between the glycine and lysine to result in a C-terminal sequence of PGPK (referred to herein as a “PGPK modification”). In certain embodiments, the solid unit may contain an anti-TNFα antibody, or antigen-binding portion thereof, comprising the full-length heavy and light chain sequences of adalimumab, but which comprise a PGPK C-terminal sequence and at least one additional sequence modification to the light or heavy chain sequences (SEQ ID NOs: 9 and 10). In certain embodiments, the additional sequence modification, or modifications, can include conservative or non-conservative substitutions, insertions, and/or deletions.

In one embodiment, a solid unit of the invention comprises antibodies containing a PGPK modification, where the antibodies comprise a heavy chain variable region (HCVR) amino acid sequence having at least 65%, 70%, 75%, 80%, 85%, 90%, 95%, 96%, 97%, 98%, 99% or more, identity to the amino acid sequence of SEQ ID NO:2. The solid units may include antibodies containing a PGPK modification comprising an HCVR amino acid sequence in which 1, 2, 3, 4, 5, 6, 7, 8, 9, or 10 amino acid residue substitutions have been made relative to SEQ ID NO:2. The substitutions may be conservative amino acid substitutions. These antibodies may have at least two or more (e.g., at least 3, 4, 5, 6, 7, 8, 9, 10 or more) of the biological characteristics described herein.

In one embodiment, a solid unit of the invention comprises antibodies containing a PGPK modification, where the antibodies comprise a light chain variable region (LCVR) domain amino acid sequence having at least 65%, 70%, 75%, 80%, 85%, 90%, 95%, 96%, 97%, 98%, 99% or more identity to SEQ ID NO:1. The solid units may include antibodies containing a PGPK modification comprising an LCVR amino acid sequence in which 1, 2, 3, 4, 5, 6, 7, 8, 9, or 10 amino acid residue substitutions have been made relative to SEQ ID NO:1. In certain embodiments, the substitutions are conservative amino acid substitutions. These antibodies have at least two or more (e.g., at least 3, 4, 5, 6, 7, 8, 9, 10 or more) of the biological characteristics described herein.

In one embodiment, a solid unit of the invention comprises antibodies containing a PGPK modification, where the antibodies comprise a HCVR amino acid sequence having at least 90%, 91%, 92%, 93%, 94%, 95%, 96%, 97%, 98%, 99% or more identity to SEQ ID NO:2, and an LCVR amino acid sequence having at least 90%, 91%, 92%, 93%, 94%, 95%, 96%, 97%, 98%, 99% or more identity to SEQ ID NO:1. These antibodies may have at least two more (e.g., at least 3, 4, 5, 6, 7, 8, 9, 10 or more) of the TNFα biological characteristics described herein.

In one embodiment, a solid unit of the invention comprises antibodies containing a PGPK modification, where the antibodies comprise an HCVR amino acid sequence in which 1, 2, 3, 4, 5, 6, 7, 8, 9, or 10 amino acid residue substitutions have been made relative to SEQ ID NO:2, and an LCVR amino acid sequence in which 1, 2, 3, 4, 5, 6, 7, 8, 9, or 10 amino acid residue substitutions have been made relative to SEQ ID NO:1. These antibodies may have at least two more (e.g., at least 3, 4, 5, 6, 7, 8, 9, 10 or more) of the TNFα biological characteristics described herein.

In one embodiment, the invention features a solid unit comprising an antibody, or antigen-binding portion thereof, e.g., an anti-TNFα antibody, wherein the solid unit comprises less than about 50% lysine variant species that lack a C-terminal lysine (Lys 0). In one embodiment, the solid unit comprises less than about 25% lysine variant species that have one C-terminal lysine (Lys 1); at least about 70% lysine variant species that have two C-terminal lysines (Lys 2); at least about 80% lysine variant species that have two C-terminal lysines (Lys 2); at least about 90% lysine variant species that have two C-terminal lysines (Lys 2); or at least about 95% lysine variant species that have two C-terminal lysines (Lys 2). The solid unit of the invention may further comprise less than about 10% acidic species, wherein the acidic species comprise a first acidic species region (AR1) and a second acidic species region (AR2). In one embodiment, the solid unit comprises about 3% acidic species; comprises less than about 1% AR1; comprises about 0% AR1; comprises less than about 4% AR2; comprises about 3% AR2; or comprises about 0% AR1 and about 3% AR2.

In one embodiment, the invention features a solid unit comprising an antibody, or antigen-binding portion thereof, e.g., an anti-TNFα antibody, wherein the solid unit comprises at least about 70% lysine variant species that have two C-terminal lysines (Lys 2). In one embodiment, the solid unit comprises at least about 75% lysine variant species that have two C-terminal lysines (Lys 2); at least about 80% lysine variant species that have two C-terminal lysines (Lys 2); at least about 85% lysine variant species that have two C-terminal lysines (Lys 2); at least about 90% lysine variant species that have two C-terminal lysines (Lys 2); at least about 100% lysine variant species that have two C-terminal lysines (Lys 2). In one embodiment, the solid unit further comprises less than about 10% acidic species, wherein the acidic species comprise a first acidic species region (AR1) and a second acidic species region (AR2); comprises about 3% acidic species; comprises less than about 1% AR1; comprises about 0% AR1; comprises less than about 4% AR2; comprises about 3% AR2; or comprises about 0% AR1 and about 3% AR2.

The present invention encompasses antibodies comprising amino acids in a sequence that is substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

The foregoing applies equally to anti-TNFα antibodies and antibody fragments of the invention. Antibodies and antibody fragments having any one or more of the foregoing functional and structural characteristics are contemplated.

In one embodiment, an antibody, or antigen-binding portion thereof, suitable for use in the solid units and methods of the invention is an antibody, or antigen-binding portion thereof, which binds human interleukin 17 (IL-17), such as those antibodies described in U.S. Patent Publication No. US20100266531, the entire contents of which are incorporated herein by reference including the amino acid sequence of IL-17 antibodies described therein. In one embodiment, an antibody, or antigen-binding portion thereof, suitable for use in the solid units and methods of the invention is an antibody, or antigen-binding portion thereof, which binds human interleukin 18 (IL-18), such as those antibodies described in U.S. Patent Publication No. 2005/0100965, the entire contents of which are incorporated herein by reference including the amino acid sequence of IL-18 antibodies described therein.

In some embodiments, a TNFα antibody, or antigen-binding protein thereof, used in the invention includes the chimeric antibody infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), or CNTO 148 (golimumab; Medarex and Centocor, see WO 02/12502). Additional TNF antibodies which may be used in the invention are described in U.S. Pat. Nos. 6,593,458; 6,498,237; 6,451,983; and 6,448,380, each of which is incorporated by reference herein.

Other examples of antibodies, or antigen-binding fragments thereof, which may be used in the methods and solid units of the invention include, but are not limited to, rituximab (RITUXAN™ Biogen Idec, Genentech/Roche) (see for example U.S. Pat. No. 5,736,137) a chimeric anti-CD20 antibody approved to treat non-Hodgkin's lymphoma; ofatumumab (HUMAX-CD20™ Genmab, GlaxoSmithKline) (described in U.S. Pat. No. 5,500,362) an anti-CD20 antibody approved to treat chronic lymphocytic leukemia that is refractory to fludarabine and alemtuzumab; AME-133v (Mentrik Biotech) an anti-CD20 antibody; veltuzumab (hA20) (Immunomedics) an anti-CD20 antibody; HumaLYM (Intracel); PRO70769 (Genentech/Roche) (PCT/US2003/040426) an anti-CD20 antibody; trastuzumab (HERCEPTIN™ Genentech/Roche) (described in U.S. Pat. No. 5,677,171) a humanized anti-Her2/neu antibody approved to treat breast cancer; pertuzumab (rhuMab-2C4, OMNITARG™ Genentech/Roche) (described in U.S. Pat. No. 4,753,894); cetuximab (ERBITUX™ Imclone) (described in U.S. Pat. No. 4,943,533; PCT WO 96/40210) a chimeric anti-EGFR antibody approved to treat colorectal and head and neck cancer; panitumumab (ABX-EGF VECTIBIX® Amgen) (described in U.S. Pat. No. 6,235,883) an anti-EGFR antibody approved to treat colorectal cancer; zalutumumab (HUMAX-EGFR™ Genmab) (described in U.S. patent application Ser. No. 10/172,317) an anti-EGFR antibody; EMD55900 (Mab 425 Merck) an anti-EGFR antibody; EMD62000 and EMD72000 (Mab 425 Merck) anti-EGFR antibodies (described in U.S. Pat. No. 5,558,864; Murthy et al. (1987) Arch. Biochem. Biophys. 252(2):549-60; Rodeck et al. (1987) J. Cell. Biochem. 35(4):315-20; Kettleborough et al. (1991) Protein Eng. 4(7):773-83; ICR62 (Institute of Cancer Research) an anti-EGFR antibody (described in PCT Publication No. WO 95/20045; Modjtahedi et al. (1993) J. Cell. Biophys. 22(1-3):129-46; Modjtahedi et al. (1993) Br. J. Cancer 67(2):247-53; Modjtahedi et al. (1996) Br. J. Cancer 73(2):228-35; Modjtahedi et al. (2003) Int. J. Cancer 105(2):273-80); nimotuzumab (TheraCIM hR3, THERALOC® YM Biosciences, Oncoscience AG) (described in U.S. Pat. No. 5,891,996; U.S. Pat. No. 6,506,883; Mateo et al. (1997) Immunotechnol. 3(1):71-81) an anti-EGFR antibody; ABT-806 (Ludwig Institute for Cancer Research, Memorial Sloan-Kettering) (Jungbluth et al. (2003) Proc. Natl. Acad. Sci. USA 100(2):639-44) an anti-EGFR antibody; KSB-102 (KS Biomedix); MR1-1 (IVAX, National Cancer Institute) (PCT Publication No. WO 0162931A2) an anti-EGFRvIII antibody; SC100 (Scancell) (PCT Publication No. WO 01/88138) an anti-EGFR antibody; alemtuzumab (CAMPATH™ Genzyme/Sanofi) an anti-CD52 antibody approved to treat B-cell chronic lymphocytic leukemia; muromonab-CD3 (Orthoclone OKT3™ Johnson and Johnson) an anti-CD3 antibody approved to treat organ transplant rejection; ibritumomab tiuxetan (ZEVALIN™ Spectrum Pharmaceuticals) an anti-CD20 antibody approved to treat non-Hodgkin's Lymphoma; gemtuzumab ozogamicin (hP67.6 MYLOTARG™ Pfizer) an anti-CD33 antibody conjugated to calicheamicin; alefacept (AMEVIVE™ Astellas Pharma) an anti-CD2 LFA-3 Fc fusion; abciximab (REOPRO™ Centocor Ortho Biotech Products, Lilly) a chimeric human-mouse anti-glycoprotein IIb/IIIa receptor and anti-vitronectic αᵥβ₃ receptor antibody approved as an adjunct to percutaneous coronary intervention to prevent cardiac ischemia; basiliximab (SIMULECT™ Novartis) an anti-CF25 antibody approved to treat organ transplant rejection; palivizumab (SYNAGIS™ Medimmune) an antibody to the A antigenic site of F protein of RSV approved to treat RSV infection; infliximab (REMICADE™ Janssen Biotech) an anti-TNF-alpha antibody approved to treat Crohn's disease, ulcerative colitis, arthritis, ankylosing spondylitis, psoriatic arthritis, and plaque psoriasis; CDP571 (HUMICADE™ Celltech, Biogen IDEC) an anti-TNFα antibody; etanercept (ENBREL™ Amgen, Pfizer) an anti-TNFα Fc fusion antibody approved to treat rheumatoid arthritis, juvenile idiopathic arthritis, psoriatic arthritis, ankylosing spondylitis, plaque psoriasis; certolizumab pegol (CIMZIA, UCB Pharma) an anti-TNFα antibody approved to treat rheumatoid arthritis and Crohn's disease; ustekinumab (STELARA Janssen Biotech) a human anti-p40 subunit of IL-12 and IL-23 antibody approved to treat plaque psoriasis; galilimomab (ABX-CBL Abgenix) a mouse anti-CD147 antibody; ABX-IL8 (Abgenix) an anti-IL8 antibody; ABX-MA1 (Abgenix) an anti-MUC18 antibody; pemtumomab (Theragyn, R1549, 90Y-muHMFG1 Antisoma) a mouse anti-MUC1-Yttrium 90 antibody conjugate; Therex (R1550 Antisoma) an anti-MUC1 antibody; AngioMab (muBC-1, AS1405 Antisoma) f; HuBC-1 (Antisoma); Thioplatin (AS1407 Antisoma); natalizumab (TYSABRI® Biogen Idec, Elan) an anti-α4 integrin antibody approved to treat multiple sclerosis and Crohn's disease; VLA-1 (Santarus) a humanized anti-VLA-1 antibody; LTBR mAb (Biogen Idec) an anti-lymphotoxin β receptor antibody; lerdelimumab (CAT-152 Cambridge Antibody Technology/Abbott) an anti-TGF-β2 antibody; briakinumab (Abbott) an anti-IL-12 and 23 antibody; metelimumab (CAT-192 Cambridge Antibody Technology, Genzyme) an anti-TGFβ1 antibody; bertilimumab (CAT-213, iCO-008 Cambridge Antibody Technology, iCo Therapeutics, Immune Pharmaceuticals) an anti-eotaxin1 antibody; belimumab (BENLYSTA® Human Genome Science, GlaxoSmithKline) an anti-B lymphocyte stimulator protein antibody approved to treat systemic lupus erythematosus; mapatumumab (HGS-ETR1 Cambridge Antibody Technology, Human Genome Sciences) an anti-TRAIL-R1 antibody; bevacizumab (AVASTIN™ Genentech/Roche) an anti-VEGF antibody approved to treat metastatic colorectal cancer, non-squamous non-small cell lung cancer, glioblastoma, metastatic renal cell cancer; anti-HER3/EGFR antibody (Genentech/Roche); an Anti-Tissue Factor antibody (Genentech/Roche); omalizumab (XOLAIR™ Genentech/Roche, Novartis) an anti-IgE antibody approved to treat severe allergic asthma; efalizumab (RAPTIVA™ Genentech/Roche, Merck Serono) an anti-CD11a antibody; MLN-02 (Millennium, Genentech/Roche) an anti-α4β7 integrin antibody; zanolimumab (HUMAX CD4™ Emergent BioSolutions) an anti-CD4 antibody; HUMAX-IL15™ (AMG-714 Genmab, Amgen) an anti-IL15 antibody; HuMax-IL8 (HUMAX-Inflam™, MDX-018 Genmab, Cormorant Pharmaceuticals) an anti-IL8 antibody; HUMAX™-Cancer, (Genmab, Medarex, Oxford GlycoSciences) an anti-Heparanase I antibody; HUMAX™-Lymphoma (Genmab) an anti-IL8 antibody; HUMAX™-TAC (Genmab) an anti-IL-2Rα, CD25 antibody; daratumumab (HuMax®-CD38, Genmab, Janssen Biotech) an anti-CD38 antibody; toralizumab (IDEC-131 Biogen Idec) an anti-CD40L antibody; clenolimimab (IDEC-151 Biogen Idec) an anti-CD4 antibody; glaiximab (IDEC-114 Biogen Idec) an anti-CD80 antibody; lumilixmab (IDEC-152 Biogen Idec) an anti-CD23; anti-macrophage migration factor (MIF) antibodies (Biogen Idec, Taisho Pharmaceutical); mitumomab (BEC2 Imclone) a mouse anti-idiotypic antibody; IMC-1C11 (Imclone) a chimeric anti-VEGFR2 antibody; DC101 (Imclone) murine anti-VEGFR2 antibody; anti-VE cadherin antibody (Imclone); labetuzumab (CEA-CIDE™ Immunomedics) an anti-carcinoembryonic antigen antibody; epratuzumab (LYMPHOCIDE™ Immunomedics) an anti-CD22 antibody; yttrium (⁹⁰Y) tacatuzumab tetraxetan (AFP-Cide® Immunomedics) an anti-afetoprotein antibody; milatuzumab (MyelomaCide® Immunomedics) an anti-CF74 antibody; LeukoCide® (Immunomedics); ProstaCide® (Immunomedics); ipilimumab (Yervoy™, MDX-010 Bristol-Myers Squibb) an anti-CTLA4 antibody approved to treat melanoma; iratumumab (MDX-060 Medarex) an anti-CD30 antibody; MDX-070 (Medarex) an anti-prostate specific membrane antigen; OSIDEM™ (IDM-1 Medarex, Immuno-Designed Molecules) an anti-Her2 antibody; HUMAX™-CD4, an anti-CD4 antibody being developed by Medarex and Genmab; HuMax-IL15, an anti-IL15 antibody being developed by Medarex and Genmab; golimumab (SIMPONI™ Janssen Biotech) an anti-TNFα antibody approved to treat rheumatoid arthritis, psoriatic arthritis, ankylosing spondylitis; ustekinumab (STELARA®, CNTO 1275 Janssen Biotech) an anti-IL-12 antibody approved to treat plaque psoriasis; MOR101 and MOR102 (MorphoSys) anti-intercellular adhesion molecule-1 (ICAM-1) (CD54) antibodies; MOR201 (MorphoSys) an anti-fibroblast growth factor receptor 3 antibody; visilizumab (NUVION™ PDL BioPharma) an anti-CD3 antibody; fontolizumab (HUZAF™ PDL BioPharma) an anti-INFγ antibody; volociximab (M200 PDL BioPharma, Biogen Idec) an anti-α5β1 integrin antibody; SMART® IL-12 (PDL BioPharma) an anti-IL-12; ING-1 (Xoma) an anti-Ep-CAM antibody; omalizumab (XOLAIR™ Genentech/Roche, Novartis) an anti-IgE antibody approved to treat allergic asthma; MLNO1 (Xoma) an anti-β integrin antibody; and tocilizumab (ACTEMRA™ Genentech/Roche) an anti-IL6 antibody approved to treat rheumatoid arthritis and systemic juvenile idiopathic arthritis.

Antibodies, or antigen-binding portions thereof, that can be used in the solid units of the present invention can be generated by a variety of techniques well-known in the art, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes. In addition, the variable domains of a DVD-Ig binding protein can be obtained from parent antibodies, including polyclonal and mAbs capable of binding antigens of interest as described herein.

One preferred animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody, or antigen-binding portion thereof, can be a human, a chimeric, or a humanized antibody, or antigen-binding portion thereof. Chimeric or humanized antibodies, or antigen-binding portions thereof, of the present disclosure can be prepared based on the sequence of a non-human monoclonal antibody, or antigen-binding portion thereof, prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

Antibodies, or antigen-binding portions thereof, can be generated by any suitable method known in the art. For example, monoclonal antibodies can be prepared using a wide variety of techniques including, e.g., the use of hybridoma, recombinant, and phage display technologies, or a combination thereof. Hybridoma techniques are generally discussed in, for example, Harlow et al., Antibodies: A Laboratory Manual, (Cold Spring Harbor Laboratory Press, 2nd ed., 1988); and Hammerling, et al., In Monoclonal Antibodies and T-Cell Hybridomas, pp. 563-681 (Elsevier, N. Y., 1981). Examples of phage display methods that can be used to make the anti-CD70 antibodies include, e.g., those disclosed in Brinkman et al., 1995, J Immunol Methods 182:41-50; Ames et al., 1995, J Immunol Methods 184:177-186; Kettleborough et al., 1994, Eur J Immunol 24:952-958; Persic et al., 1997, Gene 187:9-18; Burton et al., 1994, Advances in Immunology 57:191-280; PCT Application No. PCT/GB91/01 134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108 (the disclosures of which are incorporated by reference herein).

Techniques for generating antibody fragments that recognize specific epitopes are also generally known in the art. For example, Fab and F(ab′)₂ fragments can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Techniques to recombinantly produce Fab, Fab′ and F(ab′)₂ fragments can also be employed using, e.g., methods disclosed in PCT publication WO 92/22324; Mullinax et al., 1992, BioTechniques 12(6):864-869; and Sawai et al., 1995, AJRI 34:26-34; and Better et al., 1988, Science 240:1041-1043 (the disclosures of which are incorporated by reference herein).

Examples of techniques that can be used to produce single-chain Fvs and antibodies include those described in U.S. Pat. Nos. 4,946,778 and 5,258,498; Huston et al., 1991, Methods in Enzymology 203:46-88; Shu et al., 1993, Proc Natl Acad Sci USA 90:7995-7999; and Skerra et al., 1988, Science 240:1038-1040.

Antibodies may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

Mammalian host cells for expressing the recombinant antibodies include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) J. Mol. Biol. 159:601-621) and DG44 or DUXB11 cells (Urlaub et al. (1986) Som. Cell Molec. Genet. 12:555; Haynes et al. (1983) Nuc. Acid. Res. 11:687-706; Lau et al. (1984) Mol. Cell. Biol. 4:1469-1475), NS0 myeloma cells, monkey kidney line (e.g., CVI and COS, such as a COS 7 cell), SP2 cells, human embryonic kidney (HEK) cells, such as a HEK-293 cell, Chinese hamster fibroblast (e.g., R1610), human cervical carcinoma (e.g., HELA), murine fibroblast (e.g., BALBc/3T3), murine myeloma (P3×63-Ag3.653; NS0; SP2/0), hamster kidney line (e.g., HAK), murine L cell (e.g., L-929), human lymphocyte (e.g., RAJI), human kidney (e.g., 293 and 293T). Host cell lines are typically commercially available (e.g., from BD Biosciences, Lexington, Ky.; Promega, Madison, Wis.; Life Technologies, Gaithersburg, Md.) or from the American Type Culture Collection (ATCC, Manassas, Va.).

When recombinant expression vectors encoding the antibody are introduced into mammalian host cells, the antibodies are produced by culturing the host cells for a period of time sufficient to allow for expression of the antibodies in the host cells or secretion of the antibodies into the culture medium in which the host cells are grown. Antibodies can be recovered from the culture medium using standard protein purification methods.

In an exemplary system for recombinant expression of antibodies, a recombinant expression vector encoding both the antibody heavy chain and the antibody light chain is introduced into dhfr-CHO cells by calcium phosphate-mediated transfection. Within the recombinant expression vector, the antibody heavy and light chain cDNAs are each operatively linked to CMV enhancer/AdMLP promoter regulatory elements to drive high levels of transcription of the cDNAs. The recombinant expression vector also carries cDNA encoding DHFR, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further, the invention provides a method of synthesizing an antibody by culturing a host cell of the invention in a suitable culture medium until the antibody is synthesized. The method can further comprise isolating the antibody from the culture medium.

### DVD-Ig Proteins for Use in Compositions and Methods of Invention

In another embodiment, a protein suitable for use in the solid units and methods of the invention is a “Dual Variable Domain Immunoglobulin” or “DVD-Ig™.” In one embodiment, the DVD-Ig™ binds one molecular target. In another embodiment, the DVD-Ig™ binds to at least two distinct molecular targets.

A DVD-Ig protein is formed by combining two heavy chain DVD polypeptides and two light chain DVD polypeptides. The dual variable domain immunoglobulin (DVD-Ig) heavy chain comprises two heavy chain variable domains (VH) linked in tandem, directly or by a linker, followed by the constant domain CH1 and Fc region. The dual variable domain immunoglobulin (DVD-Ig) light chain is designed such that two light chain variable domains (VL) from the two parent mAbs are linked in tandem, directly or via a linker, followed by the light chain constant domain (CL). (see FIG. 1A of U.S. Pat. No. 7,612,181, incorporated by reference herein). Methods of making DVD-Ig proteins are also described in U.S. Pat. No. 7,612,181, incorporated by reference herein.

The variable domains of the DVD-Ig protein can be obtained using recombinant DNA techniques from a parent antibody generated by any one of the methods described above. In one embodiment, the variable domain is a CDR grafted or a humanized variable heavy or light chain domain. In another embodiment, the variable domain is a human heavy or light chain variable domain. The linker sequence may be a single amino acid or a polypeptide sequence. Examples of linker sequences that may be used to link variable domains include, but are not limited to, AKTTPKLEEGEFSEAR (SEQ ID NO:11); AKTTPKLEEGEFSEARV (SEQ ID NO:12); AKTTPKLGG (SEQ ID NO:13); SAKTTPKLGG (SEQ ID NO:14); SAKTTP (SEQ ID NO:15); RADAAP (SEQ ID NO:16); RADAAPTVS (SEQ ID NO:17); RADAAAAGGPGS (SEQ ID NO:18); RADAAAA(G_{4S}).₄ (SEQ ID NO:19), SAKTTPKLEEGEFSEARV (SEQ ID NO:20); ADAAP (SEQ ID NO:21); ADAAPTVSIFPP (SEQ ID NO:22); TVAAP (SEQ ID NO:23); TVAAPSVFIFPP (SEQ ID NO:24); QPKAAP (SEQ ID NO:25); QPKAAPSVTLFPP (SEQ ID NO:26); AKTTPP (SEQ ID NO:27); AKTTPPSVTPLAP (SEQ ID NO:28); AKTTAP (SEQ ID NO:29); AKTTAPSVYPLAP (SEQ ID NO:30); ASTKGP (SEQ ID NO:31); ASTKGPSVFPLAP (SEQ ID NO:32); GGGGSGGGGSGGGGS (SEQ ID NO:33); GENKVEYAPALMALS (SEQ ID NO:34); GPAKELTPLKEAKVS (SEQ ID NO:35); GHEAAAVMQVQYPAS (SEQ ID NO:37); and GGGGSGGGGS (SEQ ID NO: 37). Other examples of linkers are described in U.S. Patent Publication No. 20100226923. The choice of linker sequences may be determined based on crystal structure analysis of several antibody Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of the CL or CH1 domain. DVD Igs of the invention were generated using N-terminal 5-6 amino acid residues, or 11-12 amino acid residues, of CL or CH1 as the linker in the light chain and the heavy chain of the DVD-Ig, respectively. The N-terminal residues of the CL or the CH1 domains, particularly the first 5-6 amino acid residues, adopt a loop conformation without strong secondary structure, and therefore can act as flexible linkers between the two variable domains. The N-terminal residues of the CL or CH1 domains are natural extensions of the variable domains, as they are part of the Ig sequences, and therefore immunogenicity potentially arising from the linkers or junctions is minimized.

Other linker sequences may include a sequence of any length of the CL or CH1 domain but not all residues of a CL/CH1 domain; for example the first 5-12 amino acid residues of the CL or CH1 domain; the light chain linkers can be from Cκ or Cλ; and the heavy chain linkers can be derived from CH1 of any isotype, including Cγ1, Cγ2, Cγ3, Cγ4, Cα1, Cα2, Cδ, Cε, and Cμ. Linker sequences may also be derived from other proteins such as Ig-like proteins, (e.g., TCR, FcR, KIR); G/S based sequences (e.g., G4S repeats); hinge region-derived sequences; and other natural sequences from other proteins.

In an embodiment, a constant domain is linked to the two linked variable domains using recombinant DNA techniques. For example, a sequence comprising linked heavy chain variable domains is linked to a heavy chain constant domain and sequence comprising linked light chain variable domains is linked to a light chain constant domain. In an embodiment, the constant domains are a human heavy chain constant domain and a human light chain constant domain, respectively. In another embodiment, the DVD-Ig heavy chain is further linked to an Fc region. The Fc region may comprise a native Fc region sequence, or a variant Fc region sequence. In an embodiment, the Fc region is a human Fc region. For example, the Fc region comprises an Fc region from an IgG1, IgG2, IgG3, IgG4, IgA, IgM, IgE, or IgD.

In an embodiment, the DVD-Ig protein is a dual-specific tetravalent binding protein. In one embodiment, the DVD-Ig protein binds CD20 and CD80. In another embodiment, the DVD-Ig protein binds VEGF and HER2. In another embodiment, the DVD-Ig protein binds TNF and RANKL. In another embodiment, the DVD-Ig protein binds TNF and DKK. In another embodiment, the DVD-Ig protein binds CD20 and RANKL. In another embodiment, the DVD-Ig protein binds DLL4 and PLGF. In another embodiment, the DVD-Ig protein binds DLL4 and VEGF. In another embodiment, the DVD-Ig protein binds TNF and SOST. In another embodiment, the DVD-Ig protein binds IL-9 and IgE. In another embodiment, the DVD-Ig protein binds IL-12 and IL-18. An example of an IL-12 and IL-18 DVD-Ig protein is described in U.S. Pat. No. 7,612,181. In another embodiment, the DVD-Ig protein binds TNF and IL-17. In another embodiment, the DVD-Ig protein binds TNF and PGE2. In one embodiment, the DVD-Ig protein binds TNFα and IL-17. Examples of TNFα and IL-17 DVD-Ig protein may be found in, for example, U.S. Publication No. 20100266531, 2013/0164256, and 2014/0017246. Examples of PGE2 DVD-Ig proteins are provided in U.S. Patent Publication No. 20100074900. In another embodiment, the DVD-Ig protein binds IL-1α and IL-1(3. Examples of an IL-1α and IL-1β DVD-Ig protein is described in U.S. Pat. Nos. 7,612,181 and 8,841,417. In another embodiment, the DVD-Ig protein binds IL-4 and IL-1. An example of an IL-4 and IL-13 DVD-Ig protein is described in U.S. Publication No. 20100226923. The amino acid and nucleic acid sequences described in the aforementioned patents and patent applications are incorporated by reference herein.

In one embodiment of the invention, an antibody, or antigen-binding portion thereof, or DVD-Ig™ suitable for use in the methods and solid units of the invention may bind a target antigen selected from the group consisting of ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BLyS/BAFF; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL2? (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSF1 (M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10(IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; CGRP; C1q; C1r; C1; C4a; C4b; C2a; C2b; C3a; C3b; DAB2IP; DES; DKFZp451J0118; DNCL1; DPP4; E-selectin; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); Factor VII; Factor IX; Factor V; Factor VIIa; Factor X; Factor XII; Factor XIII; FADD; FasL; FASN; Fc gamma receptor; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); F1112584; F1125530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNAS1; GNRH1; GPR2 (CCR10); GPR31; GPR44; GPR81 (FKSG80); GRCC10 (C10); GRP; GSN (Gelsolin); GSTP1; glycoprotein (GP) IIb/IIIa; HAVCR2; HDAC4; HDAC5; HDAC7A; HDAC9; Her2; HGF; HIF1A; HIP1; histamine and histamine receptors; HLA-A; HLA-DRA; HM74; HMGB1; HMOX1; HUMCYT2A; ICEBERG; ICOSL; ID2; IFN-a; IFNA1; IFNA2; IFNA4; IFNA5; IFNA6; IFNA7; IFNB1; IFNgamma; IFNW1; IGBP1; IGF1; IGF1R; IGF2; IGFBP2; IGFBP3; IGFBP6; IL-1; IL-1α; IL-1β; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2; IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); L-selectin; LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NKG2D; NFKB1; NFKB2; NGF; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NRII2; NRII3; NR2C1; NR2xamC2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGFA; PDGFB; PECAM1; PF4 (CXCL4); PGE2; PGF; PGR; phosphacan; PIAS2; PIK3CG; plasminogen activator; PLAU (uPA); PLG; PLXDC1; PPBP (CXCL7); PPID; PR1; PRKCQ; PRKD1; PRL; PROC; Protein C; PROK2; PSAP; PSCA; PTAFR; PTEN; PTGS2 (COX-2); PTN; RAC2 (p21Rac2); RAGE; RARB; RGS1; RGS13; RGS3; RNF110 (ZNF144); ROBO2; SI00A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STATE; STEAP; STEAP2; substance P; TB4R2; TBX21; TCP10; TDGF1; TEK; TGFA; TGFB1; TGFB111; TGFB2; TGFB3; TGFBI; TGFBR1; TGFBR2; TGFBR3; TH1L; THBS1 (thrombospondin-1); THBS2; THBS4; THPO; TIE (Tie-1); TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; thrombomodulin; thrombin; VEGF; VEGFB; VEGFC; versican; VHL C5; VLA-4; XCL1 (lymphotactin); XCL2 (SCM-1b); XCR1 (GPR5/CCXCR1); YY1; and ZFPM2.

Methods for the preparation of DVD-Igs are described in U.S. Pat. No. 7,612,181, the entire contents of which are incorporated herein by reference. Briefly, a DVD-Ig is designed such that two different light chain variable domains (VL) from the two different parent monoclonal antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region.

The variable domains can be obtained using recombinant DNA techniques from a parent antibody generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

In one embodiment the first and second variable domains are linked directly to each other using recombinant DNA techniques. In another embodiment the variable domains are linked via a linker sequence. In an embodiment, two variable domains are linked. Three or more variable domains may also be linked directly or via a linker sequence. The variable domains may bind the same antigen or may bind different antigens. DVD-Ig molecules of the invention may include one immunoglobulin variable domain and one non-immunoglobulin variable domain such as ligand binding domain of a receptor, active domain of an enzyme. DVD-Ig molecules may also comprise two or more non-Ig domains.

### Other Therapeutic Proteins for Use in Compositions and Methods of Invention

In one embodiment, a solid unit or plurality of solid units of the invention includes a protein, such as a therapeutic protein or a peptide. Non-limiting examples of proteins suitable for use in the solid units and methods of the invention include mammalian proteins, such as, e.g., growth hormone, including human growth hormone and bovine growth hormone; growth hormone releasing factor; parathyroid hormone; thyroid stimulating hormone; lipoproteins; α-1-antitrypsin; insulin A-chain; insulin B-chain; proinsulin; follicle stimulating hormone; calcitonin; luteinizing hormone; glucagon; clotting factors such as factor VIIIC, factor, tissue factor, and von Willebrands factor; anti-clotting factors such as Protein C; atrial natriuretic factor; lung surfactant; a plasminogen activator, such as urokinase or tissue-type plasminogen activator (t-PA); bombazine; thrombin; tumor necrosis factor-α and -β; enkephalinase; RANTES (regulated on activation normally T-cell expressed and secreted); human macrophage inflammatory protein (MIP-1-α); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins; CD proteins such as CD3, CD4, CD8, CD19 and CD20; erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

In one embodiment, the protein is a therapeutic protein, including, but not limited to, fusion proteins and enzymes. Examples of therapeutic proteins include, but are not limited to, Pulmozyme (Dornase alfa), Regranex (Becaplermin), Activase (Alteplase), Aldurazyme (Laronidase), Amevive (Alefacept), Aranesp (Darbepoetin alfa), Becaplermin Concentrate, Betaseron (Interferon beta-1b), BOTOX (Botulinum Toxin Type A), Elitek (Rasburicase), Elspar (Asparaginase), Epogen (Epoetin alfa), Enbrel (Etanercept), Fabrazyme (Agalsidase beta), Infergen (Interferon alfacon-1), Intron A (Interferon alfa-2a), Kineret (Anakinra), MYOBLOC (Botulinum Toxin Type B), Neulasta (Pegfilgrastim), Neumega (Oprelvekin), Neupogen (Filgrastim), Ontak (Denileukin diftitox), PEGASYS (Peginterferon alfa-2a), Proleukin (Aldesleukin), Pulmozyme (Dornase alfa), Rebif (Interferon beta-1a), Regranex (Becaplermin), Retavase (Reteplase), Roferon-A (Interferon alfa-2), TNKase (Tenecteplase), and Xigris (Drotrecogin alfa), Arcalyst (Rilonacept), NPlate (Romiplostim), Mircera (methoxypolyethylene glycol-epoetin beta), Cinryze (C1 esterase inhibitor), Elaprase (idursulfase), Myozyme (alglucosidase alfa), Orencia (abatacept), Naglazyme (galsulfase), Kepivance (palifermin) and Actimmune (interferon gamma-1b).

Proteins suitable for use in the present application may be chemically synthesized or generated recombinantly using methods routine to one of ordinary skill in the art.

### III. METHODS FOR THE PREPARATION OF SOLID UNITS

Generally, solid units of the invention are prepared by freezing a liquid solution containing a therapeutic agent, such as a therapeutic agent, such as a protein, (e.g., an antibody or antigen-binding portion thereof, peptide, or DVD-Ig protein) followed by vacuum sublimation. This process is collectively referred to herein as lyophilization.

In one embodiment, a stabilizer, such as a lyoprotectant, e.g., a sugar such as sucrose, is added to the liquid solution containing the therapeutic agent, such as a protein (e.g., a peptide, antibody, or DVD-Ig protein) prior to lyophilizing. In one embodiment, the stabilizer is a sugar, such as, but not limited to, sorbitol, mannitol, sucrose or trehalose. Where the stabilizer is sorbitol, sucrose, or trehalose and the protein is an antibody, exemplary concentrations in the initial formulation of the stabilizer are about 30 mg/ml to about 100 mg/ml; about 40 mg/ml to about 90 mg/ml; about 40 mg/ml to about 80 mg/ml; about 40 mg/ml to about 70 mg/ml; about 40 mg/ml to about 60 mg/ml; or about 40 mg/ml to about 50 mg/ml of sucrose or sorbitol. In another embodiment, the concentration of sucrose in a solution for preparation of the solid unit is about 1% to about 7% sucrose. In one embodiment, the concentration of sucrose in a solution used in the preparation of the solid unit is less that 15%, less than 10%, or less than 7%.

After the therapeutic agent, such as a protein (e.g., a peptide, antibody, or DVD-Ig protein) and stabilizer (or other optional components (e.g., a surfactant)) are mixed together into a liquid solution, the liquid solution is then dispensed into discrete aliquots, such as drops (also referred to as droplets), using any suitable dispensing method, (e.g., a needle, a pipette, a robotic dispensing system). In one embodiment, the drops are dispensed using ultrasonic methods. The solution is dispensed onto a chilled surface without contacting the dispenser to the surface. In one embodiment, the solution is dispensed above the surface.

In one embodiment, the solution is not aliquoted onto a flat chilled surface.

The temperature of the surface is adjusted so that the liquid being dispensed does not freeze in the dispenser but rapidly freezes within seconds of contact with the surface so that there is no significant loss due to evaporation and no significant change in the physical shape of the dispensed solution. In certain embodiments, in which a drop of the initial formulation is dispensed, the solution freezes as discrete units. Such control is achieved based on distance from (height above) of the surface. In other embodiments, a sheet of the initial formulation having a desired thickness is prepared and after lyophilization, discrete units having a desired volume are punched using a suitable die.

The surface may be chilled by disposing it over a liquid bath containing a cryogenic agent such as liquid nitrogen or Freon. In one embodiment, the liquid is dispensed over a cold nitrogen gas. In general, the temperature of the surface is reduced to near the temperature of the cryogenic agent. The temperature of the surface may be about −150° C. or lower, or −180° C. or lower, or about −200° C. In other embodiments, the temperature of the surface is within a range of about −90° C. to about −130° C., about −110° C. to about −150° C., about −150° C. to about −195° C. or −180° C. to about −196° C. In one embodiment, the dispensor is not submerged in the liquid solution.

Notably, the lyophilization methods of the invention are distinct from spray-freeze drying and spray-drying. Further the methods of the invention do not rely on atomization of the solution. Thus, in one embodiment, the method does not include spray-freeze drying, spray-drying, or atomization of the solution comprising the therapeutic agent, such as a protein.

The solid units of the invention are preferably frozen under conditions suitable for controlling nucleation. (See Anuj (2012) Int. J. Drug Dev. & Res., 4 (3): 35-40 for a review on controlled nucleation). Using controlled nucleation, a population of solid units freezes collectively under substantially similar conditions, e.g., narrow temperature and time for freezing (see, e.g., FIG. 1 of Anuj ibid which compares controlled vs. uncontrolled nucleation of a solution). Preferably, controlled nucleation is achieved by instantaneous freezing rather than through the use of a temperature gradient. Instantaneous freezing of the solid units may be achieved by using, for example, liquid nitrogen or Freon. When a batch of solid units is lyophilized under controlled nucleation conditions, the resulting population of solid units is substantially uniform in that the microstructure of the solid units are similar, e.g., consistency among pore size. In one embodiment, a plurality of solid units have similar pore size and homogeneity within the solid unit as a result of being frozen using controlled nucleation techniques.

Following freezing, the solid units are introduced into a lyophilization apparatus and subjected to a vacuum while still frozen for a pressure and time sufficient to remove the solvent (e.g., by sublimation) from the units, thereby forming solid units. The period of residency in the lyophilizer sufficient to produce lyophilized pellets will vary according to unit size and composition.

A secondary drying stage may be carried out at about 0-40° C., depending primarily on the type and size of container and the type of protein employed. The time and pressure required for secondary drying will be that which produces a suitable solid unit, dependent, e.g., on the temperature and other parameters. The pressure may be the same as that employed during the primary drying step.

Lyophilization may be performed according to methods known in the art. For example, many different lyophilizers are available for this purpose such as HULL50 (Hull, USA) or GT20 (Leybold-Heraeus, Germany) lyophilizers. Lyophilization is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days. Optionally, a secondary drying stage may also be performed depending upon the desired residual moisture level in the product. The temperature at which the secondary drying is carried out ranges from about 0-40° C., depending primarily on the type and size of container and the type of protein employed. For example, the shelf temperature throughout the entire water removal phase of lyophilization may be from about 15-30° C. (e.g., about 20° C.). The time and pressure required for secondary drying will be that which produces a suitable lyophilized solid unit, dependent, e.g., on the temperature and other parameters. The secondary drying time is dictated by the desired residual moisture level in the product and typically takes at least about 5 hours. The pressure may be the same as that employed during the primary drying step. The underlying principles and general protocols for lyophilization are generally known to the ordinarily skilled person. See, e.g., Methods in Molecular Biology, Cryopreservation and Freeze-Drying Protocols, John G. Day (Ed.), Humana Press, totawa, NJ (2007).

Commercially available lyophilizers for use with the present invention include, but are not limited to: Virtis Advantage XL Benchtop Freeze Dryer, and the Virtis Genesis 25 Super XL Pilot Scale Freeze Dryer (both by Virtis, of Gardiner, N.Y.), Hull50™ (Hull, USA) or GT20™ (Leybold-Heraeus, Germany).

In some instances, it may be desirable to prepare the solid units, e.g., lyophilize the protein formulation, in the container in which reconstitution of the protein is to be carried out in order to avoid a transfer step. The container in this instance may, for example, be a dual-chamber container.

As a general proposition, lyophilization will result in a lyophilized formulation in which the moisture content thereof is less than about 5%, less than about 4%, or less than about 3%, less than about 2%, less than about 1%, less than about 0.5%, about 0.25%, about 0.1%, or about 0.1-5%.

The methods of the invention may be used to obtain any number of solid units. For example, the methods may be used to prepare 2 or more solid units, 2 or more solid units, 10 or more solid units, 50 or more solid units, 100 or more solid units, 1000 or more solid units, etc.

In certain embodiment, e.g., when being used for pharmaceutical purposes, the plurality of solid units are prepared under aseptic conditions.

In one embodiment, a plurality of solid units comprising a therapeutic agent, such as a protein (e.g., a peptide, an antibody, or a DVD-Ig protein) is prepared by dispensing drops of a solution comprising the therapeutic agent into a bath of liquid nitrogen or Freon (or any cryogenic solution). The drops are delivered using any suitable dispensing device and are measured such that the substantially the same volume is delivered with each drop. Drops are repeatedly placed in sequence in the liquid nitrogen or Freon such that a plurality of solid units is obtained. Once place in the cryogenic bath, the droplet solidifies to a frozen solid unit. Barriers may be placed within the bath such that each droplet is isolated from other droplets being frozen. The freezing of the droplet is instantaneous and, thus, is performed using controlled nucleation in order to provide consistency among the population of solid units being prepared. If liquid nitrogen is used as the cryogenic agent, once the droplet of solution is frozen, the solid unit generally falls below the surface of the liquid to the bottom of the container. The population of solid units can then be collected and separated from the liquid nitrogen or Freon. The plurality of solid units is next subjected to vacuum sublimation to remove residual water. Following water removal, the plurality of solid units are free-flowing and geometrically uniform in nature. This process may be repeated to obtain a plurality of solid units having different characteristics, e.g., different size or containing a different therapeutic agent, where the first batch of solid units can be combined with the second batch to provide a plurality of solid units having distinct features but maintaining the free flowing nature of the units. Further, the aforementioned process may be used to obtain a single solid unit, if desired. This process results in solid units that are spheres due to the freezing step in the liquid nitrogen or Freon, where the solid unit forms in suspension and not on the hard surface of a plate, etc.

In some embodiments, the methods further include contacting a solid unit with a polymer, such as enteric protectant, a slow release polymer, a non-pH sensitive polymer, a solvent, a bioadhesive polymer, or any combination thereof, using methods routine to one of ordinary skill in the art.

Stabilizers, including sugars, and other excipients that may be added to the solid unit(s) of the invention are described above in Section II. In one embodiment, the concentration of a sugar, such as sucrose, in a solution for preparation of the solid unit is about 10 mg/ml to about 200 mg/ml; about 30 mg/ml to about 100 mg/ml; about 40 mg/ml to about 90 mg/ml; about 40 mg/ml to about 80 mg/ml; about 40 mg/ml to about 70 mg/ml; about 40 mg/ml to about 60 mg/ml; and about 40 mg/ml to about 50 mg/ml. In one embodiment, the solid unit is prepared from a solution comprising about 10 to about 40 mg/mL of mannitol and about 60 mg/mL to about 80 mg/mL of sucrose. In one embodiment, the concentration of sucrose in a solution for preparation of the solid unit is less than 20%, less than 15%, less than 10%, less than 7%, or about 1% to about 7% sucrose.

### IV. ARTICLES OF MANUFACTURE

In another embodiment of the invention, an article of manufacture is provided which contains the solid units (or a plurality thereof) of the present invention and provides instructions for their use.

Following the lyophilization process described herein, the solid units can be transferred to an appropriate container depending on the intended use. For example, the solid units may be transferred to filling equipment or a bulk intermediate container. Such transfer may be done under aseptic and/or controlled humidity conditions to insure pharmaceutical quality material. In some instances, the solid units will be placed in a primary container where they may be combined (either before or after placement in the primary container) with a terminally sterilized diluent. A stopper is then placed in the primary container, the primary container may be steam sterilized, and sealed until ready for administration to a patient.

The article of manufacture may include a container. Suitable containers include, for example, bottles, vials (e.g., dual chamber vials), syringes (such as dual chamber syringes), autoinjector pen containing a syringe, and test tubes. The container may be formed from a variety of materials such as glass, plastic or polycarbonate. The container holds the solid units and the label on, or associated with, the container may indicate directions for use. For example, the label may indicate that the solid units are useful or intended for subcutaneous administration, parenteral administration, or oral administration. The container holding the solid units may be a multi-use vial, which allows for repeat administrations (e.g., from 2-6 administrations) of the solid units.

In certain embodiments, a plurality of solid units of the invention are provided in a dual chamber cartridge. Generally, a dual chamber cartridge comprises an active substance (e.g., a plurality of solid units containing an antibody or antigen binding portion thereof) and a sterile diluent in two separate chambers that remain unmixed until shortly before administration. Dual chamber systems decrease the risk of medication error by eliminating the use of multiple vials and needles, as well as eliminating multiple steps required to reconstitute an active substance for administration. In certain dual chamber systems, the two chambers are interconnected by an aperture, wherein a stopper, or a suitable similar means, can be engaged to prevent contact of the active substance and the diluent liquid until ready for use. In use, the diluent is brought into contact with the active substance by disengagement or puncture of the stopper by any suitable means, for example a device such as a plunger that exerts pressure or drives a needle through the stopper.

It can be appreciated that various dual-chambered reconstitution systems capable of combining the solid units with the aqueous solution in a sterile manner are within the scope of the present invention. By way of example, the two chambers may be configured serially (i.e., a front chamber and a rear chamber with respect to the dispensing end of the cartridge), separated from each other by an aperture or any similar means to divide the two chambers. In another exemplary embodiment, the two chambers may be configured side-by-side, wherein each chamber is equidistant to the dispensing end of the cartridge. In one embodiment, the active substance (i.e., the solid units) may be prepared directly in one chamber; the diluent may be filled into the second chamber upon lyophilization, and sterilized. In other embodiments, the lyophilized solid units and diluent may individually be filled into each chamber, and subsequently sterilized.

In some embodiments, the dual chamber cartridge provides instructions for its reconstitution and/or use. The cartridge holds the solid units/diluent and the label on, or associated with, the cartridge may indicate directions for reconstitution and/or use. For example, the label may indicate that the composition is reconstituted to protein concentrations as described herein. The label may further indicate that the composition is useful or intended for subcutaneous administration. The article of manufacture may further include other materials desirable from a commercial and user standpoint, including needles, syringes, and package inserts with instructions for use. U.S. Pat. Nos. 4,968,299, 4,874,381, 5,080,649, 5,728,075, 6,218,425, 7,259,233, and 7,396,347, and WO 93/14799, incorporated by reference herein, all describe arrangements for securing mixing of contents in cartridges having at least two chambers and followed by injection.

In one embodiment, the invention features a container comprising a plurality of lyophilized solid units which are free-flowing and geometrically uniform, wherein the plurality of solid units comprises a therapeutic agent and a stabilizer, and wherein the plurality of subunits is prepared by controlled nucleation. The solid units may be spherical in size and have any of the aforementioned diameters and/or protein content in Section II. In one embodiment, the speherical solid units each have a diameter which is about 0.1 to about 4 mm; about 0.1 to about 3 mm; about 0.1 to about 2 mm; about 0.1 to about 1 mm; or about 0.1 to about 0.5 mm. In another example, the solid units within a container comprise 0.02 μg to 6.0 mg of the therapeutic protein or 15 μg to 4.0 mg of the therapeutic protein.

As described above, one advantage of the invention is that the lyophilization process does not have to occur in the primary container. Thus, in one embodiment, the container containing a plurality of solid units of the invention is not the same container used to lyophilize the solid units.

A container of the invention may include any number of solid units, ranging from one solid unit to millions of solid units. For example, the containers may contain 10 or less solid units; 50 or less solid units; 100 or less solid units; 1,000 or less solid units; or 5,000 or less solid units; 10,000 or less solid units; 50,000 or less solid units; 100,000 or less solid units; 500,000 or less solid units; 1,000,000 or less solid units; or more than 1,000,000 solid units.

In one embodiment, the invention features a container which is either an intermediate container or a primary container. Examples of primary containers include, but are not limited to, an ampule, a bag, a blister, a bottle, a cartridge, an injection needle, an injection syringe, a single-dose container, a strip, a dual chamber syringe, a dual chamber cartridge, a patch pump, a dual chamber patch pump, and a vial.

In one embodiment, the invention further features a method of preparing an intermediate container comprising a bulk intermediate drug product comprising solid units described herein. The method includes first lyophilizing a solution comprising a therapeutic protein and a stabilizer under conditions suitable for controlling nucleation of the solution during freezing. This step results in a bulk intermediate drug product comprising a plurality of solid units, where the solid units will be further processed prior to becoming a drug product. Following lyophilization, the bulk intermediate drug product is placed in an intermediate container. The intermediate drug container could be store for a period of time including, but not limited to, about 1 month, about 3 months, about 1 year, or greater than 1 year. As described herein, one advantage of the solid units is that they maintain stability for extended periods, despite containing proteins. The stability is such that the protein retains activity upon reconstitution of the solid unit. Thus, intermediate containers are contemplated by the invention as they directly relate to the stability and flexibility provided by the solid units described herein.

### IV. USES OF THE SOLID UNITS OF THE INVENTION

Given their overall stability, the solid units of the invention may be used to deliver and/or store proteins, including therapeutic agents, e.g., therapeutic proteins (such as antibodies, peptides, and DVD-Ig proteins). The solid units of the invention lend themselves to uses requiring precise amounts of a protein, as they can be made to reliably in any desired shape or volume, where the amount of protein contained within the solid unit may also be controlled.

An important use of the solid units of the invention is to deliver and/or store therapeutic proteins. The ability to make reproducibly consistent populations of solid units having certain amounts of therapeutic protein, e.g., an antibody, makes the solid units ideal for delivery precise amounts of drug to a patient. The ability of the solid units to reconstitute more quickly than standard cake lyophilized formulations also makes the solid unit of the invention suitable for storing therapeutic proteins which must be stored and reconstituted immediately prior to use. The solid units of the invention provide the patient with the benefit that reconstitution times are minimized so overall care is more efficient.

The uniformity of the solid unit of the present invention allows for convenient dosing. For example, dosing may be accomplished by counting individual solid units. By way of example, if a prescription requires 20 units (or e.g., 20 mg) of an active substance, and each solid unit is determined to contain 2 units (or 2 mg) of an active substance, a pharmacist (or anyone who fills a prescription) will dispense the necessary number of solid units simply by counting the solid units (as exemplified here, 10 solid units) and filling them into a suitable container, including, but not limited to, a delivery device (e.g., dual chambered cartridge) as described herein. In certain embodiments, a delivery device is prefilled with an appropriate amount of diluent. In other embodiments, a pharmacist will fill the delivery device with an appropriate amount of diluent.

Additionally, dosing may be accomplished by measuring a length of close-packed solid units during filling of the primary delivery device (e.g., dual chambered cartridge). Again, by way of example, if a prescription requires 20 units (or, e.g., 20 mg) of an active substance, and a length of, e.g., 10 mm is equivalent to 10 units, then a pharmacist would measure a length of 20 mm of a close-packed solid units and fill a delivery device as appropriate. In certain embodiments, the delivery device is prefilled with an appropriate amount of diluent. In other embodiments, a pharmacist will fill the delivery device with an appropriate amount of diluent.

Due to their consistency in size, weight may also be used to determine the appropriate number of solid units to be dispersed for therapeutic purposes.

At the desired stage, typically when it is time to administer an antibody to a patient, the solid units may be reconstituted with a diluent such that the antibody concentration in the reconstituted formulation is at least 50 mg/mL, for example from about 50 mg/mL to about 400 mg/mL, more preferably from about 80 mg/mL to about 300 mg/mL, and most preferably from about 90 mg/mL to about 150 mg/mL. Such high antibody concentrations in the reconstituted formulation are considered to be particularly useful where subcutaneous delivery of the reconstituted formulation is intended. However, for other routes of administration, such as intravenous administration, lower concentrations of the protein in the reconstituted formulation may be desired (for example from about 5-50 mg/mL, or from about 10-40 mg/mL protein in the reconstituted formulation). In certain embodiments, the antibody concentration in the reconstituted formulation is significantly higher than that in the pre-lyophilized formulation. For example, the protein concentration in the reconstituted formulation may be about 2-40 times, preferably 3-10 times and most preferably 3-6 times (e.g. at least three fold or at least four fold) that of the pre-lyophilized formulation.

Reconstitution often takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The diluent may contain a preservative. Exemplary preservatives have been described above, with aromatic alcohols such as benzyl or phenol alcohol. The amount of preservative employed is determined by assessing different preservative concentrations for compatibility with the protein and preservative efficacy testing.

The reconstituted solid units of the present invention can be administered in a number of ways depending upon whether local or systemic treatment is desired and upon the area to be treated. Administration can be, for example, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. In another embodiment of the invention, a solid unit is suitable for use as a suppository for, e.g., rectal, vaginal, or urethral administration.

In one embodiment, the solid unit(s) of the invention are delivered to a subject subcutaneously (upon reconstitution). In one embodiment, the subject administers the formulation to himself/herself (self-administration).

In one embodiment, the effective amount of antibody may be determined according to a strictly weight based dosing scheme (e.g., mg/kg) or, alternatively, may be a total body dose (also referred to as a fixed dose) which is independent of weight. In one example, an effective amount of an anti-TNFα antibody is a total body dose of about 10 mg. In one example, an effective amount of an anti-TNFα antibody is a total body dose of about 80 mg. In another example, an effective amount of an anti-TNFα antibody is a total body dose of about 40 mg. In yet another example, an effective amount of an anti-TNFα antibody is a total body dose of about 160 mg. In a further example, an effective amount of an anti-TNFα antibody is a total body dose of about 20 mg of antibody. In yet another example, an effective amount of an anti-TNFα antibody is a total body dose of about mg. Alternatively, an effective amount may be determined according to a weight-based fixed dosing regimen (see, e.g., WO 2008/154543, incorporated by reference herein). Thus, the invention includes solid unit(s) of the invention collectively or individually comprising the foregoing amounts which provide a dose of an anti-TNFα antibody for therapeutic purposes.

In one embodiment, the TNF-alpha is human TNF-alpha and the subject is a human subject. Alternatively, the subject can be a mammal expressing a TNF-alpha with which an antibody of the invention cross-reacts. Still further the subject can be a mammal into which has been introduced hTNF-alpha (e.g., by administration of hTNF-alpha or by expression of an hTNF-alpha transgene).

The formulations of the invention may be administered according to a certain dosing schedule. For example, the formulations may be administered according to a weekly, biweekly, or monthly dosing regimen. Alternatively, the formulation may be administered once every three weeks. In one embodiment, the formulations and methods comprise administration to the subject of a human anti-TNFα antibody according to a periodicity selected from the group consisting of weekly, biweekly, every three weeks, and monthly.

In one embodiment, the solid unit(s) of the invention may be administered (either directly or upon reconstitution) to a subject via, for example, a prefilled syringe, an autoinjector pen, dual chamber syringe, a patch pump, or a needle-free administration device. Thus, the invention also features a delivery device, (e.g., an autoinjector pen, a prefilled syringe, or a needle-free administration device) comprising solid unit(s) of the invention which provide a dose of a therapeutic agent, such as a human anti-TNF-alpha antibody, or antigen-binding portion thereof.

In one embodiment, the invention features a delivery device comprising a dose (made up by a plurality of solid units) comprising 1 to 500 mg a human anti-TNF-alpha antibody, or antigen-binding portion thereof, e.g., an autoinjector pen or prefilled syringe comprises a dose of about 10 mg, about 11 mg, about 12 mg, about 13 mg, about 14 mg, about 15 mg, about 16 mg, about 17 mg, about 18 mg, 19 mg, 20, mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, or 500 mg. In one embodiment, the syringe or autoinjector contains solid unit(s) providing 60-100 mg, 70-90 mg, or about 80 mg of the antibody. In one embodiment, the delivery device contains solid unit(s) providing 10 mg of the antibody. In one embodiment, the delivery device contains solid unit(s) providing 20 mg of the antibody. In one embodiment, the delivery device contains solid unit(s) providing 40 mg of the antibody. In one embodiment, the delivery device contains solid unit(s) providing 80 mg of the antibody. In one embodiment, the delivery device contains solid unit(s) providing 160 mg of the antibody. In one embodiment, the delivery device contains solid unit(s) providing 200 mg of the antibody. In one embodiment, the delivery device contains solid unit(s) providing 220 mg of the antibody. In one embodiment, the delivery device contains solid unit(s) providing 260 mg of the antibody. In one embodiment, the delivery device contains solid unit(s) providing 280 mg of the antibody. In one embodiment, the delivery device contains solid unit(s) providing 300 mg of the antibody. In one embodiment, the delivery device contains solid unit(s) providing 320 mg of the antibody. In one embodiment, the delivery device contains solid unit(s) providing 340 mg of the antibody. In one embodiment, the delivery device contains solid unit(s) providing 360 mg of the antibody. In one embodiment, the delivery device contains solid unit(s) providing 380 mg of the antibody. In one embodiment, the delivery device contains solid unit(s) providing 400 mg of the antibody. In one embodiment, the delivery device contains solid unit(s) providing 420 mg of the antibody. In one embodiment, the delivery device contains solid unit(s) providing 440 mg of the antibody. In one embodiment, the delivery device contains solid unit(s) providing 460 mg of the antibody. In one embodiment, the delivery device contains solid unit(s) providing 480 mg of the antibody. In one embodiment, the delivery device contains solid unit(s) providing 500 mg of the antibody.

In one embodiment, the formulations of the invention may be self administered using, e.g., a preloaded syringe or an automatic injection device. Automatic injection devices offer an alternative to manually-operated syringes for delivering therapeutic agents into patients' bodies and allowing patients to self-administer injections. Automatic injection devices are described, for example, in the following publications, each of which is hereby incorporated herein by reference: WO 2008/005315, WO 2010/127146, WO 2006/000785, WO 2011/075524, WO 2005/113039, WO 2011/075524.

Accordingly, in one embodiment, the present invention provides pre-filled syringes or autoinjector devices containing the solid unit(s) of the invention, as well as use of pre-filled syringes or autoinjector devices comprising the solid unit(s) described herein in the methods of the invention.

In one embodiment, an effective amount of solid units containing an anti-TNF-alpha antibody, or antigen-binding portion thereof, are administered to a subject to inhibit detrimental TNF-alpha activity or treat a disorder in which TNF-alpha activity is detrimental.

As used herein, the term “a disorder in which TNF-alpha activity is detrimental” is intended to include diseases and other disorders in which the presence of TNF-alpha. in a subject suffering from the disorder has been shown to be or is suspected of being either responsible for the pathophysiology of the disorder or a factor that contributes to a worsening of the disorder. Accordingly, a disorder in which TNF-alpha.activity is detrimental is a disorder in which inhibition of TNF-alpha activity is expected to alleviate the symptoms and/or progression of the disorder. Such disorders may be evidenced, for example, by an increase in the concentration of TNF-alpha. in a biological fluid of a subject suffering from the disorder (e.g., an increase in the concentration of TNF-alpha. in serum, plasma, synovial fluid, etc. of the subject), which can be detected, for example, using an anti-TNF-alpha. antibody.

There are numerous examples of disorders in which TNF-alpha activity is detrimental. Examples in which TNF-alpha activity is detrimental are also described in U.S. Pat. Nos. 6,015,557; 6,177,077; 6,379,666; 6,419,934; 6,419,944; 6,423,321; 6,428,787; and 6,537,549; and PCT Publication Nos. WO 00/50079 and WO 01/49321, the entire contents of all of which are incorporated herein by reference. The formulations of the invention may also be used to treat disorders in which TNF-alpha activity is detrimental as described in U.S. Pat. Nos. 6,090,382, 6,258,562 and U.S. Patent Application Publication No. US20040126372, the entire contents of all of which are incorporated herein by reference.

The use of solid units containing anti-TNF-alpha antibodies in the treatment of specific exemplary disorders is discussed further below:

A. Sepsis

The solid units and methods of the invention may be used to treat subjects having sepsis. Tumor necrosis factor has an established role in the pathophysiology of sepsis, with biological effects that include hypotension, myocardial suppression, vascular leakage syndrome, organ necrosis, stimulation of the release of toxic secondary mediators and activation of the clotting cascade (see e.g., Tracey, K. J. and Cerami, A. (1994) Annu. Rev. Med. 45:491-503; Russell, D and Thompson, R. C. (1993) Curr. Opin. Biotech. 4:714-721). Accordingly, the formulation of the invention can be used to treat sepsis in any of its clinical settings, including septic shock, endotoxic shock, gram negative sepsis and toxic shock syndrome.

Furthermore, to treat sepsis, the solid units of the invention can be coadministered with one or more additional therapeutic agents that may further alleviate sepsis, such as an interleukin-1 inhibitor (such as those described in PCT Publication Nos. WO 92/16221 and WO 92/17583), the cytokine interleukin-6 (see e.g., PCT Publication No. WO 93/11793) or an antagonist of platelet activating factor (see e.g., European Patent Application Publication No. EP 374 510).

Additionally, in one embodiment, the solid unit of the invention is administered to a human subject within a subgroup of sepsis patients having a serum or plasma concentration of IL-6 above 500 μg/ml; or, in one embodiment, 1000 μg/ml, at the time of treatment (see PCT Publication No. WO 95/20978).

B. Autoimmune Diseases

The solid units and methods of the invention may be used to treat subjects having an autoimmune disease. Tumor necrosis factor has been implicated in playing a role in the pathophysiology of a variety of autoimmune diseases. For example, TNF-alpha has been implicated in activating tissue inflammation and causing joint destruction in rheumatoid arthritis (see e.g., Tracey and Cerami, supra; Arend, W. P. and Dayer, J-M. (1995) Arth. Rheum. 38:151-160; Fava, R. A., et al. (1993) Clin. Exp. Immunol. 94:261-266). TNF-alpha also has been implicated in promoting the death of islet cells and in mediating insulin resistance in diabetes (see e.g., Tracey and Cerami, supra; PCT Publication No. WO 94/08609). TNF-alpha also has been implicated in mediating cytotoxicity to oligodendrocytes and induction of inflammatory plaques in multiple sclerosis (see e.g., Tracey and Cerami, supra). Also included in autoimmune diseases that may be treated using the formulations and methods of the invention is juvenile idiopathic arthritis (JIA) (also referred to as juvenile rheumatoid arthritis) (see Grom et al. (1996) Arthritis Rheum. 39:1703; Mangge et al. (1995) Arthritis Rheum. 8:211).

The formulation of the invention can be used to treat autoimmune diseases, in particular those associated with inflammation, including rheumatoid arthritis, ankylosing spondylitis, osteoarthritis and gouty arthritis, allergy, multiple sclerosis, autoimmune diabetes, autoimmune uveitis, juvenile idiopathic arthritis (also referred to as juvenile rheumatoid arthritis), and nephrotic syndrome. Thus, in one embodiment, solid units (or a plurality of solid units) comprising a human anti-TNFα antibody, including adalimumab, or a biosimilar thereof, are administered to a subject to treat rheumatoid arthritis, ankylosing spondylitis, osteoarthritis and gouty arthritis, allergy, multiple sclerosis, autoimmune diabetes, autoimmune uveitis, juvenile idiopathic arthritis (also referred to as juvenile rheumatoid arthritis), and nephrotic syndrome.

C. Infectious Diseases

The solid units and methods of the invention may be used to treat subjects having an infectious disease. Tumor necrosis factor has been implicated in mediating biological effects observed in a variety of infectious diseases. For example, TNF-alpha has been implicated in mediating brain inflammation and capillary thrombosis and infarction in malaria (see e.g., Tracey and Cerami, supra). TNF-alpha also has been implicated in mediating brain inflammation, inducing breakdown of the blood-brain barrier, triggering septic shock syndrome and activating venous infarction in meningitis (see e.g., Tracey and Cerami, supra). TNF-alpha also has been implicated in inducing cachexia, stimulating viral proliferation and mediating central nervous system injury in acquired immune deficiency syndrome (AIDS) (see e.g., Tracey and Cerami, supra). Accordingly, the antibodies, and antibody portions, of the invention, can be used in the treatment of infectious diseases, including bacterial meningitis (see e.g., European Patent Application Publication No. EP 585 705), cerebral malaria, AIDS and AIDS-related complex (ARC) (see e.g., European Patent Application Publication No. EP 230 574), as well as cytomegalovirus infection secondary to transplantation (see e.g., Fietze, E., et al. (1994) Transplantation 58:675-680). The formulation of the invention, also can be used to alleviate symptoms associated with infectious diseases, including fever and myalgias due to infection (such as influenza) and cachexia secondary to infection (e.g., secondary to AIDS or ARC).

D. Transplantation

The solid units and methods of the invention may be used to treat subjects having a transplantation. Tumor necrosis factor has been implicated as a key mediator of allograft rejection and graft versus host disease (GVHD) and in mediating an adverse reaction that has been observed when the rat antibody OKT3, directed against the T cell receptor CD3 complex, is used to inhibit rejection of renal transplants (see e.g., Tracey and Cerami, supra; Eason, J. D., et al. (1995) Transplantation 59:300-305; Suthanthiran, M. and Strom, T. B. (1994) New Engl. J. Med. 331:365-375). Accordingly, the formulations of the invention can be used to inhibit transplant rejection, including rejections of allografts and xenografts and to inhibit GVHD. Although the antibody or antibody portion may be used alone, it can be used in combination with one or more other agents that inhibit the immune response against the allograft or inhibit GVHD. For example, in one embodiment, the formulations of the invention are used in combination with OKT3 to inhibit OKT3-induced reactions. In another embodiment, the formulation of the invention is used in combination with one or more antibodies directed at other targets involved in regulating immune responses, such as the cell surface molecules CD25 (interleukin-2 receptor-alpha), CD11a (LFA-1), CD54 (ICAM-1), CD4, CD45, CD28/CTLA4, CD80 (B7-1) and/or CD86 (B7-2). In yet another embodiment, the formulation of the invention is used in combination with one or more general immunosuppressive agents, such as cyclosporin A or FK506.

E. Malignancy

The solid units and methods of the invention may be used to treat subjects having cancer or a malignant tumor. Tumor necrosis factor has been implicated in inducing cachexia, stimulating tumor growth, enhancing metastatic potential and mediating cytotoxicity in malignancies (see e.g., Tracey and Cerami, supra). Accordingly, the formulations of the invention can be used in the treatment of malignancies, to inhibit tumor growth or metastasis and/or to alleviate cachexia secondary to malignancy. The formulation of the invention may be administered systemically or locally to the tumor site.

F. Pulmonary Disorders

The formulations and methods of the invention may be used to treat subjects having a pulmonary disease. Tumor necrosis factor has been implicated in the pathophysiology of adult respiratory distress syndrome, including stimulating leukocyte-endothelial activation, directing cytotoxicity to pneumocytes and inducing vascular leakage syndrome (see e.g., Tracey and Cerami, supra). Accordingly, the formulations of the invention can be used to treat various pulmonary disorders, including adult respiratory distress syndrome (see e.g., PCT Publication No. WO 91/04054), shock lung, chronic pulmonary inflammatory disease, pulmonary sarcoidosis, pulmonary fibrosis and silicosis. The formulation of the invention may be administered systemically or locally to the lung surface, for example as an aerosol.

G. Intestinal Disorders

The solid units and methods of the invention may be used to treat subjects having an intestinal disorder. Tumor necrosis factor has been implicated in the pathophysiology of inflammatory bowel disorders (see e.g., Tracy, K. J., et al. (1986) Science 234:470-474; Sun, X-M., et al. (1988) J. Clin. Invest. 81:1328-1331; MacDonald, T. T., et al. (1990) Clin. Exp. Immunol. 81:301-305). Chimeric murine anti-hTNF-alpha antibodies have undergone clinical testing for treatment of Crohn's disease (van Dullemen, H. M., et al. (1995) Gastroenterology 109:129-135). The formulation of the invention, also can be used to treat intestinal disorders, such as idiopathic inflammatory bowel disease, which includes two syndromes, Crohn's disease and ulcerative colitis. In one embodiment, the formulation of the invention is used to treat Crohn's disease. In one embodiment, the formulation of the invention is used to treat ulcerative colitis. Thus, in one embodiment, solid units (or a plurality of solid units) comprising a human anti-TNFα antibody, including adalimumab, or a biosimilar thereof, are administered to a subject to treat Crohn's disease or ulcerative colitis.

H. Cardiac Disorders

The formulations and methods of the invention, also can be used to treat various cardiac disorders, including ischemia of the heart (see e.g., European Patent Application Publication No. EP 453 898) and heart insufficiency (weakness of the heart muscle)(see e.g., PCT Publication No. WO 94/20139).

I. Spondyloarthropathies

The solid units and methods of the invention may also be used to treat subjects who have a spondyloarthropathy, including, for example, an axial spondyloarthropathy. TNFα has been implicated in the pathophysiology of a wide variety of disorders, including inflammatory diseases such as spondyloarthopathies (see e.g., Moeller, A., et al. (1990) Cytokine 2:162-169; U.S. Pat. No. 5,231,024 to Moeller et al.; European Patent Publication No. 260 610 B1 by Moeller, A). In one embodiment, the spondyloarthropathy is an axial spondyloarthropathy. Thus, in one embodiment, solid units (or a plurality of solid units) comprising a human anti-TNFα antibody, including adalimumab, or a biosimilar thereof, are administered to a subject to treat a spondyloarthropathy. Other examples of spondyloarthropathies which can be treated with the TNFα antibody of the invention are described below:

1. Psoriatic Arthritis

The solid units and methods of the invention may also be used to treat subjects who have psoriatic arthritis. Tumor necrosis factor has been implicated in the pathophysiology of psoriatic arthritis (Partsch et al. (1998) Ann Rheum Dis. 57:691; Ritchlin et al. (1998) J Rheumatol. 25:1544). As referred to herein, psoriatic arthritis (PsA) or psoriasis associated with the skin, refers to chronic inflammatory arthritis which is associated with psoriasis. Psoriasis is a common chronic skin condition that causes red patches on the body. About 1 in 20 individuals with psoriasis will develop arthritis along with the skin condition, and in about 75% of cases, psoriasis precedes the arthritis. PsA exhibits itself in a variety of ways, ranging from mild to severe arthritis, wherein the arthritis usually affects the fingers and the spine. When the spine is affected, the symptoms are similar to those of ankylosing spondylitis, as described above.

PsA is sometimes associated with arthritis mutilans. Arthritis mutilans refers to a disorder which is characterized by excessive bone erosion resulting in a gross, erosive deformity which mutilates the joint. In one embodiment, formulations and methods of the invention can be used to treat arthritis mutilans.

Thus, in one embodiment, solid units (or a plurality of solid units) comprising a human anti-TNFα antibody, including adalimumab, or a biosimilar thereof, are administered to a subject to treat psoriatic arthritis.

2. Reactive Arthritis/Reiter's Syndrome

The solid units and methods of the invention may also be used to treat subjects who have Reiter's syndrome or reactive arthritis. Tumor necrosis factor has been implicated in the pathophysiology of reactive arthritis, which is also referred to as Reiter's syndrome (Braun et al. (1999) Arthritis Rheum. 42(10):2039). Reactive arthritis (ReA) refers to arthritis which complicates an infection elsewhere in the body, often following enteric or urogenital infections. ReA is often characterized by certain clinical symptoms, including inflammation of the joints (arthritis), urethritis, conjunctivitis, and lesions of the skin and mucous membranes. In addition, ReA can occurs following infection with a sexually transmitted disease or dysenteric infection, including chlamydia, campylobacter, salmonella, or yersinia.

3. Undifferentiated Spondyloarthropathies

The solid units and methods of the invention may also be used to treat subjects who have an undifferentiated spondyloarthropathy (see Zeidler et al. (1992) Rheum Dis Clin North Am. 18:187). Other terms used to describe undifferentiated spondyloarthropathies include seronegative oligoarthritis and undifferentiated oligoarthritis. Undifferentiated spondyloarthropathies, as used herein, refers to a disorder wherein the subject demonstrates only some of the symptoms associated with a spondyloarthropathy. This condition is usually observed in young adults who do not have IBD, psoriasis, or the classic symptoms of AS or Reiter's syndrome. In some instances, undifferentiated spondyloarthropathies may be an early indication of AS.

The solid units of the invention may also be used to treat active axial spondyloarthritis (active axSpA) and non-radiographic axial spondyloarthritis (nr-axSpA). Thus, in one embodiment, solid units (or a plurality of solid units) comprising a human anti-TNFα antibody, including adalimumab, or a biosimilar thereof, may be administered to a subject to treat active axial spondyloarthritis (active axSpA) and non-radiographic axial spondyloarthritis (nr-axSpA).

J. Skin and Nail Disorders

In one embodiment, the solid units and methods of the invention are used to treat a skin and/or a nail disorder. As used herein, the term “skin and nail disorder in which TNFα activity is detrimental” is intended to include skin and/or nail disorders and other disorders in which the presence of TNF-alpha in a subject suffering from the disorder has been shown to be or is suspected of being either responsible for the pathophysiology of the disorder or a factor that contributes to a worsening of the disorder, e.g., psoriasis. An example of a skin disorder which may be treated using the formulation of the invention is psoriasis. In one embodiment, the solid units of the invention is used to treat plaque psoriasis. Tumor necrosis factor has been implicated in the pathophysiology of psoriasis (Takematsu et al. (1989) Arch Dermatol Res. 281:398; Victor and Gottlieb (2002) J Drugs Dermatol. 1(3):264).

1. Psoriasis

The solid units and methods of the invention may be used to treat subjects having psoriasis, including subjects having plaque psoriasis. Tumor necrosis factor has been implicated in the pathophysiology of psoriasis (Takematsu et al. (1989) Arch Dermatol Res. 281:398; Victor and Gottlieb (2002) J Drugs Dermatol. 1(3):264). Psoriasis is described as a skin inflammation (irritation and redness) characterized by frequent episodes of redness, itching, and thick, dry, silvery scales on the skin. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the epidermis and polymorphonuclear leukocyte and lymphocyte infiltration into the epidermis layer resulting in an increase in the basal cell cycle. Psoriasis often involves the nails, which frequently exhibit pitting, separation of the nail, thickening, and discoloration. Psoriasis is often associated with other inflammatory disorders, for example arthritis, including rheumatoid arthritis, inflammatory bowel disease (IBD), and Crohn's disease.

Evidence of psoriasis is most commonly seen on the trunk, elbows, knees, scalp, skin folds, or fingernails, but it may affect any or all parts of the skin. Normally, it takes about a month for new skin cells to move up from the lower layers to the surface. In psoriasis, this process takes only a few days, resulting in a build-up of dead skin cells and formation of thick scales. Symptoms of psoriasis include: skin patches, that are dry or red, covered with silvery scales, raised patches of skin, accompanied by red borders, that may crack and become painful, and that are usually located on the elbows, knees, trunk, scalp, and hands; skin lesions, including pustules, cracking of the skin, and skin redness; joint pain or aching which may be associated with of arthritis, e.g., psoriatic arthritis.

Treatment for psoriasis often includes a topical corticosteroids, vitamin D analogs, and topical or oral retinoids, or combinations thereof. In one embodiment, the TNF-alpha inhibitor of the invention is administered in combination with or the presence of one of these common treatments.

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

In one embodiment of the invention, a solid unit comprising an anti-TNF-alpha antibody is used to treat psoriasis, including chronic plaque psoriasis, guttate psoriasis, inverse psoriasis, pustular psoriasis, pemphigus vulgaris, erythrodermic psoriasis, psoriasis associated with inflammatory bowel disease (IBD), and psoriasis associated with rheumatoid arthritis (RA). Specific types of psoriasis included in the treatment methods of the invention are described in detail below:

a. Chronic Plaque Psoriasis

The solid units and methods of the invention may be used to treat subjects having chronic plaque psoriasis. Tumor necrosis factor has been implicated in the pathophysiology of chronic plaque psoriasis (Asadullah et al. (1999) Br J Dermatol. 141:94). Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis. Thus, in one embodiment, solid units (or a plurality of solid units) comprising a human anti-TNFα antibody, including adalimumab, or a biosimilar thereof, are administered to a subject to treat chronic plaque psoriasis.

b. Guttate Psoriasis

The solid units and methods of the invention may be used to treat subjects having guttate psoriasis. Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

c. Inverse Psoriasis

The solid units and methods of the invention may be used to treat subjects having inverse psoriasis. Inverse psoriasis is a form of psoriasis in which the patient has smooth, usually moist areas of skin that are red and inflamed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irritate the affected areas.

d. Pustular Psoriasis

The formulations and methods of the invention may be used to treat subjects having pustular psoriasis. Pustular psoriasis is a form of psoriasis that causes pus-filled blisters that vary in size and location, but often occur on the hands and feet. The blisters may be localized, or spread over large areas of the body. Pustular psoriasis can be both tender and painful, can cause fevers.

e. Other Psoriasis Disorders

Other examples of psoriatic disorders which can be treated with the formulations and methods of the invention include erythrodermic psoriasis, vulgaris, psoriasis associated with IBD, and psoriasis associated with arthritis, including rheumatoid arthritis.

2. Pemphigus Vulgaris

The solid units and methods of the invention may be used to treat subjects having pemphigus vulgaris. Pemphigus vulgaris is a serious autoimmune systemic dermatologic disease that often affects the oral mucous membrane and skin. The pathogenesis of pemphigus vulgaris is thought to be an autoimmune process that is directed at skin and oral mucous membrane desmosomes. Consequentially, cells do not adhere to each other. The disorder manifests as large fluid-filled, rupture-prone bullae, and has a distinctive histologic appearance. Anti-inflammatory agents are the only effective therapy for this disease which has a high mortality rate. Complications that arise in patients suffering from pemphigus vulgaris are intractable pain, interference with nutrition and fluid loss, and infections.

3. Atopic Dermatitis/Eczema

The solid units and methods of the invention may be used to treat subjects having atopic dermatitis. Atopic dermatitis (also referred to as eczema) is a chronic skin disorder categorized by scaly and itching plaques. People with eczema often have a family history of allergic conditions like asthma, hay fever, or eczema. Atopic dermatitis is a hypersensitivity reaction (similar to an allergy) which occurs in the skin, causing chronic inflammation. The inflammation causes the skin to become itchy and scaly. Chronic irritation and scratching can cause the skin to thicken and become leathery-textured. Exposure to environmental irritants can worsen symptoms, as can dryness of the skin, exposure to water, temperature changes, and stress.

Subjects with atopic dermatitis can be identified by certain symptoms, which often include intense itching, blisters with oozing and crusting, skin redness or inflammation around the blisters, rash, dry, leathery skin areas, raw areas of the skin from scratching, and ear discharges/bleeding.

4. Sarcoidosis

The solid units and methods of the invention may be used to treat subjects having sarcoidosis. Sarcoidosis is a disease in which granulomatous inflammation occurs in the lymph nodes, lungs, liver, eyes, skin, and/or other tissues. Sarcoidosis includes cutaneous sarcoidosis (sarcoidosis of the skin) and nodular sarcoidosis (sarcoidosis of the lymph nodes). Patients with sarcoidosis can be identified by the symptoms, which often include general discomfort, uneasiness, or an ill feeling; fever; skin lesions.

5. Erythema Nodosum

The solid units and methods of the invention may be used to treat subjects having erythema nodosum. Erythema nodosum refers to an inflammatory disorder that is characterized by tender, red nodules under the skin, typically on the anterior lower legs. Lesions associated with erythema nodosum often begin as flat, but firm, hot red painful lumps (approximately an inch across). Within a few days the lesions may become purplish, and then over several weeks fade to a brownish flat patch.

In some instances, erythema nodosum may be associated with infections including, streptococcus, coccidioidomycosis, tuberculosis, hepatitis B, syphilis, cat scratch disease, tularemia, yersinia, leptospirosis psittacosis, histoplasmosis, mononucleosis (EBV). In other instances, erythema nodosum may be associated with sensitivity to certain medications including, oral contraceptives, penicillin, sulfonamides, sulfones, barbiturates, hydantoin, phenacetin, salicylates, iodides, and progestin. Erythema nodosum is often associated with other disorders including, leukemia, sarcoidosis, rheumatic fever, and ulcerative colitis.

Symptoms of erythema nodosum usually present themselves on the shins, but lesions may also occur on other areas of the body, including the buttocks, calves, ankles, thighs and upper extremities. Other symptoms in subjects with erythema nodosum can include fever and malaise.

6. Hidradenitis Suppurativa

The solid units and methods of the invention may be used to treat subjects having hidradenitis suppurativa. Hidradenitis suppurativa refers to a skin disorder in which swollen, painful, inflamed lesions or lumps develop in the groin and sometimes under the arms and under the breasts. Hidradenitis suppurativa occurs when apocrine gland outlets become blocked by perspiration or are unable to drain normally because of incomplete gland development. Secretions trapped in the glands force perspiration and bacteria into surrounding tissue, causing subcutaneous induration, inflammation, and infection. Hidradenitis suppurativa is confined to areas of the body that contain apocrine glands. These areas are the axillae, areola of the nipple, groin, perineum, circumanal, and periumbilical regions. Thus, in one embodiment, solid units (or a plurality of solid units) comprising a human anti-TNFα antibody, including adalimumab, or a biosimilar thereof, may be administered to a subject to treat hidradenitis suppurativa.

7. Lichen Planus

The solid units and methods of the invention may be used to treat subjects having lichen planus. Tumor necrosis factor has been implicated in the pathophysiology of lichen planus (Sklavounou et al. (2000) J Oral Pathol Med. 29:370). Lichen planus refers to a disorder of the skin and the mucous membranes resulting in inflammation, itching, and distinctive skin lesions. Lichen planus may be associated with hepatitis C or certain medications.

8. Sweet's Syndrome

The formulations and methods of the invention may be used to treat subjects having Sweet's syndrome. Inflammatory cytokines, including tumor necrosis factor, have been implicated in the pathophysiology of Sweet's syndrome (Reuss-Borst et al. (1993) Br J Haematol. 84:356). Sweet's syndrome, which was described by R. D. Sweet in 1964, is characterized by the sudden onset of fever, leukocytosis., and cutaneous eruption. The eruption consists of tender, erythematous, well-demarcated papules and plaques which show dense neutrophilic infiltrates microscopically. The lesions may appear anywhere, but favor the upper body including the face. The individual lesions are often described as pseudovesicular or pseudopustular, but may be frankly pustular, bullous, or ulcerative. Oral and eye involvement (conjunctivitis or episcleritis) have also been frequently reported in patients with Sweet's syndrome. Leukemia has also been associated with Sweet's syndrome.

9. Vitiligo

The solid units and methods of the invention may be used to treat subjects having vitiligo. Vitiligo refers to a skin condition in which there is loss of pigment from areas of skin resulting in irregular white patches with normal skin texture. Lesions characteristic of vitiligo appear as flat depigmented areas. The edges of the lesions are sharply defined but irregular. Frequently affected areas in subjects with vitiligo include the face, elbows and knees, hands and feet, and genitalia.

10. Scleroderma

The solid units and methods of the invention may be used to treat subjects having scleroderma. Tumor necrosis factor has been implicated in the pathophysiology of scleroderma (Tutuncu Z et al. (2002) Clin Exp Rheumatol. 20(6 Suppl 28):S146-51; Mackiewicz Z et al. (2003) Clin Exp Rheumatol. 21(1):41-8; Murota H et al. (2003) Arthritis Rheum. 48(4):1117-25). Scleroderma refers to a diffuse connective tissue disease characterized by changes in the skin, blood vessels, skeletal muscles, and internal organs. Scleroderma is also referred to as CREST syndrome or Progressive systemic sclerosis, and usually affects people between the ages 30-50. Women are affected more often than men.

The cause of scleroderma is unknown. The disease may produce local or systemic symptoms. The course and severity of the disease varies widely in those affected. Excess collagen deposits in the skin and other organs produce the symptoms. Damage to small blood vessels within the skin and affected organs also occurs. In the skin, ulceration, calcification, and changes in pigmentation may occur. Systemic features may include fibrosis and degeneration of the heart, lungs, kidneys and gastrointestinal tract.

Patients suffering from scleroderma exhibit certain clinical features, including, blanching, blueness, or redness of fingers and toes in response to heat and cold (Raynaud's phenomenon), pain, stiffness, and swelling of fingers and joints, skin thickening and shiny hands and forearm, esophageal reflux or heartburn, difficulty swallowing, and shortness of breath. Other clinical symptoms used to diagnose scleroderma include, an elevated erythrocyte sedimentation rate (ESR), an elevated rheumatoid factor (RF), a positive antinuclear antibody test, urinalysis that shows protein and microscopic blood, a chest X-ray that may show fibrosis, and pulmonary function studies that show restrictive lung disease.

11. Nail Disorders

The solid units and methods of the invention may be used to treat subjects having a nail disorder. Nail disorders include any abnormality of the nail. Specific nail disorders include, but are not limited to, pitting, koilonychia, Beau's lines, spoon nails, onycholysis, yellow nails, pterygium (seen in lichen planus), and leukonychia. Pitting is characterized by the presence of small depressions on the nail surface. Ridges or linear elevations can develop along the nail occurring in a “lengthwise” or “crosswise” direction. Beau's lines are linear depressions that occur “crosswise” (transverse) in the fingernail. Leukonychia describes white streaks or spots on the nails. Koilonychia is an abnormal shape of the fingernail where the nail has raised ridges and is thin and concave Koilonychia is often associated with iron deficiency.

Nail disorders which can be treated with the anti-TNF-alpha antibody of the invention also include psoriatic nails. Psoriatic nails include changes in nails which are attributable to psoriasis. In some instances psoriasis may occur only in the nails and nowhere else on the body. Psoriatic changes in nails range from mild to severe, generally reflecting the extent of psoriatic involvement of the nail plate, nail matrix, i.e., tissue from which the nail grows, nail bed, i.e., tissue under the nail, and skin at the base of the nail. Damage to the nail bed by the pustular type of psoriasis can result in loss of the nail. Nail changes in psoriasis fall into general categories that may occur singly or all together. In one category of psoriatic nails, the nail plate is deeply pitted, probably due to defects in nail growth caused by psoriasis. IN another category, the nail has a yellow to yellow-pink discoloration, probably due to psoriatic involvement of the nail bed. A third subtype of psoriatic nails are characterized by white areas which appear under the nail plate. The white areas are actually air bubbles marking spots where the nail plate is becoming detached from the nail bed. There may also be reddened skin around the nail. A fourth category is evidenced by the nail plate crumbling in yellowish patches, i.e., onychodystrophy, probably due to psoriatic involvement in the nail matrix. A fifth category is characterized by the loss of the nail in its entirety due to psoriatic involvement of the nail matrix and nail bed.

The solid units and methods of the invention may also be used to treat nail disorders often associated with lichen planus. Nails in subjects with lichen planus often show thinning and surface roughness of the nail plate with longitudinal ridges or pterygium.

The solid units and methods of the invention may be used to treat nail disorders, such as those described herein. Often nail disorders are associated with skin disorders. In one embodiment, the invention includes a method of treatment for nail disorders with an anti-TNF-alpha antibody. In another embodiment, the nail disorder is associated with another disorder, including a skin disorder such as psoriasis. In another embodiment, the disorder associated with a nail disorder is arthritis, including psoriatic arthritis.

12. Other Skin and Nail Disorders

The solid units and methods of the invention may be used to treat other skin and nail disorders, such as chronic actinic dermatitis, bullous pemphigoid, and alopecia areata. Chronic actinic dermatitis (CAD) is also referred to as photosensitivity dermatitis/actinic reticuloid syndrome (PD/AR). CAD is a condition in which the skin becomes inflamed, particularly in areas that have been exposed to sunlight or artificial light. Commonly, CAD patients have allergies to certain substances that come into contact with their skin, particularly various flowers, woods, perfumes, sunscreens and rubber compounds. Bullous pemphigoid refers to A skin disorder characterized by the formation of large blisters on the trunk and extremities. Alopecia areata refers to hair loss characterized by round patches of complete baldness in the scalp or beard.

K. Metabolic Disorders

The solid units and methods of the invention may be used to treat a metabolic disease. TNFα has been implicated in the pathophysiology of a wide variety of disorders, including metabolic disorders, such as diabetes and obesity (Spiegelman and Hotamisligil (1993) Cell 73:625; Chu et al. (2000) Int J Obes Relat Metab Disord. 24:1085; Ishii et al. (2000) Metabolism. 49:1616).

Metabolic disorders affect how the body processes substances needed to carry out physiological functions. A number of metabolic disorders of the invention share certain characteristics, i.e. they are associated the insulin resistance, lack of ability to regulate blood sugar, weight gain, and increase in body mass index. Examples of metabolic disorders include diabetes and obesity. Examples of diabetes include type 1 diabetes mellitus, type 2 diabetes mellitus, diabetic neuropathy, peripheral neuropathy, diabetic retinopathy, diabetic ulcerations, retinopathy ulcerations, diabetic macrovasculopathy, and obesity. Examples of metabolic disorders which can be treated with the formulations and methods of the invention are described in more detail below:

1. Diabetes

The solid units and methods of the invention may be used to treat diabetes. Tumor necrosis factor has been implicated in the pathophysiology of diabetes. (see e.g., Navarro J. F., Mora C., Maca, Am J Kidney Dis. 2003 July; 42(1):53-61; Daimon M et al., Diabetes Care. 2003 July; 26(7):2015-20; Zhang M et al., J Tongji Med Univ. 1999; 19(3):203-5, Barbieri M et al., Am J Hypertens. 2003 July; 16(7):537-43.) For example, TNFα is implicated in the pathophysiology for insulin resistance. It has been found that serum TNF levels in patients with gastrointestinal cancer correlates with insulin resistance (see e.g., McCall, J. et al. Br. J. Surg. 1992; 79: 1361-3).

Diabetes includes the two most common types of the disorder, namely type I diabetes and type II diabetes, which both result from the body's inability to regulate insulin. Insulin is a hormone released by the pancreas in response to increased levels of blood sugar (glucose) in the blood.

The term “type 1 diabetes,” as used herein, refers to a chronic disease that occurs when the pancreas produces too little insulin to regulate blood sugar levels appropriately. Type 1 diabetes is also referred to as insulin-dependent diabetes mellitus, IDMM, juvenile onset diabetes, and diabetes-type I. Type 1 diabetes represents is the result of a progressive autoimmune destruction of the pancreatic β-cells with subsequent insulin deficiency.

The term “type 2 diabetes,” refers to a chronic disease that occurs when the pancreas does not make enough insulin to keep blood glucose levels normal, often because the body does not respond well to the insulin. Type 2 diabetes is also referred to as noninsulin-dependent diabetes mellitus, NDDM, and diabetes-type II

Diabetes is can be diagnosed by the administration of a glucose tolerance test. Clinically, diabetes is often divided into several basic categories. Primary examples of these categories include, autoimmune diabetes mellitus, non-insulin-dependent diabetes mellitus (type 1 NDDM), insulin-dependent diabetes mellitus (type 2 IDDM), non-autoimmune diabetes mellitus, non-insulin-dependent diabetes mellitus (type 2 NIDDM), and maturity-onset diabetes of the young (MODY). A further category, often referred to as secondary, refers to diabetes brought about by some identifiable condition which causes or allows a diabetic syndrome to develop. Examples of secondary categories include, diabetes caused by pancreatic disease, hormonal abnormalities, drug- or chemical-induced diabetes, diabetes caused by insulin receptor abnormalities, diabetes associated with genetic syndromes, and diabetes of other causes. (see e.g., Harrison's (1996) 14^{th} ed., New York, McGraw-Hill).

Diabetes manifests itself in the foregoing categories and can cause several complications that are discussed in the following sections. Accordingly, the antibody, or antigen-binding fragment thereof, of the invention can be used to treat diabetes. In one embodiment, the TNFα antibody, or antigen-binding fragment thereof, of the invention is used to treat diabetes associated with the above identified categories.

Diabetes is often treated with diet, insulin dosages, and various medications described herein. Accordingly, the formulations of the invention may also be administered in combination with agents commonly used to treat metabolic disorders and pain commonly associated with diabetes.

Diabetes manifests itself in many complications and conditions associated with diabetes, including the following categories:

a. Diabetic Neuropathy and Peripheral Neuropathy

The solid units and methods of the invention may be used to treat diabetic neuropathy or peripheral neuropathy. Tumor necrosis factor has been implicated in the pathophysiology of diabetic neuropathy and peripheral neuropathy. (See Benjafield et al. (2001) Diabetes Care. 24:753; Qiang, X. et al. (1998) Diabetologia. 41:1321-6; Pfeiffer et al. (1997) Horm Metab Res. 29:111).

The term “neuropathy,” also referred to as nerve damage-diabetic, as used herein, refers to a common complication of diabetes in which nerves are damaged as a result of hyperglycemia (high blood sugar levels). A variety of diabetic neuropathies are recognized, such as distal sensorimotor polyneuropathy, focal motor neuropathy, and autonomic neuropathy.

The term “peripheral neuropathy,” also known as peripheral neuritis and diabetic neuropathy, as used herein, refers to the failure of the nerves to carry information to and from the brain and spinal cord. Peripheral neuropathy produces symptoms such as pain, loss of sensation, and the inability to control muscles. In some cases, the failure of nerves to control blood vessels, intestinal function, and other organs results in abnormal blood pressure, digestion, and loss of other basic involuntary processes. Peripheral neuropathy may involve damage to a single nerve or nerve group (mononeuropathy) or may affect multiple nerves (polyneuropathy).

Neuropathies that affect small myelinated and unmyelinated fibers of the sympathetic and parasympathetic nerves are known as “peripheral neuropathies.” Furthermore, the related disorder of peripheral neuropathy, also known as peripheral neuritis and diabetic neuropathy, refers to the failure of the nerves to carry information to and from the brain and spinal cord. This produces symptoms such as pain, loss of sensation, and the inability to control muscles. In some cases, failure of nerves controlling blood vessels, intestinal function, and other organs results in abnormal blood pressure, digestion, and loss of other basic involuntary processes. Peripheral neuropathy may involve damage to a single nerve or nerve group (mononeuropathy) or may affect multiple nerves (polyneuropathy).

The term “diabetic neuropathy” refers to a common complication of diabetes (see www.nlm.nih.gov/medlineplus/ency/article/001214.htm) in which nerves are damaged as a result of hyperglycemia (high blood sugar levels). Diabetic neuropathy is also referred to as neuropathy and nerve damage-diabetic. A variety of diabetic neuropathies are recognized, such as distal sensorimotor polyneuropathy, focal motor neuropathy, and autonomic neuropathy.

b. Diabetic Retinopathy

The solid units and methods of the invention may be used to treat diabetic retinopathy. Tumor necrosis factor has been implicated in the pathophysiology of diabetic retinopathy (Scholz et al. (2003) Trends Microbiol. 11:171). The term “diabetic retinopathy” as used herein, refers to progressive damage to the eye's retina caused by long-term diabetes. Diabetic retinopathy, includes proliferative retinopathy. Proliferative neuropathy in turn includes neovascularization, pertinal hemmorrhage and retinal detachment.

In advanced retinopathy, small vessels proliferate on the surface of the retina. These blood vessels are fragile, tend to bleed and can cause peretinal hemorrhages. The hemorrhage can obscure vision, and as the hemorrhage is resorbed fibrous tissue forms predisposing to retinal detachments and loss of vision. In addition, diabetic retinopathy includes proliferative retinopathy which includes neovascularization, pertinal hemmorrhave and retinal detachment. Diabetic retinopathy also includes “background retinopathy” which involves changes occurring with the layers of the retina.

c. Diabetic Ulcerations and Retinopathy Ulcerations

The solid units and methods of the invention may be used to treat diabetic ulcerations or retinopathy ulcerations. Tumor necrosis factor has been implicated in the pathophysiology of diabetic ulcerations, (see Lee et al. (2003) Hum Immunol. 64:614; Navarro et al. (2003) Am J Kidney Dis. 42:53; Daimon et al (2003) Diabetes Care. 26:2015; Zhang et al. (1999) J Tongji Med Univ. 19:203; Barbieri et al. (2003) Am J Hypertens. 16:537; Venn et al. (1993) Arthritis Rheum. 36:819; Westacott et al. (1994) J Rheumatol. 21:1710).

The term “diabetic ulcerations,” as used herein, refers to an ulcer which results as a complication of diabetes. An ulcer is a crater-like lesion on the skin or mucous membrane caused by an inflammatory, infectious, malignant condition, or metabolic disorder. Typically diabetic ulcers can be found on limbs and extremities, more typically the feet. These ulcers, caused by diabetic conditions, such as neuropathy and a vascular insufficiency, can lead to ischemia and poor wound healing. More extensive ulcerations may progress to osteomyelitis. Once osteomyelitis develops, it may be difficult to eradicate with antibiotics alone, and amputation maybe necessary.

The term “retinopathy ulcerations,” as used herein refers to an ulcer which causes or results in damages to the eye and the eye's retina. Retinopathy ulcerations may include conditions such as retinopathic hemorrhages.

d. Diabetic Macrovasculopathy

The solid units and methods of the invention may be used to treat diabetic macrovasculopathy. Tumor necrosis factor has been implicated in the pathophysiology of diabetic macrovasculopathy (Devaraj et al. (2000) Circulation. 102:191; Hattori Y et al. (2000) Cardiovasc Res. 46:188; Clausell N et al. (1999) Cardiovasc Pathol. 8:145). The term “diabetic macrovasculopathy,” also referred to as “macrovascular disease,” as used herein, refers to a disease of the blood vessels that results from diabetes. Diabetic macrovasculopathy complication occurs when, for example, fat and blood clots build up in the large blood vessels and stick to the vessel walls. Diabetic macrovasculopathies include diseases such as coronary disease, cerebrovascular disease, and peripheral vascular disease, hyperglycaemia and cardiovascular disease, and strokes.

2. Obesity

The formulations and methods of the invention may be used to treat obesity. Tumor necrosis factor has been implicated in the pathophysiology of obesity (see e.g., Pihlajamaki J et al. (2003) Obes Res. 11:912; Barbieri et al. (2003) Am J Hypertens. 16:537; Tsuda et al. (2003) J Nutr. 133:2125). Obesity increases a person's risk of illness and death due to diabetes, stroke, coronary artery disease, hypertension, high cholesterol, and kidney and gallbladder disorders. Obesity may also increase the risk for some types of cancer, and may be a risk factor for the development of osteoarthritis and sleep apnea. Obesity can be treated with the antibody of the invention alone or in combination with other metabolic disorders, including diabetes.

L. Vasculitides

The solid units and methods of the invention may be used to treat a subject having a vasculitis. TNFα has been implicated in the pathophysiology of a variety of vasculitides, (see e.g., Deguchi et al. (1989) Lancet. 2:745). As used herein, the term “a vasculitis in which TNFα activity is detrimental” is intended to include vasculitis in which the presence of TNFα in a subject suffering from the disorder has been shown to be or is suspected of being either responsible for the pathophysiology of the disorder or a factor that contributes to a worsening of the disorder. Such disorders may be evidenced, for example, by an increase in the concentration of TNFα in a biological fluid of a subject suffering from the disorder (e.g., an increase in the concentration of TNFα in serum, plasma, synovial fluid, etc. of the subject), which can be detected, for example, using an anti-TNFα antibody as described above.

There are numerous examples of vasculitides in which TNFα activity is detrimental, including Behcet's disease. The use of the formulations and methods of the invention in the treatment of specific vasculitides are discussed further below. In certain embodiments, the antibody, or antibody portion, is administered to the subject in combination with another therapeutic agent, as described below

The solid units and methods of the invention be used to treat vasculitis in which TNFα activity is detrimental, wherein inhibition of TNFα activity is expected to alleviate the symptoms and/or progression of the vasculitis or to prevent the vasculitis. Subjects suffering from or at risk of developing vasculitis can be identified through clinical symptoms and tests. For example, subjects with vasculitides often develop antibodies to certain proteins in the cytoplasm of neutrophils, antineutrophil cytoplasmic antibodies (ANCA). Thus, in some instances, vasculitides may be evidenced by tests (e.g., ELISA), which measure ANCA presence.

Vasculitis and its consequences may be the sole manifestation of disease or it may be a secondary component of another primary disease. Vasculitis may be confined to a single organ or it may simultaneously affect several organs. and depending on the syndrome, arteries and veins of all sizes can be affected. Vasculitis can affect any organ in the body.

In vasculitis, the vessel lumen is usually compromised, which is associated with ischemia of the tissues supplied by the involved vessel. The broad range of disorders that may result from this process is due to the fact that any type, size and location of vessel (e.g., artery, vein, arteriole, venule, capillary) can be involved. Vasculitides are generally classified according to the size of the affected vessels, as described below. It should be noted that some small and large vessel vasculitides may involve medium-sized arteries; but large and medium-sized vessel vasculitides do not involve vessels smaller than arteries. Large vessel disease includes, but is not limited to, giant cell arteritis, also known as temporal arteritis or cranial arteritis, polymyalgia rheumatica, and Takayasu's disease or arteritis, which is also known as aortic arch syndrome, young female arteritis and Pulseless disease. Medium vessel disease includes, but is not limited to, classic polyarteritis nodosa and Kawasaki's disease, also known as mucocutaneous lymph node syndrome. Non-limiting examples of small vessel disease are Behcet's Syndrome, Wegner's granulomatosis, microscopic polyangitis, hypersensitivity vasculitis, also known as cutaneous vasculitis, small vessel vasculitis, Henoch-Schonlein purpura, allergic granulamotosis and vasculitis, also known as Churg Strauss syndrome. Other vasculitides include, but are not limited to, isolated central nervous system vasculitis, and thromboangitis obliterans, also known as Buerger's disease. Classic Polyarteritis nodosa (PAN), microscopic PAN, and allergic granulomatosis are also often grouped together and are called the systemic necrotizing vasculitides. A further description of vasculitis is described below:

1. Large Vessel Vasculitis

In one embodiment, the solid units and methods of the invention are used to treat subjects who have large vessel vasculitis. The term “large vessel(s)” as used herein, refers to the aorta and the largest branches directed toward major body regions. Large vessels include, for example, the aorta, and its branches and corresponding veins, e.g., the subclavian artery; the brachiocephalic artery; the common carotid artery; the innonimate vein; internal and external jugular veins; the pulmonary arteries and veins; the venae cavae; the renal arteries and veins; the femoral arteries and veins; and the carotid arteries. Examples of large vessel vasculitides are described below.

a. Giant Cell Arteritis (GCA)

The solid units and methods of the invention may be used to treat giant cell arteritis. Tumor necrosis factor has been implicated in the pathophysiology of giant cell arteritis (Sneller, M. C. (2002) Cleve. Clin. J. Med. 69:SII40-3; Schett, G., et al. (2002) Ann. Rheum. Dis. 61:463). Giant cell arteritis (GCA), refers to a vasculitis involving inflammation and damage to blood vessels, particularly the large or medium arteries that branch from the external carotid artery of the neck. GCA is also referred to as temporal arteritis or cranial arteritis, and is the most common primary vasculitis in the elderly. It almost exclusively affects individuals over 50 years of age, however, there are well-documented cases of patients 40 years and younger. GCA usually affects extracranial arteries. GCA can affect the branches of the carotid arteries, including the temporal artery. GCA is also a systemic disease which can involve arteries in multiple locations.

Histopathologically, GCA is a panarteritis with inflammatory mononuclear cell infiltrates within the vessel wall with frequent Langhans type giant cell formation. There is proliferation of the intima, granulomatous inflammation and fragmentation of the internal elastic lamina. The pathological findings in organs is the result of ischemia related to the involved vessels.

Patients suffering from GCA exhibit certain clinical symptoms, including fever, headache, anemia and high erythrocyte sedimentation rate (ESR). Other typical indications of GCA include jaw or tongue claudication, scalp tenderness, constitutional symptoms, pale optic disc edema (particularly ‘chalky white’ disc edema), and vision disturbances. The diagnosis is confirmed by temporal artery biopsy.

b. Polymyalgia Rheumatica

The solid units and methods of the invention may be used to treat polymyalgia rheumatica. Tumor necrosis factor has been implicated in the pathophysiology of polymyalgia rheumatica (Straub, R. H., et al. (2002) Rheumatology (Oxford) 41:423; Uddhammar, A., et al. (1998) Br. J. Rheumatol. 37:766). Polymyalgia rheumatica refers to a rheumatic disorder that is associated with moderate to severe muscle pain and stiffness in the neck, shoulder, and hip, most noticeable in the morning. IL-6 and IL-1β expression has also been detected in a majority of the circulating monocytes in patients with the polymyalgia rheumatica. Polymyalgia rheumatica may occur independently, or it may coexist with or precede GCA, which is an inflammation of blood vessels.

c. Takayasu's Arteritis

The solid units and methods of the invention may be used to treat Takayasu's arteritis. Tumor necrosis factor has been implicated in the pathophysiology of Takayasu's arteritis (Kobayashi, Y. and Numano, F. (2002) Intern. Med. 41:44; Fraga, A. and Medina F. (2002) Curr. Rheumatol. Rep. 0.4:30). Takayasu's arteritis refers to a vasculitis characterized by an inflammation of the aorta and its major branches. Takayasu's arteritis (also known as Aortic arch syndrome, young female arteritis and Pulseless disease) affects the thoracic and abdominal aorta and its main branches or the pulmonary arteries. Fibrotic thickening of the aortic wall and its branches (e.g., carotid, inominate, and subclavian arteries) can lead to reduction of lumen size of vessels that arise from the aortic arch. This condition also typically affects the renal arteries.

Takayasu's arteritis primarily affects young women, usually aged 20-40 years old, particularly of Asian descent, and may be manifested by malaise, arthralgias and the gradual onset of extremity claudication. Most patients have asymmetrically reduced pulses, usually along with a blood pressure differential in the arms. Coronary and/or renal artery stenosis may occur.

The clinical features of Takayasu's arteritis may be divided into the features of the early inflammatory disease and the features of the later disease. The clinical features of the early inflammatory stage of Takayasu's disease are: malaise, low grade fever, weight loss, myalgia, arthralgia, and erythema multiforme. Later stages of Takayasu's disease are characterized by fibrotic stenosis of arteries and thrombosis. The main resulting clinical features are ischemic phenomena, e.g. weak and asymmetrical arterial pulses, blood pressure discrepancy between the arms, visual disturbance, e.g. scotomata and hemianopia, other neurological features including vertigo and syncope, hemiparesis or stroke. The clinical features result from ischaemia due to arterial stenosis and thrombosis.

2. Medium Vessel Disease

The solid units and methods of the invention may be used to treat subjects who have medium vessel vasculitis. The term “medium vessel(s)” is used to refer to those blood vessels which are the main visceral arteries. Examples of medium vessels include the mesenteric arteries and veins, the iliac arteries and veins, and the maxillary arteries and veins. Examples of medium vessel vasculitides are described below.

a. Polyarteritis Nodosa

The solid units and methods of the invention may be used to treat polyarteritis nodosa. Tumor necrosis factor has been implicated in the pathophysiology of polyarteritis nodosa (DiGirolamo, N., et al. (1997) J. Leukoc. Biol. 61:667). Polyarteritis nodosa, or periarteritis nodosa refers to vasculitis which is a serious blood vessel disease in which small and medium-sized arteries become swollen and damaged because they are attacked by rogue immune cells. Polyarteritis nodosa usually affects adults more frequently than children. It damages the tissues supplied by the affected arteries because they don't receive enough oxygen and nourishment without a proper blood supply.

Symptoms which are exhibited in patients with polyarteritis nodosa generally result from damage to affected organs, often the skin, heart, kidneys, and nervous system. Generalized symptoms of polyarteritis nodosa include fever, fatigue, weakness, loss of appetite, and weight loss. Muscle aches (myalgia) and joint aches (arthralgia) are common. The skin of subjects with polyarteritis nodosa may also show rashes, swelling, ulcers, and lumps (nodular lesions).

Classic PAN (polyarteritis nodosa) is a systemic arteritis of small to medium muscular arteritis in which involvement of renal and visceral arteries is common. Abdominal vessels have aneurysms or occlusions in 50% of PAN patients. Classic PAN does not involve the pulmonary arteries although the bronchial vessels may be involved. Granulomas, significant eosinophilia and an allergic diathesis are not part of the syndrome. Although any organ system may be involved, the most common manifestations include peripheral neuropathy, mononeuritis multiplex, intestinal ischemia, renal ischemia, testicular pain and livedo reticularis.

b. Kawasaki's Disease

The solid units and methods of the invention may be used to treat Kawasaki's disease. Tumor necrosis factor has been implicated in the pathophysiology of Kawasaki's disease (Sundel, R. P. (2002) Curr. Rheumatol. Rep. 4:474; Gedalia, A. (2002) Curr. Rheumatol. Rep. 4:25). Although the cause of Kawasaki's disease is unknown, it is associated with acute inflammation of the coronary arteries, suggesting that the tissue damage associated with this disease may be mediated by proinflammatory agents such as TNFα. Kawasaki's disease refers to a vasculitis that affects the mucus membranes, lymph nodes, lining of the blood vessels, and the heart. Kawasaki's disease is also often referred to as mucocutaneous lymph node syndrome, mucocutaneous lymph node disease, and infantile polyarteritis. Subjects afflicted with Kawasaki's disease develop vasculitis often involving the coronary arteries which can lead to myocarditis and pericarditis. Often as the acute inflammation diminishes, the coronary arteries may develop aneurysm, thrombosis, and lead to myocardial infarction.

Kawasaki's disease is a febrile systemic vasculitis associated with edema in the palms and the soles of the feet, with enlargement of cervical lymph nodes, cracked lips and “strawberry tongue”. Although the inflammatory response is found in vessels throughout the body, the most common site of end-organ damage is the coronary arteries. Kawasaki's Disease predominantly affects children under the age of 5. The highest incidence is in Japan but is becoming increasingly recognized in the West and is now the leading cause of acquired heart disease in US children. The most serious complication of Kawasaki disease is coronary arteritis and aneurysm formation that occurs in a third of untreated patients. Thus, in one embodiment, solid units (or a plurality of solid units) comprising a human anti-TNFα antibody, including adalimumab, or a biosimilar thereof, are administered to a subject to treat Kawasaki's Disease.

3. Small Vessel Disease

The solid units and methods of the invention may be used to treat small vessel disease. In one embodiment, the TNFα antibody of the invention is used to treat subjects who have small vessel vasculitis. The term “small vessel(s)” is used to refer to arterioles, venules and capillaries. Arterioles are arteries that contain only 1 or 2 layers of smooth muscle cells and are terminal to and continuous with the capillary network. Venules carry blood from the capillary network to veins and capillaries connect arterioles and venules. Examples of small vessel vasculitides are described below.

a. Behcet's Disease

The solid units and methods of the invention may be used to treat Behcet's disease. Tumor necrosis factor has been implicated in the pathophysiology of Behcet's disease (Sfikakis, P. P. (2002) Ann. Rheum. Dis. 61:ii51-3; Dogan, D. and Farah, C. (2002) Oftalmologia. 52:23). Behcet's disease is a chronic disorder that involves inflammation of blood vessels throughout the body. Behcet's disease may also cause various types of skin lesions, arthritis, bowel inflammation, and meningitis (inflammation of the membranes of the brain and spinal cord). As a result of Behcet's disease, the subject with the disorder may have inflammation in tissues and organs throughout the body, including the gastrointestinal tract, central nervous system, vascular system, lungs, and kidneys. Behcet's disease is three times more common in males than females and is more common in the east Mediterranean and Japan. Thus, in one embodiment, solid units (or a plurality of solid units) comprising a human anti-TNFα antibody, including adalimumab, or a biosimilar thereof, are administered to a subject to treat Behcet's disease.

b. Wegener's Granulomatosis

The solid units and methods of the invention may be used to treat Wegener's granulomatosis. Tumor necrosis factor has been implicated in the pathophysiology of Wegener's granulomatosis (Marquez, J., et al. (2003) Curr. Rheumatol. Rep. 5:128; Harman, L. E. and Margo, C. E. (1998) Surv. Ophthalmol. 42:458). Wegener's granulomatosis refers to a vasculitis that causes inflammation of blood vessels in the upper respiratory tract (nose, sinuses, ears), lungs, and kidneys. Wegener's granulomatosis is also referred to as midline granulomatosis. Wegener's granulomatosis includes a granulomatous inflammation involving the respiratory tract, and necrotizing vasculitis affecting small to medium-sized vessels. Subjects who have Wegener's granulomatosis often also have arthritis (joint inflammation). Glomerulonephritis may also be present in affected subjects, but virtually any organ may be involved.

c. Churg-Strauss Syndrome

The solid units and methods of the invention may be used to treat Churg-Strauss syndrome. Tumor necrosis factor has been implicated in the pathophysiology of Churg-Strauss syndrome (Gross, W. L (2002) Curr. Opin. Rheumatol. 14:11; Churg, W. A. (2001) Mod. Pathol. 14:1284). Churg-Strauss syndrome refers to a vasculitis that is systemic and shows early manifestation signs of asthma and eosinophilia. Churg-Strauss syndrome is also referred to as allergic granulomatosis and angiitis, and occurs in the setting of allergic rhinitis, asthma and eosinophilia. Sinusitis and pulmonary infiltrates also occur in Churg-Strauss syndrome, primarily affecting the lung and heart. Peripheral neuropathy, coronary arteritis and gastrointestinal involvement are common.

M. Other Diseases

The solid units and methods of the invention may be used to treat various other disorders in which TNF-alpha activity is detrimental. Examples of other diseases and disorders in which TNF-alpha activity has been implicated in the pathophysiology, and thus which can be treated using an antibody, or antibody portion, of the invention, include inflammatory bone disorders and bone resorption disease (see e.g., O. D. R., et al. (1986) Nature 319:516-518; Konig, A. et al. (1988) J. Bone Miner. Res. 3:621-627; Lerner, U. H. and Ohlin, A. (1993) J. Bone Miner. Res. 8:147-155; and Shanlar, G. and Stem, P. H. (1993) Bone 14:871-876), hepatitis, including alcoholic hepatitis (see e g., McClain, C. J. and Cohen, D. A. (1989) Hepatology 9:349-351; Felver, M. E., el al. (1990) Alcohol. Clin. Exp. Res. 14:255-259; and Hansen, J., el al. (1994) Hepatology 20:461-474), viral hepatitis (Sheron, N., et al. (1991) J. Hepatol. 12:241-245; and Hussain, M. J., et al. (1994) J. Clin. Pathol. 47:1112-1115), and fulminant hepatitis; coagulation disturbances (see e.g., van der Poll, T., el al. (1990) N. Engl. J. Med. 322:1622-1627; and van der Poll, T., et al. (1991) Prog. Clin. Biol. Res. 367:55-60), burns (see e.g., Giroir, B. P., el al. (1994) Am. J. Physiol. 267:H 118-124; and Liu. X. S., el al. (1994) Burns 20:40-44), reperfusion injury (see e.g., Scales. W. E., et al. (1994) Am. J Physiol. 267:G1122-1127; Serrick, C., el al. (1994) Transplantation 58:1158-1162; and Yao, Y. M., et al. (1995) Resuscitation 29:157-168), keloid formation (see e.g., McCauley, R. L., et al. (1992) J. Clin. Immunol. 12:300-308), scar tissue formation; pyrexia; periodontal disease; obesity and radiation toxicity.

Examples of other disorders that may be treated with the formulations and methods of the invention are described in US20040126372 and U.S. Pat. No. 6,258,562, each of which is incorporated by reference herein.

In one embodiment, the solid units and methods of the invention are used to treat rheumatoid arthritis, psoriatic arthritis, or ankylosing spondylitis. The solid units of the invention comprising an isolated human anti-TNF-alpha antibody, or antigen-binding portion thereof, (e.g., adalimumab), may be administered to a human subject according to a dosing scheme and dose amount effective for treating rheumatoid arthritis, psoriatic arthritis, or ankylosing spondylitis. In one embodiment, a dose of about 40 mg of a human anti-TNF-alpha antibody, or antigen-binding portion thereof, (e.g., adalimumab) (e.g., 0.4 mL of a 100 mg/mL solution) in the solid unit(s) of the invention is administered to a human subject every other week for the treatment of rheumatoid arthritis, psoriatic arthritis, or ankylosing spondylitis. In one embodiment, a dose of about 80 mg of a human anti-TNF-alpha antibody, or antigen-binding portion thereof, (e.g., adalimumab) (e.g., 0.8 mL of a 100 mg/mL solution) in the solid unit(s) of the invention is administered to a human subject monthly for the treatment of rheumatoid arthritis, psoriatic arthritis, or ankylosing spondylitis. In one embodiment, the solid unit(s) is administered subcutaneously, every other week (also referred to as biweekly, see methods of administration described in US20030235585, incorporated by reference herein) for the treatment of rheumatoid arthritis, ankylosing spondylitis, or psoriatic arthritis. In one embodiment, the solid unit(s) is administered subcutaneously, monthly for the treatment of rheumatoid arthritis, ankylosing spondylitis, or psoriatic arthritis.

In one embodiment, the solid unit(s) of the invention is used to treat Crohn's disease or ulcerative colitis. The solid unit(s) of the invention comprising an isolated human anti-TNF-alpha antibody, or antigen-binding portion thereof, (e.g., adalimumab), may be administered to a human subject according to a dosing scheme and dose amount effective for treating Crohn's disease. In one embodiment, a dose of about 160 mg of a human anti-TNF-alpha antibody, or antigen-binding portion thereof, (e.g., adalimumab) (e.g., 1.6 mL of a 100 mg/mL solution) in the solid unit(s) of the invention is administered to a human subject initially at about day 1, followed by a subsequent dose of 80 mg of the antibody (e.g., 0.8 mL of a 100 mg/mL solution) two weeks later, followed by administration of about 40 mg (e.g., 0.4 mL of a 100 mg/mL solution) every other week for the treatment of Crohn's disease. In one embodiment, the solid unit(s) is administered subcutaneously, according to a multiple variable dose regimen comprising an induction dose(s) and maintenance dose(s) (see, for example, U.S. Patent Publication Nos. US20060009385 and US20090317399) for the treatment of Crohn's disease or ulcerative colitis, each of which are incorporated by reference herein) for the treatment of Crohn's disease or ulcerative colitis. In one embodiment, the solid unit(s) is administered subcutaneously, biweekly or monthly for the treatment of Crohn's disease or ulcerative colitis. In one embodiment, a dose of about 80 mg of a human anti-TNF-alpha antibody, or antigen-binding portion thereof, (e.g., adalimumab) (e.g., 0.8 mL of a 100 mg/mL solution) in the solid unit(s) of the invention is administered to a human subject monthly for the treatment of Crohn's disease or ulcerative colitis.

In one embodiment, the solid unit(s) of the invention is used to treat psoriasis. The formulation of the invention comprising an isolated human anti-TNF-alpha antibody, or antigen-binding portion thereof, (e.g., adalimumab), may be administered to a human subject according to a dosing scheme and dose amount effective for treating psoriasis. In one embodiment, an initial dose of about 80 mg of a human anti-TNF-alpha antibody, or antigen-binding portion thereof, (e.g., adalimumab) (e.g., 0.8 mL of a 100 mg/mL solution) in the solid unit(s) of the invention is administered to a human subject, followed by a subsequent dose of 40 mg of the antibody (e.g., 0.4 mL of a 100 mg/mL solution) every other week starting one week after the initial dose. In one embodiment, the solid unit(s) is administered subcutaneously, according to a multiple variable dose regimen comprising an induction dose(s) and maintenance dose(s) (see, for example, US 20060009385 and WO 2007/120823, each of which are incorporated by reference herein) for the treatment of psoriasis In one embodiment, the solid unit(s) is administered subcutaneously, biweekly or monthly for the treatment of psoriasis. In one embodiment, a dose of about 80 mg of a human anti-TNF-alpha antibody, or antigen-binding portion thereof, (e.g., adalimumab) (e.g., 0.8 mL of a 100 mg/mL solution) in the solid unit(s) of the invention is administered to a human subject monthly for the treatment of psoriasis.

In one embodiment, the solid unit(s) of the invention is used to treat juvenile idiopathic arthritis (JIA). The solid unit(s) of the invention comprising an isolated human anti-TNF-alpha antibody, or antigen-binding portion thereof, (e.g., adalimumab), may be administered to a human subject according to a dosing scheme and dose amount effective for treating JIA. In one embodiment, 20 mg of a human anti-TNF-alpha antibody, or antigen-binding portion thereof, in the solid unit(s) of the invention (e.g., 0.2 mL of a 100 mg/mL solution) is administered to a subject weighing 15 kg (about 33 lbs) to less than 30 kg (66 lbs) every other week for the treatment of JIA. In another embodiment, 40 mg of a human anti-TNF-alpha antibody, or antigen-binding portion thereof, in the formulation of the invention (e.g., 0.4 mL of a 100 mg/mL solution) is administered to a subject weighing more than or equal to 30 kg (66 lbs) every other week for the treatment of JIA. In one embodiment, the solid unit(s) is administered subcutaneously, according to a weight-based fixed dose (see, for example, U.S. Patent Publication No. 20090271164, incorporated by reference herein) for the treatment of JIA. In one embodiment, the solid unit(s) is administered subcutaneously biweekly or monthly for the treatment of JIA

In one embodiment, solid unit(s) containing an isolated human anti-TNF-alpha antibody, or antigen-binding portion thereof, (e.g., adalimumab), may be administered to a human subject for treatment of a disorder associated with detrimental TNF-alpha activity according to a monthly dosing schedule, whereby the antibody is administered once every month or once every four weeks. As described above, examples of disorders that may be treated according to a monthly dosing schedule using the solid unit(s) and methods of the invention include, but are not limited to, rheumatoid arthritis, ankylosing spondylitis, JIA, psoriasis, Crohn's disease, ulcerative colitis, hidradenitis suppurativa, giant cell arteritis, Behcet's disease, sarcoidosis, diabetic retinopathy, or psoriatic arthritis. Thus, the solid unit(s) of the invention comprising an isolated human anti-TNF-alpha antibody, or antigen-binding portion thereof, (e.g., adalimumab), may be administered to a human subject for treatment of a disorder associated with detrimental TNF-alpha activity according to a monthly dosing schedule. In one embodiment, 80 mg of a human anti-TNF-alpha antibody, or antigen-binding portion thereof, in the solid unit(s) of the invention (e.g., 0.8 mL of a 100 mg/mL formulation of the invention) is administered to a subject having a disorder associated with detrimental TNF-alpha activity. In one embodiment, 80 mg of a human anti-TNF-alpha antibody, or antigen-binding portion thereof, in the solid unit(s) of the invention (e.g., 0.8 mL of a 100 mg/mL formulation of the invention) is administered monthly or biweekly to a subject for the treatment of a disorder associated with detrimental TNF-alpha activity.

Solid unit(s) containing dose amounts described herein may be delivered as a single dose (e.g., a single dose of 40 mg or 80 mg), or, alternatively may be delivered as multiple doses (e.g., four 40 mg doses or two 80 mg doses for delivery of a 160 mg dose).

The solid unit(s) of the invention comprising an isolated human anti-TNF-alpha antibody, or antigen-binding portion thereof, (e.g., adalimumab) may also be administered to a subject in combination with an additional therapeutic agent. In one embodiment, the formulation is administered to a human subject for treatment of rheumatoid arthritis in combination with methotrexate or other disease-modifying anti-rheumatic drugs (DMARDs). In another embodiment, the solid unit(s) is administered to a human subject for treatment of JIA in combination with methotrexate or other disease-modifying anti-rheumatic drugs (DMARDs). Additional combination therapies are described in U.S. Pat. Nos. 6,258,562 and 7,541,031; and U.S. Patent Publication No. US20040126372, the entire contents of all of which are incorporated by reference herein.

The solid unit(s) of the invention comprising a human anti-TNF-alpha antibody, or antigen-binding portion thereof, may also be used to treat a subject who has failed previous TNF inhibitor therapy, e.g., a subject who has lost response to or is intolerant to infliximab.

The contents of all cited references, including literature references, issued patents, and published patent applications, as cited throughout this application are hereby expressly incorporated herein by reference. It should further be understood that the contents of all the figures and tables attached hereto are expressly incorporated herein by reference. The entire contents of the following applications are also expressly incorporated herein by reference: U.S. Provisional Patent Application No. 61/892,833, filed on Oct. 18, 2013; U.S. application Ser. No. 14/079,076, filed on Nov. 13, 2013; U.S. Provisional Patent Application 61/892,710 filed on Oct. 18, 2013; U.S. Patent Application Publication No. US20140271626, filed on Mar. 12, 2014; U.S. Provisional Patent Application 61/891,068, filed on Oct. 18, 2013; U.S. patent application Ser. No. 14/077,871, filed on Nov. 12, 2013; U.S. Provisional Patent Application 61/893,088, filed on Oct. 18, 2013; U.S. patent application Ser. No. 14/077,988, filed on Nov. 12, 2013; U.S. Provisional Patent Application 61/893,131, filed on Oct. 18, 2013; and U.S. patent application Ser. No. 14/077,574, filed on Nov. 12, 2013.

The present invention is further illustrated by the following examples which should not be construed as limiting in any way.

### EXAMPLES

### Example 1

### Uniform Free-Flowing Solid Unit Manufacturing Process for Holistic BioPharmaceutical Platform System

The production of uniform, free flowing solid units of the holistic platform system described herein may be produced generally by controlled nucleation freezing of a liquid solution followed by lyophilization and resulting in a uniform geometrically shaped solid unit. For example, solid units of the invention may be produced generally by freezing a solution comprising a therapeutic agent followed by lyophilization. The general process is described in detail below.

The initial step in the production of solid units comprising a therapeutic agent, such as a therapeutic protein, e.g., an antibody, was the freezing of a solution comprising the agent. The frozen solid unit (a sphere) was produced by releasing a droplet of the solution (e.g., a 20 μL droplet) into liquid nitrogen using a Cole Parmer syringe pump and a BioRad fraction collector. The droplet froze in the liquid nitrogen in the shape of a sphere and took approximately 20 seconds to freeze. Multiple droplets were released in sequence. The droplets were placed, in some instances, in a metal container with dividers submersed in the liquid nitrogen such that each solid unit was frozen individually. Once the solid unit was frozen, the sphere lost buoyancy and fell to the bottom of the container that was submersed in liquid nitrogen. Any number of solid units may be produced as needed in the initial freeze step. By freezing the solid units in suspension in the liquid nitrogen, the process provided for consistent freezing and a consistent nucleation temperature which resulted in uniform, free flowing solid units (spheres).

Following freezing, the solid units were collected from the liquid nitrogen and placed on a tray. The solid units were stored on dry ice during the transfer to the lyophilizer (an Ima Life lyophilizer). The solid units were then lyophilized in a standard commercial lyophilizer in a monoloayer. Following lyophilization, the resulting solid units were stored or used for further testing.

### Example 2

### Preparation of Adalimumab Solid Units

The following example describes the preparation of solid units comprising antibodies, exemplified by adalimumab. Solution 1 referenced below in Table 1 is a solution containing the following: 50-80 mg/ml adalimumab, mannitol (approximately 12 mg/ml), polysorbate 80 (approximately 1 mg/ml), sodium chloride, (approximately 6.15 mg/ml), and a phosphate/citrate buffer (sodium phosphate monobasic (approximately 0.86 mg/ml); sodium phosphate dibasic (approximately 1.53 mg/ml); sodium citrate (0.3 mg/ml); and citric acid monohydrate (approximately 1.3 mg/ml). Solution 2 is a solution containing 60-130 mg/ml adalimumab in water.

Specifically, the following adalimumab concentrations were used in solution 2 for the following studies: Study 31: 80 mg/ml adalimumab; Study 32: 100 mg/ml adalimumab; Study 33: 115 mg/ml adalimumab; Study 34: 130 mg/ml adalimumab; Study 34: 130 mg/ml adalimumab; and Study 47: 60 mg/ml adalimumab. Unless otherwise specified, solution 1 was used as the basis for the solid units described in Tables 1 to 5.

### General Lyophilization Process

Generally, the lyophilization process for making solid units of adalimumab included controlled nucleation utilizing liquid nitrogen to freeze a solution of adalimumab into spherical solid units (also referred to in the Examples as “pearls”), followed by vacuum sublimation of the solid units. Freezing was performed by dispensing a liquid solution containing adalimumab into a stainless steel pan with dividers filled with liquid nitrogen at approximately −190° C. The vacuum sublimation conditions included primary and secondary drying steps at 100 microns of pressure. The volume of each resulting adalimumab solid unit was about 9 to 15 μl. Table 2 provides a summary of all of the conditions that were tested. Conventional lyophilized cakes of adalimumab were also made, also described in Table 2 (referred to as “cakes”).

Studies 2, 5, 6, 7, 9, 49, 11, 12, 13, 14, 17, 20, 18, 19, 22, and 23, for example, demonstrate that solid units comprising adalimumab, as described herein, meet shelf life specifications for 24 months at 25° C./60% RH for size exclusion HPLC and cation exchange HPLC. Stability data demonstrates the proposed manufacturing system is capable of producing stable, uniform, free flowing solid units comprising a therapeutic agent, e.g., adalimumab. Additionally, the various formulations that were evaluated demonstrate the breadth of the proposed manufacturing system with controlled nucleation freezing. The processes for making each of these solid units are provided in more detail below.

### Studies 1, 3, and 4

In Study 1, no lyoprotectants were added to an adalimumab formulation which was lyophilized “as is” to evaluate if adalimumab bulk drug substance (BDS) (see Table 1) could be stabilized purely through a lyophilization process which employed controlled nucleation utilizing liquid nitrogen for a rapid freeze to produce uniform, free flowing solid units.

Studies 3 and 4 evaluated two additional freezing techniques to determine if adalimumab could be stabilized with no additional excipients in the formulation. In particular, adalimumab Bulk Drug Substance (BDS) (see Table 1, below) was subjected to controlled nucleation to produce uniform, free flowing solid units (Study 1), or subjected to standard lyophilizer freeze producing a cake (Study 3), or subjected to controlled liquid nitrogen freeze producing a cake (Study 4).

TABLE 1
Adalimumab Bulk Drug Substance (BDS)

| | Amount |
|---|---|
| Material | (mg/mL) |
| Adalimumab | ~50 |
| Mannitol | 12 |
| Tween 80 | 1 |
| Sodium chloride | 6.15 |
| Sodium phosphate, monobasic (2H₂O) | 0.86 |
| Sodium phosphate, dibasic (2H₂O) | 1.53 |
| Sodium citrate | 0.30 |
| Citric acid, monohydrate | 1.30 |

Preparation of the solid units in Study 1 was performed by dispensing the liquid solution into a stainless steel pan with dividers filled with liquid nitrogen (at approximately −190° C.). A syringe pump was used to dispense the solution with flow rate ranging from between approximately 2.0 to 2.2 mL/minute using a 23G needle. Frozen solid units (spherical) approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of 100 microns. Following evacuation, the solid units were subjected to a primary drying step at about 10° C. and 100 microns of pressure. Finally, the solid units went through a secondary drying step at 25° C. under 100 microns of pressure. This lyophilization process resulted in solid units containing adalimumab.

For Study 3, the standard lyophilization process was performed as follows. A stainless steel tray was loaded with vials. The vials were filled with approximately 1 mL of Adalimumab BDS solution using a syringe pump. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After freezing, the lyophilization chamber was evacuated to a pressure of approximately 100 microns. Following evacuation, the vials were subjected to a primary drying step at about 10° C. and 100 microns of pressure. Lastly, the vials were subjected to a secondary drying step at 25° C. under approximately 100 microns of pressure.

For Study 4, controlled liquid nitrogen freezing was performed as follows. A stainless steel tray was loaded with vials. The vials were filled with approximately 1 mL of Adalimumab BDS solution using the syringe pump. Lyo stoppers were inserted into the vials (lyo stoppers were partially inserted to allow sublimation venting). The stainless steel tray loaded with vials was placed in a Styrofoam box. Liquid nitrogen was poured onto the stainless steel tray to rapidly freeze the solution in the vials. The vials were loaded into the lyophilizer at shelf temperature about −50° C. After being held at −50° C., the lyophilization chamber was evacuated to a pressure of approximately 100 microns. Following evacuation, the vials were subjected to a primary drying step at about 10° C. and 100 microns of pressure. Lastly, the vials were subjected to a secondary drying step at 25° C. under approximately 100 microns of pressure.

### Studies 2 and 12

Study 2 evaluated the impact of sucrose on the stability of adalimumab in solid units comprising a citrate/phosphate buffer. Study 12 tested the impact of trehalose on the stability of adalimumab solid units comprising a citrate/phosphate buffer.

In particular, one challenge with lyophilizing a protein contained in a buffer matrix is the potential for specific buffer salts to precipitate during the freezing step, resulting in large pH modifications that can negatively affect protein stability. For example, a pH<4 can result from the crystallization of the dibasic form of sodium. Therefore, sodium phosphate buffer is not recommended in the development of lyophilized protein formulations.

Accordingly. adalimumab in the study is contained in a citrate/phosphate buffer matrix that could have a negative impact on stability due to possible pH modifications. The objective of the study was to evaluate if sucrose or trehalose could effectively stabilize within solid units comprising adalimumab in a citrate/phosphate buffer matrix by utilizing controlled nucleation employing liquid nitrogen to produce uniform, free flowing solid units. For Study 2, prior to freezing, sucrose was added to an adalimumab liquid solution. Specifically, trehalose at a concentration of approximately 46 mg/ml was added to a solution having a pH of 5 and containing the following ingredients (concentration in parentheses); adalimumab (approximately 50 mg/ml, but ranging from 50-80 mg/ml), mannitol (approximately 12 mg/ml); tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (0.3 mg/ml); and citric acid monohydrate (approximately 1.3 mg/ml).

For study 12, prior to freezing, trehalose was added to an adalimumab liquid solution. Specifically, trehalose at a concentration of approximately 46 mg/ml was added to a solution having a pH of 5 and containing the following ingredients (concentration in parentheses); adalimumab (approximately 50 mg/ml, but ranging from 50-80 mg/ml), mannitol (approximately 12 mg/ml); tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (0.3 mg/ml); and citric acid monohydrate (approximately 1.3 mg/ml).

Following the addition of sucrose or trehalose, freezing of the solid units was performed by dispensing the liquid solution into a stainless steel pan with dividers filled with liquid nitrogen (at approximately −190° C.). A syringe pump was used to dispense the solution with flow rate ranging from between approximately 2.0 to 2.2 mL/minute using a 23G needle. Frozen solid units (spherical) approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of 100 microns. Following evacuation, the solid units were subjected to a primary drying step at about 10° C. and 100 microns of pressure. Finally, the solid units went through a secondary drying step at 25° C. under 100 microns of pressure. This lyophilization process resulted in stable solid units containing adalimumab.

The solid units contained a sugar to protein mass ratio (sugar:protein ratio, e.g., sucrose:antibody ratio) at a 50 mg/ml adalimumab concentration of 0.92:1.

The process was successfully performed (see Study 2 and Study 12 in Table 2) using the above conditions.

### Studies 2, 6, and 7

Studies 2, 6, and 7 tested the impact of sucrose on the stability of the adalimumab solid units. Studies 6 and 7 also tested the impact of pH on the stability of adalimumab solid units.

For studies 2, 6, and 7, prior to freezing, sucrose was added to an adalimumab liquid solution. Specifically, sucrose at a concentration of approximately 46 mg/ml was added to a solution having a pH of 5 or 6 and containing the following ingredients (concentration in parentheses); adalimumab (approximately 50 mg/ml, but ranging from 50-80 mg/ml), mannitol (approximately 12 mg/ml); tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (0.3 mg/ml); and citric acid monohydrate (approximately 1.3 mg/ml).

Following the addition of sucrose, freezing of the solid units was performed by dispensing the liquid solution into a stainless steel pan with dividers filled with liquid nitrogen (at approximately −190° C.). A syringe pump was used to dispense the solution with flow rate ranging from between approximately 2.0 to 2.2 mL/minute using a 23G needle. Frozen solid units (spherical) approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of 100 microns. Following evacuation, the solid units were subjected to a primary drying step at about 10° C. and 100 microns of pressure. Finally, the solid units went through a secondary drying step at 25° C. under 100 microns of pressure. This lyophilization process resulted in stable solid units containing adalimumab.

The solid units contained a sugar to protein mass ratio (sugar:protein ratio, e.g., sucrose:antibody ratio) at a 50 mg/ml adalimumab concentration of 0.92:1. At an antibody concentration of 80 mg/ml, the solid units contained a sugar:protein ratio of 0.575:1.

The process was successfully performed (see Studies 2, 6, and 7 in Table 2) using the above conditions. Studies 2 and 6 included lyophilizing an adalimumab solution having a pH of 5, and Study 7 included lyophilizing an adalimumab solution having a pH of 6.

### Studies 6, 7, 9, 10, and 11

Studies 6, 7, 9, 10, and 11 tested the impact of pH on the stability of adalimumab solid units comprising sucrose.

For studies 6, 7, 9, 10, and 11 prior to freezing, sucrose was added to an adalimumab liquid solution. Specifically, sucrose at a concentration of approximately 46 mg/ml was added to a solution having a pH of 5 (Study 6), a solution having a pH of 6 (Study 7), a solution having a pH of 4 (Study 9), a solution having a pH of 3 (Study 10), or a solution having a pH of 7 (Study 11) and, each containing the following ingredients (concentration in parentheses); adalimumab (approximately 50 mg/ml, but ranging from 50-80 mg/ml), mannitol (approximately 12 mg/ml); tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (0.3 mg/ml); and citric acid monohydrate (approximately 1.3 mg/ml).

Following the addition of sucrose, freezing of the solid units was performed by dispensing the liquid solution into a stainless steel pan with dividers filled with liquid nitrogen (at approximately −190° C.). A syringe pump was used to dispense the solution with flow rate ranging from between approximately 2.0 to 2.2 mL/minute using a 23G needle. Frozen solid units (spherical) approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of 100 microns. Following evacuation, the solid units were subjected to a primary drying step at about 10° C. and 100 microns of pressure. Finally, the solid units went through a secondary drying step at 25° C. under 100 microns of pressure. This lyophilization process resulted in stable solid units containing adalimumab.

### Studies 13 and 14

Additional solid units were made using a similar process as described above but with a different amount of sucrose as a stabilizer. Studies 13 and 14 tested the impact of sucrose on the stability of the adalimumab solid units.

Prior to freezing, sucrose was added to an adalimumab liquid solution. Specifically, sucrose at a concentration of approximately 70 or 90 mg/ml (Studies 13 and 14, respectively) was added to a solution having a pH of 5 and containing the following ingredients (concentration in parentheses); adalimumab (approximately 50 mg/ml, but ranging from 50-80 mg/ml), mannitol (approximately 12 mg/ml); Tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (approximately 0.3 mg/ml); and citric acid monohydrate (approximately 1.3 mg/ml).

Following the addition of sucrose, freezing of the solid units was performed by dispensing the liquid solution into a stainless steel pan with dividers filled with liquid nitrogen (at approximately −190° C.). A syringe pump was used to dispense the solution with flow rate of about 2.0 ml/minute using a 23G needle. Frozen solid units (spherical) were approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of approximately 100 microns. Following evacuation, the solid units were subjected to a first primary drying step at about −29° C. and 100 microns of pressure and a second primary drying step at about 10° C. and 100 microns of pressure. Finally the solid units went through a secondary drying step at 25° C. under approximately 100 microns of pressure. This lyophilization process resulted in stable solid units containing adalimumab. The process was successfully performed using the above conditions (see Studies 13 and 14).

### Studies 17 and 18

Additional solid units were made using a similar process as described above but using sucrose and glycine as stabilizers.

Prior to lyophilization, sucrose and glycine were added to an adalimumab liquid solution. Sucrose and glycine were added to a final concentration of 4% and 2.5%, respectively (Study 17) or to a final concentration of 4% and 5%, respectively (Study 18). The liquid solution had a pH of 5 and contained the following ingredients (concentration in parentheses); adalimumab (approximately 50 mg/ml, but ranging from 50-80 mg/ml), mannitol (approximately 12 mg/ml); tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (approximately 0.3 mg/ml); citric acid monohydrate (approximately 1.3 mg/ml); sucrose (approximately 40 mg/ml); and glycine (approximately 25 mg/ml).

Following the addition of sucrose and glycine, freezing of the solid units was performed by dispensing the liquid solution into a stainless steel pan with dividers filled with liquid nitrogen (at approximately −190° C.). A syringe pump was used to dispense the solution with flow rate of about 2.0 ml/minute using a 23G needle. Frozen solid units (spherical) were approximately 2 to 3 mm in diameter, and had a volume of about 9 to 15 microliters.

Following freezing, the solid units were subjected to vacuum sublimation. The vacuum sublimation cycle conditions included a loading step at about −50° C., following by evacuation at a pressure of approximately 100 microns. Following evacuation, the solid units were subjected to a first primary drying step at about −29° C. and 100 microns of pressure and a second primary drying step at about 10° C. and 100 microns of pressure. Finally the solid units went through a secondary drying step at 25° C. under 100 microns of pressure. This lyophilization process resulted in stable solid units containing adalimumab (see Study 17 and Study 18).

### Studies 19 and 20

Additional solid units were made using a similar process as described above but using trehalose and glycine as stabilizers.

Prior to freezing, trehalose and glycine was added to an adalimumab liquid solution. Trehalose and glycine were added to the antibody solution to a final concentration of approximately 4% and approximately 2.5%, respectively (Study 19), or to a final concentration of approximately 4% and approximately 5%, respectively (Study 20). The liquid solution had a pH of 5 and contained the following ingredients (concentration in parentheses); adalimumab (approximately 50 mg/ml, but ranging from 50-80 mg/ml), mannitol (approximately 12 mg/ml); tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (approximately 0.3 mg/ml); citric acid monohydrate (approximately 1.3 mg/ml); trehalose (approximately 40 mg/ml); and glycine (approximately 25 mg/ml).

Following the addition of trehalose and glycine, freezing of the solid units was performed by dispensing the liquid solution into a stainless steel pan with dividers filled with liquid nitrogen (at approximately −190° C.). A syringe pump was used to dispense the solution with flow rate of about 2.0 ml/minute using a 23G needle. Frozen solid units (spherical) were approximately 2 to 3 mm in diameter, and had a volume of about 9 to 15 microliters.

Following freezing, the solid units were subjected to vacuum sublimation. The vacuum sublimation cycle conditions included a loading step at about −50° C., following by evacuation at a pressure of approximately 100 microns. Following evacuation, the solid units were subjected to a first primary drying step at about −29° C. and 100 microns of pressure and a second primary drying step at about 10° C. and 100 microns of pressure. Finally the solid units went through a secondary drying step at 25° C. under 100 microns of pressure. This lyophilization process resulted in stable solid units containing adalimumab (see Study 19 and Study 20).

In these studies, the solid units contained a sugar to protein mass ratio (sugar:protein ratio, e.g., sucrose:antibody ratio) at a 50 mg/ml adalimumab concentration of 0.92:1. At an antibody concentration of 80 mg/ml, the solid units contained a sugar:protein ratio of 0.575:1.

### Studies 21, 22, 23, 24, 25, and 26

Additional solid units were made using a similar process as described above but with different stabilizers. Studies 21 and 22 tested the impact of sucrose and dextran on the stability of the adalimumab solid units. Study 23 tested the impact of trehalose and dextran on the stability of the adalimumab solid units. Study 24 tested the impact of sucrose and PEG on the stability of the adalimumab solid units. Study 25 tested the impact of trehalose and PEG on the stability of the adalimumab solid units. Study 26 tested the impact of hydroxypropyl beta cyclodextrin on the stability of the adalimumab solid units.

For Studies 21 and 22, prior to freezing, sucrose at a concentration of approximately 1% and dextran at a concentration of 1% (Study 21) or sucrose at a concentration of 5% and dextran at a concentration of 1% (Study 22) were was added to an adalimumab solution having a pH of 5. For Study 23, prior to freezing trehalose at a concentration of about 5% and dextran at a concentration of about 1% were was added to an adalimumab solution having a pH of 5. For Study 24, prior to freezing sucrose at a concentration of about 10 mM and PEG at a concentration of about 1% were was added to an adalimumab solution having a pH of 5. For Study 25, prior to freezing trehalose at a concentration of about 10 mM and PEG at a concentration of about 1% were was added to an adalimumab solution having a pH of 5. For Study 26, prior to freezing hydroxypropyl beta cyclodextrin at a concentration of about 5% was added to an adalimumab solution having a pH of 5.

The adalimumab solution to which each of the stabilizers (or combination of stabilizers) was added for Studies 21-26 containing the following ingredients (concentration in parentheses); adalimumab (approximately 50 mg/ml, but ranging from 50-80 mg/ml), mannitol (approximately 12 mg/ml); Tween 80 (approximately 1 mg/ml); sodium chloride (approximately 6.15 mg/ml); sodium phosphate monobasic (approximately 0.86 mg/ml); sodium phosphate dibasic (approximately 1.53 mg/ml); sodium citrate (approximately 0.3 mg/ml); and citric acid monohydrate (approximately 1.3 mg/ml).

Following the addition of the stabilizer, or combination of stabilizers, freezing of the solid units was performed by dispensing the liquid solution into a stainless steel pan with dividers filled with liquid nitrogen (at approximately −190° C.). A syringe pump was used to dispense the solution with flow rate of about 2.0 ml/minute using a 23G needle. Frozen solid units (spherical) were approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of approximately 100 microns. Following evacuation, the solid units were subjected to a first primary drying step at about −29° C. and 100 microns of pressure and a second primary drying step at about 10° C. and 100 microns of pressure. Finally the solid units went through a secondary drying step at 25° C. under approximately 100 microns of pressure. This lyophilization process resulted in stable solid units containing adalimumab. The process was successfully performed using the above conditions (see Studies 21, 22, 23, 24, 25, and 26).

### Studies 31, 32, 33, and 34

Additional solid units were made with sucrose as a stabilizer using a similar process as described above but in the absence of additional excipients.

Prior to freezing, sucrose was added to an adalimumab liquid solution. Specifically, sucrose at a concentration of approximately 61 mg/ml was added to a solution having a pH of 5 and containing approximately 80 mg/ml adalimumab (Study 31), or sucrose at a concentration of approximately 77 mg/ml was added to a solution having a pH of 5 and containing approximately 100 mg/ml adalimumab (Study 32), or sucrose at a concentration of approximately 88 mg/ml was added to a solution having a pH of 5 and containing approximately 115 mg/ml adalimumab (Study 33), or sucrose at a concentration of approximately 100 mg/ml was added to a solution having a pH of 5 and containing approximately 130 mg/ml adalimumab (Study 34), or

Following the addition of sucrose, freezing of the solid units was performed by dispensing the liquid solution into a stainless steel pan with dividers filled with liquid nitrogen (at approximately −190° C.). A syringe pump was used to dispense the solution with flow rate of about 2.0 ml/minute using a 23G needle. Frozen solid units (spherical) were approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of approximately 100 microns. Following evacuation, the solid units were subjected to a first primary drying step at about −29° C. and 100 microns of pressure and a second primary drying step at about 10° C. and 100 microns of pressure. Finally the solid units went through a secondary drying step at 25° C. under approximately 100 microns of pressure. This lyophilization process resulted in stable solid units containing adalimumab. The process was successfully performed using the above conditions (see Studies 31-34).

### Study 47

Additional solid units were made with sucrose as a stabilizer using a similar process as described above to evaluate the stability of an adalimumab and sucrose only formulation without additional excipients prepared using controlled nucleation freezing.

Prior to freezing, sucrose was added to an adalimumab liquid solution. Specifically, sucrose at a concentration of approximately 46 mg/ml was added to a solution having a pH of about 5 and containing approximately 60 mg/ml adalimumab.

Following the addition of sucrose, freezing of the solid units was performed by dispensing the liquid solution into a stainless steel pan with dividers filled with liquid nitrogen (at approximately −190° C.). A syringe pump was used to dispense the solution with flow rate of about 2.0 ml/minute using a 23G needle. Frozen solid units (spherical) were approximately 2 to 3 mm in diameter were obtained. The volume of each solid unit was about 9 to 15 microliters.

Following freezing, the solid units were subjected to vacuum sublimation. The vacuum sublimation cycle conditions included a loading step at about −50° C., following by evacuation at a pressure of approximately 100 microns. Following evacuation, the solid units were subjected to a first primary drying step at about −29° C. and 100 microns of pressure and a second primary drying step at about 10° C. and 100 microns of pressure. Finally the solid units went through a secondary drying step at 25° C. under approximately 100 microns of pressure. This lyophilization process resulted in solid units containing adalimumab.

### Studies 49 and 50

Lyophilized cakes in vials were made with sucrose as a stabilizer using a similar process as described above to evaluate the effect of freezing with liquid nitrogen in a vial (Study 49) and freezing with liquid nitrogen and then annealing (Study 50) on the stability of adalimumab BDS.

Prior to freezing, sucrose was added to an adalimumab liquid solution. Specifically, sucrose at a concentration of approximately 46 mg/ml was added to an adalimumab BDS solution (see Table 1 above). Following the addition of sucrose, solid units were prepared by dispensing the liquid solution into vials. A syringe pump was used to dispense the solution with flow rate of about 2.0 ml/minute using a 23G needle. The vials were filled with approximately 1 mL of Adalimumab BDS with sucrose solution using the syringe pump. Lyo stoppers were inserted into the vials (lyo stoppers were partially inserted to allow sublimation venting). The stainless steel tray loaded with vials was placed in a Styrofoam box. Liquid nitrogen was poured onto the stainless steel tray to rapidly freeze the solution in the vials. The vials were loaded into the lyophilizer at shelf temperature about −50° C. After being held at −50° C., the lyo chamber was evacuated to a pressure of approximately 100 microns. Following evacuation, the vials were subjected to a first primary drying step at about 29° C. and 100 microns of pressure and a second primary drying step at about 10° C. and 100 microns of pressure. Lastly, the vials were subjected to a secondary drying step at 25° C. under approximately 100 microns of pressure (Study 49)

For freezing with liquid nitrogen and then annealing (Study 50), a stainless steel tray was loaded with vials. The vials were filled with approximately 1 mL of Adalimumab BDS with sucrose solution using the syringe pump to dispense the solution with flow rate of about 2.0 ml/minute using a 23G needle. Lyo stoppers were inserted into the vials (lyo stoppers were partially inserted to allow sublimation venting). The stainless steel tray loaded with vials was placed in a Styrofoam box. Liquid nitrogen was poured onto the stainless steel tray to rapidly freeze the solution in the vials. The vials were loaded into the lyophilizer at shelf temperature about −50° C. After being held at −50° C., the shelf temperature was warmed to −15° C. and held there for about an hour. The shelf was then cooled to −50° C. After being held at −50° C., the lyophilization chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

Both processes resulted in lyophilized cakes in vials.

### Studies 51, 52, 57, and 17

Additional lyophilized cakes in vials and uniform, free flowing solid units were each made with sucrose and glycine as stabilizers to evaluate the effect of different freezing techniques on the stability of adalimumab.

In particular, adalimumab Bulk Drug Substance (BDS) (see Table 1, above) was subjected to controlled nucleation to produce uniform, free flowing solid units (Study 17), or subjected to standard lyophilizer freeze producing a cake (Study 51), or subjected to standard lyophilizer freeze producing a cake and then annealed (Study 57), or subjected to controlled liquid nitrogen freeze in a vial producing a cake (Study 52).

Prior to freezing, sucrose and glycine were added to an adalimumab liquid solution. Specifically, sucrose at a concentration of approximately 4% and glycine at a concentration of approximately 2.5% were added to an adalimumab BDS solution (see Table 1, above).

Adalimumab Bulk Drug Substance (BDS) (see Table 1, above) subjected to standard lyophilizer freeze producing a cake (Study 51) was performed as follows. A stainless steel tray was loaded with vials. The vials were filled with approximately 1 mL of Adalimumab BDS with glycine and sucrose solution using the syringe pump to dispense the solution with flow rate of about 2.0 ml/minute using a 23G needle. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After being held at −50° C., the shelf temperature was warmed to −15° C. The shelf was then cooled to −50° C. After being held at −50° C., the lyophilization chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

Adalimumab Bulk Drug Substance (BDS) (see Table 1, above) subjected to controlled liquid nitrogen freeze in a vial producing a cake (Study 52) was performed as follows. A stainless steel tray was loaded with vials. The vials were filled with approximately 1 mL of Adalimumab BDS with glycine and sucrose solution using the syringe pump to dispense the solution with flow rate of about 2.0 ml/minute using a 23G needle. Lyo stoppers were inserted into the vials (lyo stoppers were partially inserted to allow sublimation venting). The stainless steel tray loaded with vials was placed in a Styrofoam box. Liquid nitrogen (approximately −190° C.) was poured onto the stainless steel tray to rapidly freeze the solution in the vials. The vials were loaded into the lyophilizer at shelf temperature about −50° C. After being held at −50° C., the shelf temperature was warmed to −15° C. The shelf was then cooled to −50° C. After being held at −50° C., the lyo chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

Adalimumab Bulk Drug Substance (BDS) (see Table 1, above) subjected to standard lyophilizer freeze producing a cake and then annealed (Study 57) was performed as follows. A stainless steel tray was loaded with vials. The vials were filled with approximately 1 mL of Adalimumab BDS with glycine and sucrose solution using the syringe pump. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After being held at −50° C., the lyo chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

### Study 54

The following study investigated whether the addition of trehalose to a solution of adalimumab bulk drug substance is sufficient to confer stability to the adalimumab when freeze dried using a different freezing technique, such as the standard lyo freeze.

In particular, prior to freezing, trehalose was added to an adalimumab BDS liquid solution at 46 mg/ml.

Following the addition of trehalose, the solution was dispensed into vials and a stainless steel tray was loaded with the vials. The vials were filled with approximately 1 mL of Adalimumab BDS with trehalose solution using the syringe pump. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After being held at −50° C., the lyo chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

### Studies 55 and 56

The following study investigated whether the addition of sucrose to a solution of adalimumab BDS was sufficient to confer stability to the adalimumab when freeze dried using a different freezing technique, such as the standard lyo freeze.

In particular, prior to freezing, sucrose at a concentration of 70 mg/ml (Study 55) or 90 mg/ml (Study 56) was added to an adalimumab BDS liquid solution.

Following the addition of sucrose, the solution was loaded into vials and a stainless steel tray was loaded with the vials. The vials were filled with approximately 1 mL of Adalimumab BDS with sucrose solution using the syringe pump. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After being held at −50° C., the lyo chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

### Study 58

The following study investigated whether the addition of hydroxypropyl beta cyclodextrin to a solution of adalimumab BDS was sufficient to confer stability to the adalimumab when freeze dried using the standard lyo freeze technique.

In particular, prior to freezing hydroxypropyl beta cyclodextrin was added to an adalimumab BDS liquid solution at a concentration of about 5%.

Following the addition of hydroxypropyl beta cyclodextrin, the solution was dispensed into vials and a stainless steel tray was loaded with vials. The vials were filled with approximately 1 mL of adalimumab BDS with hydroxypropyl beta cyclodextrin solution using the syringe pump. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After being held at −50° C., the lyophilization chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

### Study 59

The following study investigated whether the addition of trehalose and glycine to a solution of adalimumab bulk drug substance was sufficient to confer stability to the adalimumab when freeze dried using the standard lyo freeze technique.

In particular, prior to freezing, trehalose was added to an adalimumab BDS liquid solution at a concentration of about 4% and glycine was added to the same solution at a concentration of about 2.5%.

Following the addition of trehalose and glycine, the solution was dispensed into vials and a stainless steel tray was loaded with the vials. The vials were filled with approximately 1 mL of Adalimumab BDS with glycine and trehalose solution using the syringe pump. Lyo stoppers were inserted into the vials (lyo stoppers were partially inserted to allow sublimation venting). The vials were loaded into the lyophilizer at shelf temperature about 20° C. The shelf temperature was reduced to −50° C. for freezing. After being held at −50° C., the lyo chamber was evacuated to a pressure of approximately 100 microns. Following evacuation, the vials were subjected to a first primary drying step at about −29° C. and 100 microns of pressure and a second primary drying step at about 10° C. and 100 microns of pressure. Lastly, the vials were subjected to a secondary drying step at 25° C. under approximately 100 microns of pressure.

Table 2 below provides a summary of the solid units that were made, including those described above, as well as the processes by which they were made. Table 2 also provides alternative conditions under which the lyophilized stable solid units may be made. The conditions for the above studies are presented in bold in Table 2. The spherical subunits are referred to as “pearls” in Table 2 below. “Cakes” described in Table 2 below refer to the traditional lyophilization cakes which were used as controls.

TABLE 2
Formulation Summary for Adalimumab Solid Units and Cakes.

| | | | | | | Storage | | PD1 | PD2 | SD | |
|---|---|---|---|---|---|---|---|---|---|---|---|
| | | Additional | | | | Condition | Pressure | Temp | Temp | Temp | Fill |
| Study | Sample | Excipients | Form | pH | Freezing | (s) | (microns) | (° c.) | (° C.) | (° C.) | Volume |
| 1 | Adalimumab | N/A | Pearls | 5 | Liquid Nitrogen | 40 C., 25 C., | 100 | 10 | N/A | 25 | 1 mL |
| | Solution 1 | | | | | 5 C. | | | | | |
| 2 | Adalimumab | 46 mg/mL Sucrose | Pearls | 5 | Liquid Nitrogen | 40 C., 25 C., | 100 | 10 | N/A | 25 | 1 mL |
| | Solution 1 | | | | | 5 C. | | | | | |
| 3 | Adalimumab | N/A | Cake | 5 | Lyo Freeze | 40 C., 25 C., | 100 | 10 | N/A | 25 | 1 mL |
| | Solution 1 | | | | | 5 C. | | | | | |
| 4 | Adalimumab | N/A | Cake | 5 | Liquid Nitrogen | 40 C., 25 C., | 100 | 10 | N/A | 25 | 1 mL |
| | Solution 1 | | | | | 5 C. | | | | | |
| 5 | Adalimumab | 46 mg/mL Sucrose | Cake | 5 | Lyo Freeze | 40 C., 25 C. | 100 | −29 | 10 | 25 | 1 mL |
| | Solution 1 | | | | | | | | | | |
| 6 | Adalimumab | 46 mg/mL Sucrose | Pearls | 5 | Liquid Nitrogen | 40 C., 25 C. | 100 | −29 | 10 | 25 | 1 mL |
| | Solution 1 | | | | | | | | | | |
| 7 | Adalimumab | 46 mg/mL Sucrose | Pearls | 6 | Liquid Nitrogen | 40 C., 25 C. | 100 | −29 | 10 | 25 | 1 mL |
| | Solution1 | | | | | | | | | | |
| 8 | Adalimumab | N/A | Pearls | 5 | Liquid Nitrogen | 40 C., 25 C. | 100 | −29 | 10 | 25 | 1 mL |
| | Solution 1 | | | | | | | | | | |
| 9 | Adalimumab | 46 mg/mL Sucrose | Pearls | 4 | Liquid Nitrogen | 40 C., 25 C. | 100 | −29 | 10 | 25 | 1 mL |
| | Solution1 | | | | | | | | | | |
| 10 | Adalimumab | 46 mg/mL Sucrose | Pearls | 3 | Liquid Nitrogen | N/A | N/A | N/A | N/A | N/A | 1 mL |
| | Solution 1 | | | | | | | | | | |
| 11 | Adalimumab | 46 mg/mL Sucrose | Pearls | 7 | Liquid Nitrogen | 40 C., 25 C. | 100 | −29 | 10 | 25 | 1 mL |
| | Solution 1 | | | | | | | | | | |
| 12 | Adalimumab | 46 mg/mL Trehalose | Pearls | 5 | Liquid Nitrogen | 40 C., 25 C. | 100 | −29 | 10 | 25 | 1 mL |
| | Solution 1 | | | | | | | | | | |
| 13 | Adalimumab | 70 mg/mL Sucrose | Pearls | 5 | Liquid Nitrogen | 40 C., 25 C. | 100 | −29 | 10 | 25 | 1 mL |
| | Solution 1 | | | | | | | | | | |
| 14 | Adalimumab | 90 mg/mL Sucrose | Pearls | 5 | Liquid Nitrogen | 40 C., 25 C. | 100 | −29 | 10 | 25 | 1 mL |
| | Solution 1 | | | | | | | | | | |
| 17 | Adalimumab | 2.5% glycine, 4% sucrose | Pearls | 5 | Liquid Nitrogen | 40 C., 25 C. | 100 | −29 | 10 | 25 | 1 mL |
| | Solution 1 | | | | | | | | | | |
| 18 | Adalimumab | 5% glycine, 4% sucrose | Pearls | 5 | Liquid Nitrogen | 40 C., 25 C. | 100 | −29 | 10 | 25 | 1 mL |
| | Solution 1 | | | | | | | | | | |
| 19 | Adalimumab | 2.5% glycine, 4% trehalose | Pearls | 5 | Liquid Nitrogen | 40 C., 25 C. | 100 | −29 | 10 | 25 | 1 mL |
| | Solution 1 | | | | | | | | | | |
| 20 | Adalimumab | 5% glycine, 4% trehalose | Pearls | 5 | Liquid Nitrogen | 40 C., 25 C. | 100 | −29 | 10 | 25 | 1 mL |
| | Solution 1 | | | | | | | | | | |
| 21 | Adalimumab | 1% sucrose, 1% dextran | Pearls | 5 | Liquid Nitrogen | 40 C., 25 C. | 100 | −29 | 10 | 25 | 1 mL |
| | Solution 1 | | | | | | | | | | |
| 22 | Adalimumab | 5% sucrose, 1% dextran | Pearls | 5 | Liquid Nitrogen | 40 C., 25 C. | 100 | −29 | 10 | 25 | 1 mL |
| | Solution 1 | | | | | | | | | | |
| 23 | Adalimumab | 5% trehalose, 1% dextran | Pearls | 5 | Liquid Nitrogen | 40 C., 25 C. | 100 | −29 | 10 | 25 | 1 mL |
| | Solution 1 | | | | | | | | | | |
| 24 | Adalimumab | 1% PEG, 10 mM sucrose | Pearls | 5 | Liquid Nitrogen | N/A | 100 | −29 | 10 | 25 | 1 mL |
| | Solution 1 | | | | | | | | | | |
| 25 | Adalimumab | 1% PEG, 10 mM trehalose | Pearls | 5 | Liquid Nitrogen | 40 C., 25 C. | 100 | −29 | 10 | 25 | 1 mL |
| | Solution 1 | | | | | | | | | | |
| 26 | Adalimumab | 5% hydroxypropyl | Pearls | 5 | Liquid Nitrogen | 40 C., 25 C. | 100 | −29 | 10 | 25 | 1 mL |
| | Solution 1 | beta cyclodextrin | | | | | | | | | |
| 31 | Adalimumab | 61 mg/mL Sucrose | Pearls | 5 | Liquid Nitrogen | N/A | 100 | −29 | 10 | 25 | 1 mL |
| | Solution 2 | | | | | | | | | | |
| 32 | Adalimumab | 77 mg/mL Sucrose | Pearls | 5 | Liquid Nitrogen | N/A | 100 | −29 | 10 | 25 | 1 mL |
| | Solution 2 | | | | | | | | | | |
| 33 | Adalimumab | 88 mg/mL Sucrose | Pearls | 5 | Liquid Nitrogen | N/A | 100 | −29 | 10 | 25 | 1 mL |
| | Solution 2 | | | | | | | | | | |
| 34 | Adalimumab | 100 mg/mL Sucrose | Pearls | 5 | Liquid Nitrogen | N/A | 100 | −29 | 10 | 25 | 1 mL |
| | Solution 2 | | | | | | | | | | |
| 36 | Adalimumab | 70 mg/mL Sucrose | Pearls | 5 | Liquid Nitrogen | 40 C., 25 C. | 100 | −20 | 10 | 25 | 1 mL |
| | Solution 1 | | | | | | | | | | |
| 40 | Adalimumab | 70 mg/mL Sucrose | Pearls | 5 | Liquid Nitrogen | 40 C., 25 C. | 100 | −20 | 10 | 25 | 1 mL |
| | Solution 1 | | | | | | | | | | |
| 41 | Adalimumab | 70 mg/mL Sucrose | Pearls | 5 | Liquid Nitrogen | 40 C., 25 C. | 100 | −20 | 10 | 25 | 1 mL |
| | Solution 1 | | | | | | | | | | |
| 42 | Adalimumab | 70 mg/mL Sucrose | Pearls | 5 | Liquid Nitrogen | 40 C., 25 C. | 100 | −20 | 10 | 25 | 1 mL |
| | Solution 1 | | | | | | | | | | |
| 43 | Adalimumab | 70 mg/mL Sucrose | Pearls | 5 | Liquid Nitrogen | 40 C., 25 C. | 100 | −20 | 10 | 25 | 1 mL |
| | Solution 1 | | | | | | | | | | |
| 44 | Adalimumab | 70 mg/mL Sucrose | Pearls | 5 | Liquid Nitrogen | 40 C., 25 C. | 100 | −20 | 10 | 25 | 1 mL |
| | Solution 1 | | | | | | | | | | |
| 47 | Adalimumab | 46 mg/mL Sucrose | Pearls | 5 | Liquid Nitrogen | N/A | 100 | −29 | 10 | 25 | 1 mL |
| | Solution 2 | | | | | | | | | | |
| 49 | Adalimumab | 46 mg/mL Sucrose | Cake | 5 | Liquid Nitrogen | 40 C., 25 C. | 100 | −29 | 10 | 25 | 1 mL |
| | Solution 1 | | | | | | | | | | |
| 54 | Adalimumab | 46 mg/mL Trehalose | Cake | 5 | Std. Lyo Freeze | 40 C., 25 C. | 100 | −29 | 10 | 25 | 1 mL |
| | Solution 1 | | | | | | | | | | |
| 55 | Adalimumab | 70 mg/mL Sucrose | Cake | 5 | Std. Lyo Freeze | 40 C., 25 C. | 100 | −29 | 10 | 25 | 1 mL |
| | Solution 1 | | | | | | | | | | |
| 56 | Adalimumab | 90 mg/mL Sucrose | Cake | 5 | Std. Lyo Freeze | 40 C., 25 C. | 100 | −29 | 10 | 25 | 1 mL |
| | Solution 1 | | | | | | | | | | |
| 57 | Adalimumab | 2.5% glycine, 4% sucrose | Cake | 5 | Std. Lyo Freeze | 40 C., 25 C. | 100 | −29 | 10 | 25 | 1 mL |
| | Solution 1 | | | | | | | | | | |
| 58 | Adalimumab | 5% hydroxypropyl | Cake | 5 | Std. Lyo Freeze | 40 C., 25 C. | 100 | −29 | 10 | 25 | 1 mL |
| | Solution 1 | beta cyclodextrin | | | | | | | | | |
| 59 | Adalimumab | 5% glycine, 4% trehalose | Cake | 5 | Std. Lyo Freeze | 40 C., 25 C. | 100 | −29 | 10 | 25 | 1 mL |
| | Solution 1 | | | | | | | | | | |

### Example 3

### Stability Analysis of Adalimumab Solid Units

The stability of the antibody adalimumab within the solid units and cakes prepared according to the methods described in Example 2 was assessed by cation exchange chromatography (CEX) and size exclusion chromatography (SEC) following specific storage conditions. The levels of aggregates, monomers, and fragments of adalimumab in the reconstituted solution was determined by SEC HPLC. The levels of acidic species and other charged variants of adalimumab in the reconstituted solution were quantified using a CEX HPLC method.

For SEC and CEX testing, approximately 100 solid units comprising adalimumab prepared as described above in Table 2 per study were reconstituted with 100 ml of water as the diluent. One cake comprising adalimumab as described above per study was also reconstituted with water as the diluent.

For the SEC HPLC testing, a Superose 6 HR 10/30 column, 10×300 mm highly cross-linked agarose, 11-15 μm particle size (Amersham Biosciences) was used along with an Agilent HPLC system 1200 Series. Injections were made under isocratic elution conditions using a mobile phase consisting of 20 mM Na₂HPO₄*2H₂O/150 mM NaCl, pH 7.5, and detected with UV absorbance at 214 nm.

For the CEX HPLC testing, a 4 mm×250 mm analytical Dionex ProPac WCX-10 column (Dionex, CA) was used along with an Agilent HPLC system 1200 Series. The mobile phases were 10 mM Sodium Phosphate dibasic pH 7.5 buffer (Mobile phase A) and 10 mM Sodium Phosphate dibasic, 500 mM Sodium Chloride pH 5.5 buffer (Mobile phase B). A binary gradient (6% B: 0 min; 6-16% B: 0-20 min; 16-100% B: 20-22 min; 100% B: 22-26 min; 100-6% B: 26-28 min; 6% B: 28-35 min) was used with detection at 280 nm. Quantitation was based on the relative area percentage of detected peaks. The peaks that elute at residence time less than about 7 min, together, represent the acidic species of adalimumab.

### Stability Testing Under Accelerated Conditions

The stability of adalimumab in the solid units prepared in Example 1 was tested using SEC and CEX HPLC methods following accelerated storage conditions, i.e., storage of the solid units at 40° C. for up to 6 months. Solid units were resuspended in water as described above and subsequently tested. Tests were performed at the initial start of the experiment, and subsequently following 1 month, 3 months, or 6 months of storage.

Results from the SEC and CEX tests of the solid units stored under accelerated conditions for up to 6 months are described in Tables 3 and 4. The study numbers referred to in Tables 3 and 4 correspond to Table 2. Tables 3 and 4 below describe results from the SEC and CEX analysis of the uniform, free flowing solid units (and cakes) containing adalimumab and stored under accelerated conditions for up to 6 months. Table 3 describes the % monomer detected by SEC analysis following storage of the various solid units (and cakes) at the indicated time period at 40° C. at 75% relative humidity. Table 4 provides the results of CEX analysis (sum of lysine variants) of the various solid units (and cakes) at the indicated time period at 40° C. at 75% relative humidity.

TABLE 3
Stability of Solid Units and Cakes of Adalimumab Stored
at 40° C. As Determined by Size Exclusion Chromatography

| | Additional | | | | | 1 | 3 | 6 |
|---|---|---|---|---|---|---|---|---|
| Study | Excipients | Form | pH | Freezing | Initial | Month | Month | Month |
| 1 | N/A | Pearls | 5 | Liquid Nitrogen | 99.4 | 97.9 | 96.1 | N/A |
| 2 | 46 mg/mL Sucrose | Pearls | 5 | Liquid Nitrogen | 99.6 | 99.2 | 98.7 | 98 |
| 3 | N/A | Cake | 5 | Std. Lyo Freeze | 99.4 | 97.9 | 96.4 | N/A |
| 4 | N/A | Cake | 5 | Liquid Nitrogen | 99.4 | 97.8 | 96.2 | N/A |
| 5 | 46 mg/mL Sucrose | Cake | 5 | Std. Lyo Freeze | 99.6 | 99.3 | 99 | 98.5 |
| 6 | 46 mg/mL Sucrose | Pearls | 5 | Liquid Nitrogen | 99.6 | 99.3 | 98.9 | 98.1 |
| 7 | 46 mg/mL Sucrose | Pearls | 6 | Liquid Nitrogen | 99.5 | 99.1 | 98.6 | 98 |
| 9 | 46 mg/mL Sucrose | Pearls | 4 | Liquid Nitrogen | 99.3 | 98.8 | 98.3 | N/A |
| 49 | 46 mg/mL Sucrose | Cake | 5 | Liquid Nitrogen | 99.7 | 99.3 | 98.9 | 98.4 |
| 11 | 46 mg/mL Sucrose | Pearls | 7 | Liquid Nitrogen | 99.6 | 98.6 | 98.1 | N/A |
| 12 | 46 mg/mL Trehalose | Pearls | 5 | Liquid Nitrogen | 99.6 | 99.1 | 98.5 | N/A |
| 13 | 70 mg/mL Sucrose | Pearls | 5 | Liquid Nitrogen | 99.6 | 99.5 | 99.2 | 98.7 |
| 14 | 90 mg/mL Sucrose | Pearls | 5 | Liquid Nitrogen | 99.6 | 99.6 | 99.4 | 99 |
| 17 | 2.5% glycine, 4% sucrose | Pearls | 5 | Liquid Nitrogen | 99.7 | 99.4 | 99.2 | 98.5 |
| 26 | 5% hydroxypropyl | Pearls | 5 | Liquid Nitrogen | 99.4 | 97.1 | 95.7 | N/A |
| | beta cyclodextrin | | | | | | | |
| 20 | 5% glycine, 4% trehalose | Pearls | 5 | Liquid Nitrogen | 99.6 | 99.1 | 98.8 | N/A |
| 8 | N/A | Pearls | 5 | Liquid Nitrogen | 99.5 | 98.2 | 96.6 | N/A |
| 54 | 46 mg/mL Trehalose | Cake | 5 | Std. Lyo Freeze | 99.6 | 99.2 | 98.7 | 97.9 |
| 55 | 70 mg/mL Sucrose | Cake | 5 | Std. Lyo Freeze | 99.7 | 99.5 | 99.3 | 98.9 |
| 56 | 90 mg/mL Sucrose | Cake | 5 | Std. Lyo Freeze | 99.7 | 99.6 | 99.4 | 99.1 |
| 57 | 2.5% glycine, 4% sucrose | Cake | 5 | Std. Lyo Freeze | 99.7 | 97.6 | N/A | N/A |
| 58 | 5% hydroxypropyl | Cake | 5 | Std. Lyo Freeze | 99.4 | 96.9 | N/A | N/A |
| | beta cyclodextrin | | | | | | | |
| 59 | 5% glycine, 4% trehalose | Cake | 5 | Std. Lyo Freeze | 99.7 | 99.4 | 99.2 | N/A |
| 16 | 46 mg/mL Sucrose | Cake | 5 | Annealing with initial | 99.6 | 99.4 | 99 | 98.5 |
| | | | | std. lyo freeze | | | | |
| 51 | 2.5% glycine, 4% sucrose | Cake | 5 | Annealing with initial | 99.7 | 99.5 | 99.2 | (7.5) 98.7 |
| | | | | std. lyo freeze | | | | |
| 50 | 46 mg/mL Sucrose | Cake | 5 | Annealing with initial | 99.7 | 99.4 | 99 | 98.4 |
| | | | | liquid nitrogen freeze | | | | |
| 52 | 2.5% glycine, 4% sucrose | Cake | 5 | Annealing with initial | 99.7 | 99.4 | 99.2 | (7.5) 98.6 |
| | | | | liquid nitrogen freeze | | | | |
| 32 | 77 mg/mL Sucrose | Pearls | 5 | Liquid Nitrogen | 99.0 | 97.7 | N/A | N/A |
| 33 | 88 mg/mL Sucrose | Pearls | 5 | Liquid Nitrogen | 98.9 | 97.7 | N/A | N/A |
| 34 | 100 mg/mL Sucrose | Pearls | 5 | Liquid Nitrogen | 98.9 | 97.7 | N/A | N/A |
| 18 | 5% glycine, 4% sucrose | Pearls | 5 | Liquid Nitrogen | 99.7 | 99.4 | N/A | 98.7 |
| 19 | 2.5% glycine, 4% trehalose | Pearls | 5 | Liquid Nitrogen | 99.6 | 99.3 | N/A | 98.4 |
| 22 | 5% sucrose, 1% dextran | Pearls | 5 | Liquid Nitrogen | 99.6 | 99.1 | 98.3 | 97.8 |
| 23 | 5% trehalose, 1% dextran | Pearls | 5 | Liquid Nitrogen | 99.6 | 98.8 | N/A | 96.9 |
| 25 | 1% PEG, 10 mM trehalose | Pearls | 5 | Liquid Nitrogen | 99.5 | 97.7 | N/A | N/A |

TABLE 4
Stability of Solid Units and Cakes of Adalimumab Stored
at 40° C. As Determined by Cation Exchange Chromatography

| | Additional | | | | | 1 | 3 | 6 |
|---|---|---|---|---|---|---|---|---|
| Study | Excipients | Form | pH | Freezing | Initial | Month | Month | Month |
| 1 | N/A | Pearls | 5 | Liquid Nitrogen | 83.4 | 79.9 | 76.5 | N/A |
| 2 | 46 mg/mL Sucrose | Pearls | 5 | Liquid Nitrogen | 82 | 81.8 | 80.4 | 78.8 |
| 3 | N/A | Cake | 5 | Std. Lyo Freeze | 83.6 | 79.9 | 76.8 | N/A |
| 4 | N/A | Cake | 5 | Liquid Nitrogen | 83.5 | 79.9 | 76.3 | N/A |
| 5 | 46 mg/mL Sucrose | Cake | 5 | Std. Lyo Freeze | 84.8 | 83.5 | 82.5 | 80.4 |
| 6 | 46 mg/mL Sucrose | Pearls | 5 | Liquid Nitrogen | 84.6 | 83.2 | 81.9 | 79.3 |
| 7 | 46 mg/mL Sucrose | Pearls | 6 | Liquid Nitrogen | 84.6 | 83.2 | 82.2 | 80.8 |
| 9 | 46 mg/mL Sucrose | Pearls | 4 | Liquid Nitrogen | 84.3 | 82.0 | 79.3 | N/A |
| 49 | 46 mg/mL Sucrose | Cake | 5 | Liquid Nitrogen | 84.6 | 83.3 | 82 | 80.2 |
| 11 | 46 mg/mL Sucrose | Pearls | 7 | Liquid Nitrogen | 84.6 | 83.2 | 82.4 | N/A |
| 12 | 46 mg/mL Trehalose | Pearls | 5 | Liquid Nitrogen | 84.5 | 82.3 | 80.3 | N/A |
| 13 | 70 mg/mL Sucrose | Pearls | 5 | Liquid Nitrogen | 84.6 | 83.5 | 82 | 80.5 |
| 14 | 90 mg/mL Sucrose | Pearls | 5 | Liquid Nitrogen | 84.6 | 83.7 | 82.4 | 80.8 |
| 17 | 2.5% glycine, 4% sucrose | Pearls | 5 | Liquid Nitrogen | 84.3 | 82.0 | 80.2 | 75.9 |
| 26 | 5% hydroxypropyl | Pearls | 5 | Liquid Nitrogen | 83.3 | 74.7 | 69.2 | N/A |
| | beta cyclodextrin | | | | | | | |
| 20 | 5% glycine, 4% trehalose | Pearls | 5 | Liquid Nitrogen | 84.3 | 80.5 | 77.3 | N/A |
| 8 | N/A | Pearls | 5 | Liquid Nitrogen | 84.4 | 81.3 | 78.8 | N/A |
| 54 | 46 mg/mL Trehalose | Cake | 5 | Std. Lyo Freeze | 84.3 | 82.8 | 81.5 | 78.8 |
| 55 | 70 mg/mL Sucrose | Cake | 5 | Std. Lyo Freeze | 84.4 | 83.7 | 82.8 | 81.1 |
| 56 | 90 mg/mL Sucrose | Cake | 5 | Std. Lyo Freeze | 84.5 | 83.8 | 82.9 | 81.3 |
| 57 | 2.5% glycine, 4% sucrose | Cake | 5 | Std. Lyo Freeze | 84.2 | 69 | N/A | N/A |
| 58 | 5% hydroxypropyl | Cake | 5 | Std. Lyo Freeze | 82.5 | 74.7 | N/A | N/A |
| | beta cyclodextrin | | | | | | | |
| 59 | 5% glycine, 4% trehalose | Cake | 5 | Std. Lyo Freeze | 84.1 | 80.4 | 77.7 | N/A |
| 16 | 46 mg/mL Sucrose | Cake | 5 | Annealing with initial | 84.4 | 83.5 | 82.3 | 80.4 |
| | | | | std. lyo freeze | | | | |
| 51 | 2.5% glycine, 4% sucrose | Cake | 5 | Annealing with initial | 84.0 | 81.7 | 79.8 | (7.5) 76.7 |
| | | | | std. lyo freeze | | | | |
| 50 | 46 mg/mL Sucrose | Cake | 5 | Annealing with initial | 84.5 | 83.4 | 82.4 | 80.2 |
| | | | | liquid nitrogen freeze | | | | |
| 52 | 2.5% glycine, 4% sucrose | Cake | 5 | Annealing with initial | 84.2 | 81.3 | 78.8 | (7.5) 76.7 |
| | | | | liquid nitrogen freeze | | | | |
| 24 | 1% PEG, 10 mM sucrose | Pearls | 5 | Liquid Nitrogen | 84.6 | 79 | N/A | N/A |
| 21 | 1% sucrose, 1% dextran | Pearls | 5 | Liquid Nitrogen | 84.5 | 81.6 | 79.7 | N/A |
| 47 | 46 mg/mL Sucrose | Pearls | 5 | Liquid Nitrogen | 85.3 | 82.5 | N/A | N/A |
| 31 | 61 mg/mL Sucrose | Pearls | 5 | Liquid Nitrogen | 85.4 | 82.1 | N/A | N/A |
| 32 | 77 mg/mL Sucrose | Pearls | 5 | Liquid Nitrogen | 85.2 | 82.5 | N/A | N/A |
| 33 | 88 mg/mL Sucrose | Pearls | 5 | Liquid Nitrogen | 85.2 | 82.6 | N/A | N/A |
| 34 | 100 mg/mL Sucrose | Pearls | 5 | Liquid Nitrogen | 85.2 | 82.8 | N/A | N/A |
| 18 | 5% glycine, 4% sucrose | Pearls | 5 | Liquid Nitrogen | 83.9 | 80 | N/A | 74.4 |
| 19 | 2.5% glycine, 4% trehalose | Pearls | 5 | Liquid Nitrogen | 83.9 | 81.7 | N/A | 77 |
| 22 | 5% sucrose, 1% dextran | Pearls | 5 | Liquid Nitrogen | 84.5 | 83 | 80.7 | 79.7 |
| 23 | 5% trehalose, 1% dextran | Pearls | 5 | Liquid Nitrogen | 84.4 | 82.3 | N/A | 77.9 |
| 25 | 1% PEG, 10 mM trehalose | Pearls | 5 | Liquid Nitrogen | 84.2 | 79.6 | N/A | N/A |

The results of Table 3 show that Studies 2, 6, 7, 13, 14, 17, and 19 demonstrate substantial stability of the antibody adalimumab within the solid units for six months stored at 40° C. The native structure of the protein was preserved and the protein aggregation was reduced by the addition of the lyoprotectant sucrose or trehalose at or above 40 mg/mL and/or in combination with the bulking agent glycine at 25 mg/mL. Study 1 showed significant aggregation over time in the solid units without sucrose in the starting formulation. Studies 13 and 14 demonstrate as the amount of sucrose is increased the aggregation is reduced and the physical stability is improved. Study 17 reveals that the freezing of the solid unit enables improved stability over standard lyophilization freezing in the form of a cake shown in Study 57, where comparative studies 17 and 57 contained sucrose and glycine.

The results described in Table 4 show that Studies 2, 6, 7, 13, 14, 17, and 19 demonstrate substantial stability of the antibody adalimumab within the solid units for six months stored at 40° C. The degradation was reduced by the addition of the lyoprotectant sucrose or trehalose at or above 40 mg/mL and/or in combination with the bulking agent glycine at 25 mg/mL. Study 1 showed significant chemical degradation over time in the solid units without sucrose in the starting formulation. Studies 13 and 14 demonstrate as the amount of sucrose is increased the overall stability is improved. Study 17 reveals that the freezing of the dosage unit enables improved stability over standard lyophilization freezing in the form of a cake shown in Study 57, where comparative studies 17 and 57 both contained sucrose and glycine.

Tables 5 and 6 below describe results from the SEC and CEX analysis, respectively, of the free-flowing solid units containing adalimumab and cakes containing adalimumab and stored at 25° C. (60% relative humidity) for up to 24 months.

TABLE 5
Stability of Solid Units of Adalimumab Stored at 25° C. As Determined by Size Exclusion Chromatography (SEC)

| | Additional | | | | | 3 | 6 | 9 | 12 | 18 | 24 |
|---|---|---|---|---|---|---|---|---|---|---|---|
| Study | Excipients | Form | pH | Freezing | Initial | Month | Month | Month | Month | Month | Month |
| 1 | N/A | Pearls | 5 | Liquid Nitrogen | 99.4 | 98.8 | 98.3 | N/A | N/A | N/A | N/A |
| 2 | 46 mg/mL Sucrose | Pearls | 5 | Liquid Nitrogen | 99.6 | 99.4 | 99.2 | N/A | 99.2 | N/A | 98.9 |
| 3 | N/A | Cake | 5 | Std. Lyo Freeze | 99.4 | 98.8 | 98.2 | N/A | N/A | N/A | N/A |
| 4 | N/A | Cake | 5 | Liquid Nitrogen | 99.4 | 98.8 | 98.2 | N/A | N/A | N/A | N/A |
| 5 | 46 mg/mL Sucrose | Cake | 5 | Std. Lyo Freeze | 99.6 | N/A | 99.3 | N/A | N/A | N/A | 99.1 |
| 6 | 46 mg/mL Sucrose | Pearls | 5 | Liquid Nitrogen | 99.6 | N/A | 99.2 | (9.5) 99.1 | 99.3 | N/A | 98.9 |
| 7 | 46 mg/mL Sucrose | Pearls | 6 | Liquid Nitrogen | 99.5 | N/A | 99 | (9.5) 98.9 | N/A | N/A | 98.8 |
| 9 | 46 mg/mL Sucrose | Pearls | 4 | Liquid Nitrogen | 99.3 | N/A | N/A | N/A | N/A | N/A | 98.3 |
| 49 | 46 mg/mL Sucrose | Cake | 5 | Liquid Nitrogen | 99.7 | N/A | 99.3 | N/A | N/A | N/A | 99 |
| 11 | 46 mg/mL Sucrose | Pearls | 7 | Liquid Nitrogen | 99.6 | N/A | N/A | 98.1 | N/A | N/A | 98.4 |
| 12 | 46 mg/mL Trehalose | Pearls | 5 | Liquid Nitrogen | 99.6 | N/A | N/A | 98.9 | N/A | N/A | 98.7 |
| 13 | 70 mg/mL Sucrose | Pearls | 5 | Liquid Nitrogen | 99.6 | N/A | 99.3 | 99.3 | 99.4 | N/A | 99.3 |
| 14 | 90 mg/mL Sucrose | Pearls | 5 | Liquid Nitrogen | 99.6 | N/A | 99.4 | 99.4 | 99.6 | N/A | 99.5 |
| 17 | 2.5% glycine, 4% sucrose | Pearls | 5 | Liquid Nitrogen | 99.7 | N/A | 99.4 | 99.3 | 99.5 | N/A | 99.3 |
| 26 | 5% hydroxypropyl | Pearls | 5 | Liquid Nitrogen | 99.4 | N/A | N/A | N/A | N/A | N/A | N/A |
| | beta cyclodextrin | | | | | | | | | | |
| 20 | 5% glycine, 4% trehalose | Pearls | 5 | Liquid Nitrogen | 99.6 | N/A | N/A | 99 | N/A | N/A | 99 |
| 8 | N/A | Pearls | 5 | Liquid Nitrogen | 99.5 | N/A | N/A | N/A | N/A | N/A | N/A |
| 54 | 46 mg/mL Trehalose | Cake | 5 | Std. Lyo Freeze | 99.6 | N/A | 99.1 | N/A | 99.1 | N/A | N/A |
| 55 | 70 mg/mL Sucrose | Cake | 5 | Std. Lyo Freeze | 99.7 | N/A | 99.3 | N/A | 99.5 | N/A | N/A |
| 56 | 90 mg/mL Sucrose | Cake | 5 | Std. Lyo Freeze | 99.7 | N/A | 99.4 | N/A | 99.5 | N/A | N/A |
| 57 | 2.5% glycine, 4% sucrose | Cake | 5 | Std. Lyo Freeze | 99.7 | N/A | N/A | N/A | N/A | N/A | N/A |
| 58 | 5% hydroxypropyl | Cake | 5 | Std. Lyo Freeze | 99.4 | N/A | N/A | N/A | N/A | N/A | N/A |
| | beta cyclodextrin | | | | | | | | | | |
| 59 | 5% glycine, 4% trehalose | Cake | 5 | Std. Lyo Freeze | 99.7 | N/A | N/A | N/A | 99.4 | N/A | N/A |
| 16 | 46 mg/mL Sucrose | Cake | 5 | Annealing with | 99.6 | N/A | 99.3 | N/A | N/A | N/A | N/A |
| | | | | initial std. lyo | | | | | | | |
| 51 | 2.5% glycine, 4% sucrose | Cake | 5 | Annealing with | 99.7 | N/A | N/A | N/A | N/A | N/A | N/A |
| | | | | initial std. lyo | | | | | | | |
| 50 | 46 mg/mL Sucrose | Cake | 5 | Annealing with | 99.7 | N/A | 99.2 | N/A | N/A | N/A | N/A |
| | | | | initial liquid | | | | | | | |
| 52 | 2.5% glycine, 4% sucrose | Cake | 5 | Annealing with initial | 99.7 | N/A | N/A | N/A | N/A | N/A | N/A |
| | | | | liquid nitrogen freeze | | | | | | | |
| 24 | 1% PEG, 10 mM sucrose | Pearls | 5 | Liquid Nitrogen | 99.6 | N/A | N/A | N/A | N/A | N/A | N/A |
| 21 | 1% sucrose, 1% dextran | Pearls | 5 | Liquid Nitrogen | 99.6 | N/A | N/A | N/A | N/A | N/A | N/A |
| 47 | 46 mg/mL Sucrose | Pearls | 5 | Liquid Nitrogen | 99.1 | N/A | N/A | N/A | N/A | N/A | N/A |
| 31 | 61 mg/mL Sucrose | Pearls | 5 | Liquid Nitrogen | 99.0 | N/A | N/A | N/A | N/A | N/A | N/A |
| 32 | 77 mg/mL Sucrose | Pearls | 5 | Liquid Nitrogen | 99.0 | N/A | N/A | N/A | N/A | N/A | N/A |
| 33 | 88 mg/mL Sucrose | Pearls | 5 | Liquid Nitrogen | 98.9 | N/A | N/A | N/A | N/A | N/A | N/A |
| 34 | 100 mg/mL Sucrose | Pearls | 5 | Liquid Nitrogen | 98.9 | N/A | N/A | N/A | N/A | N/A | N/A |
| 18 | 5% glycine, 4% sucrose | Pearls | 5 | Liquid Nitrogen | 99.7 | N/A | 99.4 | N/A | N/A | N/A | 99.4 |
| 19 | 2.5% glycine, 4% trehalose | Pearls | 5 | Liquid Nitrogen | 99.6 | N/A | 99.3 | N/A | N/A | N/A | 99.1 |
| 22 | 5% sucrose, 1% dextran | Pearls | 5 | Liquid Nitrogen | 99.6 | N/A | 99.1 | N/A | N/A | N/A | 98.6 |
| 23 | 5% trehalose, 1% dextran | Pearls | 5 | Liquid Nitrogen | 99.6 | N/A | 98.8 | N/A | N/A | N/A | 98 |
| 25 | 1% PEG, 10 mM trehalose | Pearls | 5 | Liquid Nitrogen | 99.5 | N/A | N/A | N/A | N/A | N/A | N/A |

TABLE 6
Stability of Solid Units of Adalimumab Stored at 25° C. As Determined by Cation Exchange Chromatography (CEX)

| | Additional | | | | | 3 | 6 | 9 | 12 | 18 | 24 |
|---|---|---|---|---|---|---|---|---|---|---|---|
| Study | Excipients | Form | pH | Freezing | Initial | Month | Month | Month | Month | Month | Month |
| 1 | N/A | Pearls | 5 | Liquid Nitrogen | 83.4 | 81.7 | 81.5 | N/A | N/A | N/A | N/A |
| 2 | 46 mg/mL Sucrose | Pearls | 5 | Liquid Nitrogen | 82 | 82.7 | 82.4 | N/A | 80.6 | N/A | 81.5 |
| 3 | N/A | Cake | 5 | Std. Lyo Freeze | 83.6 | 82 | 81.2 | N/A | N/A | N/A | N/A |
| 4 | N/A | Cake | 5 | Liquid Nitrogen | 83.5 | 81.8 | 81.3 | N/A | N/A | N/A | N/A |
| 5 | 46 mg/mL Sucrose | Cake | 5 | Std. Lyo Freeze | 84.8 | N/A | 83.6 | N/A | N/A | N/A | 83.9 |
| 6 | 46 mg/mL Sucrose | Pearls | 5 | Liquid Nitrogen | 84.6 | N/A | 83.1 | 83.2 | 82 | N/A | 84 |
| 7 | 46 mg/mL Sucrose | Pearls | 6 | Liquid Nitrogen | 84.6 | N/A | 83.3 | 83.1 | N/A | N/A | 84 |
| 9 | 46 mg/mL Sucrose | Pearls | 4 | Liquid Nitrogen | 84.3 | N/A | N/A | N/A | N/A | N/A | 82.1 |
| 49 | 46 mg/mL Sucrose | Cake | 5 | Liquid Nitrogen | 84.6 | N/A | 83.2 | N/A | N/A | N/A | 83.6 |
| 11 | 46 mg/mL Sucrose | Pearls | 7 | Liquid Nitrogen | 84.6 | N/A | N/A | 82.9 | N/A | N/A | 84.1 |
| 12 | 46 mg/mL Trehalose | Pearls | 5 | Liquid Nitrogen | 84.5 | N/A | N/A | 82.5 | N/A | N/A | 81.3 |
| 13 | 70 mg/mL Sucrose | Pearls | 5 | Liquid Nitrogen | 84.6 | N/A | 83.5 | 83.2 | 82 | N/A | 82.4 |
| 14 | 90 mg/mL Sucrose | Pearls | 5 | Liquid Nitrogen | 84.6 | N/A | 83.5 | 83.5 | 81.3 | N/A | 82.5 |
| 17 | 2.5% glycine, 4% sucrose | Pearls | 5 | Liquid Nitrogen | 84.3 | N/A | 82.4 | 82.2 | 80.8 | N/A | 80.8 |
| 26 | 5% hydroxypropyl | Pearls | 5 | Liquid Nitrogen | 83.3 | N/A | N/A | N/A | N/A | N/A | N/A |
| | beta cyclodextrin | | | | | | | | | | |
| 20 | 5% glycine, 4% trehalose | Pearls | 5 | Liquid Nitrogen | 84.3 | N/A | N/A | 81.1 | N/A | N/A | 80.1 |
| 8 | N/A | Pearls | 5 | Liquid Nitrogen | 84.4 | N/A | N/A | N/A | N/A | N/A | N/A |
| 54 | 46 mg/mL Trehalose | Cake | 5 | Std. Lyo Freeze | 84.3 | N/A | 82.9 | N/A | 81.8 | N/A | N/A |
| 55 | 70 mg/mL Sucrose | Cake | 5 | Std. Lyo Freeze | 84.4 | N/A | 83.7 | N/A | 82.7 | N/A | N/A |
| 56 | 90 mg/mL Sucrose | Cake | 5 | Std. Lyo Freeze | 84.5 | N/A | 83.7 | N/A | 82.7 | N/A | N/A |
| 57 | 2.5% glycine, 4% sucrose | Cake | 5 | Std. Lyo Freeze | 84.2 | N/A | N/A | N/A | N/A | N/A | N/A |
| 58 | 5% hydroxypropyl | Cake | 5 | Std. Lyo Freeze | 82.5 | N/A | N/A | N/A | N/A | N/A | N/A |
| | beta cyclodextrin | | | | | | | | | | |
| 59 | 5% glycine, 4% trehalose | Cake | 5 | Std. Lyo Freeze | 84.1 | N/A | N/A | N/A | 80.8 | N/A | N/A |
| 16 | 46 mg/mL Sucrose | Cake | 5 | Annealing with initial | 84.4 | N/A | 83.3 | N/A | N/A | N/A | N/A |
| | | | | std. lyo freeze | | | | | | | |

| | Additional | | | | | 3 | 6 | 9 | 12 | 18 | |
|---|---|---|---|---|---|---|---|---|---|---|---|
| Study | Excipients | Form | pH | Freezing | Initial | Month | Month | Month | Month | Month | N/A |
| 51 | 2.5% glycine, 4% sucrose | Cake | 5 | Annealing with initial | 84.0 | N/A | N/A | N/A | N/A | N/A | 80.8 |
| | | | | std. lyo freeze | | | | | | | |

| | Additional | | | | | 3 | 6 | 9 | 12 | 18 | 24 |
|---|---|---|---|---|---|---|---|---|---|---|---|
| Study | Excipients | Form | pH | Freezing | Initial | Month | Month | Month | Month | Month | Month |
| 50 | 46 mg/mL Sucrose | Cake | 5 | Annealing with initial | 84.5 | N/A | 83.4 | N/A | N/A | N/A | N/A |
| | | | | liquid nitrogen freeze | | | | | | | |
| 52 | 2.5% glycine, 4% sucrose | Cake | 5 | Annealing with initial | 84.2 | N/A | N/A | N/A | N/A | N/A | N/A |
| | | | | liquid nitrogen freeze | | | | | | | |
| 24 | 1% PEG, 10 mM sucrose | Pearls | 5 | Liquid Nitrogen | 84.6 | N/A | N/A | N/A | N/A | N/A | N/A |
| 21 | 1% sucrose, 1% dextran | Pearls | 5 | Liquid Nitrogen | 84.5 | N/A | N/A | N/A | N/A | N/A | N/A |
| 47 | 46 mg/mL Sucrose | Pearls | 5 | Liquid Nitrogen | 85.3 | N/A | N/A | N/A | N/A | N/A | N/A |
| 31 | 61 mg/mL Sucrose | Pearls | 5 | Liquid Nitrogen | 85.4 | N/A | N/A | N/A | N/A | N/A | N/A |
| 32 | 77 mg/mL Sucrose | Pearls | 5 | Liquid Nitrogen | 85.2 | N/A | N/A | N/A | N/A | N/A | N/A |
| 33 | 88 mg/mL Sucrose | Pearls | 5 | Liquid Nitrogen | 85.2 | N/A | N/A | N/A | N/A | N/A | N/A |
| 34 | 100 mg/mL Sucrose | Pearls | 5 | Liquid Nitrogen | 85.2 | N/A | N/A | N/A | N/A | N/A | N/A |
| 18 | 5% glycine, 4% sucrose | Pearls | 5 | Liquid Nitrogen | 83.9 | N/A | 81.9 | N/A | N/A | N/A | 80.9 |
| 19 | 2.5% glycine, 4% trehalose | Pearls | 5 | Liquid Nitrogen | 83.9 | N/A | 82.2 | N/A | N/A | N/A | 81.7 |
| 22 | 5% sucrose, 1% dextran | Pearls | 5 | Liquid Nitrogen | 84.5 | N/A | 83.2 | N/A | N/A | N/A | 82.4 |
| 23 | 5% trehalose, 1% dextran | Pearls | 5 | Liquid Nitrogen | 84.4 | N/A | 82.6 | N/A | N/A | N/A | 81.4 |
| 25 | 1% PEG, 10 mM trehalose | Pearls | 5 | Liquid Nitrogen | 84.2 | N/A | N/A | N/A | N/A | N/A | N/A |

As described in Table 5, Studies 2, 6, and 7 demonstrated substantial stability of the antibody adalimumab within the solid units for twenty four months stored at 25° C. Studies 13, 14, 17, and 19 demonstrate substantial stability of the antibody adalimumab within the solid units for twelve months stored at 25° C. The native structure of the protein was preserved and the protein aggregation was reduced by the addition of the lyoprotectant sucrose or trehalose at or above 40 mg/mL and/or in combination with the bulking agent glycine at 25 mg/mL. Study 1 showed significant aggregation over time in the solid units without sucrose in the starting formulation.

The results provided in Table 6 show that Studies 2, 6, and 7 demonstrated substantial stability of the antibody adalimumab within the solid units for twenty four months stored at 25° C. Studies 13, 14, 17, and 19 demonstrate substantial stability of the antibody adalimumab within the solid units for twelve months stored at 25° C. The degradation was reduced by the addition of the lyoprotectant sucrose or trehalose at or above 40 mg/mL and/or in combination with the bulking agent glycine at 25 mg/mL. Study 1 showed significant aggregation over time in the solid units without sucrose in the starting formulation.

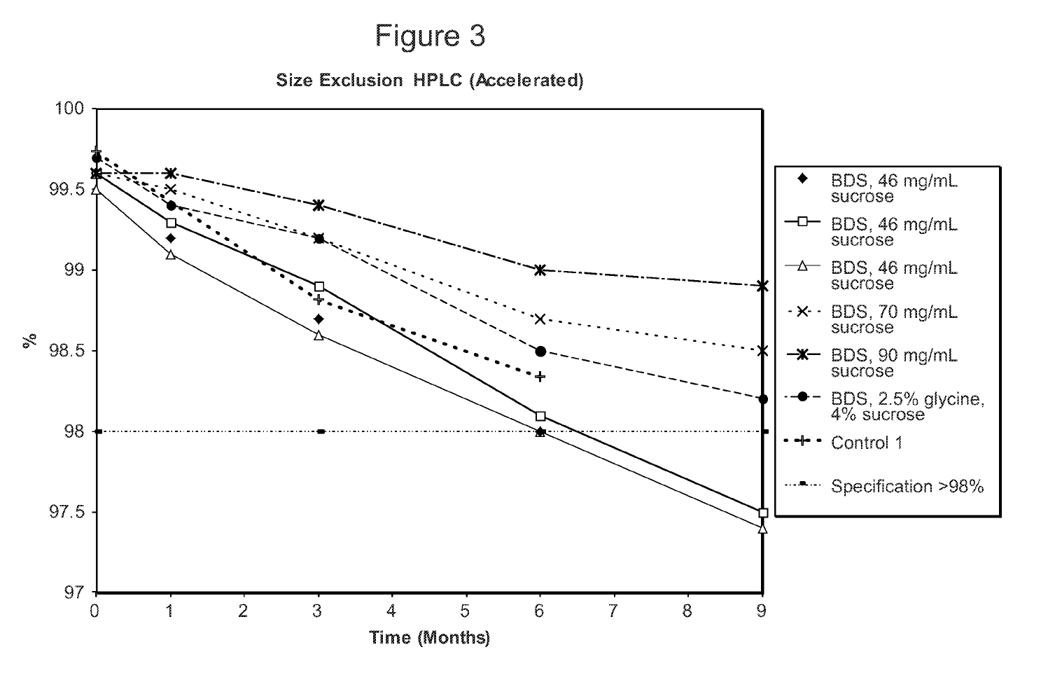

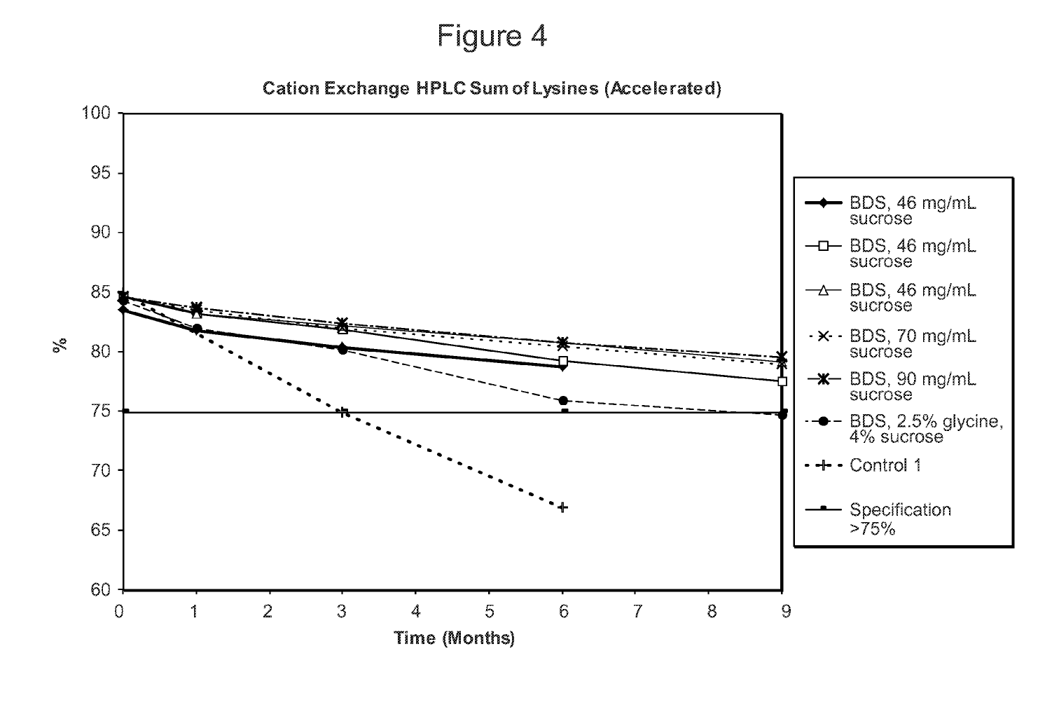

In addition to Tables 3 to 6 above, FIGS. 1 to 4 provide comparative data for adalimumab formulated in solid units containing 46 to 90 mg/ml of sucrose (concentrations referring to the starting solution which was lyophilized; see Example 1), as well as a solid unit having a sucrose and glycine combination, stored at 25° C. for up to 18 months (FIGS. 1 and 2) or 40° C. for up to 9 months (FIGS. 3 and 4). FIG. 1 describes the % monomer in the reconstituted solution using SEC-HPLC, while FIG. 2 describes results from CEX-HPLC analysis. Control 1 in FIGS. 1 to 4 is equivalent to Solution 1 described in Example 1.

As demonstrated in Tables 3-6, solid units prepared utilizing a controlled nucleation freezing process of a solution comprising sucrose or trehalose provided a stabilizing effect to adalimumab within the solid units. Furthermore, even in the presence of additional excipients (e.g., buffer and NaCl) which are traditionally omitted for lyophilization, the adalimumab within the solid units remained stable (see, e.g., Studies 2 and 12). Moreover, these solid units meet the specifications for Humira at 25° C./60% RH for 24 months.

### Example 4

### Adalimumab Solid Units Formulated with an Enteric Protectant

The following example describes a solid unit containing adalimumab and an exemplary enteric protectant, i.e., hydroxypropylmethylcellulose (HPMC).

A 10% HPMC solution (polymer solution) was made with water. Adalimumab solid units were dissolved in the 10% HPMC solution to obtain an antibody concentration of about 50 mg/ml. The resulting solution contained the following ingredients (concentrations in parentheses): adalimumab (50 mg/ml, although ranging from 50-80 mg/ml), mannitol (12 mg/ml); tween 80 (1 mg/ml); sodium chloride (6.15 mg/ml); sodium phosphate monobasic (0.86 mg/ml); sodium phosphate dibasic (1.53 mg/ml); sodium citrate (0.3 mg/ml); citric acid monohydrate (1.3 mg/ml); sucrose (46 mg/ml); hypromellose acetate succinate NF (HPMC AS-LF) (10 mg/ml) and NaOH (6 mg/ml).

After the adalimumab solid units were fully dissolved and mixed with the polymer solution, the resulting solution was used to manufacture HPMC+adalimumab solid units. More specifically, the adalimumab/HPMC solution was lyophilized to obtain adalimumab solid units containing an enteric protectant i.e., HPMC. The lyophilization process conditions included a loading step at about −50° C., followed by a freezing at about −15° C. and subsequently at −50° C. Evacuation was the performed at a pressure of 100 microns, followed by a primary drying step at about −15° C. and 100 microns of pressure and a second primary drying step at about 30° C. and 100 microns of pressure. This lyophilization process resulted in stable solid units containing adalimumab and an enteric protectant, i.e. HPMC.

Combining adalimumab with HPMC resulted in a stable solid unit. SEC and CEX HPLC analysis showed that adalimumab maintained stability in the presence of HPMC. Specifically, solid units made from an initial solution of 10% HPMC, 50 mg/ml adalimumab resulted in a CEX profile (sum of lysines) of 83.8 and an SEC profile (% monomer) of 99.3. Adalimumab solid units (made from solution 1 of Table 2) rinsed in acetone (described in more detail in Example 4) also resulted in stable solid units, where the CEX profile (sum of lysines) of 85.6 and an SEC profile (% monomer) of 99.7.

### Example 5

### Adalimumab Solid Units Formulated with Additional Polymers

In addition to HPMC, additional polymers and solvents were tested in adalimumab solid units.

The following solvents were evaluated for physical appearance after placing pearls in each solvent: chloroform, methanol, isopropanol, ethanol, acetone, petroleum ether, tert-butanol, and reagent alcohol. For the initial observations of the pearls soaked in solvent, the following pearls remained intact: chloroform, methanol, ethanol, acetone, petroleum ether, tert-butanol, and reagent alcohol. The pearls soaked in the isopropanol slowly dissolved (pearls failed to remain in-tact for the initial solvent soak). The pearls were soaked in the solvent for approximately 5 minutes. The solvent was then drained from the scintillation vial and capped. The vials were then uncapped and placed in the dessicator under vacuum. The next day the pearls that still remained intact and spherical were the pearls soaked in chloroform, acetone, and petroleum ether. Therefore, further evaluation with HPLC analysis was executed with pearls soaked in chloroform, acetone, and petroleum ether.

Chloroform, acetone, and petroleum ether were all tested to determine the impact of each solvent on a solid unit containing adalimumab.

About 2 mls of acetone was placed in a vial containing approximately 0.068 g of adalimumab solid units. The solid units were swirled in the acetone for about 20 second (single rinse) and then immediately dried under nitrogen for about 10 minutes. The dried solid units were then analyzed by SEC and CEX HPLC. Chloroform and petroleum ether were applied in a similar manner.

Results are described in Table 7 and show that each of the solvents tested were comparable to the control with respect to maintaining stability of adalimumab. The control in Tables 7 and 8 was an unrinsed solid unit containing adalimumab (see solution 1 from Example 1). The data demonstrates physical and chemical stability of the solid units rinsed in chloroform, petroleum ether, and acetone.

TABLE 7
SEC HPLC Analysis of Adalimumab Solid
Units Using Various Solvents

| | Control | | Petroleum | |
|---|---|---|---|---|
| | (Unrinsed) | Chloroform | Ether | Acetone |
| SEC (% | 99.8 | 99.6 | 99.7 | 99.7 |
| Monomer) | | | | |
| Sum of | 85.7 | 85.4 | 85.8 | 85.6 |
| Lysines | | | | |
| Acidic I | 2.5 | 2.6 | 2.6 | 2.6 |
| Acidic II | 10.2 | 10.4 | 10.1 | 10.3 |

Solid units were also made using the combination of adalimumab and a polymer, including 1% methocellulose, 1% kollicoat, or 0.5% copovidone. Solid units containing adalimumab and each of these polymers were made in a manner similar to that described in Example 3 for HPMC. Following the production of a solid unit containing adalimumab and methocellulose, kollicoat, or copovidone, the solid units were rinsed once in acetone. The results from this study are described in Table 8, which shows stability of the combinations of polymers and adalimumab in a solid unit, and also that rinsing the solid units with acetone did not have a significant impact on the stability of adalimumab, as determined by SEC and CEX HPLC.

TABLE 8
SEC and CEX HPLC Analysis of Adalimumab
Combined with Various Polymers

| | | 1% | 1% | 0.5% |
|---|---|---|---|---|
| | Control | Methocel/ | Kollicoat | Copovidone/ |
| | (unrinsed) | Acetone | IR/Acetone | Acetone |
| SEC | 99.8 | 99.8 | 99.8 | 94.3 |
| (monomer %) | | | | |
| Sum of | 85.7 | 85.4 | 85.2 | 85.5 |
| Lysines | | | | |
| Acidic I | 2.5 | 2.6 | 2.6 | 2.6 |
| Acidic II | 10.2 | 10.4 | 10.6 | 10.3 |

The data presented in Table 8 demonstrates physical and chemical stability of the solid units prepared with 1% methocel/acetone, 1% kollicoat IR/acetone, and 0.5% copovidone/acetone.

In sum, the studies in Examples 4 and 5 show that solid units may be made with an enteric protectant and other polymers in combination with adalimumab, such that the stability of the antibody is maintained.

### Example 6

### Stability Analysis of Adalimumab Solid Units Comprising Varying Amounts of Sucrose

As described above, sucrose concentrations of 46 mg/mL and above were evaluated and showed effective stabilization of adalimumab within solid units prepared utilizing controlled nucleation freezing in liquid nitrogen. This example describes the effect of lower concentrations of sucrose on the stability of adalimumab in solid units prepared using controlled nucleation employing liquid nitrogen for a rapid freeze.

The following sucrose concentrations were evaluated in the study: 10 mg/mL, 20 mg/mL, 30 mg/mL, 40 mg/mL, and 50 mg/mL at accelerated storage conditions. All solid units were made from the same adalimumab BDS solution, described in the Table 9 below, and were prepared as described in Example 1.

TABLE 9
Adalimumab Bulk Drug Substance

| | Amount |
|---|---|
| Material | (mg/mL) |
| Adalimumab | 50 |
| Mannitol | 12 |
| Tween 80 | 1 |
| Sodium chloride | 6.15 |
| Sodium phosphate, monobasic (2H2O) | 0.86 |
| Sodium phosphate, dibasic (2H2O) | 1.53 |
| Sodium citrate | 0.30 |
| Citric acid, monohydrate | 1.30 |

The monomer stability of the protein within the solid units was determined by SEC following storage for 0, 1, 2, and 3 months at 40° C./75% RH storage conditions.

Table 10 shows that adalimumab in the solid units with sucrose concentrations at 40 mg/mL or above have the greatest stability. The monomer stability trend for the 20 and 30 mg/mL sucrose formulations show the formulations will be out of specification (OOS) after 6 months. The 10 mg/mL sucrose formulation is OOS for monomer after 3 months at 40° C./75% RH. The monomer specification is >98%.

TABLE 10
Size Exclusion HPLC Stability Data for Adalimumab BDS
with Varying Sucrose Concentrations at 40° C./75% RH

| | | | | | | Sodium | Sodium |
|---|---|---|---|---|---|---|---|
| | | | | | Sodium | Phosphate, | Phosphate, |
| | | Adalimumab | Mannitol | Tween 80 | Chloride | monobasic | dibasic |
| Description | Specification | (mg/mL) | (mg/mL) | (mg/mL) | (mg/mL) | (mg/mL) | (mg/mL) |
| Monomer | ≧98% | 50 | 12 | 1 | 6.15 | 0.86 | 1.53 |
| | | 50 | 12 | 1 | 6.15 | 0.86 | 1.53 |
| | | 50 | 12 | 1 | 6.15 | 0.86 | 1.53 |
| | | 50 | 12 | 1 | 6.15 | 0.86 | 1.53 |
| | | 50 | 12 | 1 | 6.15 | 0.86 | 1.53 |

| | | Sodium | Citric Acid, | | | | | |
|---|---|---|---|---|---|---|---|---|
| | | Citrate | monohydrate | Sucrose | t | 1 | 2 | 3 |
| Description | Specification | (mg/mL) | (mg/mL) | (mg/mL) | zero | month | month | month |
| Monomer | ≧98% | 0.3 | 1.3 | 50 | 99.9 | 99.5 | 99.3 | 98.9 |
| | | 0.3 | 1.3 | 40 | 99.8 | 99.5 | 99.3 | 98.7 |
| | | 0.3 | 1.3 | 30 | 99.9 | 99.3 | 99.1 | 98.5 |
| | | 0.3 | 1.3 | 20 | 99.8 | 99.2 | 99.1 | 98.1 |
| | | 0.3 | 1.3 | 10 | 99.8 | 99.1 | 98.5 | 97.3 |

### Example 7

### Room Temperature Stability of Reconstituted Solid Unit Solutions

The following example investigates the solution stability of reconstituted solid units of adalimumab BDS (see Table 1) with sucrose stored at room temperature.

Solid units comprising adalimumab BDS with 46 mg/mL sucrose prepared as described in Example 2 were transferred to a dual chamber cartridge. After reconstitution with high purity water, the cartridge with the reconstituted solution was maintained at room temperature for 18 hours and then analyzed by SEC-HPLC. The results were compared to a control sample of solid units comprising adalimumab BDS with 46 mg/mL sucrose that were stored at 4° C. and reconstituted with high purity water immediately before HPLC analysis.

The results are presented in Tables 11 and 12 and show that the reconstituted solid unit solution is stable at room temperature for at least 18 hours.

TABLE 11
Size Exclusion Stability Data for Reconstituted BioPearl Solution at Room Temperature

| | | | | | | Sodium | Sodium |
|---|---|---|---|---|---|---|---|
| | | | | | Sodium | Phosphate, | Phosphate, |
| | | Adalimumab | Mannitol | Tween 80 | Chloride | monobasic | dibasic |
| Description | Specification | (mg/mL) | (mg/mL) | (mg/mL) | (mg/mL) | (mg/mL) | (mg/mL) |
| Monomer | ≧98% | 50 | 12 | 1 | 6.15 | 0.86 | 1.53 |
| | | 50 | 12 | 1 | 6.15 | 0.86 | 1.53 |

| | | Sodium | Citric Acid, | | | |
|---|---|---|---|---|---|---|
| | | Citrate | monohydrate | Sucrose | | t |
| Description | Specification | (mg/mL) | (mg/mL) | (mg/mL) | Sample | zero |
| Monomer | ≧98% | 0.3 | 1.3 | 46 | Control | 99.9 |
| | | 0.3 | 1.3 | 46 | Reconstituted so- | 99.8 |
| | | | | | lution 18 hours at | |
| | | | | | room temperature | |

TABLE 12
Cation Exchange Sum of Lysines Stability Data for
Reconstituted BioPearl Solution at Room Temperature

| | | | | | | Sodium | Sodium |
|---|---|---|---|---|---|---|---|
| | | | | | Sodium | Phosphate, | Phosphate, |
| | | Adalimumab | Mannitol | Tween 80 | Chloride | monobasic | dibasic |
| Description | Specification | (mg/mL) | (mg/mL) | (mg/mL) | (mg/mL) | (mg/mL) | (mg/mL) |
| Sum of Lysines | ≧75% | 50 | 12 | 1 | 6.15 | 0.86 | 1.53 |
| | | 50 | 12 | 1 | 6.15 | 0.86 | 1.53 |

| | | Sodium | Citric Acid, | | | |
|---|---|---|---|---|---|---|
| | | Citrate | monohydrate | Sucrose | | t |
| Description | Specification | (mg/mL) | (mg/mL) | (mg/mL) | Sample | zero |
| Sum of Lysines | ≧75% | 0.3 | 1.3 | 46 | Control | 85.8 |
| | | 0.3 | 1.3 | 46 | Reconstituted so- | 85.9 |
| | | | | | lution 18 hours at | |
| | | | | | room temperature | |

### Example 8

### Stability of Solid Units Comprising Low Ionic Adalimumab and Sucrose Formulations

The stability of solid units comprising low ionic formulations, i.e., little to no ionic excipients, of adalimumab was evaluated.

Specifically, in the study, the excipients sodium chloride, sodium phosphate, sodium citrate, and citric acid were removed from a BDS solution of adalimumab to evaluate the stability of low ionic formulations. Sucrose was added as a lyoprotectant to help stabilize the protein during lyophilization.

The following formulations were studied:
Formulation 1: 50 mg/ml adalimumab, 12 mg/ml mannitol, 1 mg/ml tween-80, 6.15 mg/ml NaCl, 0.86 mg/ml sodium phosphate monobasic, 1.53 mg/ml sodium phosphate dibasic, 0.3 mg/ml sodium citrate, 1.3 mg/ml citric acid monohydrate, and 46 mg/ml sucrose.
Formulation 2: 50 mg/ml adalimumab, 12 mg/ml mannitol, 1 mg/ml tween-80, 0.86 mg/ml sodium phosphate monobasic, 1.53 mg/ml sodium phosphate dibasic, 0.3 mg/ml sodium citrate, 1.3 mg/ml citric acid monohydrate, and 60 mg/ml sucrose.
Formulation 3: 50 mg/ml adalimumab, 12 mg/ml mannitol, 1 mg/ml tween-80, 0.65 mg/ml sodium phosphate monobasic, 1.15 mg/ml sodium phosphate dibasic, 0.23 mg/ml sodium citrate, 0.98 mg/ml citric acid monohydrate, and 60 mg/ml sucrose.
Formulation 4: 50 mg/ml adalimumab, 12 mg/ml mannitol, 1 mg/ml tween-80, and 65 mg/ml sucrose.
Formulation 5: 100 mg/ml adalimumab, 1 mg/ml mannitol, 1 mg/ml tween-80, and 90 mg/ml sucrose.
Formulation 6: 100 mg/ml adalimumab, 3 mg/ml mannitol, 1 mg/ml tween-80, and 75 mg/ml sucrose.

The monomer content and the sum of lysines stability of the protein within the solid units was determined by SEC and CEX, respectively, following storage for 0, 1, 2, and 3 months at 40° C./75% RH storage conditions.

Tables 13 and 14 show that the adalimumab (particularly Formulation 4) in the solid units remained stable following accelerated storage conditions.

TABLE 13
Size Exclusion HPLC Stability Data for Adalimumab Low Ionic Formulations at 40° C./75% RH

| | | | | | | | Sodium | Sodium | |
|---|---|---|---|---|---|---|---|---|---|
| | | | | | | Sodium | Phosphate, | Phosphate, | Sodium |
| | | | Adalimumab | Mannitol | Tween 80 | Chloride | monobasic | dibasic | Citrate |
| Description | Specification | Formulation | (mg/mL) | (mg/mL) | (mg/mL) | (mg/mL) | (mg/mL) | (mg/mL) | (mg/mL) |
| Monomer | ≧98% | 1 | 50 | 12 | 1 | 6.15 | 0.86 | 1.53 | 0.3 |
| | | 2 | 50 | 12 | 1 | 0 | 0.86 | 1.53 | 0.3 |
| | | 3 | 50 | 12 | 1 | 0 | 0.65 | 1.15 | 0.23 |
| | | 4 | 50 | 12 | 1 | 0 | 0 | 0 | 0 |
| | | 5 | 100 | 1 | 1 | 0 | 0 | 0 | 0 |
| | | 6 | 100 | 3 | 1 | 0 | 0 | 0 | 0 |

| | | | Citric Acid, | | | | | | |
|---|---|---|---|---|---|---|---|---|---|
| | | | monohydrate | Sucrose | | | | | |
| Description | Specification | Formulation | (mg/mL) | (mg/mL) | 0 | 2 WK | 1 M | 2 M | 3 M |
| Monomer | ≧98% | 1 | 1.3 | 46 | 99.4 | 99.3 | 99.1 | 98.7 | 98.5 |
| | | 2 | 1.3 | 60 | 99.6 | 99.3 | 98.8 | 98.8 | 98.5 |
| | | 3 | 0.98 | 60 | 99.4 | 99.3 | 98.9 | 98.8 | 98.5 |
| | | 4 | 0 | 65 | 99.6 | 99.5 | 99.1 | 99.1 | 98.9 |
| | | 5 | 0 | 90 | 99.5 | 98.9 | 98.7 | 98.4 | 98 |
| | | 6 | 0 | 75 | 99.5 | 98.8 | 98.7 | 98.1 | 97.7 |

TABLE 14
Cation Exchange Sum of Lysines Stability Data for Adalimumab Low Ionic Formulations at 40° C./75% RH

| | | | | | | | Sodium | Sodium | |
|---|---|---|---|---|---|---|---|---|---|
| | | | | | | Sodium | Phosphate, | Phosphate, | Sodium |
| | | | Adalimumab | Mannitol | Tween 80 | Chloride | monobasic | dibasic | Citrate |
| Description | Specification | Formulation | (mg/mL) | (mg/mL) | (mg/mL) | (mg/mL) | (mg/mL) | (mg/mL) | (mg/mL) |
| Sum of Lysines | ≧75% | 1 | 50 | 12 | 1 | 6.15 | 0.86 | 1.53 | 0.3 |
| | | 2 | 50 | 12 | 1 | 0 | 0.86 | 1.53 | 0.3 |
| | | 3 | 50 | 12 | 1 | 0 | 0.65 | 1.15 | 0.23 |
| | | 4 | 50 | 12 | 1 | 0 | 0 | 0 | 0 |
| | | 5 | 100 | 1 | 1 | 0 | 0 | 0 | 0 |
| | | 6 | 100 | 3 | 1 | 0 | 0 | 0 | 0 |

| | | | Citric Acid, | | | | | | |
|---|---|---|---|---|---|---|---|---|---|
| | | | monohydrate | Sucrose | | | | | |
| Description | Specification | Formulation | (mg/mL) | (mg/mL) | 0 | 2 WK | 1 M | 2 M | 3 M |
| Sum of Lysines | ≧75% | 1 | 1.3 | 46 | 86.2 | 84.5 | 83.7 | 82.4 | 80.3 |
| | | 2 | 1.3 | 60 | 85.7 | 84.9 | 81.6 | 79.9 | 75.3 |
| | | 3 | 0.98 | 60 | 85.3 | 84.7 | 83.2 | 79.6 | 76.1 |
| | | 4 | 0 | 65 | 86.8 | 85.7 | 83.7 | 83.1 | 82.4 |
| | | 5 | 0 | 90 | 86.1 | 83.4 | 83.3 | 82.2 | 82.4 |
| | | 6 | 0 | 75 | 85.2 | 83.3 | 83.7 | 81.6 | 81.5 |

Table 15 also shows the percent change in the SEC monomer content at 1, 2, and 3 months as compared to time zero and Table 16 shows the percent change in the Sum of Lysines data at 1, 2, and 3 months as compared to time zero.

In summary, the low ionic formulation with 50 mg/mL adalimumab, 12 mg/mL mannitol, 1 mg/mL tween 80, and 65 mg/mL sucrose was the most stable formulation and within shelf life specifications after 3 months at the accelerated (40° C./75% RH) storage condition.

TABLE 15
% Change for Size Exclusion HPLC Data

| Description | Specification | 1 M change | 2 M change | 3 M change |
|---|---|---|---|---|
| Monomer | ≧98% | −0.3 | −0.7 | −0.9 |
| | | −0.8 | −0.8 | −1.1 |
| | | −0.5 | −0.6 | −0.9 |
| | | −0.5 | −0.5 | −0.7 |
| | | −0.8 | −1.1 | −1.5 |
| | | −0.8 | −1.4 | −1.8 |

TABLE 16
% Change for Sum of Lysines Data

| Description | Specification | 1 M change | 2 M change | 3 M change |
|---|---|---|---|---|
| Sum of | ≧75% | −2.5 | −3.8 | −5.9 |
| Lysines | | −4.1 | −5.8 | −10.4 |
| | | −2.1 | −5.7 | −9.2 |
| | | −3.1 | −3.7 | −4.4 |
| | | −2.8 | −3.9 | −3.7 |
| | | −1.5 | −3.6 | −3.7 |

### Example 9

### Stability of Solid Units Comprising Low Ionic, Low Acidic Adalimumab Formulations Stability at Accelerated Storage Conditions

The following example describes the evaluation of the stability of solid units comprising low ionic formulations of low acidic adalimumab subjected to accelerated (40° C./75% RH) storage conditions. In addition, the goal of the study was to evaluate if mannitol has any additional stabilizing effect on the low ionic formulations of low acidic adalimumab.

The low acidic adalimumab formulation comprises adalimumab which has been further purified resulting in less acidic regions (AR). Prior to freezing, sucrose was added to a solution comprising low acidic adalimumab which was subsequently subjected to freezing utilizing controlled nucleation freezing in liquid nitrogen as described above in Example 1. The stability of the low acidic adalimumab within the solid units was determined by SEC analysis following storage for 0, 1, 2, 3, and 6 months at 40° C./75% RH storage conditions. The sum of lysines stability of the protein within the solid units was also determined by CEX following storage for 0, 1, 2, 3, and 6 months at 40° C./75% RH storage conditions.

Tables 17 and 18 provide the components and amounts of the low acidic adalimumab, mannitol, tween 80, and sucrose or mannitol in the formulations tested. Tables 17 and 18 also provide the stability data for each of the tested formulations. Of the low ionic formulations tested, the formulation comprising 50 mg/mL low acidic adalimumab, 12 mg/mL mannitol, 1 mg/mL tween 80, and 65 mg/mL sucrose was the most stable formulation and within shelf life specifications after 6 months at the accelerated (40° C./75% RH) storage condition.

TABLE 17
Size Exclusion Stability Data for Low Acidic Adalimumab Low Ionic Formulations at 40° C./75% RH

| | | Low Acidic | | | | | | | | |
|---|---|---|---|---|---|---|---|---|---|---|
| | | Adalimumab | Mannitol | Tween 80 | Sucrose | t | 1 | 2 | 3 | 6 |
| Description | Specification | (mg/mL) | (mg/mL) | (mg/mL) | (mg/mL) | zero | month | month | month | month |
| Monomer | ≧98% | 50 | 12 | 1 | 65 | 99.7 | 99.6 | 99.4 | 99.5 | 98.7 |
| | | 100 | 1 | 1 | 90 | 99.8 | 99 | 99.4 | 98.4 | 96.8 |
| | | 50 | 12 | 1 | 65 | 99.8 | 99.6 | 99.5 | 98.7 | 99.1 |
| | | 50 | 0 | 1 | 65 | 99.8 | 99.4 | 99.1 | 98.2 | 98.5 |
| | | 100 | 0 | 1 | 80 | 99.8 | 98.9 | 98.3 | 96.8 | 96.7 |

TABLE 18
Cation Exchange Sum of Lysines Stability Data for Low Acidic Adalimumab Low Ionic Formulations at 40° C./75% RH

| | | Low Acidic | | | | | | | | |
|---|---|---|---|---|---|---|---|---|---|---|
| | | Adalimumab | Mannitol | Tween 80 | Sucrose | t | 1 | 2 | 3 | 6 |
| Description | Specification | (mg/mL) | (mg/mL) | (mg/mL) | (mg/mL) | zero | month | month | month | month |
| Sum of Lysines | ≧75% | 50 | 12 | 1 | 65 | 96.4 | 94.8 | 94.9 | 92.3 | 90.9 |
| | | 100 | 1 | 1 | 90 | 96.5 | 93.8 | 91.8 | 89.9 | 88.8 |
| | | 50 | 12 | 1 | 65 | 95.2 | 93.1 | 94.7 | 92.9 | 91.7 |
| | | 50 | 0 | 1 | 65 | 94.7 | 92.5 | 93.7 | 92.1 | 90.2 |
| | | 100 | 0 | 1 | 80 | 94.4 | 93.6 | 90.6 | 90.4 | 88.4 |

### Example 10

### Stability of Antibody B within Solid Units Subjected to Accelerated Storage Conditions

The following example describes the evaluation of the stability of Antibody B (an anti-IL-8 antibody) in solid units prepared using solutions of Antibody B and varying mannitol-sucrose ratios, with and without tween subjected to accelerated (40° C./75% RH) storage conditions.

In this study, ten different formulations comprising 50 mg/ml of Antibody B and 2.33 mg/ml histidine with varying mannitol-sucrose ratios and with and without tween 80 (see Tables 19 and 20) were subjected to the lyophilization process employing controlled nucleation utilizing liquid nitrogen (described above in Example 1).

The stability of the Antibody B within the solid units was determined by SEC analysis following storage for 0, 1, 2, and 4 weeks at 40° C./75% RH storage conditions. The sum of lysines stability of the protein within the solid units was also determined by CEX following storage for 0, 1, 2, and 4 weeks at 40° C./75% RH storage conditions.

The results are presented in Tables 19 and 20 and demonstrate that Antibody B within the solid units is stable in all formulations tested following the controlled nucleation lyophilization process (the monomer specification for Antibody B is not less than 90%). The Antibody B formulations without tween and with mannitol (formulations 9 and 10) show the greatest monomer stability at accelerated storage conditions. Surprisingly, the formulations without Tween also maintained the stability of Antibody B. Furthermore, the combination of sucrose and mannitol in the formulation stabilized the Antibody B molecule.

TABLE 19
Size Exclusion HPLC Stability Data for Antibody B Formulations at 40° C./75% RH

| | | Protein | Tween | Histidine | Mannitol | Sucrose | | | | |
|---|---|---|---|---|---|---|---|---|---|---|
| Description | Formulation | (mg/mL) | (mg/mL) | (mg/mL) | (mg/mL) | (mg/mL) | 0 | 1 wk | 2 wk | 4 wk |
| Monomer | 1 | 50 | 0.1 | 2.33 | 0 | 50 | 98.1 | 97.6 | 97.5 | 96.7 |
| | 2 | 50 | 0.1 | 2.33 | 10 | 50 | 98.1 | 97.8 | 97.9 | 97.2 |
| | 3 | 50 | 0.1 | 2.33 | 20 | 50 | 98.2 | 87.9 | 97.7 | 97.6 |
| | 4 | 50 | 0.1 | 2.33 | 10 | 25 | 97.8 | 97.2 | 96.6 | 96.3 |
| | 5 | 50 | 0.1 | 2.33 | 20 | 25 | 98.3 | 97.4 | 97.2 | 96.7 |
| | 6 | 50 | 0.1 | 2.33 | 10 | 10 | 97.6 | 96.6 | 95.5 | 94.3 |
| | 7 | 50 | 0.1 | 2.33 | 20 | 10 | 97.5 | 95.9 | 95.3 | 94.0 |
| | 8 | 50 | 0 | 2.33 | 0 | 50 | 98.1 | 98.0 | 97.8 | 97.6 |
| | 9 | 50 | 0 | 2.33 | 10 | 50 | 98.3 | 98.0 | 97.9 | 97.8 |
| | 10 | 50 | 0 | 2.33 | 20 | 50 | 98.4 | 98.3 | 98.0 | 98.1 |

TABLE 20
Cation Exchange HPLC Stability Data for Antibody B Formulations at 40° C./75% RH

| | | Protein | Tween | Histidine | Mannitol | Sucrose | | | | |
|---|---|---|---|---|---|---|---|---|---|---|
| Description | Formulation | (mg/mL) | (mg/mL) | (mg/mL) | (mg/mL) | (mg/mL) | 0 | 1 wk | 2 wk | 4 wk |
| Main Peak | 1 | 50 | 0.1 | 2.33 | 0 | 50 | 70.5 | 69.7 | 68.9 | 67.1 |
| | 2 | 50 | 0.1 | 2.33 | 10 | 50 | 70.8 | 70.1 | 69.6 | 68.8 |
| | 3 | 50 | 0.1 | 2.33 | 20 | 50 | 71.3 | 70.6 | 70.0 | 56.7 |
| | 4 | 50 | 0.1 | 2.33 | 10 | 25 | 70.9 | 70.1 | 68.8 | 67.0 |
| | 5 | 50 | 0.1 | 2.33 | 20 | 25 | 71.3 | 69.9 | 69.2 | 69.0 |
| | 6 | 50 | 0.1 | 2.33 | 10 | 10 | 70.8 | 67.6 | 65.4 | 68.8 |
| | 7 | 50 | 0.1 | 2.33 | 20 | 10 | 69.8 | 64.8 | 61.5 | 66.6 |
| | 8 | 50 | 0 | 2.33 | 0 | 50 | 71.2 | 70.0 | 68.7 | 66.7 |
| | 9 | 50 | 0 | 2.33 | 10 | 50 | 71.6 | 70.5 | 70.6 | 69.1 |
| | 10 | 50 | 0 | 2.33 | 20 | 50 | 71.4 | 70.6 | 70.9 | 60.8 |
| Acidic | 1 | 50 | 0.1 | 2.33 | 0 | 50 | 13.1 | 14.0 | 15.0 | 16.3 |
| | 2 | 50 | 0.1 | 2.33 | 10 | 50 | 13.1 | 14.2 | 14.9 | 15.1 |
| | 3 | 50 | 0.1 | 2.33 | 20 | 50 | 12.6 | 13.6 | 14.1 | 24.1 |
| | 4 | 50 | 0.1 | 2.33 | 10 | 25 | 12.9 | 13.8 | 15.0 | 16.9 |
| | 5 | 50 | 0.1 | 2.33 | 20 | 25 | 13.0 | 14.1 | 14.8 | 15.0 |
| | 6 | 50 | 0.1 | 2.33 | 10 | 10 | 13.1 | 15.6 | 18.1 | 14.9 |
| | 7 | 50 | 0.1 | 2.33 | 20 | 10 | 13.9 | 17.6 | 20.5 | 16.9 |
| | 8 | 50 | 0 | 2.33 | 0 | 50 | 12.9 | 13.8 | 15.1 | 17.0 |
| | 9 | 50 | 0 | 2.33 | 10 | 50 | 12.8 | 13.8 | 13.6 | 14.8 |
| | 10 | 50 | 0 | 2.33 | 20 | 50 | 13.0 | 13.6 | 13.1 | 21.2 |
| Basic | 1 | 50 | 0.1 | 2.33 | 0 | 50 | 16.4 | 16.3 | 16.1 | 16.5 |
| | 2 | 50 | 0.1 | 2.33 | 10 | 50 | 16.1 | 15.7 | 15.5 | 16.1 |
| | 3 | 50 | 0.1 | 2.33 | 20 | 50 | 16.1 | 15.9 | 15.9 | 19.2 |
| | 4 | 50 | 0.1 | 2.33 | 10 | 25 | 16.2 | 16.1 | 16.2 | 16.1 |
| | 5 | 50 | 0.1 | 2.33 | 20 | 25 | 15.8 | 16.0 | 16.1 | 16.0 |
| | 6 | 50 | 0.1 | 2.33 | 10 | 10 | 16.1 | 16.9 | 16.5 | 16.2 |
| | 7 | 50 | 0.1 | 2.33 | 20 | 10 | 16.2 | 17.6 | 18.0 | 16.4 |
| | 8 | 50 | 0 | 2.33 | 0 | 50 | 15.9 | 16.1 | 16.2 | 16.3 |
| | 9 | 50 | 0 | 2.33 | 10 | 50 | 15.7 | 15.7 | 15.9 | 16.2 |
| | 10 | 50 | 0 | 2.33 | 20 | 50 | 15.6 | 15.8 | 16.0 | 18.0 |

### Example 11

### Stability of Antibody C within Solid Units Subjected to Accelerated Storage Conditions

The following example describes the evaluation of the stability of Antibody C (an anti-IL-17 antibody) in solid units prepared using solutions of Antibody C and varying mannitol-sucrose ratios, with and without tween and subjected to accelerated (40° C./75% RH) storage conditions.

In this study, ten different formulations comprising 50 mg/ml of Antibody C and 2.33 mg/ml histidine with varying mannitol-sucrose ratios and with and without tween 80 (see Tables 21 and 22) were subjected to the lyophilization process employing controlled nucleation utilizing liquid nitrogen (described above in Example 1).

The stability of the Antibody C within the solid units was determined by SEC analysis following storage for 0, 1, 2, 3, and 4 weeks at 40° C./75% RH storage conditions. The sum of lysines stability of the protein within the solid units was also determined by CEX following storage for 0, 1, 2, 3, and 4 weeks at 40° C./75% RH storage conditions.

The results are also presented in Tables 21 and 22 and demonstrate that Antibody C (within the solid units) is stable in all formulations tested following the controlled nucleation lyophilization process (the monomer specification for Antibody C is not less than 90%). The Antibody C formulations without tween and with mannitol (formulations 9 and 10) show the greatest monomer stability at accelerated storage conditions. Surprisingly, the formulations without Tween also maintained the stability of Antibody C. Furthermore, the combination of sucrose and mannitol in the formulation stabilized the Antibody C molecule.

TABLE 21
Size Exclusion HPLC Stability Data for Antibody C Formulations at 40° C./75% RH

| | | Protein | Tween | Histidine | Mannitol | Sucrose | | | | |
|---|---|---|---|---|---|---|---|---|---|---|
| Description | Formulation | (mg/mL) | (mg/mL) | (mg/mL) | (mg/mL) | (mg/mL) | 0 | 1 wk | 2 wk | 4 wk |
| Monomer | 1 | 50 | 0.1 | 2.33 | 0 | 50 | 97.7 | 97.4 | 97.1 | 96.7 |
| | 2 | 50 | 0.1 | 2.33 | 10 | 50 | 97.9 | 97.5 | 97.3 | 97.1 |
| | 3 | 50 | 0.1 | 2.33 | 20 | 50 | 97.2 | 94.8 | 93.4 | 91.1 |
| | 4 | 50 | 0.1 | 2.33 | 10 | 25 | 98.0 | 97.2 | 96.8 | 96.9 |
| | 5 | 50 | 0.1 | 2.33 | 20 | 25 | 97.8 | 97.3 | 96.9 | 97.2 |
| | 6 | 50 | 0.1 | 2.33 | 10 | 10 | 97.6 | 97.0 | 96.4 | 95.7 |
| | 7 | 50 | 0.1 | 2.33 | 20 | 10 | 97.3 | 96.1 | 95.1 | 93.8 |
| | 8 | 50 | 0 | 2.33 | 0 | 50 | 98.1 | 97.6 | 97.4 | 97.0 |
| | 9 | 50 | 0 | 2.33 | 10 | 50 | 98.1 | 98.0 | 97.9 | 97.6 |
| | 10 | 50 | 0 | 2.33 | 20 | 50 | 98.2 | 98.1 | 98.0 | 97.9 |

TABLE 22
Cation Exchange HPLC Stability Data for Antibody C Formulations at 40° C./75% RH

| | | Protein | Tween | Histidine | Mannitol | Sucrose | | | | |
|---|---|---|---|---|---|---|---|---|---|---|
| Description | Formulation | (mg/mL) | (mg/mL) | (mg/mL) | (mg/m) | (mg/mL) | 0 | 1 wk | 2 wk | 4 wk |
| Main Isoform | 1 | 50 | 0.1 | 2.33 | 0 | 50 | 54.0 | 53.9 | 53.5 | 52.9 |
| | 2 | 50 | 0.1 | 2.33 | 10 | 50 | 54.2 | 53.8 | 53.9 | 53.8 |
| | 3 | 50 | 0.1 | 2.33 | 20 | 50 | 53.3 | 45.1 | 39.2 | 36.1 |
| | 4 | 50 | 0.1 | 2.33 | 10 | 25 | 53.8 | 53.6 | 53.6 | 52.9 |
| | 5 | 50 | 0.1 | 2.33 | 20 | 25 | 54.7 | 53.9 | 53.5 | 53.7 |
| | 6 | 50 | 0.1 | 2.33 | 10 | 10 | 54.1 | 53.3 | 52.5 | 52.8 |
| | 7 | 50 | 0.1 | 2.33 | 20 | 10 | 54.2 | 52.3 | 51.0 | 51.3 |
| | 8 | 50 | 0 | 2.33 | 0 | 50 | 54.7 | 53.9 | 53.7 | 54.0 |
| | 9 | 50 | 0 | 2.33 | 10 | 50 | 54.8 | 54.5 | 54.4 | 54.6 |
| | 10 | 50 | 0 | 2.33 | 20 | 50 | 54.8 | 54.6 | 54.6 | 54.5 |
| Acidic | 1 | 50 | 0.1 | 2.33 | 0 | 50 | 8.4 | 8.5 | 8.9 | 9.2 |
| | 2 | 50 | 0.1 | 2.33 | 10 | 50 | 8.5 | 9.0 | 8.9 | 8.9 |
| | 3 | 50 | 0.1 | 2.33 | 20 | 50 | 8.3 | 14.3 | 18.4 | 18.9 |
| | 4 | 50 | 0.1 | 2.33 | 10 | 25 | 8.3 | 9.1 | 9.0 | 9.3 |
| | 5 | 50 | 0.1 | 2.33 | 20 | 25 | 8.7 | 9.1 | 9.3 | 9.1 |
| | 6 | 50 | 0.1 | 2.33 | 10 | 10 | 8.9 | 8.8 | 8.9 | 8.8 |
| | 7 | 50 | 0.1 | 2.33 | 20 | 10 | 8.7 | 8.7 | 9.1 | 8.8 |
| | 8 | 50 | 0 | 2.33 | 0 | 50 | 8.4 | 8.6 | 9.0 | 8.5 |
| | 9 | 50 | 0 | 2.33 | 10 | 50 | 8.5 | 8.7 | 8.7 | 8.6 |
| | 10 | 50 | 0 | 2.33 | 20 | 50 | 8.4 | 8.8 | 8.5 | 8.8 |
| Basic | 1 | 50 | 0.1 | 2.33 | 0 | 50 | 37.6 | 37.5 | 37.6 | 38.0 |
| | 2 | 50 | 0.1 | 2.33 | 10 | 50 | 37.2 | 37.2 | 37.2 | 37.2 |
| | 3 | 50 | 0.1 | 2.33 | 20 | 50 | 38.5 | 40.5 | 42.4 | 45.1 |
| | 4 | 50 | 0.1 | 2.33 | 10 | 25 | 37.9 | 37.4 | 37.5 | 37.9 |
| | 5 | 50 | 0.1 | 2.33 | 20 | 25 | 36.6 | 37.1 | 37.2 | 37.3 |
| | 6 | 50 | 0.1 | 2.33 | 10 | 10 | 37.0 | 37.9 | 38.5 | 38.5 |
| | 7 | 50 | 0.1 | 2.33 | 20 | 10 | 37.1 | 38.9 | 39.9 | 39.9 |
| | 8 | 50 | 0 | 2.33 | 0 | 50 | 36.9 | 37.5 | 37.4 | 37.5 |
| | 9 | 50 | 0 | 2.33 | 10 | 50 | 36.7 | 36.9 | 36.9 | 36.9 |
| | 10 | 50 | 0 | 2.33 | 20 | 50 | 36.9 | 36.6 | 36.9 | 36.7 |

### Example 12

### Stability of DVD-Ig a within Solid Units Subjected to Accelerated Storage Conditions

The following example describes the evaluation of the stability of DVD-Ig A in solid units prepared using solutions of DVD-Ig A and varying mannitol-sucrose ratios or varying glycine-sucrose ratios subjected to accelerated (40° C./75% RH) storage conditions.

In this study, six different formulations comprising 50 mg/ml of DVD-Ig A and 2.33 mg/ml histidine with varying mannitol-sucrose ratios or varying sucrose-glycine ratios (see Tables 23 and 24) were subjected to the lyophilization process employing controlled nucleation utilizing liquid nitrogen (described above in Example 1).

TABLE 23
Size Exclusion HPLC Stability Data for DVD-Ig A Formulations at 40° C./75% RH

| | | Protein | Tween | Histidine | Mannitol | Sucrose | Glycine | | | | |
|---|---|---|---|---|---|---|---|---|---|---|---|
| Description | Formulation | (mg/mL) | (mg/mL) | (mg/mL) | (mg/mL) | (mg/mL) | (mg/mL) | 0 | 1 wk | 2 wk | 4 wk |
| Monomer | 1 | 50 | 0.1 | 2.33 | 0 | 75 | 0 | 98.0 | 97.8 | 97.5 | 97.4 |
| | 2 | 50 | 0.1 | 2.33 | 10 | 75 | 0 | 97.8 | 96.9 | 97.6 | 97.0 |
| | 3 | 50 | 0.1 | 2.33 | 20 | 75 | 0 | 97.9 | 98.0 | 96.9 | 97.7 |
| | 4 | 50 | 0.1 | 2.33 | 0 | 75 | 0 | 96.8 | 96.8 | 96.4 | 97.2 |
| | 5 | 50 | 0.1 | 2.33 | 0 | 75 | 10 | 97.3 | 97.1 | 97.2 | 97.6 |
| | 6 | 50 | 0.1 | 2.33 | 0 | 75 | 20 | 98.1 | 97.1 | 97.3 | 97.1 |

TABLE 24
Cation Exchange HPLC Stability Data for DVD-Ig A Formulations at 40° C./75% RH

| | | Protein | Tween | Histidine | Mannitol | Sucrose | Glycine | | | | |
|---|---|---|---|---|---|---|---|---|---|---|---|
| Description | Formulation | (mg/mL) | (mg/mL) | (mg/mL) | (mg/mL) | (mg/mL) | (mg/mL) | 0 | 1 wk | 2 wk | 4 wk |
| Main Species | 1 | 50 | 0.1 | 2.33 | 0 | 75 | 0 | 70.3 | 68.0 | 67.7 | 66.8 |
| | 2 | 50 | 0.1 | 2.33 | 10 | 75 | 0 | 70.3 | 68.6 | 67.9 | 66.9 |
| | 3 | 50 | 0.1 | 2.33 | 20 | 75 | 0 | 70.5 | 68.9 | 68.3 | 67.4 |
| | 4 | 50 | 0.1 | 2.33 | 0 | 75 | 0 | 69.7 | 68.2 | 67.7 | 66.5 |
| | 5 | 50 | 0.1 | 2.33 | 0 | 75 | 10 | 70.2 | 68.8 | 68.5 | 67.7 |
| | 6 | 50 | 0.1 | 2.33 | 0 | 75 | 20 | 70.1 | 68.8 | 68.5 | 67.7 |
| Acidic | 1 | 50 | 0.1 | 2.33 | 0 | 75 | 0 | 11.0 | 11.2 | 11.2 | 11.2 |
| | 2 | 50 | 0.1 | 2.33 | 10 | 75 | 0 | 11.1 | 11.2 | 11.2 | 11.3 |
| | 3 | 50 | 0.1 | 2.33 | 20 | 75 | 0 | 11.2 | 11.3 | 11.3 | 11.3 |
| | 4 | 50 | 0.1 | 2.33 | 0 | 75 | 0 | 11.2 | 11.2 | 11.2 | 11.3 |
| | 5 | 50 | 0.1 | 2.33 | 0 | 75 | 10 | 11.3 | 11.5 | 11.5 | 11.6 |
| | 6 | 50 | 0.1 | 2.33 | 0 | 75 | 20 | 11.4 | 11.6 | 11.7 | 11.8 |
| Basic | 1 | 50 | 0.1 | 2.33 | 0 | 75 | 0 | 18.7 | 20.8 | 21.1 | 22.0 |
| | 2 | 50 | 0.1 | 2.33 | 10 | 75 | 0 | 18.6 | 20.2 | 20.8 | 21.8 |
| | 3 | 50 | 0.1 | 2.33 | 20 | 75 | 0 | 18.3 | 19.8 | 20.4 | 21.3 |
| | 4 | 50 | 0.1 | 2.33 | 0 | 75 | 0 | 19.0 | 20.6 | 21.1 | 22.2 |
| | 5 | 50 | 0.1 | 2.33 | 0 | 75 | 10 | 18.5 | 19.8 | 20.1 | 20.7 |
| | 6 | 50 | 0.1 | 2.33 | 0 | 75 | 20 | 18.6 | 19.5 | 19.8 | 20.6 |

The stability of the DVD-Ig A within the solid units was determined by SEC analysis following storage for 0, 1, 2, 3, and 4 weeks at 40° C./75% RH storage conditions. The sum of lysines stability of the protein within the solid units was also determined by CEX following storage for 0, 1, 2, 3, and 4 weeks at 40° C./75% RH storage conditions.

The results are also presented in Tables 23 and 24 and demonstrate that DVD-Ig A within the solid units is stable in all formulations tested following the controlled nucleation lyophilization process (the monomer specification for DVD-Ig A is not less than 90%). The DVD-Ig A formulations with glycine and sucrose (formulations 5 and 6) showed the greatest basic species stability at accelerated storage conditions. Therefore, glycine in combination with sucrose has a stabilizing effect on the stability of the DVD-Ig A molecule.

### Example 13

### Pharmacokinetic Properties of Different Adalimumab Preparations

The aim of this study was to compare the pharmacokinetic properties of three different adalimumab formulations dosed subcutaneously in rats. Specifically, the serum concentration of adalimumab was assayed using a ligand binding assay following administration of a frozen solution of adalimumab bulk drug substance (Group 1), solid units comprising adalimumab bulk drug substance (Group 2), and solid units comprising adalimumab bulk drug substance and 46 mg/ml sucrose (Group 3). Adalimumab BDS solution was prepared with the formulation in Table 25.

TABLE 25
Adalimumab Bulk Drug Substance (ada BDS)

| | Amount |
|---|---|
| Material | (mg/mL) |
| Adalimumab | 50 |
| Mannitol | 12 |
| Tween 80 | 1 |
| Sodium chloride | 6.15 |
| Sodium phosphate, monobasic (2H2O) | 0.86 |
| Sodium phosphate, dibasic (2H2O) | 1.53 |
| Sodium citrate | 0.30 |
| Citric acid, monohydrate | 1.30 |

For group one, 0.5 mL aliquots of the 50 mg/mL adalimumab BDS solution was transferred to vials and sealed with a rubber stopper and aluminum cap. The sealed vials were transferred to a CryoPro Box and stored in −70° C. freezer. The vials were kept frozen on dry ice and thawed at room temperature before use.

For group two, the solid units were prepared with the 50 mg/mL adalimumab BDS solution. Freezing was performed by dispensing the liquid solution into a stainless steel pan with dividers filled with liquid nitrogen at approximately −190° C. After a bulk of frozen solid units was manufactured, the solid units were transferred to pre-cooled vials on dry ice. The frozen solid units were transferred into each of the vial and then stoppered with lyo stoppers (lyo stoppers were partially inserted to allow sublimation venting) and loaded into the lyophilizer at shelf temperature about −45° C. After being held at −45° C., the shelf temperature was warmed to −15° C. The shelf was then cooled to −45° C. After being held at −45° C., the lyo chamber was evacuated to a pressure of approximately 100 microns. Following evacuation, the vials were subjected to a primary drying step at about −15° C. and 100 microns of pressure. Lastly, the vials were subjected to a secondary drying step at 30° C. under approximately 100 microns of pressure. The lyophilized solid units were stored refrigerated until use. The solid units in vials were reconstituted with water before use.

For group three, sucrose was added to the 50 mg/mL adalimumab BDS solution to obtain 46 mg/mL sucrose concentration. The solid units were prepared utilizing the adalimumab BDS with sucrose solution. Freezing was performed by dispensing the liquid solution using syringe pump into a stainless steel pan with dividers filled with liquid nitrogen. After a bulk of frozen solid units was manufactured, the solid units were transferred to pre-cooled vials on dry ice. The frozen solid units were transferred into each of the vial and then stoppered with lyo stoppers (lyo stoppers were partially inserted to allow sublimation venting) and loaded into the lyophilizer at shelf temperature about −45° C. After being held at −45° C., the shelf temperature was warmed to −15° C. The shelf was then cooled to −45° C. After being held at −45° C., the lyophilizer chamber was evacuated to a pressure of approximately 100 microns. Following evacuation, the vials were subjected to a primary drying step at about −15° C. and 100 microns of pressure. Lastly, the vials were subjected to a secondary drying step at 30° C. under approximately 100 microns of pressure. The lyophilized solid units were stored refrigerated until use. The solid units in vials were reconstituted with water before use.

Groups of five male Sprague Daley rats were subcutaneously administered a single 5 mg/kg adalimumab dose (1 ml/kg). Serum was collected over a period of 10 days. At about 0, 1, 3, 5, 10, 20, 40, 80, 100, 120, 140, 160, 180, 200, 220, and 240 hours after administration, serum was collected. Serum adalimumab concentrations were determined using a ligand-binding assay with biotinylated human anti-TNF as a capture reagent and goat anti-human sulfotag as detection reagent. The lower limit of quantitation of the assay was about 0.14 μg/ml.

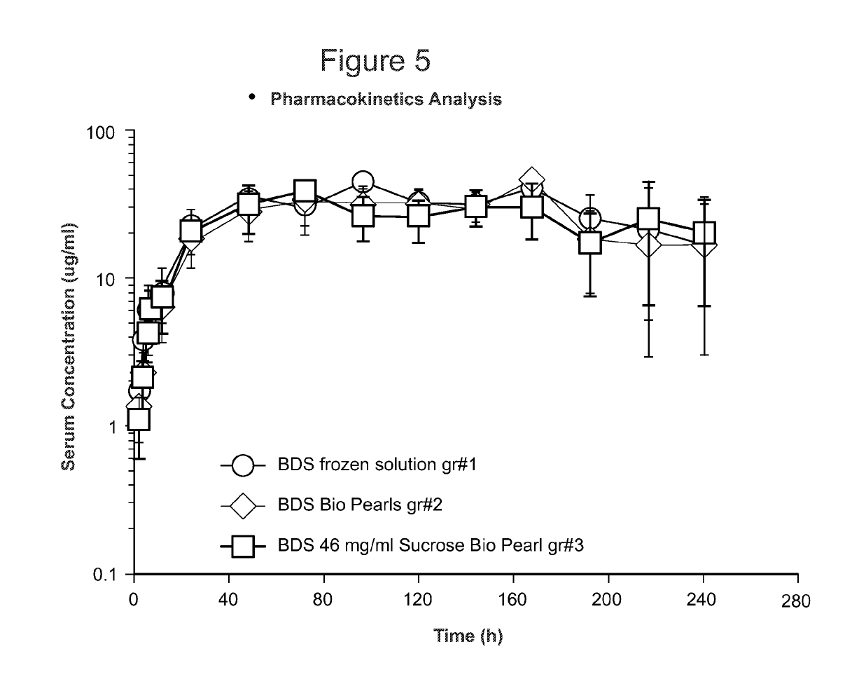

As shown in FIG. 5, the concentration of adalimumab in the serum of rats administered the different formulations were similar over the period of 10 days. Table 26 below summarizes the Cₘₐₓ, Tₘₐₓ, and AUC₀₋₁₆₈ₕᵣ calculations from FIG. 5 and demonstrate that the different preparations of adalimumab result in comparable pharmacokinetics.

TABLE 26
Summary of Pharmacokinetics Analyses

| | Cₘₐₓ | Tₘₐₓ | AUC_{0-168 hr} |
|---|---|---|---|
| Formulation | (μg/ml) | (hr) | (mg · hr/ml) |
| Group 1 | 46.7 (+/−6.1) | 168 (+/−61) | 5134 (+/−610) |
| Group 2 (ada BDS | 47.2 (+/−2.9) | 158 (+/−21) | 4752 (+/−842) |
| solid units) | | | |
| Group 3 (ada BDS + | 41.9 (+/−5.5) | 120 (+/−70) | 4332 (+/−386) |
| sucrose solid units) | | | |

The pharmacokinetics of control sample, uniform free flowing solid units with the same composition of control sample, and uniform free flowing solid units with sucrose in the composition are comparable. The manufacturing process with controlled nucleation producing uniform, free flowing solid units does not alter the pharmacokinetics. Furthermore, the addition of sucrose to the formulation does not alter the pharmacokinetics.

### Example 14

### Stability Analysis of Adalimumab in Solid Units Comprising Adalimumab and Sucrose

A detailed analysis of protein stability within solid units comprising adalimumab and sucrose (46 mg/mL) was assessed by comparing freshly made solid units and solid units stored at s 25° C./60% RH for 23 months. Two liquid controls (adalimumab reference standards) were also used in the comparative study.

Solid units and liquid formulations comprising adalimumab BDS were prepared as described above. Analysis of the antibody in each formulation was performed using a number of methods known in the art, including Mass Spectrometry (MS), Circular Dichronism (CD), Size Exclusion Chromatography—Multi Angle Light Scattering (SEC-MALS), Sedimentation Velocity Analytical Ultracentrifugation (SV-AUC), Hydrophobic Interaction Chromatography (HIC), Differential Scanning calorimetry (DSC), Weak Cation Exchange (WCX), Capillary Electrophoresis-Sodium-Dodecyl Sulfate Polyacrylamide Gel Electrophoresis (CE-SDS-PAGE), Dynamic Light Scattering and Surface Plasmon Resonance (SPR) spectroscopy.

Table 27 provides a summary of the results and shows that all of the tested parameters were comparable between the freshly made and stored solid units comprising adalimumab. In addition, the primary structure of adalimumab within both the freshly made solid units and the solid units stored for 23 months was in agreement with the theoretical amino acid sequence.

TABLE 27
Summary of Stability Studies of Adalimumab
with Sucrose Solid Units (Pearls).

| Assay | Purpose | Results |
|---|---|---|
| Intact/reduced MS | Confirm sequence, some | Comparable |
| | oligosaccharide information | |
| CD | Confirm 2°, 3° structure | Comparable |
| SEC-MALS | Confirm purity and molecular | Comparable |
| | weight, assess aggregation | |
| SV-AUC | Orthogonal confirmation of SEC | Comparable |
| | results, more precise information | |
| | on aggregates | |
| HIC | Assess distribution of | Comparable |
| | hydrophobic species | |
| DSC | Assess thermodynamic stability | Comparable |
| WCX-10 | Assess charge heterogeneity | Comparable |
| Free SH | Assess integrity of disulfide | Comparable |
| | bonds | |
| CE-SDS | Assess purity | Comparable |
| Dynamic light scattering | Measure high-order aggregation | Comparable |

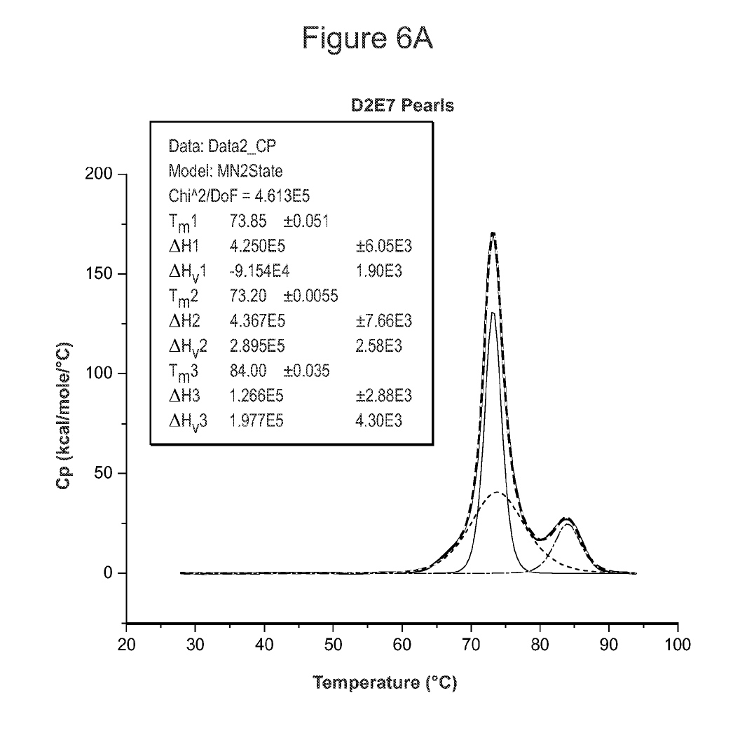

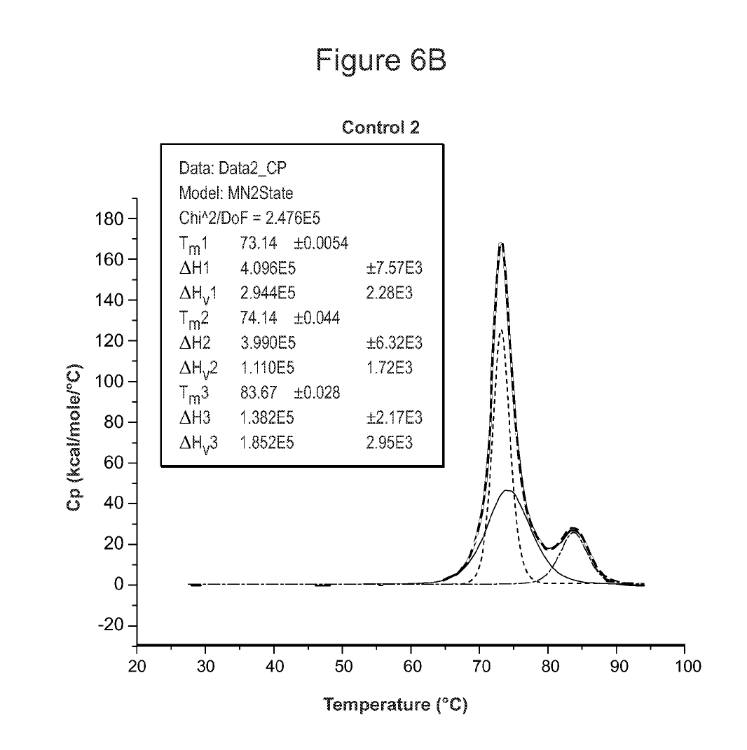

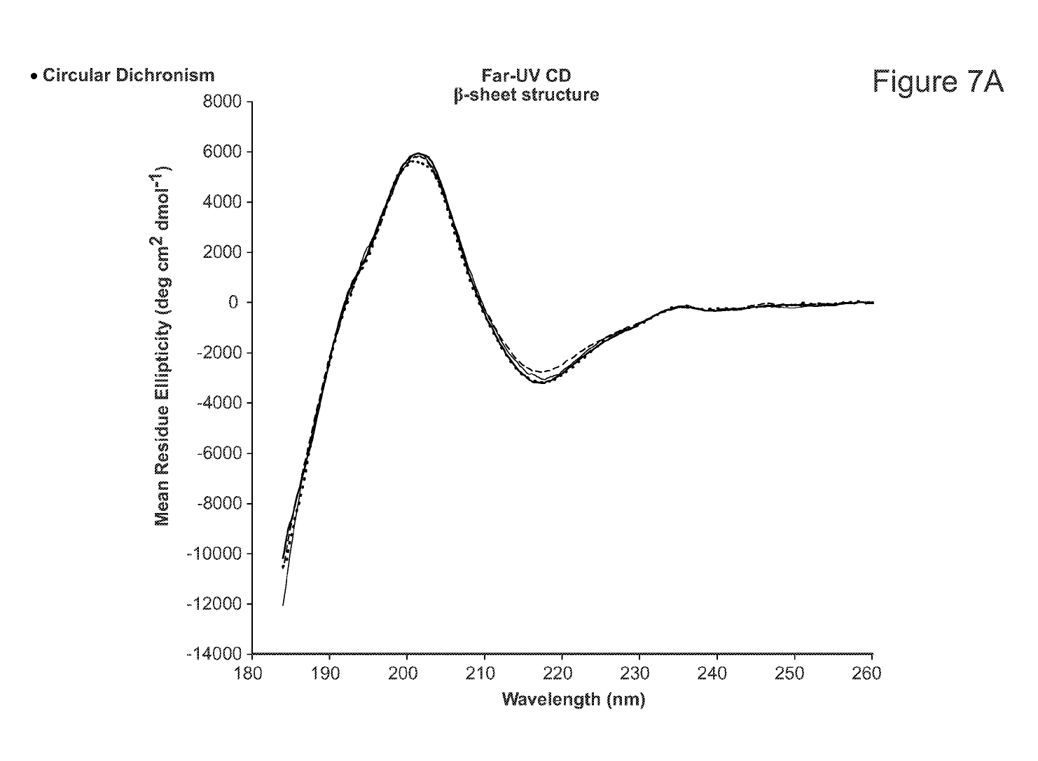

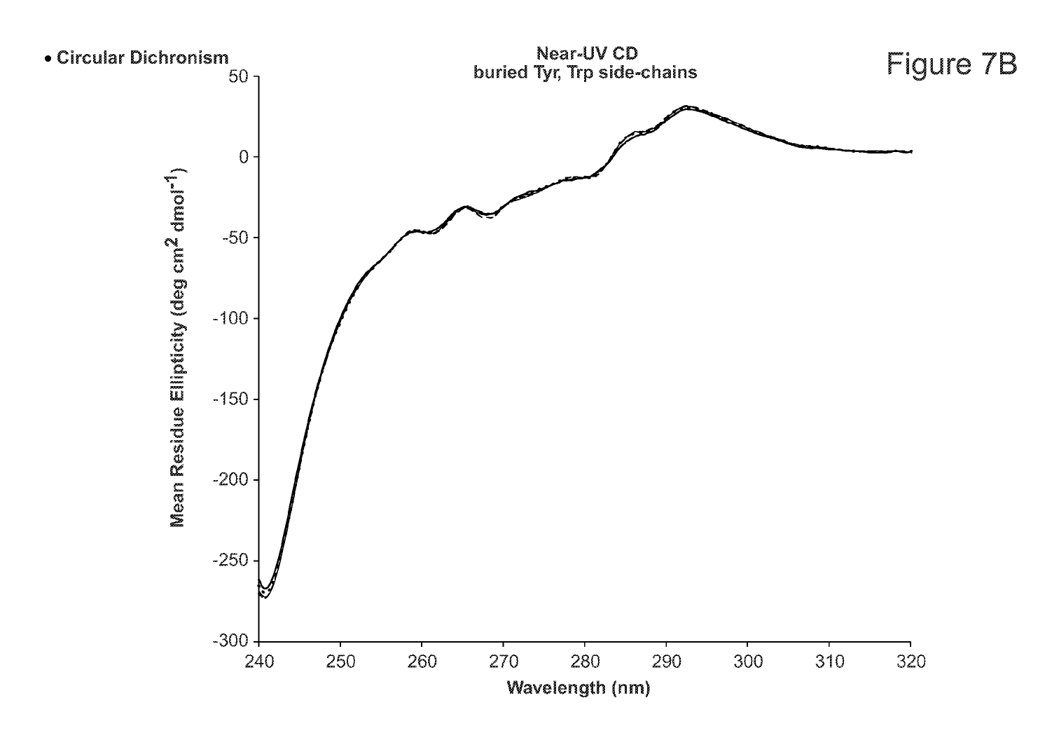

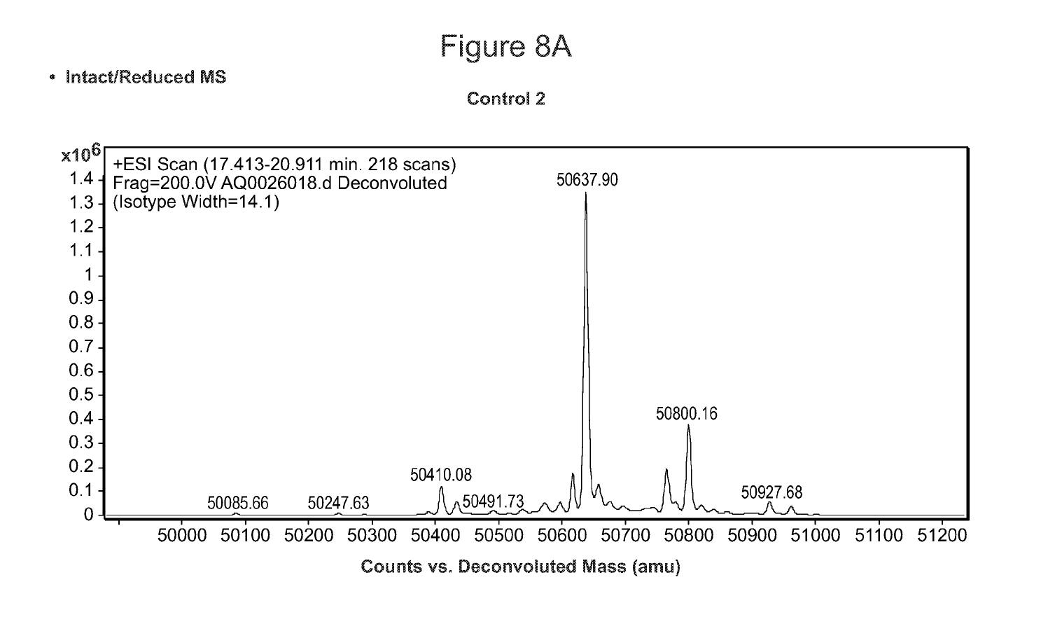

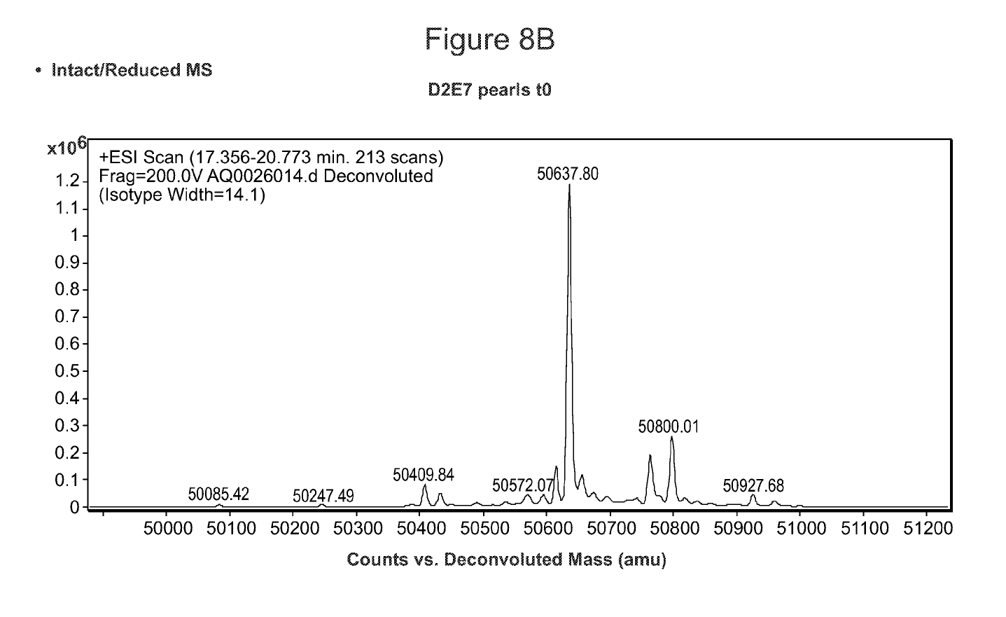

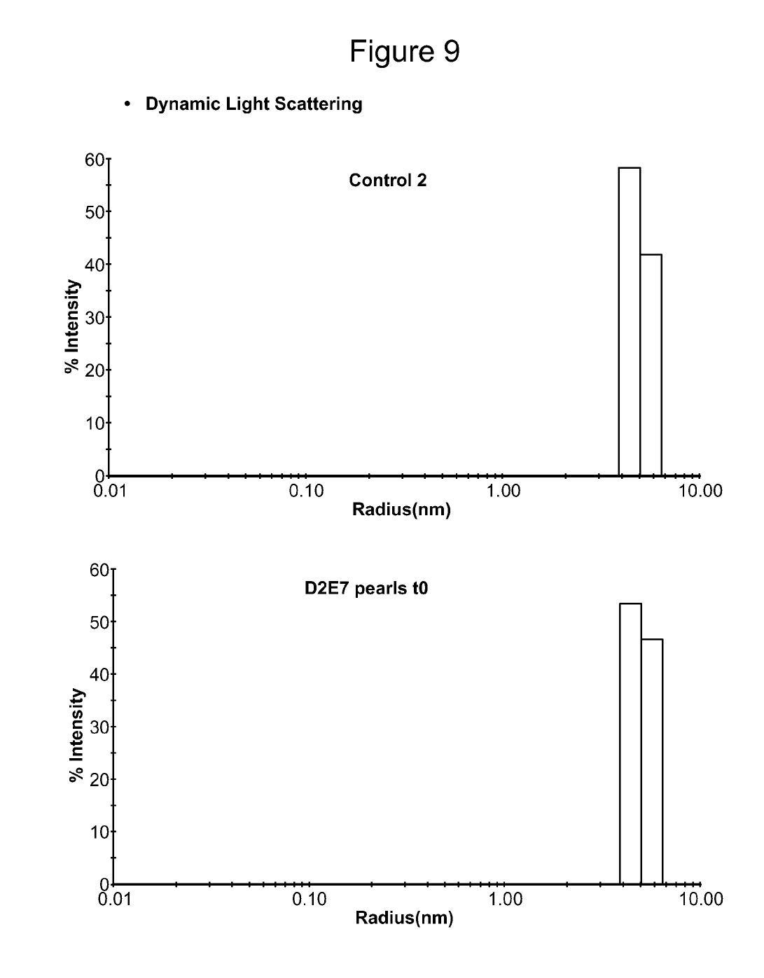

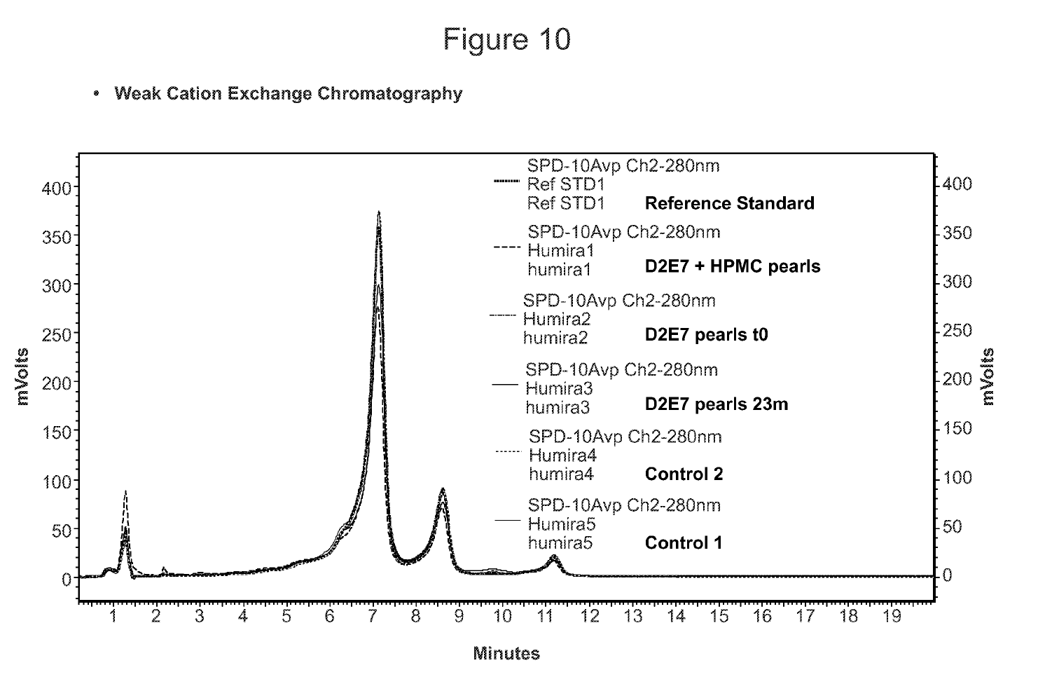

Tables 28 to 34 provide more detailed analysis of the summary provided in Table 27. Table 28 provides the results of the assessment of TNF Binding by Surface Plasmon Resonance (SPR); Table 29 provides the results of Differential Scanning calorimetry (DSC) analyses (see also FIGS. 6A and 6B); Table 30 provides the results of the Intact/Reduced MS analyses; Table 31 provides the results of the Size Exclusion Chromatography—Multi Angle Light Scattering (SEC-MALS) analyses; Table 32 provides the results of the Dynamic Light Scattering (DLS analyses); Table 33 provides the results of the Weak Cation Exchange Chromatography (WCX-10) analyses; and Table 34 provides the results of the Sedimentation Velocity Analytical Ultracentrifugation (SV-AUC) analyses. In each of Tables 28-34, the freshly made solid units are referred as D2E7 pearls t0 and the 23 month old solid units are referred as D2E7 pearls 23m. FIGS. 7A and 7B show the results of the circular dichroism analyses.

The batch referred to in the Tables below as Control 1 is liquid standard used in these analyses having a pH of about 5.2 and including about 100 mg/mL of Adalimumab, about 42 mg/ml mannitol, and about 1 mg/ml Tween-80. The batch referred to in the Tables below as Control 2 is also a liquid standard used in these analyses having a pH of about 5.2 and including about 50 mg/mL of Adalimumab, about 12 mg/ml mannitol, about 1 mg/ml Tween-80, about 6.15 mg/ml Sodium Chloride, about 0.86 mg/ml Sodium Phosphate, monobasic (2H₂O), about 1.53 mg/ml Sodium Phosphate, dibasic (2H₂O), about 0.3 mg/ml sodium citrate, and about 1.30 mg/ml citric acid, monohydrate.

TABLE 28
Results from TNF Binding by SPR (Biacore T-100).

| | kₐ | k_{d} | K_{D} |
|---|---|---|---|
| Batch | (×10⁶ M⁻¹ s⁻¹) | (×10⁻⁴ s⁻¹) | (pM) |
| Control 1 | 1.98 ± 0.06 | 1.17 ± 0.02 | 59.08 ± 2.80 |
| Control 2 | 1.83 ± 0.08 | 1.12 ± 0.04 | 61.32 ± 0.93 |
| D2E7 pearls t0 | 2.13 ± 0.06 | 1.17 ± 0.02 | 54.80 ± 1.52 |
| D2E7 pearls 23 m | 2.31 ± 0.01 | 1.25 ± 0.06 | 53.89 ± 2.23 |

TABLE 29
Results from Differential Scanning Calorimetry

| Batch | T_{M, 1} (° C.) | ΔH₁ | T_{M, 2} (° C.) | ΔH₂ | T_{M, 3} (° C.) | ΔH₃ |
|---|---|---|---|---|---|---|
| D2E7 pearls t0 | 73.20 | 4.37E+05 | 73.85 | 4.25E+05 | 84.00 | 1.27E+05 |
| D2E7 pearls 23 m | 73.10 | 4.30E+05 | 74.09 | 4.09E+05 | 83.73 | 1.34E+05 |
| Control 2 | 73.14 | 4.10E+05 | 74.14 | 3.99E+05 | 83.67 | 1.38E+05 |
| Control 1 | 73.14 | 3.84E+05 | 74.07 | 3.83E+05 | 83.69 | 1.30E+05 |

TABLE 30
Results from Intact/Reduced MS

| | Intact Mass | Heavy Chain Mass | Light Chain Mass |
|---|---|---|---|
| Sample | (Da) | (Da) | (Da) |
| D2E7 pearls t0 | 148086 | 50638 | 23409 |
| D2E7 pearls 23 m | 148086 | 50638 | 23409 |
| Control 2 | 148086 | 50638 | 23409 |
| Control 1 | 148086 | 50638 | 23408 |

TABLE 31
Results from SEC-MALS

peak1

| | Mass | MWapp | Rn | |
|---|---|---|---|---|
| Sample | % | (kD) | (nm) | Pd |
| D2E7 pearls t0 | 100 | 147.2 | 5.6 | 1.001 |
| D2E7 pearls 23 m | 100 | 147.4 | 6.8 | 1.002 |
| Control 2 | 100 | 147.4 | 6.5 | 1.002 |
| Control 1 | 100 | 147 | 6.3 | 1.001 |

TABLE 32
Results from Dynamic Light Scattering

| | Radius | MW | % mass |
|---|---|---|---|
| Sample | (main peak) | (main peak) | (main peak) |
| D2E7 pearls t0 | 5.22 nm | 161 kDa | 100 |
| D2E7 pearls 23 m | 5.36 nm | 171 kDa | 100 |
| Control 2 | 5.15 nm | 156 kDa | 100 |
| Control 1 | 5.16 nm | 157 kDa | 100 |

TABLE 33
Results from Weak Cation Exchange Chromatography

| | | | | | | | Σ |
|---|---|---|---|---|---|---|---|
| Sample | AR1 | AR2 | Lys0 | Lys1 | Lys2 | Lys1.5 | lysines |
| Reference | 3.59 | 10.34 | 63.52 | 17.64 | 4.49 | 0.42 | 86.07 |
| standard | | | | | | | |
| D2E7 pearls t0 | 3.56 | 11.05 | 62.30 | 17.72 | 4.52 | 0.85 | 85.39 |
| D2E7 pearls 23 m | 6.37 | 12.90 | 59.15 | 17.66 | 3.28 | 0.64 | 80.73 |
| Control 2 | 2.14 | 10.95 | 64.45 | 16.98 | 4.16 | 1.32 | 86.91 |
| Control 1 | 3.14 | 11.71 | 63.97 | 16.34 | 3.76 | 1.08 | 85.15 |

TABLE 34
Results from Analytical Ultracentrifugation

| Lot | s (S) | s20, w (S) | f/f0 | MW (kDa) | % Monomer | % LMW | % HMW |
|---|---|---|---|---|---|---|---|
| D2E7 pearls t0 | 6.47 ± 0.06 | 6.65 ± 0.09 | 1.51 ± 0.03 | 146.4 ± 1.5 | 97.5 ± 0.7 | 0.1 ± 0.0 | 2.4 ± 0.7 |
| D2E7 pearls 23 m | 6.45 ± 0.07 | 6.63 ± 0.10 | 1.50 ± 0.02 | 144.9 ± 1.2 | 97.1 ± 0.2 | 0.0 ± 0.0 | 2.9 ± 0.2 |
| Control 2 | 6.47 ± 0.06 | 6.65 ± 0.09 | 1.50 ± 0.01 | 145.2 ± 1.3 | 99.9 ± 0.0 | 0.1 ± 0.0 | 0.0 ± 0.0 |
| Control 1 | 6.47 ± 0.06 | 6.64 ± 0.09 | 1.49 ± 0.01 | 144.0 ± 1.5 | 97.4 ± 0.7 | 0.0 ± 0.0 | 2.5 ± 0.7 |

This full detailed protein analysis shows comparability between freshly made uniform, flowable solid units and 23 month old uniform, flowable solid units with 2 liquid standards. No stability liabilities were observed in the uniform, flowable solid units and the 23 month old peptide mapping data was in good agreement with the freshly made uniform, flowable solid unit sample and liquid standard.

### Example 15

### Reconstitution of Antibody A Solid Units

The following example compares the reconstitution time of solid units comprising Antibody A (an IgG antibody) and sucrose, and lyophilized cakes comprising Antibody A and sucrose.

Antibody A solutions with varying protein and sucrose concentrations were prepared with 15 mM histidine at pH 6 and 0.02% (w/v) polysorbate 80. The protein concentration range was 50-150 mg/mL. The sucrose concentration range was 0-10% (w/v). The study plan constructed was a full factorial DOE design with 2 factors, 3 protein levels, and 4 sucrose levels creating a total of 12 formulations. Lyophilized cakes and lyophilized solid units were manufactured for each formulation in the same lyophilization load for direct stability and reconstitution comparison.

Each vial was filled with approximately 2.3 mL of solution. Lyo stoppers were inserted into the vials (lyo stoppers were partially inserted to allow sublimation venting). The vials were loaded into the lyophilizer at shelf temperature about 5° C. The shelf temperature was reduced to −35° C. for freezing. After being held at −35° C., the shelf temperature was warmed to −10° C. The shelf was then cooled to −50° C. After being held at −50° C., the lyo chamber was evacuated to a pressure of approximately 100 microns. Following evacuation, the vials were subjected to a primary drying step at about −8° C. and 100 microns of pressure. Lastly, the vials were subjected to a secondary drying step at 30° C. under approximately 100 microns of pressure. For the manufacture of solid units, freezing was performed by dispensing a liquid solution using a syringe pump into a stainless steel pan with dividers filled with liquid nitrogen. After a bulk of frozen solid units was manufactured, the solid units were transferred to pre-cooled vials on dry ice. Solid units equivalent to about 2.3 mL liquid fill solution was transferred into each of the vials. The frozen solid units in vials were then stoppered with lyo stoppers (lyo stoppers were partially inserted to allow sublimation venting) and loaded into the lyophilizer at shelf temperature about −50° C. The frozen solid units were subjected to the same lyophilization cycle and run as the corresponding liquid filled vial with same formulation.

Lyophilized cakes and lyophilized solid units were reconstituted with high purity water. Approximately 2.1 mL of high purity water was transferred to each of the vials. The vials were then swirled and mixed and the reconstitution time was recorded after all of the lyophilized cake or lyophilized solid units were dissolved.

Table 35 below summarizes the protein concentrations and sucrose concentrations of the twelve formulations used to prepare solid units and traditional lyophilized cakes of Antibody A.

TABLE 35
Summary of Antibody A Formulations

| | Protein Conc. | Sucrose |
|---|---|---|
| Formulation # | (mg/mL) | (% w/v) |
| 1 | 75 | 8 |
| 2 | 100 | 4 |
| 3 | 50 | 10 |
| 4 | 50 | 4 |
| 5 | 100 | 0 |
| 6 | 75 | 10 |
| 7 | 75 | 4 |
| 8 | 50 | 8 |
| 9 | 50 | 0 |
| 10 | 75 | 0 |
| 11 | 100 | 10 |
| 12 | 100 | 8 |

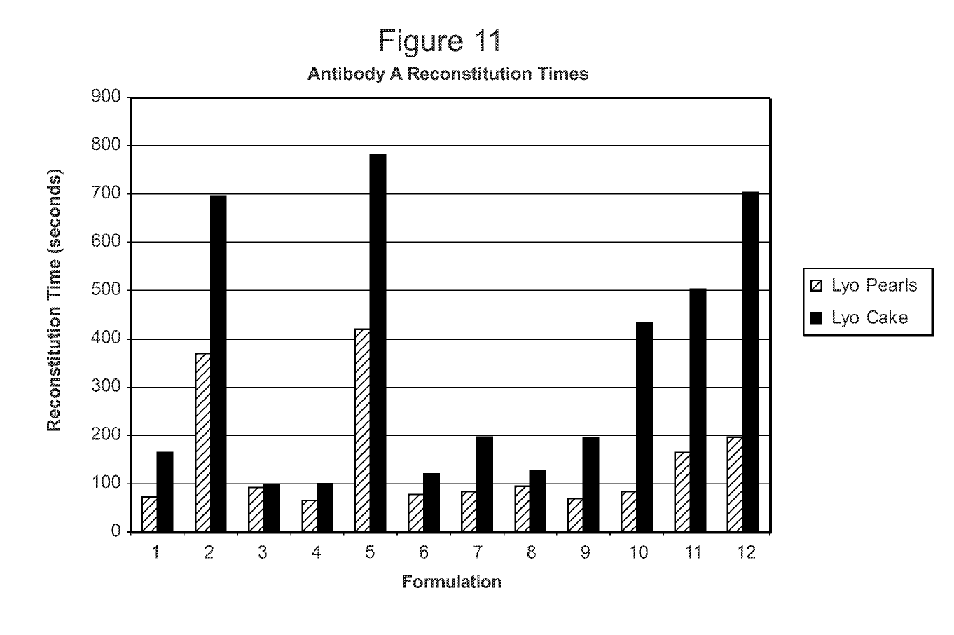

The results of thee analyses are presented in Table 36 and FIG. 11 and demonstrate that all of the formulations comprising Antibody A prepared as solid units have faster reconstitution times that the same formulations of Antibody A prepared as lyophilized cakes.

TABLE 36
Antibody A Reconstitution Time Comparison
Between BioPearl and Cake

| Antibody A | Pearl Recon Time | Cake Recon Time |
|---|---|---|
| Formulation | (secs) | (secs) |
| 1 | 73 | 164 |
| 2 | 369 | 696 |
| 3 | 92 | 96 |
| 4 | 64 | 99 |
| 5 | 420 | 782 |
| 6 | 78 | 120 |
| 7 | 84 | 197 |
| 8 | 95 | 127 |
| 9 | 70 | 195 |
| 10 | 83 | 432 |
| 11 | 165 | 503 |
| 12 | 196 | 704 |

### Example 16

### Additional Antibody a Solid Unit Studies

The following example describes the preparation of solid units comprising antibodies, exemplified by Antibody A, and further analysis of the solid units.

Solid units comprising Antibody A at 50, 75, or 100 mg/ml in a 15 mM histidine buffer, and 0.02% polysorbate 80, pH 6.0 (see Tables 37-40) were prepared by freezing using controlled nucleation as described in Example 1, above. Standard lyophilized cakes of the same formulations were also prepared.

The solid units and cakes were subjected to accelerated storage conditions for 4 weeks at which time they were reconstituted in water (see Example 15) and subjected to SEC analysis and Micro-Flow Imaging to determine the subvisible particle counts.

The data comparing the solid units versus the lyophilized cake are presented in Tables 37-40.

TABLE 37
Analysis of Antibody A Solid Units

| | Protein | | | Recon | Recon | | | |
|---|---|---|---|---|---|---|---|---|
| Form | Conc. | Sucrose | | Time | Time | Conc. | | % |
| # | (mg/mL) | (% w/v) | TimePoint↓ | min:secs | (secs) | (mg/mL) | pH | Moisture |
| 1 | 75 | 8 | FDS | NA | NA | 74.9 | 6.1 | NA |
| | | | T0_Pearl | 1:13 | 73 | 71.5 | NS | 0.4 |
| | | | 4 wks at 55° C. | 1:40 | 100 | NS | NS | NS |
| 2 | 100 | 4 | FDS | NA | NA | 102.5 | 6.0 | NA |
| | | | T0_Pearl | 6:09 | 369 | 101.5 | NS | 0.3 |
| | | | 4 wks at 55° C. | 4:40 | 280 | NS | NS | NS |
| 3 | 50 | 10 | FDS | NA | NA | 49.7 | 6.0 | NA |
| | | | T0_Pearl | 1:32 | 92 | 49.0 | NS | 0.3 |
| | | | 4 wks at 55° C. | 1:01 | 61 | NS | NS | NS |
| 4 | 50 | 4 | FDS | NA | NA | 51.1 | 6.0 | NA |
| | | | T0_Pearl | 1:04 | 64 | 51.6 | NS | 0.4 |
| | | | 4 wks at 55° C. | 1:30 | 90 | NS | NS | NS |
| 5 | 100 | 0 | FDS | NA | NA | 103.3 | 6.0 | NA |
| | | | T0_Pearl | 7:00 | 420 | 105.3 | NS | 0.3 |
| | | | 4 wks at 55° C. | 7:00 | 420 | NS | NS | NS |
| 6 | 75 | 10 | FDS | NA | NA | 75.1 | 6.0 | NA |
| | | | T0_Pearl | 1:18 | 78 | 74.1 | NS | 0.3 |
| | | | 4 wks at 55° C. | 2:59 | 179 | NS | NS | NS |
| 7 | 75 | 4 | FDS | NA | NA | 76.2 | 6.0 | NA |
| | | | T0_Pearl | 1:24 | 84 | 73.9 | NS | 0.3 |
| | | | 4 wks at 55° C. | 1:58 | 118 | NS | NS | NS |
| 8 | 50 | 8 | FDS | NA | NA | 49.5 | 6.0 | NA |
| | | | T0_Pearl | 1:35 | 95 | 47.5 | NS | 0.4 |
| | | | 4 wks at 55° C. | 1:38 | 98 | NS | NS | NS |
| 9 | 50 | 0 | FDS | NA | NA | 49.4 | 6.1 | NA |
| | | | T0_Pearl | 1:10 | 70 | 51.2 | NS | 0.5 |
| | | | 4 wks at 55° C. | 3:00 | 180 | NS | NS | NS |
| 10 | 75 | 0 | FDS | NA | NA | 76.1 | 6.1 | NA |
| | | | T0_Pearl | 1:23 | 83 | 77.4 | NS | 0.5 |
| | | | 4 wks at 55° C. | 3:52 | 232 | NS | NS | NS |
| 11 | 100 | 10 | FDS | NA | NA | 102.6 | 6.0 | NA |
| | | | T0_Pearl | 2:45 | 165 | 96.3 | NS | 0.3 |
| | | | 4 wks at 55° C. | 10:00 | 600 | NS | NS | NS |
| 12 | 100 | 8 | FDS | NA | NA | 102.9 | 6.0 | NA |
| | | | T0_Pearl | 3:16 | 196 | 98.2 | NS | 0.2 |
| | | | 4 wks at 55° C. | 12:00 | 720 | NS | NS | NS |

TABLE 38
Analysis of Antibody A Solid Units

| | Protein | | | SEC | | | Subvisible Particle Count | | | | |
|---|---|---|---|---|---|---|---|---|---|---|---|
| Form | Conc. | Sucrose | | % | % Main | % | by MFI (Particle Counts/mL) | | | | |
| # | (mg/mL) | (% w/v) | TimePoint↓ | Aggregates | Peak | Fragments | ≧1 μm | ≧2 μm | ≧5 μm | ≧10 μm | ≧25 μm |
| 1 | 75 | 8 | FDS | 1.6 | 98.3 | 0.1 | 944 | 186 | 56 | 26 | 12 |
| | | | T0_Pearl | 1.6 | 98.3 | 0.1 | 42703 | 7520 | 648 | 28 | 8 |
| | | | 4 wks at 55° C. | 3.9 | 96.0 | 0.1 | 139236 | 25688 | 3671 | 412 | 40 |
| 2 | 100 | 4 | FDS | 1.6 | 98.3 | 0.0 | 422 | 46 | 2 | 2 | 0 |
| | | | T0_Pearl | 2.0 | 98.0 | 0.0 | 203798 | 43527 | 4409 | 184 | 8 |
| | | | 4 wks at 55° C. | 13.7 | 86.2 | 0.1 | 4687961 | 580707 | 65932 | 6886 | 185 |
| 3 | 50 | 10 | FDS | 1.6 | 98.4 | 0.1 | 832 | 110 | 24 | 4 | 2 |
| | | | T0_Pearl | 1.5 | 98.4 | 0.1 | 74638 | 19151 | 2515 | 110 | 8 |
| | | | 4 wks at 55° C. | 2.1 | 97.8 | 0.1 | 60429 | 15286 | 2535 | 314 | 28 |
| 4 | 50 | 4 | FDS | 1.6 | 98.4 | 0.1 | 862 | 56 | 4 | 0 | 0 |
| | | | T0_Pearl | 1.6 | 98.3 | 0.1 | 116819 | 33008 | 3727 | 210 | 8 |
| | | | 4 wks at 55° C. | 5.4 | 94.5 | 0.1 | 134713 | 26629 | 3717 | 376 | 18 |
| 5 | 100 | 0 | FDS | 1.6 | 98.3 | 0.0 | 162 | 22 | 0 | 0 | 0 |
| | | | T0_Pearl | 6.4 | 93.5 | 0.1 | 604527 | 119405 | 10124 | 372 | 16 |
| | | | 4 wks at 55° C. | 44.1 | 55.6 | 0.3 | NT | NT | NT | NT | NT |
| 6 | 75 | 10 | FDS | 1.6 | 98.4 | 0.0 | 174 | 62 | 6 | 2 | 2 |
| | | | T0_Pearl | 1.6 | 98.3 | 0.1 | 73864 | 16152 | 1436 | 72 | 2 |
| | | | 4 wks at 55° C. | 3.0 | 96.9 | 0.1 | 77481 | 13859 | 1804 | 230 | 8 |
| 7 | 75 | 4 | FDS | 1.6 | 98.4 | 0.0 | 3219 | 400 | 42 | 6 | 0 |
| | | | T0_Pearl | 1.8 | 98.2 | 0.1 | 148815 | 29267 | 1758 | 28 | 4 |
| | | | 4 wks at 55° C. | 9.6 | 90.4 | 0.1 | 1212654 | 135820 | 16274 | 1934 | 46 |
| 8 | 50 | 8 | FDS | 1.6 | 98.3 | 0.1 | 254 | 34 | 0 | 0 | 0 |
| | | | T0_Pearl | 1.6 | 98.4 | 0.1 | 101323 | 21463 | 1094 | 64 | 4 |
| | | | 4 wks at 55° C. | 2.4 | 97.5 | 0.1 | 51477 | 13333 | 1768 | 128 | 2 |
| 9 | 50 | 0 | FDS | 1.6 | 98.4 | 0.0 | 760 | 116 | 32 | 16 | 2 |
| | | | T0_Pearl | 3.7 | 96.3 | 0.1 | 105428 | 22698 | 2529 | 266 | 12 |
| | | | 4 wks at 55° C. | 37.1 | 62.7 | 0.3 | NT | NT | NT | NT | NT |
| 10 | 75 | 0 | FDS | 1.6 | 98.3 | 0.1 | 522 | 60 | 10 | 6 | 4 |
| | | | T0_Pearl | 4.5 | 95.5 | 0.1 | 275946 | 37803 | 2835 | 198 | 6 |
| | | | 4 wks at 55° C. | 39.6 | 60.2 | 0.1 | NT | NT | NT | NT | NT |
| 11 | 100 | 10 | FDS | 1.6 | 98.3 | 0.0 | 302 | 66 | 16 | 2 | 0 |
| | | | T0_Pearl | 1.7 | 98.3 | 0.1 | 133579 | 25356 | 2373 | 128 | 32 |
| | | | 4 wks at 55° C. | 4.4 | 95.5 | 0.1 | 179024 | 28521 | 3079 | 276 | 22 |
| 12 | 100 | 8 | FDS | 1.6 | 98.3 | 0.0 | 254 | 58 | 18 | 6 | 4 |
| | | | T0_Pearl | 1.7 | 98.3 | 0.0 | 224670 | 38686 | 2473 | 14 | 2 |
| | | | 4 wks at 55° C. | 5.9 | 94.0 | 0.1 | 241216 | 41412 | 4475 | 286 | 8 |

TABLE 39
Analysis of Antibody A Lyophilized Cakes

| | Protein | | | Recon | Recon | | | |
|---|---|---|---|---|---|---|---|---|
| Form | Conc. | Sucrose | | Time | Time | Concentration | | % |
| # | (mg/mL) | (% w/v) | TimePoint↓ | min:secs | (secs) | (mg/mL) | pH | Moisture |
| 1 | 75 | 8 | FDS | NA | NA | 74.9 | 6.1 | NA |
| | | | T0_Cake | 2:44 | 164 | 72.4 | NS | 0.3 |
| | | | T4 wks at 55° C. | 3:05 | 185 | NS | NS | NS |
| 2 | 100 | 4 | FDS | NA | NA | 102.5 | 6.0 | NA |
| | | | T0_Cake | 11:36 | 696 | 99.2 | NS | 0.2 |
| | | | T4 wks at 55° C. | 12:30 | 750 | NS | NS | NS |
| 3 | 50 | 10 | FDS | NA | NA | 49.7 | 6.0 | NA |
| | | | T0_Cake | 1:36 | 96 | 49.1 | NS | 0.6 |
| | | | T4 wks at 55° C. | 1:30 | 90 | NS | NS | NS |
| 4 | 50 | 4 | FDS | NA | NA | 51.1 | 6.0 | NA |
| | | | T0_Cake | 1:33 | 99 | 50.8 | NS | 0.5 |
| | | | T4 wks at 55° C. | 1:35 | 95 | NS | NS | NS |
| 5 | 100 | 0 | FDS | NA | NA | 103.3 | 6.0 | NA |
| | | | T0_Cake | 13:02 | 782 | 101.1 | NS | 0.3 |
| | | | T4 wks at 55° C. | 30.00 | 1800 | NS | NS | NS |
| 6 | 75 | 10 | FDS | NA | NA | 75.1 | 6.0 | NA |
| | | | T0_Cake | 2:00 | 120 | 72.9 | NS | 0.5 |
| | | | T4 wks at 55° C. | 5:37 | 337 | NS | NS | NS |
| 7 | 75 | 4 | FDS | NA | NA | 76.2 | 6.0 | NA |
| | | | T0_Cake | 3:17 | 197 | 74.9 | NS | 0.2 |
| | | | T4 wks at 55° C. | 5:30 | 330 | NS | NS | NS |
| 8 | 50 | 8 | FDS | NA | NA | 49.5 | 6.0 | NA |
| | | | T0_Cake | 2:07 | 127 | 49.3 | NS | 0.5 |
| | | | T4 wks at 55° C. | 1:17 | 77 | NS | NS | NS |
| 9 | 50 | 0 | FDS | NA | NA | 49.4 | 6.1 | NA |
| | | | T0_Cake | 3:15 | 195 | 51.2 | NS | 0.4 |
| | | | T4 wks at 55° C. | 6:49 | 409 | NS | NS | NS |
| 10 | 75 | 0 | FDS | NA | NA | 76.1 | 6.1 | NA |
| | | | T0_Cake | 7:12 | 432 | 75.4 | NS | 0.4 |
| | | | T4 wks at 55° C. | 20 | 1200 | NS | NS | NS |
| 11 | 100 | 10 | FDS | NA | NA | 102.6 | 6.0 | NA |
| | | | T0_Cake | 8:23 | 503 | 97.5 | NS | 0.5 |
| | | | T4 wks at 55° C. | 13:29 | 809 | NS | NS | NS |
| 12 | 100 | 8 | FDS | NA | NA | 102.9 | 6.0 | NA |
| | | | T0_Cake | 11:44 | 704 | 97.8 | NS | 0.4 |
| | | | T4 wks at 55° C. | 13:30 | 810 | NS | NS | NS |

TABLE 40
Analysis of Antibody A Lyophilized Cakes

| | Protein | | | SEC | | | Subvisible Particle Count | | | | |
|---|---|---|---|---|---|---|---|---|---|---|---|
| Form | Conc. | Sucrose | | % | % Main | % | by MFI (Particle Counts/mL) | | | | |
| # | (mg/mL) | (% w/v) | TimePoint↓ | Aggregates | Peak | Fragments | ≧1 μm | ≧2 μm | ≧5 μm | ≧10 μm | ≧25 μm |
| 1 | 75 | 8 | FDS | 1.6 | 98.3 | 0.1 | 944 | 186 | 56 | 26 | 12 |
| | | | T0_Cake | 1.6 | 98.3 | 0.1 | 168382 | 36177 | 3783 | 100 | 0 |
| | | | T4 wks at 55° C. | 3.4 | 96.6 | 0.0 | 112634 | 26905 | 3569 | 314 | 6 |
| 2 | 100 | 4 | FDS | 1.6 | 98.3 | 0.0 | 422 | 46 | 2 | 2 | 0 |
| | | | T0_Cake | 1.9 | 98.1 | 0.0 | 363470 | 49994 | 3455 | 50 | 0 |
| | | | T4 wks at 55° C. | 12.7 | 87.2 | 0.1 | 679984 | 105590 | 7780 | 358 | 4 |
| 3 | 50 | 10 | FDS | 1.6 | 98.4 | 0.1 | 832 | 110 | 24 | 4 | 2 |
| | | | T0_Cake | 1.6 | 98.4 | 0.1 | 120068 | 33538 | 4807 | 240 | 6 |
| | | | T4 wks at 55° C. | 1.9 | 98.0 | 0.0 | 45513 | 7310 | 660 | 82 | 6 |
| 4 | 50 | 4 | FDS | 1.6 | 98.4 | 0.1 | 862 | 56 | 4 | 0 | 0 |
| | | | T0_Cake | 1.6 | 98.3 | 0.1 | 63172 | 12305 | 978 | 36 | 4 |
| | | | T4 wks at 55° C. | 4.8 | 95.2 | 0.1 | 101565 | 22810 | 2445 | 276 | 12 |
| 5 | 100 | 0 | FDS | 1.6 | 98.3 | 0.0 | 162 | 22 | 0 | 0 | 0 |
| | | | T0_Cake | 7.3 | 92.6 | 0.0 | 702468 | 137234 | 11177 | 488 | 4 |
| | | | T4 wks at 55° C. | 43.8 | 56.0 | 0.2 | NT | NT | NT | NT | NT |
| 6 | 75 | 10 | FDS | 1.6 | 98.4 | 0.0 | 174 | 62 | 6 | 2 | 2 |
| | | | T0_Cake | 1.6 | 98.3 | 0.0 | 106412 | 22954 | 3249 | 122 | 0 |
| | | | T4 wks at 55° C. | 2.6 | 97.3 | 0.1 | 176172 | 30834 | 3359 | 148 | 6 |
| 7 | 75 | 4 | FDS | 1.6 | 98.4 | 0.0 | 3219 | 400 | 42 | 6 | 0 |
| | | | T0_Cake | 1.7 | 98.2 | 0.1 | 181331 | 32566 | 2739 | 118 | 4 |
| | | | T4 wks at 55° C. | 8.6 | 91.4 | 0.1 | 410491 | 99305 | 14412 | 1612 | 34 |
| 8 | 50 | 8 | FDS | 1.6 | 98.3 | 0.1 | 254 | 34 | 0 | 0 | 0 |
| | | | T0_Cake | 1.6 | 98.4 | 0.1 | 158883 | 38804 | 5267 | 178 | 8 |
| | | | T4 wks at 55° C. | 2.2 | 97.8 | 0.1 | 55284 | 10529 | 958 | 44 | 6 |
| 9 | 50 | 0 | FDS | 1.6 | 98.4 | 0.0 | 760 | 116 | 32 | 16 | 2 |
| | | | T0_Cake | 3.6 | 96.4 | 0.1 | 320431 | 91745 | 11385 | 868 | 8 |
| | | | T4 wks at 55° C. | 35.0 | 64.9 | 0.1 | NT | NT | NT | NT | NT |
| 10 | 75 | 0 | FDS | 1.6 | 98.3 | 0.1 | 522 | 60 | 10 | 6 | 4 |
| | | | T0_Cake | 5.1 | 94.9 | 0.1 | 635571 | 134667 | 14398 | 1502 | 68 |
| | | | T4 wks at 55° C. | 40.2 | 59.5 | 0.2 | NT | NT | NT | NT | NT |
| 11 | 100 | 10 | FDS | 1.6 | 98.3 | 0.0 | 302 | 66 | 16 | 2 | 0 |
| | | | T0_Cake | 1.7 | 98.3 | 0.1 | 196373 | 38716 | 6210 | 220 | 14 |
| | | | T4 wks at 55° C. | 3.6 | 96.4 | 0.0 | 252808 | 38031 | 2887 | 134 | 14 |
| 12 | 100 | 8 | FDS | 1.6 | 98.3 | 0.0 | 254 | 58 | 18 | 6 | 4 |
| | | | T0_Cake | 1.7 | 98.3 | 0.1 | 398416 | 71374 | 8888 | 288 | 8 |
| | | | T4 wks at 55° C. | 5.2 | 94.8 | 0.0 | 162866 | 24222 | 1600 | 16 | 0 |

### Example 17

### Manufacturing of Solid Units for Enteric Coating

A low acidic adalimumab solution was prepared with the components in Table 41, below.

TABLE 41
Low Acidic Adalimumab Formulation

| | Amount |
|---|---|
| Material | (mg/mL) |
| Low Acidic Adalimumab | 100 |
| Sorbitol | 100 |

The solution was transferred to a syringe and pumped through a 0.2 mm stainless steel nozzle at a flow rate of about 4 mL/min. A sonication head vibrated the stainless steel nozzle to produce uniform, spherical liquid units about 0.4 mm in diameter. The liquid units passed through an electrical field where a voltage was applied to separate the units. The liquid units were placed into a stainless steel tray filled with liquid nitrogen. The tray containing the frozen solid units was loaded into the lyophilizer at shelf temperature about −50° C. After being held at −50° C., the shelf temperature was warmed to −30° C. and held there for a couple hours. The shelf was then cooled to −50° C. After being held at −50° C., the lyophilization chamber was evacuated to a pressure of approximately 100 microns. Following evacuation, the solid units were subjected to a primary drying step at about −15° C. and 100 microns of pressure. Lastly, the solid units were subjected to a secondary drying step at 25° C. under approximately 100 microns of pressure.

The lyophilized solid units were sieved to NMT 0.4 mm in size and mixed with SiO₂ to prevent static. The solid units were then exposed to room temperature humidity for about 1.5 hours. A Wurster coater was used to coat the solid units with Eudragit 5100 enteric coating. The seal coats in Table 42 were utilized.

TABLE 42
Seal Coat Compositions

| | Eudragit | TEC | Talc | Acetone | IPA |
|---|---|---|---|---|---|
| Coating # | (%) | (%) | (%) | (%) | (%) |
| 1^{st} | 3.49 | 1.3 | 3.99 | 72.83 | 18.38 |
| 2^{nd} | 1.99 | 0.72 | 2.30 | 72.80 | 22.18 |
| 3^{rd} | 2.00 | 0.44 | 1.04 | 77.89 | 18.61 |

The coated solid units were then tested with an in-vitro test method. The coated solid units were tested using a SoloVPE equipment to determine the amount of protein released at variable pH. The SoloVPE equipment is a UV-Vis spectrophotometer that has the ability to dynamically vary the measurement path length and enables accurate measurements of highly concentrated samples without any further dilution.

The coated solid units were transferred to a scintillation vial and 1 mL pH 1.68 buffer was added. After 30 minutes, the sample was tested on the SoloVPE to measure the amount of protein released. In the same vial, NaOH was added to raise the pH to 7 and after 30 minutes the sample was tested using the SoloVPE to measure the amount of protein released. The % burst is calculated based on the ratio between the amount of protein released at pH 1.68 and pH 7. The coated solid units in the study had a burst effect of about 26%.

The stability of the coated solid units stored at 25° C. for about 24 hours was determined by SEC and CEX HPLC, as described above and is presented in Table 43.

TABLE 43
Stability of Coated Solid Units Stored at 25°
C. As Determined by Size Exclusion Chromatography
and Cation Exchange Chromatography

| | Control Minipearls | Coated Minipearls |
|---|---|---|
| Aggregates | 0.37 | 6.49 |
| Monomer | 99.63 | 93.26 |
| Fragments | 0 | 0.25 |
| Acidic Region 1 | 2.12 | 2.01 |
| Acidic Region 2 | 10.37 | 10.48 |
| Sum of lysines | 85.88 | 85.83 |
| Peak between lys 1 and lys 2 | 1 | 1 |
| Peak after lys 2 | 0.63 | 0.69 |

### Example 18

### Preparation of DVD-Ig Protein DVD-Ig C Solid Units

Solid units comprising DVD-Ig C were prepared as described above using controlled nucleation and assessed for stability following storage under accelerated storage conditions (40° C./75% humidity) for 2 weeks, 1 month, 6 weeks, and 2 months by SEC analysis.

A total of six DVD-Ig C formulations were evaluated (Table 44). Solid units prepared from each of the six formulations were prepared as described above. In particular, freezing was performed by dispensing the liquid solution into a stainless steel pan with dividers filled with liquid nitrogen at approximately −190° C. After a bulk of frozen solid units was manufactured, the solid units were transferred to pre-cooled vials on dry ice. The frozen solid units were transferred into each of the vial and then stoppered with lyo stoppers (lyo stoppers were partially inserted to allow sublimation venting) and loaded into the lyophilizer at shelf temperature about −50° C. After being held at −50° C., the shelf temperature was warmed to −30° C. The shelf was then cooled to −50° C. After being held at −50° C., the lyophilization chamber was evacuated to a pressure of approximately 100 microns. Following evacuation, the vials were subjected to a primary drying step at about −15° C. and 100 microns of pressure. Lastly, the vials were subjected to a secondary drying step at 25° C. under approximately 100 microns of pressure. The lyophilized solid units were stored refrigerated until use. The solid units in vials were reconstituted with water before use.

TABLE 44
DVD-Ig C Formulations

| Run # | DVD-Ig C | EXCIPIENTS |
|---|---|---|
| Run 1 | 100.85 mg/mL | 20 mM acetate, 7% sucrose, 0.01% |
| | | tween 80, pH 5 |
| Run 2 | 105.04 mg/mL | 7.5% sucrose, 0.01% tween 80, pH 5 |
| Run 3 | 102.67 mg/mL | 15 mM Histidine, 7% sucrose, 0.01% |
| | | tween 80, pH 5 |
| Run 4 | 108.48 mg/mL | 15 mM Histidine, 50 mM arginine- |
| | | HCl, 5% sucrose, 0.01% tween 80, |
| | | pH 5.4 |
| Run 5 | 105.33 mg/mL | 15 mM Histidine, 50 mM glycine- |
| | | HCl, 5% sucrose, 0.01% tween 80, |
| | | pH 5.4 |
| Run 6 | 56.15 mg/mL | 15 mM Histidine, 7% sucrose, 0.01% |
| | | tween 80, pH 5.4 |

The results of the stability analyses are shown in Table 45.

TABLE 45
Stability of DVD-Ig C Solid Units as Determined by SEC Analysis

| | | | | SEC | | | |
|---|---|---|---|---|---|---|---|
| | | | | Mono- | | | |
| Run | DVD- | | Time | mer | Acidic | Main | Basic |
| # | Ig C | Excipients | point | (%) | (%) | (%) | (%) |
| Run | 101 | 20 mM | t zero | 95.5 | 11.8 | 56.1 | 32.1 |
| 1 | mg/mL | acetate, | 2 weeks | 93.7 | 12.5 | 54.9 | 32.6 |
| | | 7% | 1 month | 92.9 | 13.7 | 53.3 | 33.0 |
| | | sucrose, | 6 weeks | 91.5 | 14.0 | 51.8 | 34.2 |
| | | 0.01% | 2 month | 90.9 | 14.8 | 50.8 | 34.5 |
| | | PS80 | | | | | |
| Run | 105 | 7% | t zero | 96.3 | 11.2 | 57.5 | 31.3 |
| 2 | mg/mL | sucrose, | 2 weeks | 94.7 | 11.6 | 55.0 | 33.3 |
| | | 0.01% | 1 month | 93.7 | 12.0 | 53.8 | 34.3 |
| | | PS80 | 6 weeks | 93.2 | 11.9 | 52.6 | 35.4 |
| | | | 2 month | 92.4 | 12.5 | 50.9 | 36.6 |
| Run | 103 | 15 mM | t zero | 96.3 | 11.3 | 57.9 | 30.8 |
| 3 | mg/mL | Histidine, | 2 weeks | 94.9 | 11.6 | 55.3 | 33.1 |
| | | 7% | 1 month | 94.0 | 11.1 | 54.3 | 34.5 |
| | | sucrose, | 6 weeks | 93.4 | 11.4 | 52.9 | 35.8 |
| | | 0.01% | 2 month | 92.8 | 11.6 | 51.7 | 36.7 |
| | | PS80 | | | | | |
| Run | 109 | 15 mM | t zero | 96.0 | 11.7 | 57.1 | 31.2 |
| 4 | mg/mL | Histidine, | 2 weeks | 93.9 | 11.7 | 54.7 | 33.6 |
| | | 50 mM | 1 month | 93.6 | 12.0 | 53.3 | 34.8 |
| | | Arginine | 6 weeks | 93.5 | 12.0 | 52.6 | 35.5 |
| | | HCl, 5% | 2 month | 92.9 | 12.3 | 51.3 | 36.4 |
| | | sucrose, | | | | | |
| | | 0.01% | | | | | |
| | | PS80 | | | | | |
| Run | 105 | 15 mM | t zero | 96.8 | 10.7 | 55.0 | 34.3 |
| 5 | mg/mL | Histidine, | 2 weeks | 95.2 | 11.6 | 52.6 | 35.8 |
| | | 50 mM | 1 month | 94.2 | 11.9 | 51.4 | 36.7 |
| | | glycine | 6 weeks | 93.6 | 12.5 | 50.4 | 37.1 |
| | | HCl, 5% | 2 month | 93.0 | 12.8 | 49.2 | 38.1 |
| | | sucrose, | | | | | |
| | | 0.01% | | | | | |
| | | PS80 | | | | | |
| Run | 56 | 15 mM | t zero | 97.2 | 11.3 | 57.6 | 31.2 |
| 6 | mg/mL | Histidine, | 2 weeks | 96.7 | 11.3 | 55.9 | 32.8 |
| | | 7% | 1 month | 96.3 | 11.3 | 54.8 | 33.9 |
| | | sucrose, | 6 weeks | 96.2 | 11.6 | 54.5 | 33.9 |
| | | 0.01% | 2 month | 95.9 | 11.4 | 53.8 | 34.8 |
| | | PS80 | | | | | |

### Example 19

### Preparation of DVD-Ig B Protein Solid Units

Solid units comprising DVD-Ig B were prepared as described above using controlled nucleation and assessed for stability following storage under accelerated storage conditions (40° C./75% humidity) for 3 weeks by SEC analysis. In particular, DVD-Ig B solutions with varying sucrose, polysorbate 80, glycine and mannitol concentrations were prepared with 15 mM Histidine at pH 6. A total of 5 formulations were evaluated (Table 46). Solid units of all five formulations were manufactured in as described above.

Specifically, freezing was performed by dispensing the liquid solution into a stainless steel pan with dividers filled with liquid nitrogen at approximately −190° C. After a bulk of frozen solid units was manufactured, the solid units were transferred to pre-cooled vials on dry ice. The frozen solid units were transferred into each of the vial and then stoppered with lyo stoppers (lyo stoppers were partially inserted to allow sublimation venting) and loaded into the lyophilizer at shelf temperature about −45° C. After being held at −45° C., the shelf temperature was warmed to −15° C. The shelf was then cooled to −45° C. After being held at −45° C., the lyophilization chamber was evacuated to a pressure of approximately 100 microns. Following evacuation, the vials were subjected to a primary drying step at about −15° C. and 100 microns of pressure. Lastly, the vials were subjected to a secondary drying step at 25° C. under approximately 100 microns of pressure. The lyophilized solid units were stored refrigerated until use. The solid units in vials were reconstituted with water before use. The stability of these solid units is described in Table 47.

TABLE 46
DVD-Ig B Formulations

| Run # | DVD-Ig B | EXCIPIENTS |
|---|---|---|
| Run 1 | 50 mg/mL | 15 mM Histidine, 1 mg/mL tween 80, |
| | | 65 mg/mL sucrose, 12 mg/mL |
| | | Mannitol |
| Run 2 | 50 mg/mL | 15 mM Histidine, 1 mg/mL tween 80, |
| | | 100 mg/mL sucrose, 12 mg/mL |
| | | Mannitol |
| Run 3 | 50 mg/mL | 15 mM Histidine, 1 mg/mL tween 80, |
| | | 30 mg/mL sucrose, 12 mg/mL |
| | | Mannitol |
| Run 4 | 50 mg/mL | 15 mM Histidine, 65 mg/mL sucrose, |
| | | 12 mg/mL Mannitol |
| Run 5 | 50 mg/mL | 15 mM Histidine, 1 mg/mL tween 80, |
| | | 65 mg/mL sucrose, 25 mg/mL Glycine |

TABLE 47
Stability of DVD-Ig B Solid Units as Determined by SEC Analysis

| | | | | SEC | | | |
|---|---|---|---|---|---|---|---|
| | | | | Mono- | | | |
| Run | DVD- | | Time | mer | Acidic | Main | Basic |
| # | IgB | Excipients | point | (%) | (%) | (%) | (%) |
| Run | 50 | 15 mM | t zero | 96.62 | 24.93 | 61.42 | 13.66 |
| 1 | mg/mL | histidine, | 3 weeks | 96.28 | 25.34 | 61.38 | 13.27 |
| | | 1 mg/mL | | | | | |
| | | tween 80, | | | | | |
| | | 65 mg/mL | | | | | |
| | | sucrose, | | | | | |
| | | 12 mg/mL | | | | | |
| | | mannitol | | | | | |
| Run | 50 | 15 mM | t zero | 96.78 | 25.10 | 61.65 | 13.24 |
| 2 | mg/mL | histidine, | 3 weeks | 96.54 | 25.29 | 61.11 | 13.60 |
| | | 1 mg/mL | | | | | |
| | | tween 80, | | | | | |
| | | 100 mg/mL | | | | | |
| | | sucrose, | | | | | |
| | | 12 mg/mL | | | | | |
| | | mannitol | | | | | |
| Run | 50 | 15 mM | t zero | 96.59 | 25.23 | 62.07 | 12.70 |
| 3 | mg/mL | histidine, | 3 weeks | 95.29 | 25.20 | 60.76 | 14.04 |
| | | 1 mg/mL | | | | | |
| | | tween 80, | | | | | |
| | | 30 mg/mL | | | | | |
| | | sucrose, | | | | | |
| | | 12 mg/mL | | | | | |
| | | mannitol | | | | | |
| Run | 50 | 15 mM | t zero | 96.73 | 25.17 | 61.78 | 13.05 |
| 4 | mg/mL | histidine, | 3 weeks | 96.17 | 25.10 | 61.88 | 13.03 |
| | | 65 mg/mL | | | | | |
| | | sucrose, | | | | | |
| | | 12 mg/mL | | | | | |
| | | mannitol | | | | | |
| Run | 50 | 15 mM | t zero | 96.57 | 25.43 | 61.88 | 12.69 |
| 5 | mg/mL | histidine, | 3 weeks | 96.38 | 26.37 | 60.79 | 12.85 |
| | | 1 mg/mL | | | | | |
| | | tween 80, | | | | | |
| | | 65 mg/mL | | | | | |
| | | sucrose, | | | | | |
| | | 25 mg/mL | | | | | |
| | | glycine | | | | | |

### Example 20

### Study Evaluating Mannitol:Sucrose Relationship in Lyophilized Formulations

The following studies evaluated the impact of the excipients tween, mannitol and sucrose on the stability of adalimumab and low acidic adalimumab antibodies in lyophilized solid units. The studies were designed to determine both if a co-founding stability relationship between mannitol and sucrose exists, and the impact of the surfactant polysorbate (tween) for the stability of adalimumab and low acidic adalimumab antibodies.

To determine the impact of mannitol and sucrose on solid unit stability, a response surface design with a 16 run D-Optimal design evaluation was employed. Three levels for each factor (mannitol and sucrose) were utilized. Tween was treated as a blocking factor (4 blocks) allowing detailed studies of the linear and quadratic effects of mannitol and sucrose and their interaction while retaining capability for detecting the effect of tween on stability. A single center point with the D-optimal design places more points on the vertex to achieve higher D-efficiency and therefore greater power for detecting significant factor effects. The center point utilized the control solid unit formulation. Blocks 2 and 4 were combined for Tween 1 mg/mL, whereas block 1 was assigned to tween 0 mg/mL and block 3 to 0.1 mg/mL to achieve a better balance between factors (mannitol and sucrose) and levels (−1, 0, and 1). Table 48 provides a summary of Runs 1 to 16 and describes the various combinations of tween, mannitol, and sucrose.

Sixteen formulations (Runs 1 to 16) were prepared according to Table 48 with 50 mg/mL adalimumab. Another sixteen formulations were prepared according to Table 48 with 50 mg/mL low acidic adalimumab. Freezing was performed by dispensing the liquid solution through a syringe and freezing the spherical droplets into a stainless steel pan with dividers filled with liquid nitrogen. After a bulk of frozen solid units was manufactured for each of the formulations for adalimumab and low acidic adalimumab, the solid units were transferred to pre-cooled vials on dry ice. Solid units equivalent to about 0.5 mL liquid fill solution was transferred into each of the vials. The frozen solid units in vials were then stoppered with lyo stoppers (lyo stoppers were partially inserted to allow sublimation venting) and loaded into the lyophilizer at shelf temperature about −45° C. to −50° C. After being held at about −45° C. to −50° C., the shelf temperature was warmed to −15° C. The shelf was then cooled to about −45° C. to −50° C. After being held at about −45° C. to −50° C., the lyophilization chamber was evacuated to a pressure of approximately 100 microns. Following evacuation, the vials were subjected to a primary drying step at about −15° C. and 100 microns of pressure. Lastly, the vials were subjected to a secondary drying step at about 25° C. under approximately 100 microns of pressure. At the end of the lyophilization cycle, the lyo door was opened and all vials were immediately stoppered. The vials were then sealed with aluminum flip off caps and stored at 2-8° C. until placed into 40° C./75% RH storage conditions.

TABLE 48
Summary of Runs 1 to 16
Design

| | | | | Sum of | |
|---|---|---|---|---|---|
| Run | Mannitol | Sucrose | Tween | Lysines | Monomer |
| 1 | −1 | 1 | 1 | — | — |
| 2 | 1 | 0 | 1 | — | — |
| 3 | −1 | −1 | 1 | — | — |
| 4 | 0 | −1 | 1 | — | — |
| 5 | 1 | 1 | 2 | — | — |
| 6 | 0 | −1 | 2 | — | — |
| 7 | −1 | 0 | 2 | — | — |
| 8 | 1 | −1 | 2 | — | — |
| 9 | −1 | −1 | 3 | — | — |
| 10 | 0 | 1 | 3 | — | — |
| 11 | 1 | −1 | 3 | — | — |
| 12 | −1 | 0 | 3 | — | — |
| 13 | 0 | 0 | 4 | — | — |
| 14 | 1 | 1 | 4 | — | — |
| 15 | −1 | −1 | 4 | — | — |
| 16 | −1 | 1 | 4 | — | — |

Key to Table 48:
Mannitol:
−1 = 1 mg/mL
0 = 12 mg/mL
1 = 50 mg/mL
Sucrose:
−1 = 1 mg/mL
0 = 65 mg/mL
1 = 100 mg/mL
Tween (Block):
1 = 0 mg/mL
3 = 0.05 mg/mL
2 & 4 = 1 mg/mL

Each combination of excipients was analyzed for both monomer (reflective of aggregates) and lysine (reflective of degradation) content. Analysis of variance (ANOVA) was used to evaluate the significance of the mannitol and sucrose (linear, quadratic and interaction terms) on the response variables at the 0.05 level of statistical significance. The effect of block factor Tween 80 was also evaluated. The data were imported into a JMP table. All analyses were conducted using SAS JMP version 10. The JMP analysis output and script and selected are provided in Appendices A and B respectively.

A control formulation having low levels of aggregation and lysine content was first analyzed at time periods up to 3 months. Up to two months of data was collected for two sets of adalimumab and low acidic adalimumab from the 16-runs mentioned in Table 48 with varying concentrations of mannitol, sucrose and Tween 80. A formulation containing adalimumab, 12 mg/ml of mannitol and 65 mg/ml of sucrose was set as the center point conditions; Tween 80 was designed as block factor. The excipients and response variables involved in these experiments are listed in Table 49.

TABLE 49
List of Controlled Excipients and Response Variables

| | Concentration |
|---|---|
| Excipients | (mg/ml) |
| Sucrose | 1, 65, 100 |
| Mannitol | 1, 12, 50 |
| Tween 80 | 0, 0.05, 1 |

| Response | Specification |
|---|---|
| Monomer | NLT 98% |
| Sum of Lysine | NLT 75% |

For the control pearl formulation containing 50 mg/ml of adalimumab, 12 mg/ml mannitol, 1 mg/ml Tween 80, and 65 mg/ml sucrose, monomer content (with a specification of greater than 98%) was 99.6% at week 0; 99.5% monomer at week 2; 99.1% monomer at week 5; 99.1% monomer content at 2 months; and 98.9% monomer content at month 3. For the same control pearl formulation, the sum of lysines (with a specification of greater than 75%) was 86.8% at week 0; 85.7% at week 2; 83.7% at week 5; 83.1% at 2 months; and 82.4% at month 3. Weeks/months indicate the time period of storage of the pearl.

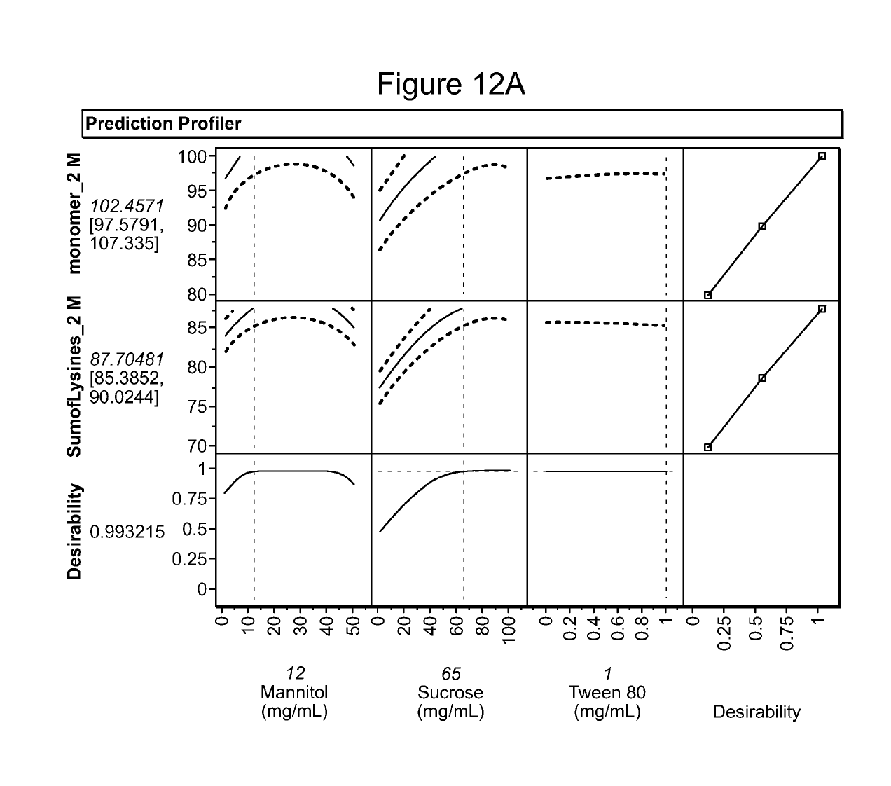

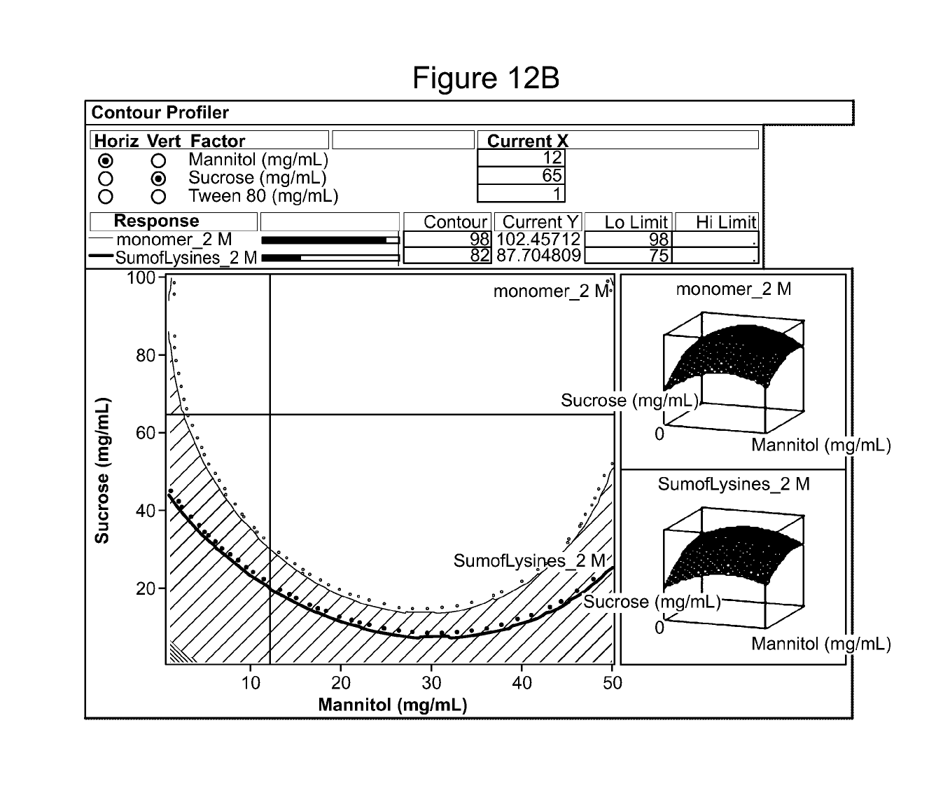

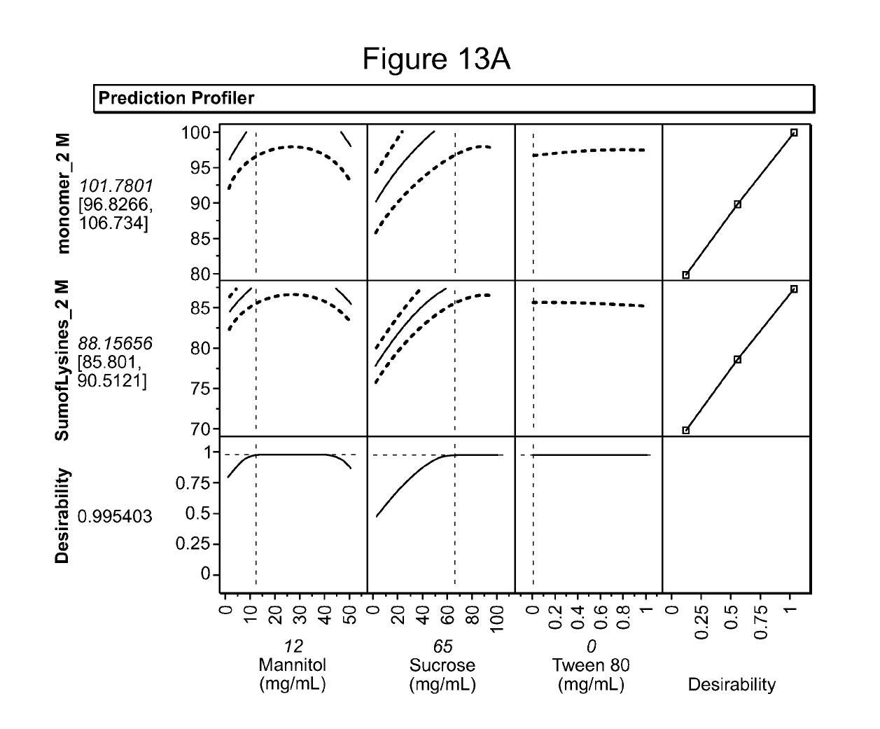

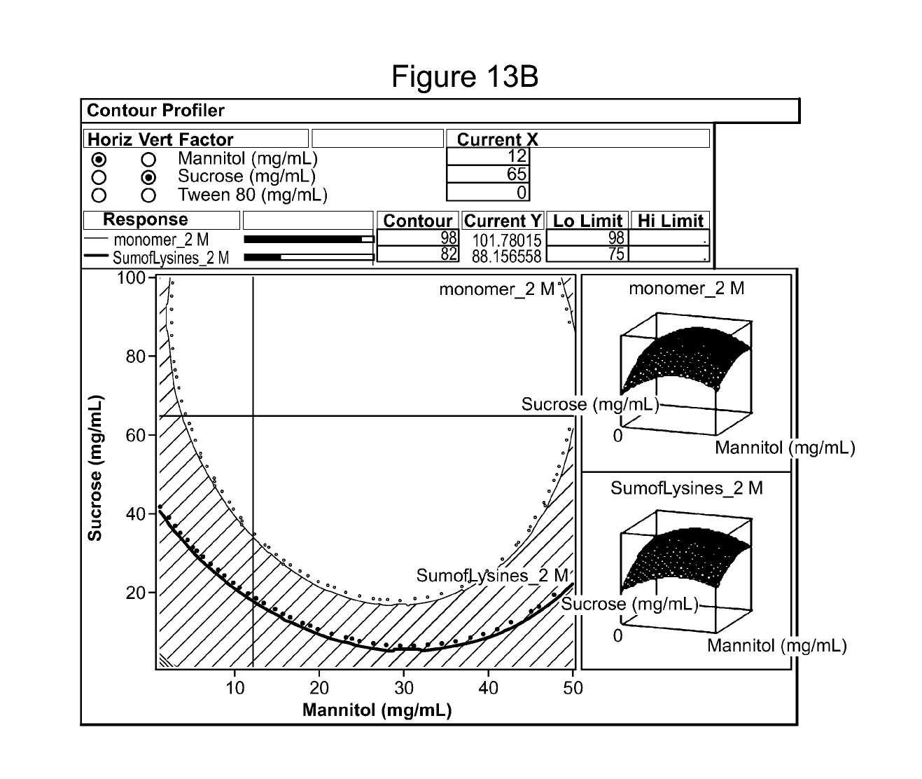

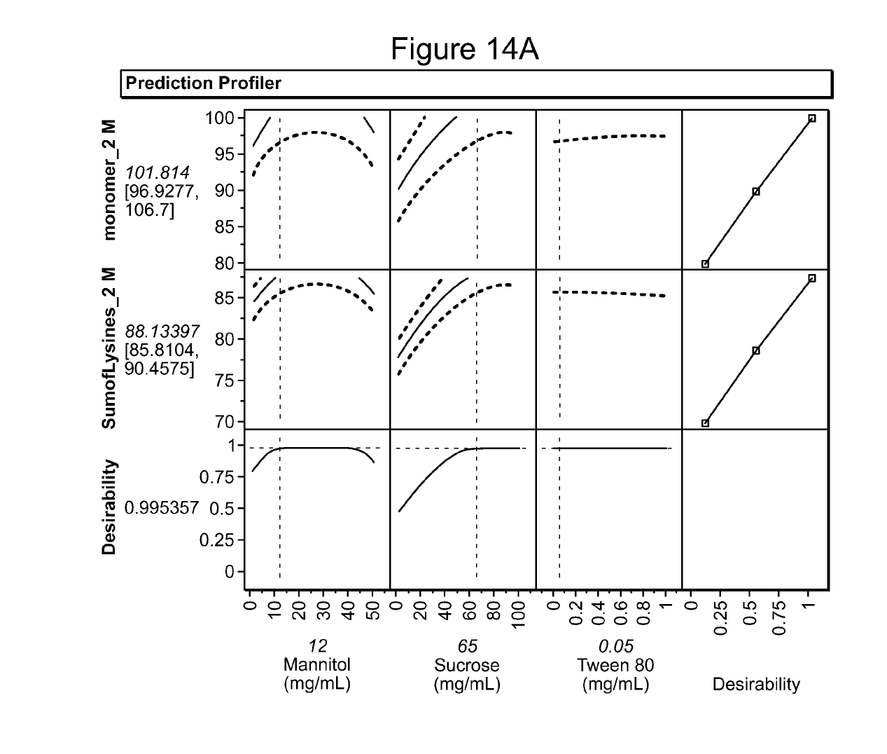

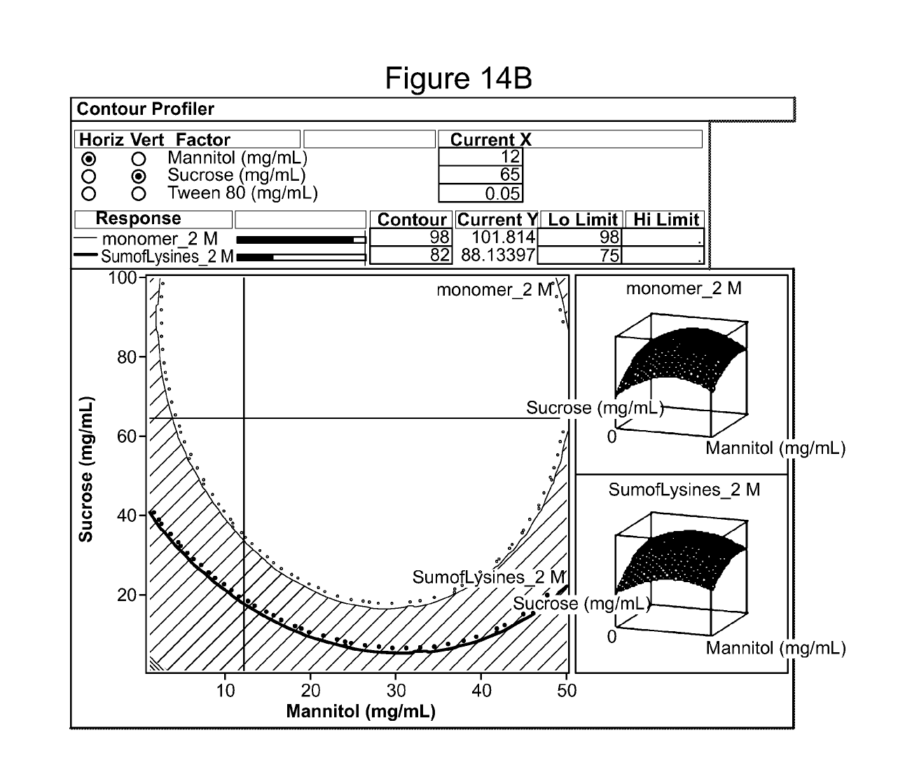

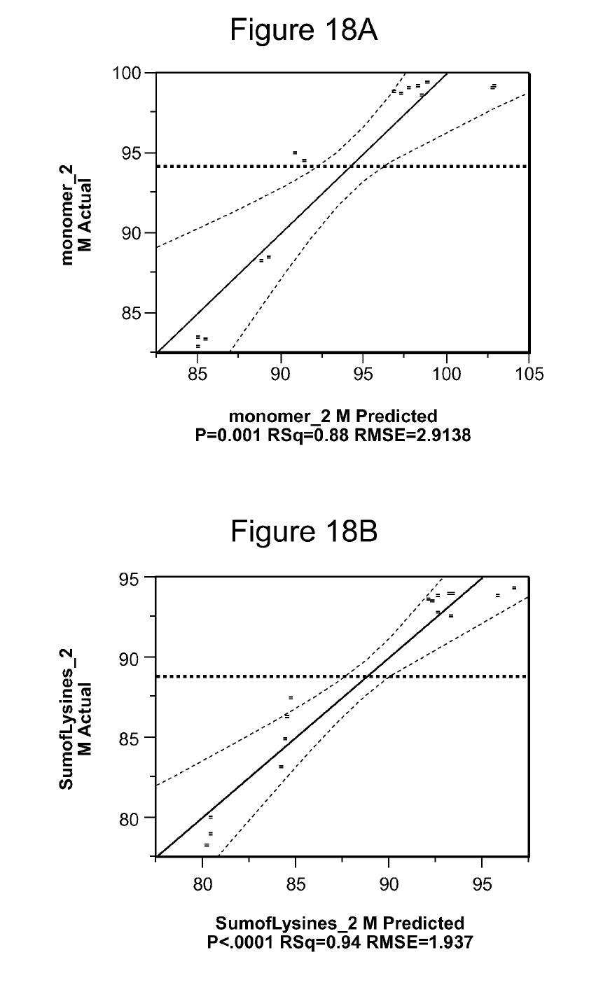

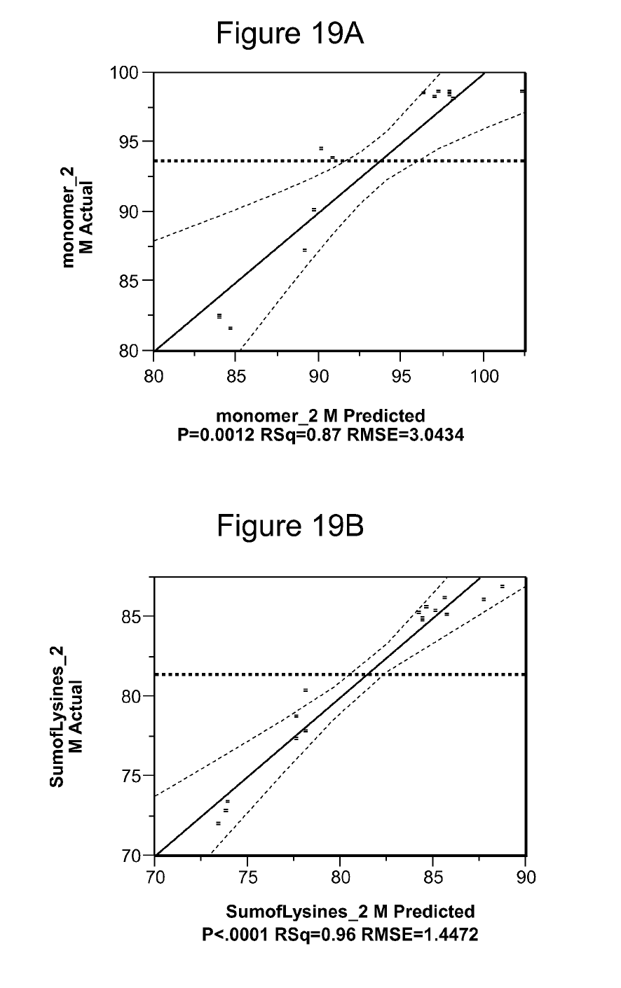

Studies using pearls containing adalimumab (both adalimumab and low acidic adalimumab) in the various combinations described in Tables 48 and 49 were analyzed by SEC analysis for monomer content and lysine content according to standard methods. Results from the adalimumab pearls stored for two months are provided in FIGS. 12 to 14. Sorted parameter estimates from the analysis are described below in Tables 50 to 54. FIGS. 18 and 19 provide sorted parameter estimates for both adalimumab (FIG. 19) and low acidic adalimumab (FIG. 18).

TABLE 50
Sorted Parameter Estimates for Low Acidic Adalimumab

| Term | Estimate | Std Error | t Ratio | Prob > \|t\| |
|---|---|---|---|---|
| Sucrose (mg/mL) | 0.124814 | 0.017036 | 7.33 | <.0001* |
| (Mannitol (mg/mL) − | −0.012085 | 0.004113 | −2.94 | 0.0165* |
| 19.0625)*(Mannitol | | | | |
| (mg/mL) − 19.0625) | | | | |
| Mannitol (mg/mL) | 0.2017156 | 0.069047 | 2.92 | 0.0170* |
| (Sucrose (mg/mL) − | −0.001594 | 0.000778 | −2.05 | 0.0706 |
| 47.9375)*(Sucrose | | | | |
| (mg/mL) − 47.9375) | | | | |
| (Mannitol (mg/mL) − | −0.000661 | 0.000787 | −0.84 | 0.4226 |
| 19.0625)*(Sucrose | | | | |
| (mg/mL) − 47.9375) | | | | |
| Tween 80 (mg/mL) | 0.5254745 | 1.534545 | 0.34 | 0.7399 |

TABLE 51
Sorted Parameter Estimates for Low Acidic Adalimumab

| Term | Estimate | Std Error | t Ratio | Prob > \|t\| |
|---|---|---|---|---|
| Sucrose (mg/mL) | 0.1227647 | 0.011325 | 10.84 | <.0001* |
| Mannitol (mg/mL) | 0.1428724 | 0.045901 | 3.11 | 0.0125* |
| (Sucrose (mg/mL) − | −0.001554 | 0.000517 | −3.01 | 0.0148* |
| 47.9375)*(Sucrose | | | | |
| (mg/mL) − 47.9375) | | | | |
| (Mannitol (mg/mL) − | −0.0082 | 0.002734 | −3.00 | 0.0150* |
| 19.0625)*(Mannitol | | | | |
| (mg/mL) − 19.0625) | | | | |
| (Mannitol (mg/mL) − | −0.00095 | 0.000523 | −1.82 | 0.1028 |
| 19.0625)*(Sucrose | | | | |
| (mg/mL) − 47.9375) | | | | |
| Tween 80 (mg/mL) | −0.194911 | 1.020124 | −0.19 | 0.8527 |

TABLE 52
Sorted Parameter Estimates for Adalimumab

| Term | Estimate | Std Error | t Ratio | Prob > \|t\| |
|---|---|---|---|---|
| Sucrose (mg/mL) | 0.1235574 | 0.017794 | 6.94 | <.0001* |
| Mannitol (mg/mL) | 0.2119536 | 0.07212 | 2.94 | 0.0165* |
| (Mannitol (mg/mL) − | −0.012199 | 0.004296 | −2.84 | 0.0194* |
| 19.0625)*(Mannitol | | | | |
| (mg/mL) − 19.0625) | | | | |
| (Sucrose (mg/mL) − | −0.001685 | 0.000812 | −2.07 | 0.0678 |
| 47.9375)*(Sucrose | | | | |
| (mg/mL) − 47.9375) | | | | |
| (Mannitol (mg/mL) − | −0.001058 | 0.000822 | −1.29 | 0.2303 |
| 19.0625)*(Sucrose | | | | |
| (mg/mL) − 47.9375) | | | | |
| Tween 80 (mg/mL) | 0.6769722 | 1.602824 | 0.42 | 0.6827 |

TABLE 53
Sorted Parameter Estimates for Adalimumab

| Term | Estimate | Std Error | t Ratio | Prob > \|t\| |
|---|---|---|---|---|
| Sucrose (mg/mL) | 0.1073776 | 0.008461 | 12.69 | <.0001* |
| Mannitol (mg/mL) | 0.1387143 | 0.034294 | 4.04 | 0.0029* |
| (Mannitol (mg/mL) − | −0.007836 | 0.002043 | −3.84 | 0.0040* |
| 19.0625)*(Mannitol | | | | |
| (mg/mL) − 19.0625) | | | | |
| (Sucrose (mg/mL) − | −0.001432 | 0.000386 | −3.71 | 0.0049* |
| 47.9375)*(Sucrose | | | | |
| (mg/mL) − 47.9375) | | | | |
| (Mannitol (mg/mL) − | −0.001025 | 0.000391 | −2.62 | 0.0277* |
| 19.0625)*(Sucrose | | | | |
| (mg/mL) − 47.9375) | | | | |
| Tween 80 (mg/mL) | −0.451748 | 0.762178 | −0.59 | 0.5680 |

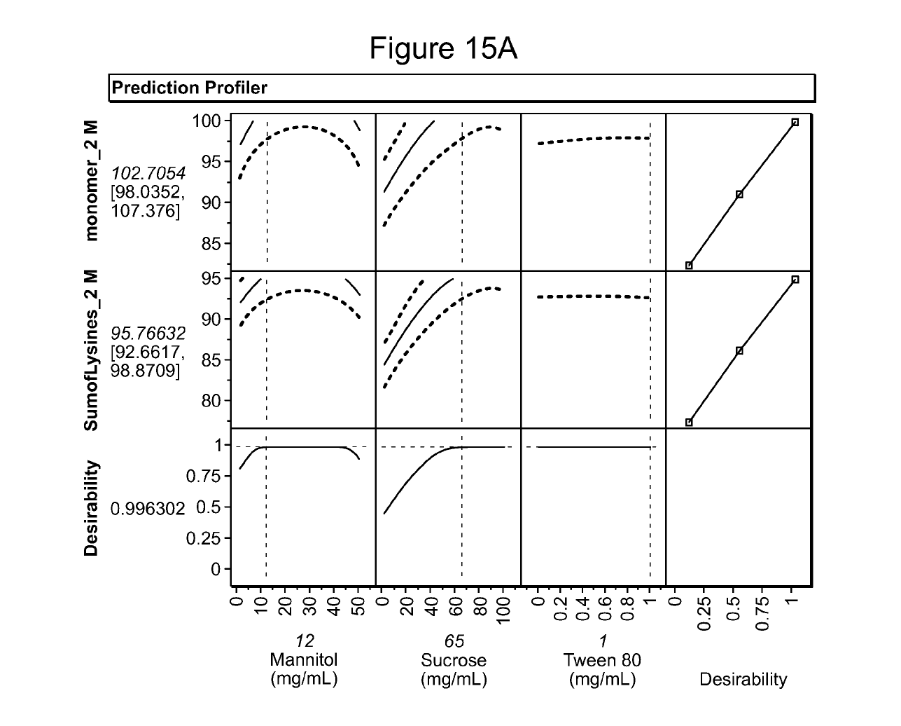

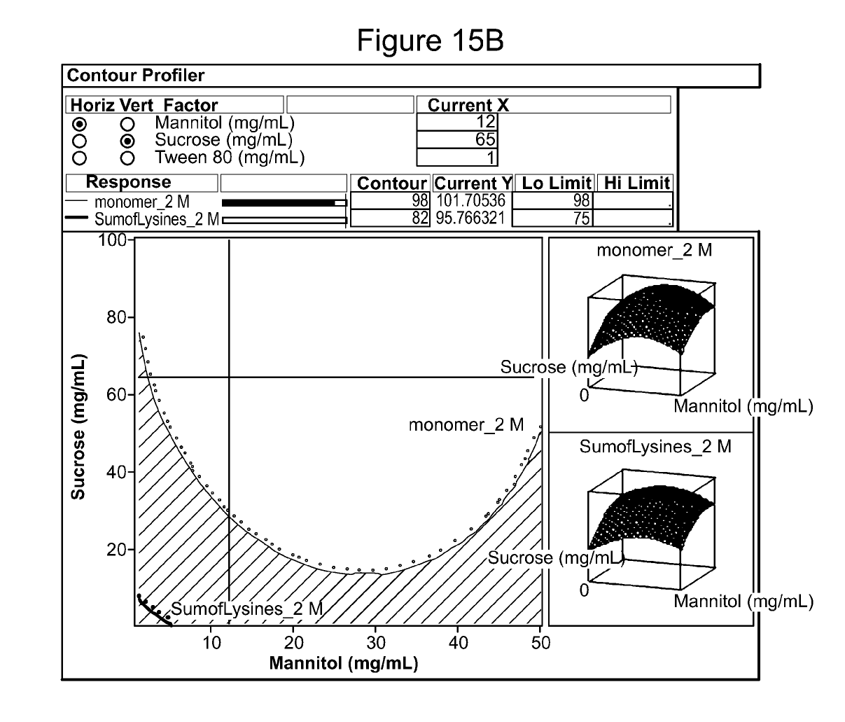

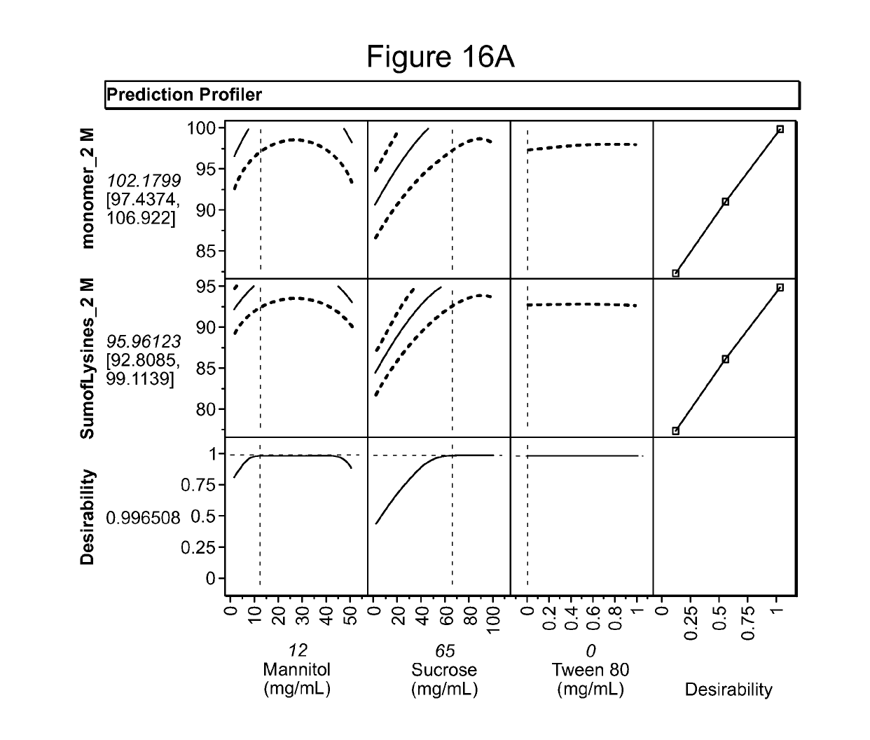

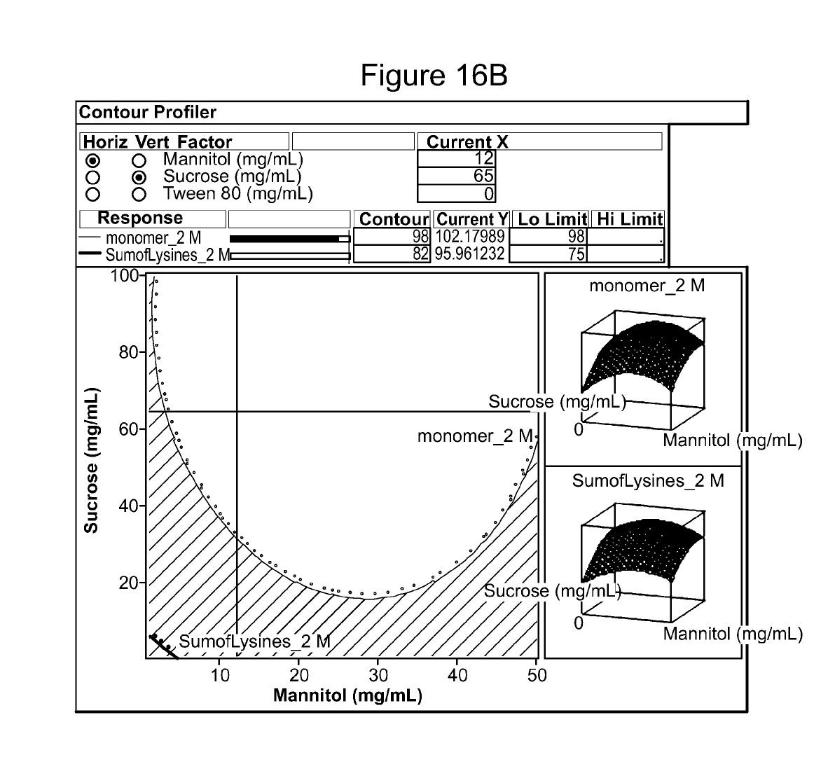

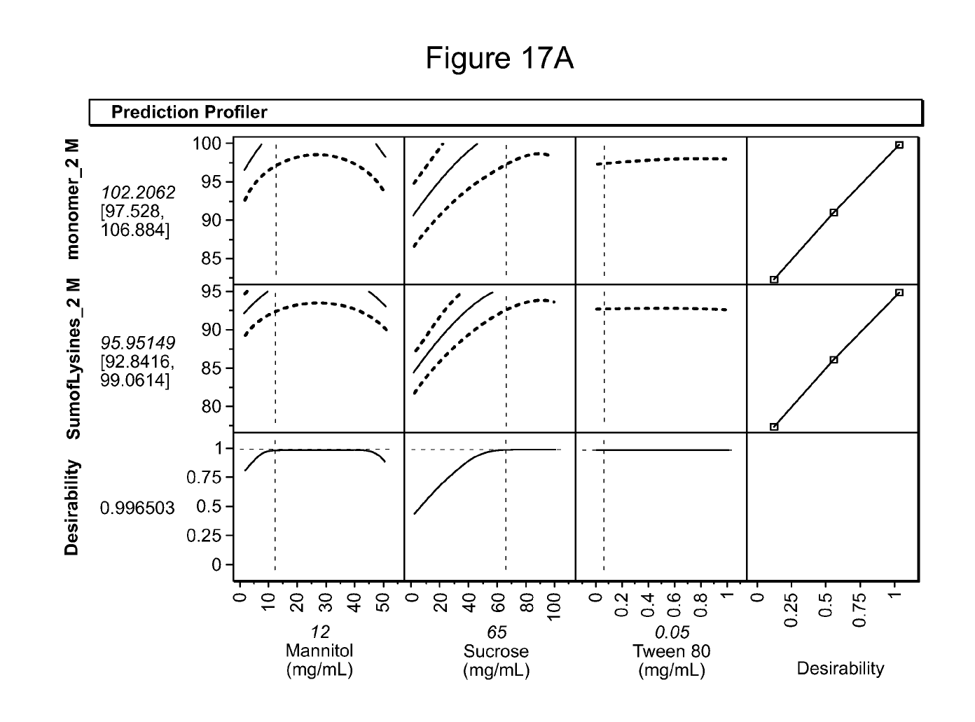

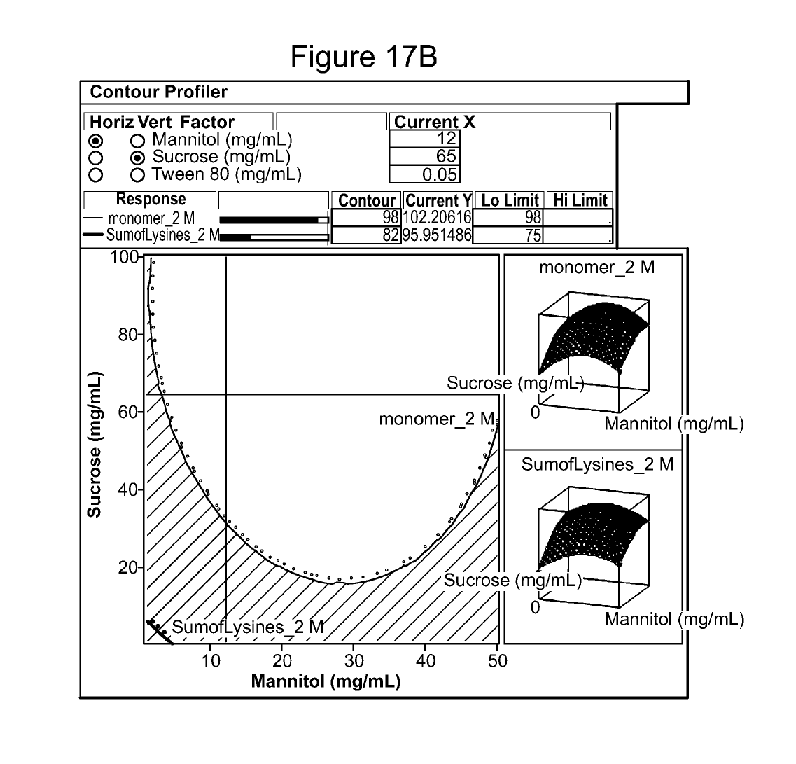

Results from the low acidic adalimumab pearls stored for two months are provided in FIGS. 15 to 17.

Results described in FIGS. 12 to 17 relate to stability at an elevated temperature of 40° C. and 75% relative humidity (accelerated conditions).

Overall, the results suggest that solution conditions that satisfy specification limits in 2 months are an eclipse area centered in the neighborhood of 26 mg/ml mannitol and 84 mg/ml sucrose. The level of Tween 80 did not have a significant effect on stability. Both sucrose and mannitol were found to have effects on both responses, for adalimumab and low acidic adalimumab. Some of the quadratic and interacation terms also had effectx. Tween was always not significant for either response. The result is shown in Table 55, terms with significant effect are indicated by star.

TABLE 55
Significant Effect Test Result

| | | | | | Mannitol | |
|---|---|---|---|---|---|---|
| | | Sucrose | | Mannitol | Sucrose | |
| Response | Sucrose | Quadratic | Mannitol | Quadratic | Interaction | Tween 80 |
| Adalimumab | * | | * | * | | |
| Monomer | | | | | | |
| Adalimumab | * | * | * | * | * | |
| Sum of Lysine | | | | | | |
| LAR Monomer | * | | * | * | | |
| LAR Sum of | * | * | * | * | | |
| Lysine | | | | | | |

In conclusion, the contour plots demonstrate mannitol and sucrose provide exceptional stability for pearl formulations containing antibodies, including adalimumab. The results also establish that there is a co-founding relationship between mannitol and sucrose and the curved plots display the boundaries of the formulation to maintain product stability. The graphs show that a mannitol concentration range of about 10 to 40 mg/mL and a sucrose concentration range of about 60 mg/mL to 80 mg/mL provides particularly stable solid unit formulations.

With respect to the above examples (Examples 1 to 20), it should be noted that reconstitution of solid units and cakes was done with water, unless otherwise indicated. In addition, low acidic adalimumab described the examples above refers to adalimumab that is obtained from a composition having low levels, e.g., less than 10%, of acidic species (AR) of adalimumab. Specifically the starting material for solid units made from low acidic adalimumab had <3% acidic species. Such examples of compositions having low levels of acidic species of adalimumab (used as source material for certain exemplary solid units described herein) and methods for making such compositions can be found in U.S. patent application Ser. No. 14/077,871, incorporated by reference herein.

### INCORPORATION BY REFERENCE

The contents of all cited references (including, for example, literature references, patents, patent applications, and websites) that are cited throughout this application are hereby expressly incorporated by reference in their entirety for any purpose.

### EQUIVALENTS

It will be clear that the invention may be practiced otherwise than as particularly described in the foregoing description and examples. Numerous modifications and variations of the present invention are possible in light of the above teachings and, therefore, are within the scope of the appended claims.

1.-18. (canceled)

19. A drug product comprising a plurality of lyophilized, spherical solid units which are free-flowing and geometrically uniform, wherein each solid unit of said plurality of solid units comprises a therapeutic protein and a sugar.

20. The drug product of claim 19, wherein the solid units each have a diameter selected from the group consisting of about 0.1 to about 4 mm; about 0.1 to about 3 mm; about 0.1 to about 2 mm; about 0.1 to about 1 mm; and about 0.1 to about 0.5 mm.

21. (canceled)

22. The drug product of claim 19, wherein each of the solid units comprises 0.01 μg to 6.0 mg of the therapeutic protein or 15 μg to 4.0 mg of the therapeutic protein.

23.-25. (canceled)

26. The drug product of claim 19, wherein the therapeutic protein is selected from the group consisting of a peptide, a DVD-Ig protein, and an antibody, or an antigen-binding portion thereof.

27.-29. (canceled)

30. The drug product of claim 26, wherein the antibody is adalimumab, or a biosimilar thereof.

31. (canceled)

32. A capsule comprising the drug product of claim 19.

33.-35. (canceled)

36. A method of treating a subject having a disorder, said method comprising combining the drug product of claim 19 with a diluent to form a reconstituted solution; and
administering the reconstituted solution to the subject having the disorder.

37. (canceled)

38. (canceled)

39. A stable solid unit suitable for pharmaceutical administration, comprising a therapeutic protein and sucrose or trehalose,
wherein the amount of sucrose or trehalose is sufficient to maintain the stability of the therapeutic protein for at least 12 months of storage at about 25° C. storage or for at least 3 months of storage at about 40° C., and
wherein the solid unit is free flowing when placed in a plurality of the solid units.

40.-42. (canceled)

43. The solid unit of claim 39, wherein the concentration of sucrose in a solution for preparation of the solid unit is selected from the group consisting of about 10 mg/ml to about 200 mg/ml; about 30 mg/ml to about 100 mg/ml; about 40 mg/ml to about 90 mg/ml; about 40 mg/ml to about 80 mg/ml; about 40 mg/ml to about 70 mg/ml; about 40 mg/ml to about 60 mg/ml; and about 40 mg/ml to about 50 mg/ml.

44. The solid unit of claim 39, wherein the concentration of sucrose in a solution for preparation of the solid unit is less than 20%, less than 15%, less than 10%, less than 7%, or about 1% to about 7% sucrose.

45. The solid unit of claim 39, wherein the solid unit is prepared from a solution comprising about 10 to about 40 mg/mL of mannitol and about 60 mg/mL to about 80 mg/mL of sucrose.

46.-48. (canceled)

49. The solid unit of claim 39, wherein the solid unit is in a shape of a sphere.

50. The solid unit of claim 49, wherein the sphere has a diameter selected from the group consisting of about 0.1 mm to about 4 mm; about 0.1 mm to about 3 mm; about 0.1 mm to about 2 mm; about 0.1 mm to about 1 mm; and about 0.1 mm to about 0.5 mm.

51. (canceled)

52. The solid unit of claim 39, wherein the solid unit is suitable for parenteral or oral administration.

53. (canceled)

54. (canceled)

55. A stable solid unit suitable for oral administration to a human subject, said solid unit comprising
a therapeutic agent,
a stabilizer, and
a polymer selected from the group consisting of an enteric protectant, a non-pH-sensitive polymer, a slow-release polymer, a bioadhesive polymer, or any combination thereof,
wherein the solid unit is free flowing when placed in a plurality of the solid units.

56.-76. (canceled)

77. The solid unit of claim 39
wherein the sucrose:therapeutic protein ratio ranges from about 0.8 to 3.5:1 weight/weight (w/w); or from about 0.9 to 2.0:1 w/w; or about 1:1 w/w.

78.-82. (canceled)

83. The solid unit of claim 39,
wherein the trehalose:therapeutic protein ratio ranges from a ratio selected from the group consisting of about 0.1 to 10:1 weight/weight (w/w), 0.1 to 3.5:1 w/w, and 0.8 to 3.5:1 w/w; or from about 0.9 to 2.0:1 w/w; or about 1:1 w/w.

84.-88. (canceled)

89. The solid unit of claim 39, wherein the solid unit has a volume ranging from about 0.0005 μl to about 20 μl.

90. (canceled)

91. (canceled)

92. The solid unit of claim 39, wherein the therapeutic protein is an anti-human TNFα antibody, or an antigen-binding portion thereof.

93.-102. (canceled)

103. The solid unit of claim 92, wherein the anti-human TNFα antibody, or an antigen-binding portion thereof, is adalimumab, or a biosimilar thereof.

104.-108. (canceled)

109. A plurality of solid units comprising the solid unit of claim 39.

110. The plurality of solid units of claim 109, wherein the solid units have a uniform size distribution and/or a volume ranging from about 0.0005 μl to about 40 μl; or a uniform size distribution and/or a volume ranging from about 0.1 μl to about 20 μl; or a volume ranging from about 0.5 μl to about 10 μl.

111.-113. (canceled)

114. The plurality of solid units of claim 109, wherein the therapeutic proteins are selected from the group consisting of a peptide, a DVD-Ig protein, an antibody, or an antigen-binding portion thereof, or any combination thereof.

115.-130. (canceled)

131. A dual-chambered delivery device comprising the plurality of solid units of claim 109.

132. The dual-chambered delivery device of claim 131, wherein the device comprises one chamber comprising the plurality of solid units and one chamber comprising a diluent.

133. (canceled)

134. A method of treating a subject in need thereof, comprising administering to the subject a therapeutically effective amount of the solid unit of claim 39.

135.-157. (canceled)
